(19) United States
(12) Patent Application Publication
(10) Pub. No.: US 2026/0273045 A1
(43) Pub. Date: Sep. 17, 2026

(54) ANTIGEN-SPECIFIC PHYSIOLOGIC DENDRITIC CELLS FOR VACCINATION

(71) Applicants: Yale University, New Haven, CT (US); Transimmune AG, Düsseldorf (DE); Emory University, Atlanta, GA (US)

(72) Inventors: Olga SOBOLEV, Shelton, CT (US); Richard EDELSON, Westport, CT (US); Aaron VASSALL, West Hartford, CT (US); Kazuki TATSUNO, Hamden, CT (US); Philip J. SANTANGELO, Smyrna, GA (US); Douglas HANLON, Branford, CT (US)

(21) Appl. No.: 19/480,134

(22) PCT Filed: Apr. 30, 2024
(86) PCT No.: PCT/EP2024/061958

§ 371 Date: Oct. 30, 2025

Related U.S. Application Data
(60) Provisional application No. 63/499,343, filed on May 1, 2023.

(30) Foreign Application Priority Data
Aug. 28, 2023 (EP) 23193757.4

(51) Int. Cl. A61K 39/215; A61K 9/1271; A61K 9/51; A61K 35/15; A61K 39/00; A61K 39/21; A61P 31/14; A61P 35/00; C12N 5/0784; C12N 7/00

(52) U.S. Cl. CPC A61K 39/215; A61K 9/1271; A61K 9/5123; A61K 35/15; A61K 39/0011; A61K 39/21; A61P 31/14; A61P 35/00; C12N 5/0639; C12N 7/00; A61K 2039/5154; A61K 2039/53; A61K 2039/55555; C12N 2527/00; C12N 2740/16034; C12N 2770/20034

(57) ABSTRACT

This invention relates to compositions comprising physiologic dendritic cells and at least one miRNA. The invention further relates to compositions comprising physiologic dendritic cells and at least one mRNA for use in vaccination.

### FIELD OF THE INVENTION

This invention relates to the field of prophylactic vaccination against diseases based on physiologic dendritic cells. The invention further relates to vaccine compositions comprising physiologic dendritic cells and at least one mRNA, as well as vaccine compositions for use in vaccination against infectious diseases and hyper-proliferative diseases such as cancers or tumors.

### BACKGROUND OF THE INVENTION

Dendritic cells (DCs) have the ability to take up, process, and present antigens on their cell surface to T-cells and B-cells. As DCs thereby activate naïve, effector, and memory immune cells, they are a promising therapeutic agent against diseases such as cancer or infectious diseases. The potent activation of de novo T-cell and B-cell responses also suggests dendritic cells as promising agents for prophylactic purposes. However, dendritic cells in the human body represent only about 0.3% of total circulating leukocytes, and consist of a heterogeneous population consisting of different maturation levels and capabilities of stimulating tolerogenic or stimulatory immune responses.

Techniques for the isolation and maturation of dendritic cells are described in a number of documents, and there are various methods, including bringing monocytes into contact with hematopoietic growth factors and cytokines such as IFN-α, GM-CSF, TNF-α, IL-3 or combinations thereof (see e.g. EP 922,758 or EP 663,930, WO 95/28479).

Extracorporeal Photopheresis (ECP) has been used successfully to treat cutaneous T-cell lymphoma (CTCL) in subsets of patients. In classical ECP, patient peripheral blood mononuclear cells (PBMC) are isolated and passed through a transparent plastic plate while being exposed to 8-methoxypsoralen and UVA (8-MOP UVA), before being returned to the patient. It has been assumed that the underlying mechanism of action includes dendritic cells taking up antigens that are being released by the exposure of malignant T cells to 8-MOP UVA and presenting these to the patient's immune system. According to their mode of action, these DCs have been termed immuno-stimulatory DCs. In the course of studying the principles of ECP, it has been learned that ECP leads to the conversion of passaged blood monocytes to DC. However, during this process it is not known, which antigens are exactly taken up and presented. Accordingly, there is a continuing need to selectively provide immuno-stimulatory DCs, which are moreover, equipped with defined antigens in order to provoke a desired and targeted immuno-stimulatory response.

### OBJECTIVES AND SUMMARY OF THE INVENTION

One objective of the present invention is to provide a vaccine composition comprising phDCs and at least one mRNA.

Another objective of the present invention is to provide a vaccine composition comprising antigen-specific phDCs and at least one mRNA. A further objective of the invention is to provide a vaccine composition comprising antigen-specific monocytes and at least one mRNA. It is another objective of the present invention to use the vaccine compositions of the invention for vaccination.

It is still another objective to provide vaccine compositions for use in a method of preventing or reducing a disease in a subject.

Finally, it is an objective of the invention to provide a kit comprising phDCs and at least one mRNA, wherein the at least one mRNA encodes for at least one antigenic protein.

These and other objectives as they will become apparent from the ensuing description hereinafter are solved by the subject matter of the independent claims. Some of the preferred embodiments of the present invention form the subject matter of the dependent claims. Yet other embodiments of the present invention may be taken from the ensuing description.

The present invention as illustratively described in the following may suitably be practiced in the absence of any element or elements, limitation or limitations, not specifically disclosed herein.

The present invention is described with respect to particular embodiments below and with reference to certain figures but the invention is not limited thereto but only by the claims.

The present invention is based to some extent on data and experiments presented hereinafter, which lead to the insight that dendritic cells loaded with selected disease antigens can be used effectively for vaccination against diseases such as cancer or infectious diseases. As key prerequisites for obtaining antigen-specific DCs, dendritic cells need to be (i) obtained and (ii) equipped with disease specific antigens. As regards (i), so-called physiologic dendritic cells (phDCs) can be used. phDCs can inter alia be produced in vitro by applying a shear force to monocytes (e.g. from a blood sample), see e.g. Ventura et al., 2018 “Extracorporeal Photochemotherapy Drives Monocyte-to-Dendritic Cell Maturation to Induce Anticancer Immunity”, WO 2014/106629, WO 2016/001405 or Hanlon et al., 2020 “Rapid Production of Physiologic Dendritic Cells (phDC) for Immunotherapy”). phDC can be characterized by marker expression including markers such as HLA-DR, CD83, CD86, ICAM or PLAUR and/or no increased expression of GILZ (see, e.g., Ventura et al., 2018 “Extracorporeal Photochemotherapy Drives Monocyte-to-Dendritic Cell Maturation to Induce Anticancer Immunity” or WO 2014/106629). As the monocytes do not need to be subjected to cytokines to induce differentiation into dendritic cells, it is assumed that dendritic cells which are obtained by subjecting monocytes to a physical force more closely resemble the properties of naturally-occurring dendritic cells which is why dendritic cells which are obtained by subjecting monocytes to a physical force will be designated as physiological dendritic cells (phDCs) hereinafter.

As described previously (Ventura et al., 2018 “Extracorporeal Photochemotherapy Drives Monocyte-to-Dendritic Cell Maturation to Induce Anticancer Immunity” or WO 2014/106629), a physical force to induce differentiation of monocytes into phDCs can be applied by passing monocytes through a flow chamber which can take the form of, e.g., a plate or, e.g., a bag (e.g. flexible bag or plastic bag). Such a bag is described, e.g., in Buechler et al., 2004 “Generation of Dendritic Cells Using Cell Culture Bags—Description of a Method and Review of Literature”. It is assumed that such passing through, e.g., a plate or placing in, e.g., a flexible bag or a combination thereof (e.g., a hybrid of a bag and a chamber as disclosed herein), exposes the monocytes to shear stress, inducing differentiation into dendritic cells.

If platelets are present, the maturation process can be improved. As mentioned, the monocytes can be matured into dendritic cells using this method without the need for adding expensive cytokine cocktails. As also mentioned, the above described process of passing monocytes through, e.g., a plate or placing them in, e.g., a bag or a hybrid of a chamber and a bag, mimics some of the aspects which are assumed to take place in vivo which is why the dendritic cells generated by plate-passing (or placement in a bag and movement) are termed “physiological dendritic cells” (phDC) throughout this disclosure. Although phDCs have been found to work especially well for loading with disease specific antigens (e.g. with at least one mRNA encoding for at least one antigenic protein), any population of DCs can in principle be used for generating the antigen-specific DCs of the invention, such as e.g. DCs obtained after incubation of blood derived monocytes with cytokines. As has also previously been shown (Ventura et al., 2018 “Extracorporeal Photochemotherapy Drives Monocyte-to-Dendritic Cell Maturation to Induce Anticancer Immunity” or WO 2016/001405), phDCs which are preferably produced in the absence of apoptotic signals and subsequently loaded with antigens will be effective in stimulating an immune response. It needs to be understood that transfection (with e.g. at least one mRNA encoding for at least one antigenic protein), when subjecting monocytes to a physical force, may also occur at the monocyte stage. Thus, monocytes can be combined with disease specific antigens (in the form of e.g. at least one mRNA encoding for at least one antigenic protein) and subjected to a physical force such as shear forces. As described in WO 2016/001405, monocytes activated by a physical force (so-called globally activated monocytes), may inter alia be identified by increased expression of at least HLA-DR, PLAUR or ICAM-1.

As regards (ii), the inventors found that phDC are capable of internalizing mRNA-containing LNP and expressing encoded antigens such as disease associated proteins or peptides. The loaded antigen specific phDCs (e.g. vaccine composition comprising phDCs and at least one mRNA) have been found to be useful in targeted immunotherapy against different diseases such as e.g. cancer or infectious diseases. It appears that the antigen specific phDCs of the invention are capable of eliciting a disease-preventing immune response by their unique ability of presenting the specific antigen on MHC molecules leading to activation, proliferation and differentiation of T cells into armed effector T cells. One important function of effector T cells is the killing of infected cells by CD8+ cytotoxic T cells and the activation of macrophages by Th1 cells which together make up cell-mediated immunity, and the activation of B cells by both Th2 and Th1 cells to produce different classes of antibody, thus driving the humoral immune response. Importantly, T cells (such as e.g. cytotoxic T cells) are transmissible to the brain, leading to superior vaccination effects as opposed to classical vaccines, or specifically mRNA vaccines, which primarily rely on eliciting a B cell response including the production of antibodies, which are, however, not able to pass the blood-brain-barrier. The direct use of antigen-specific phDCs (e.g. vaccine composition comprising phDCs and at least one mRNA) can additionally be considered a safer vaccination approach as the immune system is activated by a very defined set of cells as compared to e.g. mRNA vaccination, which randomly scatters the antigenic peptides from muscle tissue into systemic circulation providing a larger probability of missing target cells and eliciting unwanted side effects. Furthermore, the use of adjuvants may be lower for the vaccines of the invention as compared to conventional vaccines, including mRNA vaccines. As the antigen-specific phDCs (e.g. vaccine composition comprising phDCs and at least one mRNA) of the invention are highly efficient, the present invention also provides for significant dose sparing possibilities.

Moreover, the direct use of antigen-specific phDCs (e.g. vaccine composition comprising phDCs and at least one mRNA) allows for lower LNP doses to induce an immune response. Still another advantage of antigen-specific phDCs (e.g. vaccine composition comprising phDCs and at least one mRNA) is a faster onset of immunoprotection after administration. For vaccines such as mRNA vaccines, clonal expansion of memory B cells takes around 10 to 14 days. However, the antigen-specific phDCs are able to directly trigger clonal expansion of the subject's already preexisting T-cells into effector cytotoxic T-cells not depending on a 10-14 day maturation lag period. Memory T cells, unlike naiive T cells, can be triggered for cytotoxic function as soon as an antigen is encountered. Thus, vaccination with antigen-specific phDCs (e.g. vaccine composition comprising phDCs and at least one mRNA) corresponds to a vaccination approach with immediate efficacy backed by its additional potency to induce a subsequent humoral immune response as a follow-up.

### First Aspect: Vaccine Composition

In a first aspect, the present invention relates to a vaccine composition comprising a pharmaceutically effective amount of phDCs and at least one RNA, which comprises a coding sequence encoding for at least one antigenic protein.

In one embodiment, the RNA is a single stranded RNA, an mRNA, a circular RNA, a self-amplifying RNA, and/or a synthetic RNA. In a preferred embodiment, the RNA is self-amplifying mRNA. In a preferred embodiment, the RNA is an mRNA. Thus, the present invention relates to a vaccine composition comprising a pharmaceutically effective amount of phDCs and at least one mRNA (e.g. one mRNA molecule or more than one mRNA molecule), which comprises a coding sequence encoding for at least one antigenic protein.

In one embodiment, the RNA (e.g. mRNA) comprises at least one chemical modification. If more than one RNA (e.g. mRNA) is used, each RNA (e.g mRNA) independently comprises at least one chemical modification (i.e. the chemical modification from one RNA to another can be different). For the purposes of the present disclosure, chemical modification means that one of the four naturally-occurring standard nucleosides which occur in RNA (adenosine (A), guanosine (G), uridine (U), and cytidine (C)) are replaced by modified forms thereof wherein the modification affects the base moiety within the nucleoside. Modified nucleosides can be naturally-occurring or non-naturally-occurring modified nucleosides. Naturally-occurring modified nucleosides are preferred.

Such replacement of A, C, U, and G by naturally-occurring or non-naturally-occurring modified nucleosides is assumed to reduce the Toll-like receptor (TLR)-mediated immune response of the recipient dendritic cells and/or to increase expression of the antigen encoded by the RNA (e.g. mRNA).

In one embodiment, the chemical modification is a substitution of one or more nucleosides of the RNA (e.g. mRNA) by one or more modified nucleosides.

Naturally occurring modified nucleosides comprise 1-methyladenosine (m¹A), N⁶-methyladenosine (m⁶A), 2′-O-methyladenosine (Am), 5-methylcytidine (m⁵C), 2′-O-methylcytidine (Cm), 2-thiocytidine (s²C), N⁴-acetylcytidine (ac⁴C), 5-formylcytidine (f⁵C), 2′-O-methylguanosine (Gm), inosine (I), pseudouridine (Ψ), 5-methyluridine (m⁵U), 2′-O-methyluridine (Um). 1-methylpseudouridine (m1Ψ), 2-thiouridine (s2U), 4-thiouridine (s4U), 5-methoxyuridine (mo5U) and 3-methyluridine (m3U).

Within the four nucleosides A, U, C, and G, it is preferred to substitute at least one or more uridine nucleosides of the RNA (e.g. mRNA) by naturally occurring modified uridine nucleosides. It is assumed that the above-mentioned effects on the TLR-mediated immune reaction and/or the expression level of the RNA (e.g. mRNA) encoded antigen depend on the extent of replacement of uridine by naturally-occurring modified uridine nucleosides. In a preferred embodiment, all uridine nucleosides within an antigen-encoding RNA (e.g. mRNA) are thus replaced with naturally-occurring modified uridine nucleosides. However, the present invention also considers less than 90%, less than 80%, less than 70%, less than 60%, or less than 50% of the uridine nucleosides present in the RNA (e.g. mRNA) encoding the antigenic protein are replaced with naturally-occurring modified uridine nucleosides. Where uridine nucleosides are replaced by naturally-occurring modified forms thereof, it is preferred to use pseudouridine and more preferred to use N1-methylpseudouridine or N1-ethylpseudouridine. N1-methylpseudouridine is most preferred. The invention thus considers as a particularly preferred embodiment antigen-encoding RNAs (e.g. mRNAs) in which all uridines are replaced N1-methylpseudouridine.

The invention also considers to replace, instead of and preferably in addition to uridine nucleosides, A, C, and/or G nucleosides by modified forms thereof. It is preferred to use naturally-occurring modified forms of A, C, and/or G for such replacements. If such additional replacements are considered, it is preferred that all of A, C, and/or G nucleosides are replaced by modified forms thereof.

In one embodiment, the RNA (e.g. mRNA) comprises structural elements comprising a 5′ untranslated region (UTR), a 3′ UTR, a 5′ cap and/or a poly(A) tail. In one embodiment, the RNA (e.g. mRNA) contains all of these elements. In one embodiment, the 5′ cap is a Cap1 structure or a m7GpppG cap. Preferably, the 5′cap is a Cap1 structure. In one embodiment, the sequence of the RNA (e.g. mRNA) is optimized. In one embodiment, the sequence of the 5′ UTR, 3′UTR, and/or coding sequence for the antigenic protein is optimized. In one embodiment, the sequence of the RNA (e.g. mRNA) has optimized codon usage or optimized G/C content. In one embodiment, codon usage, G/C content and structural elements are optimized. Optimization of the sequence of the structural elements 3′ and/or 5′UTR can additionally comprise using one or more of a heterologous UTR, a Kozak sequence, a FI element, removed AURES elements and an enzymatically added tail (e.g. poly(A) tail). In one embodiment, a poly(A) tail is added enzymatically.

The at least one RNA (e.g., mRNA) can be comprised in a nanoparticle. Nanoparticles comprise lipid nanoparticles, poly(amine-co-ester) particles (PACE), poly-beta-amino-ester particles, PACE polyplex particles, lipoplexes and poly(N,N-cystaminebis(acrylamide)-co-4-amino-1-butanol) (pABOL) particles. In one embodiment, the nanoparticle is a lipid nanoparticle (LNP).

In one more preferred embodiment, the RNA (e.g. mRNA) is comprised in a lipid nanoparticle (LNP). A lipid nanoparticle can comprise a cationic lipid, a PEG-modified lipid, a cholesterol, a DSPE-PEG-maleimide, DSPN-PEG-azide and/or a non-cationic lipid. In one embodiment, a lipid nanoparticle comprises a cationic lipid, a PEG-modified lipid, a cholesterol, and/or a non-cationic lipid. In one embodiment, the lipid nanoparticle comprises a cationic lipid, a PEG-modified lipid, a cholesterol and a non-cationic lipid. Cationic lipids comprise cKK-E12, cKK-E14, LP01, SM102, Lipid 5, etc. In one embodiment, the cationic lipid is a cKK-E12 lipid. In one embodiment, the cationic lipid is a SM102 lipid. In one embodiment, the cationic lipid is a MC3 lipid (DLin-MC3-DMA). In one embodiment, the lipid nanoparticle comprises cKK-E12, cholesterol, C14-PEG 2000-PE and DOPE. In one embodiment, the lipid nanoparticle comprises cKK-E12, cholesterol, C14-PEG 2000-PE and DOPE at a ratio of 30-40:41-51:1.0-4.0:12-21. In one embodiment, the lipid nanoparticle comprises cKK-E12, cholesterol, C14-PEG 2000-PE and DOPE at a ratio of 33-37:44-48:2.0-3.0:14-18. In one embodiment, the lipid nanoparticle comprises cKK-E12, cholesterol, C14-PEG 2000-PE and DOPE at a ratio of 34-36:45-47:2.2-2.8:15-17. In one embodiment, the lipid nanoparticle comprises cKK-E12, cholesterol, C14-PEG 2000-PE and DOPE at a ratio of 35:46.5:2.5:16.

In one embodiment, the lipid nanoparticle comprises SM-102, cholesterol, C14-PEG 2000-PE and DOPE. In one embodiment, the lipid nanoparticle comprises SM-102, cholesterol, C14-PEG 2000-PE and DOPE at a ratio of 30-40:41-51:1.0-4.0:12-21. In one embodiment, the lipid nanoparticle comprises SM-102, cholesterol, C14-PEG 2000-PE and DOPE at a ratio of 33-37:44-48:2.0-3.0:14-18. In one embodiment, the lipid nanoparticle comprises SM102, cholesterol, C14-PEG 2000-PE and DOPE at a ratio of 34-36:45-47:2.2-2.8:15-17. In one embodiment, the lipid nanoparticle comprises SM-102, cholesterol, C14-PEG 2000-PE and DOPE at a ratio of 35:46.5:2.5:16. In some embodiments, the lipid nanoparticle comprises SM102, cholesterol, DMG-PEG-2K, DSPC. In one embodiment, the lipid nanoparticle comprises SM102, cholesterol at a ratio of 30-50:35-45:1.0-4.0:10-20, DMG-PEG-2K, DSPC at a ratio of 50:38.5:1.5:10.

In one embodiment, the lipid nanoparticle comprises MC3, cholesterol, C14-PEG 2000-PE and DOPE. In one embodiment, the lipid nanoparticle comprises MC3, cholesterol, C14-PEG 2000-PE and DOPE at a ratio of 30-40:41-51:1.0-4.0:12-21. In one embodiment, the lipid nanoparticle comprises MC3, cholesterol, C14-PEG 2000-PE and DOPE at a ratio of 33-37:44-48:2.0-3.0:14-18. In one embodiment, the lipid nanoparticle comprises MC3, cholesterol, C14-PEG 2000-PE and DOPE at a ratio of 34-36:45-47:2.2-2.8:15-17. In one embodiment, the lipid nanoparticle comprises MC3, cholesterol, C14-PEG 2000-PE and DOPE at a ratio of 35:46.5:2.5:16.

In one embodiment, the at least one mRNA encoding for at least one antigenic protein is heterologous or exogenous to the phDCs. In one embodiment, the antigenic protein is exogenous to the phDCs. The terms “heterologous” or “exogenous” as used herein, in relation to a specific cell type, e.g., phDCs, can refer to a nucleic acid sequence, e.g. mRNA, or a protein, that originates from a source other than the specified cell type, e.g., the phDC. A heterologous mRNA or exogenous mRNA to phDCs thus refers to an mRNA that is not endogenous to the phDC, but has been introduced to the phDCs, e.g. by transfection or other means.

In one embodiment, the antigenic protein is an infectious disease associated antigen or a tumor associated antigen. In one embodiment, the antigenic protein is an infectious disease associated antigen. Thus, the vaccine composition comprises phDCs and at least one mRNA, which comprises a coding sequence encoding at least one infectious disease associated antigenic protein. In one embodiment, the infectious disease associated antigen is a viral antigen, a bacterial antigen, a fungal antigen or a parasite antigen. In one embodiment, the infectious disease associated antigen is a bacterial antigen. In one embodiment, the bacterial antigen is derived from Borrelia spp. or Mycobacteria spp. In another embodiment, the infectious disease associated antigen is a fungal antigen. In one embodiment, the fungal antigen is derived from Cryptococcus neoformans, Histoplasma capsulatum, Coccidioides immitis, Blastomyces dermatitidis, Chlamydia trachomatis or Candida albicans. In another embodiment, the infectious disease associated antigen is a parasite antigen. In one embodiment, the parasite antigen is derived from Plasmodium malariae.

In one embodiment, the infectious disease associated antigen is a viral antigen. Thus, the vaccine composition comprises phDCs and at least one mRNA, which comprises a coding sequence encoding at least one viral antigenic protein. In one embodiment, the viral antigen is a coronavirus antigenic protein or HIV antigenic protein. In one embodiment, the viral antigen is a coronavirus antigenic protein. In one embodiment, the viral antigen is a betacoronavirus antigenic protein. In another embodiment, the viral antigen is a SARS-CoV-2 antigenic protein. In one embodiment, the coronavirus antigenic protein or betacoronavirus antigenic protein is an antigenic protein of the spike protein, envelope protein, nucleocapsid protein, membrane protein and/or Orf1ab polyprotein or a fragment of each of the aforementioned. In one embodiment, the SARS-CoV-2 antigen is an antigenic protein of the spike protein, envelope protein, nucleocapsid protein, membrane protein and/or Orf1ab polyprotein or a fragment of each of the aforementioned. In one embodiment, the SARS-CoV-2 antigen is an antigenic protein of the spike protein or a fragment thereof. A fragment comprises at least 10, at least 50, at least 100, at least 200, at least 400 or at least 800 amino acid residues.

In one embodiment, the RNA (e.g. mRNA) comprises a sequence which is at least 75%, 85%, 90%, 95%, 98% or 99% or a 100% identical to a sequence or a part of a sequence selected from the group comprising SEQ ID NO: 1 (spike protein transcript), SEQ ID NO: 2 (envelope protein transcript), SEQ ID NO: 3 (nucleocapsid protein transcript), SEQ ID NO: 4 (membrane protein transcript) and/or SEQ ID NO: 5 (Orf1ab polyprotein transcript). In one embodiment, the RNA (e.g. mRNA) comprises a sequence or a part of a sequence which is at least 75%, 85%, 90%, 95%, 98% or 99% or a 100% identical to SEQ ID NO: 1. In one embodiment, the antigenic protein, for which the RNA (e.g. mRNA) encodes, comprises at least 100, at least 200, at least 300, at least 400, at least 500, at least 600, at least 700, at least 800, at least 900, at least 1000, at least 1200, at least 2000 or at least 3000 amino acids. In one embodiment, the antigenic protein, for which the RNA (e.g. mRNA) encodes, comprises at least 50 amino acids. In one embodiment, the antigenic protein, for which the RNA (e.g. mRNA) encodes, comprises at least 500 amino acids. In one embodiment, the antigenic protein, for which the RNA (e.g. mRNA) encodes, comprises at least 1000 amino acids.

In one embodiment, the SARS-CoV-2 antigen is a protein or peptide comprising a sequence which is at least 75%, 85%, 90%, 95%, 98% or 99% or a 100% identical to a sequence or a part of a sequence selected from the group comprising SEQ ID NO: 6 (spike amino acid sequence), SEQ ID NO: 7 (envelope amino acid sequence), SEQ ID NO: 8 (nucleocapsid amino acid sequence), SEQ ID NO: 9 (membrane amino acid sequence) and/or SEQ ID NO: 10 (Orf1ab amino acid sequence). In one embodiment, the protein or peptide comprises a sequence or part of a sequence which is at least 75%, 85%, 90%, 95%, 98% or 99% or a 100% identical to SEQ ID NO: 6.

In one embodiment, the at least one antigenic protein is a SARS-CoV-2 hexapro spike protein, with a foldon domain in its ectodomain right above the transmembrane domain (RNA sequence of which is depicted as bold and underlined in Table 3) or a RNA (e.g. mRNA) encoding therefore. The corresponding RNA (e.g. mRNA) may be modified with one or more of N1-methylpseudouridine, a cap1 structure and a poly-A tail. In one embodiment, the RNA (e.g. mRNA) comprises N1-methylpseudouridine (e.g. each uridine is replaced by N1-methylpseudouridine), a cap1 structure and a poly-A tail. In one embodiment, the codons may be optimized to GC enrich the RNA (e.g. mRNA) and optimized 5′ and 3′ UTRs may be used. In one embodiment, the RNA (e.g. mRNA) comprises a sequence or part of a sequence corresponding to SEQ ID NO: 19 or a sequence having at least 99%, 98%, 97%, 96%, 95%, 90%, 85%, or 80% identity to SEQ ID NO: 19. In one embodiment, the RNA (e.g. mRNA) comprises a sequence or part of a sequence corresponding to SEQ ID NO: 19 or a sequence having at least 99%, 98%, 97%, 96%, 95%, 90%, 85%, or 80% identity to SEQ ID NO: 19; and a poly-A tail (e.g. of about 200 to 400 nucleotides).

In one embodiment, the viral antigen is derived from HIV. In one embodiment, the HIV antigen is derived from the envelope protein (env), group antigens polyprotein (gag), reverse transcriptase (pol) and/or negative factor protein (nef). In one embodiment, the RNA (e.g. mRNA) which encodes for an antigenic protein comprises a sequence which is at least 75%, 85%, 90%, 95%, 98% or 99% or a 100% identical to a sequence or a part of a sequence selected from the group comprising SEQ ID NO: 11 (env protein transcript), SEQ ID NO: 12 (gag protein transcript), SEQ ID NO: 13 (pol protein transcript) and/or SEQ ID NO: 14 (nef protein transcript).

In one embodiment, the HIV antigen is a protein or peptide comprising a sequence which is at least 75%, 85%, 90%, 95%, 98% or 99% or a 100% identical to a sequence or apart of a sequence selected from the group comprising SEQ ID NO: 15 (env amino acid sequence), SEQ ID NO: 16 (gag amino acid sequence), SEQ ID NO: 17 (pol amino acid sequence) and/or SEQ ID NO: 18 (nef amino acid sequence).

The vaccine composition of the present invention may comprise a pharmaceutically effective amount of physiological DCs (phDCs) and at least one mRNA, which comprises a coding sequence encoding for at least one antigenic protein, wherein, the mRNA may be at least partially comprised in the phDCs. In some embodiments, substantially all of the mRNA is comprised in the phDCs. The mRNA not incorporated into the phDCs may be removed by washing the phDCs or other means known in the art. The resulting vaccine composition may thus comprise phDCs, wherein the phDCs have taken up mRNA encoding for at least one antigenic protein. The mRNA may be translated to the antigenic protein. In some embodiments, the phDCs express the antigenic protein. The antigenic protein may be exogenous or heterologous to the phDCs. In some embodiments, the free, i.e., the non-incorporated mRNA is not removed from the vaccine composition, and the composition thus comprises unincorporated mRNA. In another embodiment, the vaccine composition of the invention may comprise a pharmaceutically effective amount of physiological DCs (phDCs) and at least one mRNA, which comprises a coding sequence encoding for at least one antigenic protein, wherein the mRNA is at least partially not incorporated into the phDCs. In some embodiments, only a subset of the mRNA comprised in the vaccine composition is taken up by the phDCs present in the composition. In some embodiments, more than 30%, 40%, 50%, 60%, 70%, 80%, or 90% of the mRNA is not incorporated into the phDCs. In some embodiments, more than 30%, 40%, 50%, 60%, 70%, 80%, or 90% of the mRNA comprised in the vaccine composition of the invention is incorporated into the phDCs.

In one embodiment, the at least one antigenic protein is a tumor associated antigen. Thus, the vaccine composition comprises phDCs and at least one mRNA, which comprises a coding sequence encoding at least one tumor associated antigenic protein. In one embodiment, the tumor associated antigen is a leukemia antigen, melanoma antigen, lymphoma antigen, endometrial cancer antigen, kidney cancer antigen, brain cancer antigen, cervical cancer antigen, liver cancer antigen, head and neck cancer antigen, gastrointestinal cancer antigen, lymph node cancer antigen, pancreas cancer antigen, ear, nose and throat (ENT) cancer antigen, breast cancer antigen, prostate cancer antigen, ovarian cancer antigen or lung cancer antigen. In one embodiment, the at least one antigenic protein is a blood cancer associated antigen. A blood cancer antigenic protein can be a leukemia antigenic protein, lymphoma antigenic protein or myeloma antigenic protein.

In one embodiment, phDCs are obtainable by subjecting monocytes (e.g. obtained from a donor) to a physical force. The physical force can be applied to the monocytes by passing said monocytes through a flow chamber. The flow chamber can be a plate, a bag or a flow chamber of a device, e.g. such as a large-scale ECP device, e.g., a clinical ECP device (e.g., a THERAKOS® CELLEX® device; Combination of Terumo's Spectra Optia with UVA PIT; Combination of apheresis device from Fresenius and Macogenix from Macopharma; Single-Needle Option for the Amicus® Extracorporeal Photopheresis Protocol), or a miniaturized ECP device, e.g., a Transimmunization plate as described in WO2017/005700 A1, or a combination thereof (e.g. a hybrid of a bag and a chamber as disclosed herein). In one embodiment of the present invention, phDCs are obtainable by plate-passage of monocytes using an extracorporeal photopheresis (ECP) derived process. Thus, in one embodiment, the flow chamber is a plate. Monocytes (e.g. obtained from a donor) are passed through a plate such that the monocytes are exposed to shear forces. Preferably, platelets are present in the plate which can be either derived from the donor's blood sample or a fraction thereof or provided separately. Additionally or alternatively, plasma components can be present in the plate which can be either derived from the donor's blood sample or a fraction thereof or provided separately. The process of preparing phDCs, and also the term itself, has been described previously in, for example, Hanlon et al, 2020 (“Rapid Production of Physiologic Dendritic Cells (phDC) for Immunotherapy”). In one embodiment, all method steps are carried out in vitro.

In another embodiment, the flow chamber is a bag. Optionally, the bag is a flexible bag or a plastic bag. In one embodiment, the flow chamber is a flexible bag. In one embodiment, the bag is a plastic bag. In one embodiment, the material of the flow chamber (e.g. plate, flexible bag or plastic bag) is plastic. In one embodiment, the material of the flow-chamber is non-plastic such as glass, ceramic or silicone. If plastic materials are considered, one may use acrylics, polycarbonate, polyetherimide, polysulfone, polyphenylsulfone, styrenes, polyurethane, polyethylene, teflon or any other appropriate medical grade plastic. In a preferred embodiment of the present invention, the flow chamber is made from an acrylic plastic. If a bag (e.g. flexible bag) is considered, the material may be plastic, rubber or silicone. In a preferred embodiment, the material is plastic. Plastic materials comprise polyolefin, polyethylene, fluoropolymer, polyvinyl chloride, ethylene-vinyl acetate-copolymer, ethylene vinyl alcohol, polyvinylidene fluoride, and/or other plastic comprising materials approved for medical use.

In some embodiments, the flow chamber comprises or consists of a plate. The plate can be made of various materials, including but not limited to a plastic material. In one embodiment, the material of the plate is plastic. In one embodiment, the material of the plate is non-plastic such as glass, ceramic or silicone. Non limiting examples of materials for the plate comprise acrylics, polycarbonate, polyetherimide, polysulfone, polyphenylsulfone, styrenes, polyurethane, polyethylene, teflon or any other appropriate medical grade plastic. The plate may be rigid or flexible. In some embodiment, the material of the plate may comprise or consist of plastic, rubber or silicone. In some embodiments, the plate is elastic, i.e. is made of an elastic material. The elastic material may comprise cyclic olefin copolymer (COC), polyolefin, polyethylene, fluoropolymer, polyvinyl chloride, ethylene-vinyl acetate-copolymer, ethylene vinyl alcohol, polyvinylidene fluoride, polydimethylsiloxane (PDMS), dimethicone, and/or other plastic comprising materials approved for medical use. In one embodiment, the plate is made of PDMS, e.g., PDMS RTV-615 or PDMS Sylgard 184. In another preferred embodiment of the present invention, the plate is made from an acrylic plastic.

In some embodiments, the flow chamber is a hybrid flow chamber. The hybrid flow chamber may comprise a chamber and a bag, a chamber and a plate, or a bag and a plate. The individual components of the hybrid flow chamber, i.e. the bag, plate or chamber, are as defined herein.

In one embodiment, human AB serum, autologous (e.g. human) serum, autologous (e.g. human) plasma, allogeneic (e.g. human) serum, allogeneic (e.g. human) plasma, mouse serum, mouse plasma or FBS is added additionally to the composition. In one embodiment, autologous (e.g. human) serum, autologous (e.g. human) plasma, allogeneic (e.g. human) serum, allogeneic (e.g. human) plasma, mouse serum, mouse plasma or FBS is added additionally to the composition. In one embodiment, mouse serum, mouse plasma or FBS is added additionally to the composition. Alternatively, the flow chamber, bag, plate or hybrid flow chamber can be coated with human AB serum, autologous (e.g. human) serum, autologous (e.g. human) plasma, allogeneic (e.g. human) serum, allogeneic (e.g. human) plasma, mouse serum, mouse plasma or FBS before the monocytes are added. In one embodiment, the flow chamber, bag, plate or hybrid flow chamber is coated with autologous (e.g. human) serum, autologous (e.g. human) plasma, allogeneic (e.g. human) serum, allogeneic (e.g. human) plasma, mouse serum, mouse plasma or FBS. In another embodiment, the flow chamber, bag, plate or hybrid flow chamber is coated with mouse serum, mouse plasma or FBS.

In one embodiment, the phDCs are incubated with the at least one antigenic protein or the at least one mRNA encoding for the at least one antigenic protein. Incubation can be performed under standard conditions for culturing of human cells, e.g. at 37° C. and 5% CO2 in standard mediums such as in RPMI-1640 medium. Alternatively or additionally, an incubation step can also be inserted after generation of phDCs from monocytes. Incubation can be performed for 0.5 h, 1 h, 2 h, 3 h, 4 h, 5 h, 6 h, 8 h, 12 h or 24 h. In another embodiment, the incubation step can be performed for at least 0.5 h, at least 1 h, at least 2 h, at least 3 h, at least 4 h, at least 5 h, at least 6 h, at least 8 h, at least 12 h or at least 20 h. In one embodiment, incubation is performed for 6 h. In one embodiment, incubation is performed for 12 h. In one embodiment, incubation is performed for 20 h. In one embodiment, incubation is performed for 6 to 20 hours. In one embodiment, incubation is performed for 8 to 20 hours. In general, phDCs have a higher transfection capability as compared to other DCs. The incubation step increases transfection efficiency.

Monocytes may be obtained by any suitable means, e.g., from a blood sample obtained from a donor or a fraction thereof. The blood sample or fraction thereof may be, e.g., a buffy coat including white blood cells and platelets. Alternatively, the blood sample or fraction thereof may be isolated peripheral blood mononuclear cells (PMBC). In one embodiment, the monocytes are autologous.

In one embodiment, the vaccine composition additionally comprises human AB serum, autologous (e.g. human) serum, autologous (e.g. human) plasma, allogeneic (e.g. human) serum, allogeneic (e.g. human) plasma, mouse serum, mouse plasma or FBS. In one embodiment, the vaccine composition additionally comprises autologous serum, autologous plasma, allogeneic serum, allogeneic plasma, mouse serum, mouse plasma or FBS. In one embodiment, the vaccine composition additionally comprises mouse serum, mouse plasma or FBS.

In one embodiment, the vaccine composition additionally comprises human AB serum, autologous (e.g. human) serum, autologous (e.g. human) plasma, allogeneic (e.g. human) serum, allogeneic (e.g. human) plasma, mouse serum, mouse plasma or FBS. In one embodiment, the vaccine composition additionally comprises autologous serum, autologous plasma, allogeneic serum, allogeneic plasma, mouse serum, mouse plasma or FBS. In one embodiment, the vaccine composition additionally comprises mouse serum, mouse plasma or FBS.

In one embodiment, the present invention relates to a vaccine composition comprising phDCs and at least one mRNA, which comprises a coding sequence encoding for at least one infectious disease associated antigenic protein or at least one tumor associated antigenic protein.

In one embodiment, the present invention relates to a vaccine composition comprising phDCs and at least one mRNA, which comprises a coding sequence encoding for at least one viral antigenic protein, optionally a coronavirus (e.g. betacoronavirus) antigenic protein or HIV antigenic protein.

In one embodiment, the present invention relates to a vaccine composition comprising phDCs and at least one mRNA, which comprises a coding sequence encoding for at least one SARS-CoV-2 antigenic protein.

In one embodiment, the present invention relates to a vaccine composition comprising phDCs and at least one mRNA, which comprises a coding sequence encoding for at least one tumor associated antigenic protein, optionally a blood cancer antigenic protein.

In one embodiment, the present invention relates to a vaccine composition comprising phDCs and at least one mRNA, which comprises a coding sequence encoding for at least one infectious disease associated antigenic protein or at least one tumor associated antigenic protein, wherein the at least one mRNA is comprised in nanoparticles, and wherein the at least one mRNA is optionally modified with one or more naturally occurring modified nucleosides.

In one embodiment, the present invention relates to a vaccine composition comprising phDCs and at least one mRNA, which comprises a coding sequence encoding for at least one infectious disease associated antigenic protein or at least one tumor associated antigenic protein, wherein the at least one mRNA is comprised in lipid nanoparticles, and wherein the at least one mRNA is optionally modified with one or more naturally occurring modified nucleosides.

In one embodiment, the present invention relates to a vaccine composition comprising phDCs and at least one mRNA, which comprises a coding sequence encoding for at least one viral antigenic protein, optionally a SARS-CoV-2 antigenic protein, wherein the at least one mRNA is comprised in lipid nanoparticles, and wherein the at least one mRNA is optionally modified with one or more naturally occurring modified nucleosides. In a preferred embodiment, the at least one mRNA encodes a SARS-COV-2 spike protein or a fragment thereof. In one embodiment, the fragment has a length of at least 100, at least 200, at least 400, at least 600 or at least 800 amino acids.

In one embodiment, the fragment has a length of 400 to 1200 amino acids, 600 to 1200 amino acids or 800 to 1200 amino acids.

In a preferred embodiment, the at least one mRNA, encoding a SARS-COV-2 antigenic protein, comprises a sequence of SEQ ID NO:19 or a sequence, which is at least 75%, 85%, 90%, 95%, 98% or 99% or 100% identical to SEQ ID NO:19.

In one embodiment, the present invention relates to a vaccine composition comprising phDCs and at least one mRNA, which comprises a coding sequence encoding for at least one blood cancer antigenic protein, wherein the at least one mRNA is comprised in lipid nanoparticles, and wherein the at least one mRNA is optionally modified with one or more naturally occurring modified nucleosides.

For the above embodiments, as regards the phDCs, lipid nanoparticles, mRNA and at least one antigenic protein, the respective embodiments as mentioned before and below apply mutatis mutandis. For instance, the lipid nanoparticles can comprise a cationic lipid, a polyethylene glycol (PEG) modified lipid, a cholesterol-based lipid and/or a non-cationic lipid. Optionally, the cationic lipid is present at a molar ratio between 30% and 40%, said PEG-modified lipid is present at a molar ratio between 1.5% and 4.0%, said cholesterol-based lipid is present at a molar ratio between 40% and 52%, and said non-cationic lipid is present at a molar ratio between 11% and 21%, wherein all the molar ratios are relative to the total lipid content of the LNP. Furthermore, the at least one mRNA optionally modified with one or more naturally occurring modified nucleosides, can be fully modified with N1-methyl-pseudouridine in place of every uridine. In one embodiment, the vaccine composition comprises (e.g. at least a portion of) antigen-specific phDCs. Said antigen-specific phDCs are obtainable by combining phDCs with the at least one mRNA encoding for the at least one antigenic protein or by combining monocytes with the at least one mRNA encoding for the at least one antigenic protein and then subjecting the mixture of monocytes and the at least one mRNA encoding for the at least one antigenic protein to a physical force.

The present invention further relates to a vaccine composition comprising antigen-specific monocytes and at least one mRNA encoding for at least one antigenic protein. The antigen-specific monocytes are obtainable by
combining monocytes (which have been obtained as described above) with at least one mRNA encoding for at least one antigenic protein;
subjecting the monocytes and the at least one mRNA encoding for at least one antigenic protein to a physical force.

In another embodiment, the present invention relates to a vaccine composition comprising antigen-specific monocytes, phDCs and at least one mRNA encoding for at least one antigenic protein. A physical force (e.g. shear force) can be applied by passing monocytes through a flow chamber which can take the form of, e.g., a plate or, e.g., a bag (e.g. flexible bag or plastic bag) or a combination thereof (e.g. hybrid of a plate and bag). The at least one mRNA can be comprised in nanoparticles, lipid nanoparticles in particular. All embodiments, e.g. as regards the at least one mRNA encoding for the at least one antigenic protein, or e.g. nanoparticles (in particular lipid nanoparticles), as disclosed herein apply mutatis mutandis.

### Second Aspect: Vaccine Composition Comprising phDCs for Use in Vaccination

The vaccine composition according to the first aspect can be used for vaccination. Thus, in one embodiment, the invention relates to a vaccine composition for use in a method of preventing or reducing a disease in a subject, said method comprising administering the vaccine composition to the subject. In one embodiment, the invention relates to a vaccine composition for use in a method of preventing a disease in a subject, said method comprising administering the vaccine composition to the subject.

The vaccine compositions of the invention are suitable for use in prophylactic vaccination against diseases as caused by the pathogens as listed in the first aspect or against the tumors/cancers as listed in the first aspect. Moreover, the vaccine compositions of the invention are suitable for use in prophylactic vaccination against diseases as caused by the pathogens listed in the detailed description, section “pathogens” or against the tumors/cancers as listed in the detailed description, section “tumors/cancer”. For example, the vaccine compositions of the invention are suitable for use in vaccination against AIDS, hepatitis A, hepatitis B, hepatitis C, polio, gastroenteritis, dengue fever, yellow fever, encephalitis, meningitis, or meningoencephalitis caused by West nile virus, influenza, ebola, rabies, mumps, measles, herpes, pox, Middle East respiratory syndrome, severe acute respiratory syndrome, Covid-19, borreliosis, pseudomonas, burkholderia, tuberculosis and/or malaria depending on the at least one antigen or the at least one mRNA (encoding the at least one antigenic protein), which is present in the composition. In one embodiment, the vaccine compositions of the invention are for use in vaccination against AIDS and/or Covid-19. In one embodiment, the vaccine compositions of the invention are for use in vaccination against Covid-19. In one embodiment, the vaccine compositions of the invention are for use in vaccination against Covid-19 and are administered intranasally.

In one embodiment, the subject and the donor are identical. In this embodiment, the subject is vaccinated with autologous (antigen-specific) phDCs. In one embodiment, the subject and the donor are related. In another embodiment, the subject and donor are different (i.e. not related). In this embodiment, the subject is vaccinated with allogeneic (antigen-specific) phDCs.

Administration of the vaccine compositions of the invention can be subcutaneous, intravenous, intramuscular, inhalative, intra-articular, intra-synovial, intrasternal, intrathecal, intra-hepatic, intralesional, intracranial, transdermal, intradermal, intrapulmonal, intraperitoneal, intracardial, intraarterial, intranasal or sublingual. In one embodiment, administration is inhalative, intravenous, intradermal, intranasal or intramuscular. In one embodiment, administration is intranasal. In one embodiment, administration is intradermal or intramuscular. In one embodiment, administration is intravenous. In some embodiments, the vaccine compositions of the invention are administered as a prime and/or boost. In one embodiment, the vaccine compositions of the invention are administered as a prime. In one embodiment, the vaccine compositions of the invention are administered as a boost. In one embodiment, the vaccine compositions of the invention are administered as a prime and a boost. The vaccine compositions of the invention are able to stimulate an immune response against the disease associated antigen or antigenic protein in the subject. In one embodiment, the vaccine compositions of the invention are able to stimulate an immune response against an infectious disease associated antigen or infectious disease associated antigenic protein. In one embodiment, the vaccine compositions of the invention are able to stimulate an immune response against a tumor associated antigen or a tumor associated antigenic protein.

In one embodiment, the disease is caused by disease causing particles, optionally comprising viruses, bacteria, fungi, parasites and/or tumor cells. In one embodiment, the disease causing particles comprise pathogens such as viruses, bacteria, fungi and parasites. In one embodiment, the disease causing particles comprise viruses. In one embodiment, the disease causing particles comprise tumor cells (or cancer cells).

In some embodiments, the vaccine compositions as provided herein may be characterized in that subjects who have been vaccinated with such vaccine compositions (e.g., with at least one dose, at least two doses, etc) may show reduced and/or more transient presence of disease causing particles in relevant site(s) (e.g. nose and/or lungs, and/or any other tissue susceptible to the corresponding disease or disorder) upon outbreak of the disease as compared with an appropriate control (e.g. an established expected level for a comparable subject or population not having been vaccinated and having been exposed to the disease causing particles; or a comparable subject or population having been vaccinated with a different vaccine such as a mRNA vaccine).

In one embodiment, the concentration of the disease causing particles is effectively reduced after the outbreak of the disease in the subject as compared to a concentration of the disease causing particles in a second subject, in which the outbreak of the same disease is observed (e.g. diagnosed) but which has not been administered the vaccine composition of the invention. In one embodiment, the concentration of the disease causing particles is effectively reduced upon an infection in the subject as compared to a concentration of the disease causing particles in a second infected subject which has not been administered the vaccine composition of the invention; or as compared to a concentration of the disease causing particles in a second infected subject which has been vaccinated against the same disease causing particle with a different vaccine, preferably a vaccine not comprising dendritic cells; even more preferred compared to a concentration of the disease causing particles in a second infected subject which has been vaccinated against the same disease causing particle with an mRNA vaccine.

In one embodiment, the concentration of the disease causing particles (e.g. tumor cells or tumor volume) is effectively reduced upon a tumor outbreak in the subject as compared to a concentration of the disease causing particles in a second subject, in which the same tumor outbreak is observed and which has not been administered the vaccine composition of the invention; or as compared to a concentration of the disease causing particles in a second subject, in which the same tumor outbreak is observed and which has been vaccinated against the same disease causing particle with a different vaccine, preferably a vaccine not comprising dendritic cells; even more preferred compared to a concentration of the disease causing particles in a second subject, in which the same tumor outbreak is observed and which has been vaccinated against the same disease causing particle with an RNA vaccine. In some embodiments, the subject is in remission after conventional anti-tumor therapeutic intervention. The vaccine compositions can be administered in one embodiment to such subject being in remission to act as an anti-tumor vaccine and to reduce the risk of recurrence.

In one embodiment, the anti-antigen antibody titer in the subject is lower after vaccination with the vaccine compositions of the invention as compared to the anti-antigen antibody titer in a second subject which has been vaccinated against the same disease causing particle with a different vaccine, preferably a vaccine not comprising dendritic cells. In one embodiment, the anti-antigen antibody titer in the subject is lower after vaccination and after infection as compared to a second infected subject which has been vaccinated against the same disease causing particle with a different vaccine, preferably a vaccine not comprising dendritic cells. Importantly, the subject who received the vaccine compositions is equally protected as compared to the second subject showing a higher anti-antigen antibody titer. This effect may be due to the antigen-specific phDCs of the invention favoring a T cell-based cellular immunity whereas conventional vaccines, especially RNA based vaccines lead to a strong antibody response.

In one embodiment, the concentration of the disease causing particles is effectively reduced by at least 10%, 20%, 30%, 40%, 50%, 60%, 70%, 80%, 90%, 95%, 98% or 99% in the subject. Reduction can be measured as compared to an appropriate control, e.g. as compared to the concentration of the disease causing particles before outbreak of the disease or as compared to the concentration of the disease causing particles in a second subject as mentioned above. In one embodiment, the time period in which the subject shows symptoms of the disease is reduced by at least 10%, 20%, 30%, 40%, 50%, 60%, 70%, 80%, 90%, 95%, 98% or 99% in the subjects who have been vaccinated with the vaccine compositions of the invention. Comparison can be made to an appropriate control, e.g. to subjects not having received the antigen-specific phDCs, or having received a different vaccine such as a RNA vaccine.

In one embodiment, the concentration of the disease causing particles is effectively reduced in the subject systemically upon infection. In one embodiment, the concentration of the disease causing particles is effectively reduced locally. In one embodiment, the concentration of the disease causing particles is effectively reduced in the brain. In one embodiment, the concentration of the disease causing particles is effectively reduced in mucosal tissues (i.e., the vaccine compositions of the invention induce mucosal immunity). The reduction may be at least 5%, at least 10%, at least 20%, at least 30%, at least 40%, at least 50%, at least 60%, at least 70%, at least 80%, or at least 90%. Comparison can be made to the concentration of the disease causing particles in a second subject not having received the vaccine composition of the invention, or having received a different vaccine such as a RNA vaccine.

In one embodiment, after vaccination with the vaccine compositions of the invention, the subject shows an increased proportion of central memory T cells (Tcm) and/or stem-like T cells, which are specific to the at least one disease associated antigen (or at least one antigenic protein), as compared to a second subject vaccinated against the same disease causing particle with a different vaccine, preferably a vaccine not comprising dendritic cells; even more preferred compared to a second subject which has been vaccinated against the same disease causing particle with an RNA vaccine. In one embodiment, after vaccination with the vaccine compositions of the invention, the subject shows a decreased proportion of effector dominant T cells (Teff), which are specific to the at least one disease associated antigen (or at least one antigenic protein), as compared to a second subject vaccinated against the same disease causing particle with a different vaccine, preferably a vaccine not comprising dendritic cells; even more preferred compared to a second subject which has been vaccinated against the same disease causing particle with an RNA vaccine. In one embodiment, after vaccination with the vaccine compositions of the invention, the subject shows an increased proportion of natural killer cells (NK) as compared to a second subject vaccinated against the same disease causing particle with a different vaccine, preferably a vaccine not comprising dendritic cells; even more preferred compared to a second subject which has been vaccinated against the same disease causing particle with an mRNA vaccine. In one embodiment, after vaccination with the vaccine compositions of the invention, the subject shows an increased level of IFN-7 as compared to a second subject vaccinated against the same disease causing particle with a different vaccine, preferably a vaccine not comprising dendritic cells; even more preferred compared to a second subject which has been vaccinated against the same disease causing particle with an mRNA vaccine. In one embodiment, after vaccination with the vaccine compositions of the invention, the subject shows a decreased proportion of exhausted effector T cells as compared to a second subject vaccinated against the same disease causing particle with a different vaccine, preferably a vaccine not comprising dendritic cells; even more preferred compared to a second subject which has been vaccinated against the same disease causing particle with an mRNA vaccine. These effects can be observed after a certain time following initial and/or booster administration, such as e.g. at least 2, at least 3, at least 4, at least 5, at least 6, at least 7, at least 8, at least 9, at least 10, at least 11, or at least 12 weeks after initial and/or booster administration. Comparison can also be made to other appropriate controls such as the same subject before administration of the vaccine composition of the invention (and not to a second subject). The increase in proportion of central memory T cells and/or stem-like T cells and/or natural killer cells can be at least 5%, at least 10%, at least 20%, at least 30%, at least 40%, at least 50%, at least 60%, at least 70%, at least 80%, or at least 90%. The decrease in proportion of effector dominant T cells and/or exhausted effector T cells can be at least 5%, at least 10%, at least 20%, at least 30%, at least 40%, at least 50%, at least 60%, at least 70%, at least 80%, or at least 90%. The increase in level of IFN-7 secretion can be at least 2-fold, at least 10-fold, at least 50-fold, at least 100-fold, at least 500-fold or at least 5000-fold. The proportion of Tcm can be determined, e.g., by measuring markers which are associated with a central memory T cell phenotype such as CD44/CD62L. Teff show reduced expression of CD62L as compared to Tcm. The proportion of stem-like T cells can be determined by, e.g., measuring markers which are associated with a stem-like T cell phenotype such as IL7Ra/SCA-1. The expression of both markers is increased for stem-like T cells. NK cells can e.g. be determined by positive selection using a NK1.1 cell isolation method. The proportion of exhausted effector T cells can be determined by, e.g., measuring expression markers which are associated with an exhausted state such as PD1. Vaccination can refer to initial vaccination and/or booster administration, i.e. one dose or more doses. In one embodiment, vaccination refers to initial and booster administration.

As mentioned, the present invention in one embodiment, relates to a vaccine composition for use in a method of prophylactic vaccination. By prohylactic vaccination, the invention refers to the administration of the vaccine composition described herein to subjects which have not been diagnosed with respect to the above-mention conditions such as, e.g., an infectious disease (e.g., Covid-19) or a tumor, against which the vaccine composition is directed. Prophylactic vaccination thus relates to the prevention or reduction of a condition that has not been diagnosed yet.

In one embodiment, the present invention relates to a vaccine composition of the first aspect for use in a porphylactic method of preventing or reducing a disease in a subject, said method comprising administering the vaccine composition to the subject.

In one embodiment, the vaccine composition is for use in a prophylactic method of preventing or reducing an infectious disease or a tumor in a subject, said method comprising administering the vaccine composition to the subject.

In one embodiment, the vaccine composition is for use in a prophylactic method of preventing or reducing a viral disease in a subject, said method comprising administering the vaccine composition to the subject.

In one embodiment, the vaccine composition is for use in a prophylactic method of preventing or reducing Covid-19 (or one of the symptoms of Covid-19) in a subject, said method comprising administering the vaccine composition to the subject.

In one embodiment, the vaccine composition is for use in a prophylactic method of preventing or reducing a blood cancer in a subject, said method comprising administering the vaccine composition to the subject.

In one embodiment, the vaccine composition is for use in a prophylactic method of preventing or reducing a solid tumor in a subject, said method comprising administering the vaccine composition to the subject.

### Third Aspect: Method of Prophylactically Preventing or Reducing a Disease

In a third aspect, the present invention relates to a method of prophylactically preventing or reducing a disease in a subject, said method comprising administering the vaccine composition of the first aspect (including all embodiments of the first and second aspect as described above) to the subject.

### Fourth Aspect: Use of a Pharmaceutical Composition for Vaccination

In a fourth aspect, the present invention relates to the use of a pharmaceutical composition for vaccination, wherein the pharmaceutical composition is the vaccine composition of the first aspect. Thus, as regards the pharmaceutical composition, phDCs, nanoparticles, RNA (e.g. mRNA) and at least one antigenic protein, the respective embodiments of the first, second and third aspect apply mutatis mutandis.

### Fifth Aspect: Kit Comprising phDCs and mRNA

In a fifth aspect, the present invention relates to a kit comprising phDCs and at least one RNA (e.g. mRNA), which encodes for at least one antigenic protein. The kit may further comprise nanoparticles (e.g. lipid nanoparticles). In case the kit comprises nanoparticles, the nanoparticles comprise the RNA (e.g. mRNA), which encodes for the at least one antigenic protein. As regards the phDCs, nanoparticles (e.g. lipid nanoparticles), RNA (e.g. mRNA) and at least one antigenic protein, the respective embodiments of the first aspect apply mutatis mutandis.

The present invention also relates to a kit comprising monocytes and at least one RNA (e.g. mRNA), which encodes for at least one antigenic protein. The kit may further comprise nanoparticles (e.g. lipid nanoparticles). In case the kit comprises nanoparticles, the nanoparticles comprise the RNA (e.g. mRNA), which encodes for the at least one antigenic protein. As regards the monocytes, nanoparticles (e.g. lipid nanoparticles), RNA (e.g. mRNA) and at least one antigenic protein, the respective embodiments of the first aspect apply mutatis mutandis.

### FIGURE LEGENDS

FIG. 1 Murine phDC specifically internalize and express Spike protein encoded by mRNA-containing LNPs. A, B, FACS plot showing spike protein positivity in CD11b positive phDCs without LNP transfection (A) and with LNP transfection (B). C, D, Confocal microscopy analysis using same sample showing spike protein expression in CD11b+Ly6G-phDCs (projected Z-stack images). E. Z-plane slice image of the LNP transfected phDC.

FIG. 2 A, C57BL/6 mice were administered i.v. treatment with phDCs transduced with OVA mRNA LNP, or treated with OVA mRNA LNP via i.m. route (no phDCs), 2 days post EG7-OVA subcutaneous tumor implant (3×10⁶ cells per mouse). Approximately 10 ng of LNP were used to transduce the phDCs (per mouse), and same amount was injected via i.m. per mouse (0.5 ug/kg of LNP, dose equivalent to COVID-19 Pfizer mRNA LNP vaccine in current human vaccine setting). Treatments were given twice per week, with each mouse receiving a total of 5 treatments. Tumors were visible and palpable (>50 mm³) at the time of treatment initiation. B, EG7-OVA tumor growth was monitored throughout the experiment.

FIG. 3 A, phDC[ova] or IM[ova] mice were given five total treatments at 10 ng [ova] per mouse, starting two days following EG7-OVA subcutaneous tumor inoculation with 3*10⁶ cells per mouse (same experiment as FIG. 3). B-C, tumor growth was monitored for the duration of the experiment, with cumulative tumor growth per experimental group (B) and individual mouse tumor growth curves (C) represented. D-E, CD8+ splenic T cells were isolated from treated mice on Day 30 and immediately placed into an 18 hr IFN-g Elispot assay at 1*10⁵ cells per well in the presence or absence of 10 ug/mL SIINFEKL peptide. Representative Elispot well images (D) and IFNg spot quantitation (E) are provided for each experimental group. *[ova]=LNP-mRNA encoding ovalbumin.

FIG. 4 A, phDC[ova] or IM[ova] mice were given five total treatments at 10 ng [ova] per mouse, starting two days following EG7-OVA subcutaneous tumor inoculation with 3*10⁶ cells per mouse (same experiment as FIG. 3). Splenocytes from the treated mice were harvested at the end of the 28-day observation period for T cell analysis and characterization by flow cytometry of A, antigen (SIINFEKL)-specific CD8 T cells via dextramer analysis; Tem (effector)/Tcm (central memory) type phenotype analysis via CD44/CD62L expression; detection of stem-like T cell via IL7Ra/SCA-1 expression, and B, T cell exhaustion marker evaluation via PD-1 expression. *[ova]=LNP-mRNA encoding ovalbumin.

FIG. 5 phDC[ova] or IM[ova] mice were vaccinated (prime/boost) on days −7 and 0, respectively. Undifferentiated splenocytes were harvested post vaccination and immediately placed in an 18 hr IFN-g Elispot without added antigenic stimulation (A, B and C). To identify cells responsible for splenic IFN-g, isolated CD8+ or NK1.1+ splenocytes were collected post vaccination from phDC[ova] mice and assessed for IFN-g spots (D). *[ova]=LNP-mRNA encoding ovalbumin.

FIG. 6 A, C57BL/6 mice were administered i.v. treatment with phDCs transduced with 1 ug/mL OVA mRNA LNP, or treated i.v. with soluble OVA protein (50 ug/mouse, no phDCs), on days −14 and −7 prior to EG7-OVA subcutaneous tumor implant (1×10⁶ cells per mouse). B, EG7-OVA tumor growth was monitored throughout the experiment.

FIG. 7 Mouse phDCs were transduced with OVA mRNA LNP (CKK) at 1 μg/ml, and stained for intracellular OVA protein after over-night culture. PhDCs transduced with SP LNP (1 ug/ml) placed as control. (n=3) OVA detection with Rockland OVA ab (FITC conjugated).

FIG. 8 Mouse phDCs were incubated overnight with OVA in various antigen form; soluble, expressed in tumor cells or OVA encoding mRNA containing LNPs. Cells were then harvested, and stained for surface CD11b, Ly6G and 25.D1 (ab against SIINFEKL bound MHC I in H-2Kb strain). Ly6G positive cells were selected out for neutrophil exclusion. FACs plot showing 25.D1 positivity on CD11b+ phDCs.

FIG. 9 PhDC (PP PBMCs from B6 mice) were transduced with OVA mRNA LNPs (1 ug/ml) in standard overnight culture protocol. Cells were harvested, washed and fixed (intraprep kit) at various time points, and stained for surface CD11b and SIINFEKL-MHC I complex (25.D1 ab). 25.D1 expression level show in CD11b+ cells at various time points shown in FACs plot (upper panel). Percentage of 25.D1 positive cells and MFI level of 25.D1 in CD11b+ subset shown in bar graph (below).

FIG. 10 Enriched phDC (monocytes purified from plate passed PBMCs from mice) or BMDC (cytokine induced DC from bone marrow of mice) were incubated over-night with/without LNPs containing OVA mRNA 0.1 ug/ml in standard overnight protocol. Cells were harvested and stained for surface CD11b and 25.D1 (ab against SIINFEKL bound MHC I in H-2Kb strain) for phDC samples, and CD11c and 25.D1 for BMDC sample. FACs plot showing 25.D1 positivity on CD11b+ phDC and CD11c+ BMDCs.

FIG. 11 Mouse phDC were transduced with either SIINFEKL peptide mRNA LNP or OVA protein mRNA LNP. After over-night incubation, cells were harvested and stained with 25.D1 ab for detection of SIINFEKL MHC I complex signal. FACS plot showing 25.D1 signal on CD11b+ phDC subset.

FIG. 12 phDC from mouse were pulsed with OVA mRNA LNPs at differing concentration, then cultured with OT1 T cells. Proliferation of the OT1 CD8 T cells (CFSE labeled) was evaluated as shown by reduction in CFSE signal.

FIG. 13 phDC from mouse (B6) were pulsed with 1 μg/ml OVA mRNA, then cultured with 100K OT1 T cells at differing phDC number (96 well U bottom plate). Proliferation of the OT1 CD8 T cells (CFSE labeled) were evaluated as readout.

FIG. 14 C57BL/6 mice were administered i.v. treatment with phDCs transduced with OVA mRNA LNP* or injected with with mock (SP) mRNA LNP* or OVA mRNA LNP via i.m.* route (no phDC), 4 days post EG7 tumor implant (3×106 cells per mouse). Treatments were given twice per week (Tx on day 4, 8, 11, 15, 18 and 25). Tumors were visible and palpable (>50 mm3) at the time of treatment initiation. N=10 for each groups.

FIG. 15 (a) Splenocytes from mice in experiment 21-1008 (Therapeutic exp, HB) were analyzed for SIINFEKL tetramer positivity at the end of the in vivo tumor monitoring period (day 32). H-2Kb OVA SIINFEKL tetramer positivity on CD8+ subset shown in FACs plot. Splenocytes from 10 mice pooled prior to analysis. Last treatment given 1 week prior to analysis. (b) T cells isolated from the splenocytes of the three experimental groups were transferred (15M T cells/mouse) into EG7 tumor bearing mice, and tumor development was monitored over the course of 19 days.

### DETAILED DESCRIPTION

### I. Exemplary Preparation of phDCs

In one example, phDCs can be prepared from monocytes (e.g. obtained from a donor). For instance, phDCs can be generated by subjecting monocytes (e.g. obtained from a donor) to a physical force. A physical force can e.g. be generated by passing the monocytes through a flow chamber. In one embodiment of the present invention, phDC are obtained by plate-passage of monocytes using an extracorporeal photopheresis (ECP) derived process. Thus, the flow chamber can be a plate in one embodiment. A plate for preparing phDCs has been previously described in the literature, see e.g. Durazzo et al., 2014 (“Induction of Monocyte-to-Dendritic Cell Maturation by Extracorporeal Photochemotherapy: Initiation via Direct Platelet Signaling”) or Ventura et al., 2018 (“Extracorporeal Photochemotherapy Drives Monocyte-to-Dendritic Cell Maturation to Induce Anticancer Immunity”). Methods and devices for extracorporeal activation of monocytes and generation of dendritic cells therefrom are described in WO2014/106629 A1, WO2014/106631 A1, WO2016/001405 A1, and WO2017/005700 A1, each of which is incorporated herein by reference in its entirety. ECP describes a process in which monocytes derived from a blood sample or a fraction thereof are exposed to mechanical stress (e.g., a physical force in the form of shear force) and plasma components (e.g., platelets) or derivatives or mimics thereof, thereby activating the monocytes to differentiate into healthy, physiologic dendritic cells which are also termed phDC herein. However, providing monocytes and subjecting the monocytes to a physical force is sufficient for activation and differentiation of the monocytes into phDCs. ECP and ECP derived processes, including the differentiation of monocytes into phDC, may be performed in a flow chamber (e.g., a THERAKOS® CELLEX® device); or in a miniaturized ECP device, e.g., a Transimmunization plate as described in WO2017/005700 A1; or in a bag (e.g. flexible bag or plastic bag); or in a combination of any of the afore-mentioned).

The bag can be made of any material that does not leak liquids such as e.g. rubber, silicone or plastic. Preferably, the material is able to bend easily without breaking.

Optionally, the bag or plate is made of a plastic material. Suitable plastic materials comprise polyolefin, polyethylene, fluoropolymer, polyvinyl chloride, ethylene-vinyl acetate-copolymer, ethylene vinyl alcohol, polyvinylidene fluoride, and/or other plastic comprising materials approved for medical use. The preferred plastic material is ethylen-vinyl acetate-copolymer. The bag or plate may be made of a material that provides a degree of transparency such that the sample or cell mixture can be irradiated with visible or UV light.

In one embodiment, the flow chamber can be hybrid flow chamber or a combination of a chamber and a bag, wherein each of the components are as described herein.

The inventors found that phDC obtained by the method described above are advantageous as compared to DC obtained by other methods such as incubation of blood monocytes with cytokines or direct isolation from a donor, as phDC are generated physiologically (without the need for chemicals such as cytokines and/or apoptotic agents) with greater reproducibility and controllability under precise in vitro laboratory conditions. Benefits of the above described method for generating phDCs compared to other methods such as incubation with cytokines include one or more of higher yields, a faster process, better intracellular antigen processing and more effective priming of disease-specific cytotoxic T cells.

phDC of a donor are obtained by subjecting monocytes contained in a blood sample (obtained from the donor) to a shear force by passing the blood sample or fraction thereof through a flow chamber. Preferably, platelets are present in the flow chamber which can be either derived from the donor's blood sample or a fraction thereof or provided separately. Additionally or alternatively, plasma components can be present in the flow chamber which can be either derived from the donor's blood sample or a fraction thereof or provided separately. After phDCs have been obtained they can be stored frozen until further use, e.g. combination with the at least one disease associated antigen or at least one mRNA encoding for at least one antigenic protein.

A monocyte of a donor may be obtained by any suitable means, e.g., from a blood sample or a fraction thereof. The fraction of the blood sample may be, e.g., a buffy coat including white blood cells and platelets. Alternatively, the fraction of the blood sample may be an isolated peripheral blood mononuclear cell (PMBC). PMBCs may be isolated from a blood sample using, e.g., centrifugation over a Ficoll-Hypaque gradient (Isolymph, CTL Scientific). In another example, the fraction of the blood sample may be a purified or enriched monocyte preparation. Monocytes may be enriched from PBMCs using, e.g., one, two, or all three of plastic adherence; CD14 magnetic bead positive selection (e.g., from Miltenyi Biotec); and a Monocyte Isolation Kit II (Miltenyi Biotec).

Any suitable volume of blood can be used. The blood sample (e.g., the blood sample from which the fraction is derived) may be between about 1 μL and about 500 mL, e.g., between about 1 μL and about 10 mL, between about 1 μL and about 5 mL, between about 1 μL and about 1 mL, between about 1 μL and about 750 μL, between about 1 μL and about 500 μL, between about 1 μL and about 250 μL, between about 10 mL and about 450 mL, about 20 mL and about 400 mL, about 30 mL and about 350 mL, about 40 mL and about 300 mL, about 50 mL and about 200 mL, or about 50 mL and about 100 mL. In some embodiments, the blood sample or the fraction thereof or the additional blood sample or the fraction thereof is less than or equal to about 100 mL (e.g., about 50 mL to about 100 mL). In one embodiment, the monocytes are derived from a blood sample obtained from the donor. However, monocytes can be derived from other sources in the donor that provide monocytes, e.g. bone marrow, spleen or other peripheral tissues. In one embodiment, monocytes are derived from peripheral blood mononuclear cells (PBMC) obtained from the donor.

The skilled person is well aware of methods to distinguish dendritic cells including phDC from monocytes, such as e.g. by assessing gene expression. For example, CD80, CD83, and CD86 are markers expressed in higher levels by mature dendritic cells as compared to monocytes. In one embodiment, the donor is mammalian. Mammals include for example, but are not limited to, humans, non-human primates, pigs, dogs, cats and rodents. In a preferred embodiment, the donor is human.

### II. The Antigenic Protein

phDCs are combined with at least one mRNA encoding at least one antigenic protein. The antigenic protein may be a disease associated antigen or a fragment thereof. If it is referred to a “disease associated antigen” or “antigenic protein”, “a fragment thereof” is included in the meaning. Upon combination of phDCs with a disease associated antigen, phDCs are able to take up, process and present the antigen on their surface. This process is also referred to as “loading”. Such loaded phDCs can elicit potent anti-disease, e.g. anti-infectious disease or anti-tumor, immune responses.

The at least one disease associated antigen can be loaded as the antigen itself (e.g., proteins, peptides, epitopes, cells, cell or tissue lysates, viruses, viral particles, etc., including fragments of each of the aforementioned) or can be loaded (loaded in the meaning of the phDCs taking up the antigen or antigenic protein) as a nucleic acid encoding antigen. Therefore, in one embodiment, the phDCs comprise a nucleic acid, preferably a RNA, more preferably an mRNA, encoding the antigenic protein or disease associated antigen. In one embodiment, the at least one disease associated antigen is loaded as a nucleic acid encoding the antigen. The at least one disease associated antigen can be provided directly or stored in frozen or a lyophilized form until use. Methods for loading dendritic cells with antigens are known to those of skill in the art. In one example, the phDCs are loaded by combination, e.g. incubation, of the phDCs with the at least one disease associated antigen. In some embodiments, the phDCs may be loaded with different antigens to give rise to multi-valent antigen-specific phDCs. In other examples, phDCs are loaded by electroporation, polymer or lipid based nanoparticles or the cell squeeze method. In one embodiment, phDCs are loaded by encapsulating the at least one disease associated antigen (e.g. in the form of an mRNA) in lipid nanoparticles and then combining the phDCs with the encapsulated at least one disease associated antigen. In case, the at least one antigen is encoded by RNA, in particular mRNA, the at least one disease associated antigen is referred to as an antigenic protein for the purpose of this invention.

As regards the cell squeeze method, phDCs are flowed in a solution containing the at least one disease associated antigen or fragment thereof. For loading, phDCs are squeezed through a microfluidic constriction that is smaller than the diameter of the cells. This results in temporary disruption of the cell membrane, enabling the at least one disease-associated antigen to enter the cytosol of the phDCs.

Once phDCs have been obtained and combined with the at least one disease associated antigen, the mixture can for example be incubated under standard conditions. Culturing can be performed under standard conditions, e.g. at 37° C. and 5% CO₂ in standard mediums for culturing of human cells such as in RPMI-1640 medium (obtainable e.g. from GIBCO), supplemented with 15% AB serum (obtainable from e.g. Gemini Bio-Products). An incubation step can be added after combining the phDCs with at least one disease associated antigen. Alternatively or additionally, an incubation step can also be inserted after generation of phDCs from monocytes. The incubation step can be performed for 0.5 h, 1 h, 2 h, 3 h, 4 h, 5 h, 6 h, 12 h, 20 h or 24 h. In another embodiment, the incubation step can be performed for at least 0.5 h, at least 1 h, at least 2 h, at least 3 h, at least 4 h, at least 5 h, at least 6 h, at least 12 h, at least 20 h or at least 24 h. It needs to be understood that all method steps are performed in vitro.

### II.A Disease Causing Particles

The at least one disease associated antigen or the at least one antigenic protein can be derived from disease causing particles, which comprise pathogens and tumor cells. Pathogens are viruses, bacteria, fungi, prions and parasites. Thus, in one embodiment, disease causing particles comprise viruses, bacteria, fungi, prions, parasites and tumor cells. In the following sections on pathogens and tumors/cancer, the term “antigen” is used interchangeably with the term “antigenic protein”. For example, if an infectious disease antigen or tumor antigen is mentioned, it is also referred to an infectious disease associated antigenic protein or a tumor associated antigenic protein. If a viral antigen is mentioned, it is also referred to a viral antigenic protein. If a HIV or SARS-CoV-2 antigen is mentioned, it is also referred to a HIV or SARS-CoV-2 antigenic protein.

### II.A.1 Pathogens

In some embodiments, the disease associated antigen is an infectious disease associated antigen. In some embodiments, the disease associated antigen is a viral antigen. In some embodiments, the disease associated antigenic protein is a viral antigenic protein. Examples of viruses, from which the disease associated antigen or antigenic protein can be derived include: Retroviridae (e.g. human immunodeficiency viruses, such as HIV-1 (also referred to as HTLV-III, LAV or HTLV-III/LAV, or HIV-III; and other isolates, such as HIV-LP, HIV-2; Picornaviridae (e.g. polio viruses, hepatitis A virus; enteroviruses, human coxsackie viruses, rhinoviruses, echoviruses); Calciviridae (e.g. strains that cause gastroenteritis); Togaviridae (e.g. equine encephalitis viruses, rubella viruses); Flaviridae (e.g. dengue viruses, encephalitis viruses, yellow fever viruses); Coronaviridae (e.g. coronaviruses); Rhabdoviridae (e.g. vesicular stomatitis viruses, rabies viruses); Filoviridae (e.g. ebola viruses); Paramyxoviridae (e.g. parainfluenza viruses, mumps virus, measles virus, respiratory syncytial virus); Orthomyxoviridae (e.g. influenza viruses); Bungaviridae (e.g. Hantaan viruses, bunga viruses, phleboviruses and Nairo viruses); Arena viridae (hemorrhagic fever viruses); Reoviridae (e.g. reoviruses, orbiviruses and rotaviruses); Birnaviridae; Hepadnaviridae (Hepatitis B virus); Parvoviridae (parvoviruses); Papovaviridae (papilloma viruses, polyoma viruses); Adenoviridae (most adenoviruses); Herpesviridae (herpes simplex virus (HSV) 1 and 2, varicella zoster virus, cytomegalovirus (CMV), herpes virus); Poxviridae (variola viruses, vaccinia viruses, pox viruses); and Iridoviridae (e.g. African swine fever virus); and unclassified viruses (e.g. the infectious agents of spongiform encephalopathies, the agent of delta hepatitis (thought to be a defective satellite of hepatitis B virus), the agents of non-A, non-B hepatitis (enterically transmitted; parenterally transmitted (i.e. hepatitis C); Norwalk and related viruses and astroviruses).

Preferably, the disease associated antigen is a viral antigen, more preferably a Retroviridae or Coronaviridae antigen, and most preferably a HIV or SARS-CoV-2 antigen. In an even more preferred embodiment, the disease associated antigen is derived from SARS-CoV-2.

In some embodiments, the disease associated antigen is a bacterial antigen. Examples of bacteria, from which the disease associated antigen can be derived include: Helicobacter pyloris, Borrelia spp. (e.g. Borrelia burgdorferi), Legionella pneumophilia, Mycobacteria spp. (e.g. M. tuberculosis, M avium, M. intracellulare, M kansasii, M gordonae), Staphylococcus aureus, Neisseria gonorrhoeae, Neisseria meningitidis, Listeria monocytogenes, Streptococcus pyogenes (Group A Streptococcus), Streptococcus agalactiae (Group B Streptococcus), Streptococcus (viridans group), Streptococcus faecalis, Streptococcus bovis, Streptococcus (anaerobic spp.), Streptococcus pneumoniae, pathogenic Campylobacter spp., Enterococcus spp., Haemophilus influenzae, Bacillus anthracis, Corynebacterium diphtheriae, Corynebacterium spp., Erysipelothrix rhusiopathiae, Clostridium perfringens, Clostridium tetani, Enterobacter aerogenes, Klebsiella pneumoniae, Pasteurella multocida, Bacteroides spp., Fusobacterium nucleatum, Streptobacillus moniliformis, Treponema pertenue, Leptospira and Actinomyces israelii, Burkholderia pseudomallei, Burkholderia mallei, Pseudomonas aeruginosa.

Preferably the bacterial antigen is a Borrelia spp. or Mycobacteria spp. antigen.

In some embodiments, the disease associated antigen is a fungal antigen. Examples of fungi, from which the disease associated antigen can be derived include: Cryptococcus neoformans, Histoplasma capsulatum, Coccidioides immitis, Blastomyces dermatitidis, Chlamydia trachomatis, Candida albicans.

In some embodiments, the disease associated antigen is a parasite antigen. Parasite antigens can be derived from protozoa, helminths or ectoparasites. Examples of parasites, from which the disease associated antigen can be derived include: Plasmodium falciparum, Plasmodium vivax, Plasmodium ovale, Plasmodium malariae, Leishmania species, Trypanosome species (African and American), cryptosporidiums, isospora species, Naegleria fowleri, Acanthamoeba species, Balamuthia mandrillaris, Toxoplasma gondii or Pneumocystis carinii.

### II.A.2 Tumors Cancer

In some embodiments, the disease associated antigen is a tumor or cancer antigen, e.g. a tumor associated antigen (TAA) or a tumor specific antigen (TSA). For the purpose of this disclosure, TAAs comprise the group of TSAs, if not mentioned otherwise. Tumor or cancer antigens can be found on tumor or cancer cells. The tumor or cancer antigen can be derived from a solid tumor or a blood cancer.

Examples of tumors or cancers, from which the disease associated antigen can be derived comprise leukemia, melanoma, lymphoma, endometrial cancer, kidney cancer, brain cancer, cervical cancer, liver cancer, head and neck cancer, gastrointestinal cancer, lymph node cancer, pancreas cancer, ear, nose and throat (ENT) cancer, breast cancer, prostate cancer, ovarian cancer or lung cancer.

In certain embodiments, the disease associated antigen is a tumor-associated antigen. Tumor-associated antigens comprise Her2, prostate stem cell antigen (PSCA), PSMA (prostate-specific membrane antigen), B cell maturation antigen (BCMA), ERK5, alpha-fetoprotein (AFP), carcinoembryonic antigen (CEA), cancer antigen-125 (CA-125), CA19-9, calretinin, MUC-1, epithelial membrane protein (EMA), epithelial tumor antigen (ETA), tyrosinase, melanoma-associated antigen (MAGE), CD34, CD45, CD99, CD117, chromogranin, cytokeratin, desmin, glial fibrillary acidic protein (GFAP), gross cystic disease fluid protein (GCDFP-15), HMIB-45 antigen, protein melan-A (melanoma antigen recognized by T lymphocytes; MART-1), myo-D1, muscle-specific actin (MSA), neurofilament, neuron-specific enolase (NSE), placental alkaline phosphatase, synaptophysin, thyroglobulin, thyroid transcription factor-1, the dimeric form of the pyruvate kinase isoenzyme type M2 (tumor M2-PK), an abnormal ras protein, or an abnormal p53 protein. In certain embodiments, the tumor-associated antigen is CD19, CD22, CD27, CD30, CD70, GD2 (ganglioside G2), EGFRvIII (epidermal growth factor variant III), sperm protein 17 (Sp17), mesothelin, PAP (prostatic acid phosphatase), prostein, TARP (T cell receptor gamma alternate reading frame protein), Trp-p8, or STEAP1 (six-transmembrane epithelial antigen of the prostate 1). In certain embodiments, the TAA is a cancer/testis (CT) antigen, e.g., BAGE, CAGE, CTAGE, FATE, GAGE, HCA661, HOM-TES-85, MAGEA, MAGEB, MAGEC, NA88, NY-ESO-1, NY-SAR-35, OY-TES-1, SPANXB1, SPA17, SSX, SYCP1, or TPTE. In certain other embodiments, the TAA is a carbohydrate or ganglioside, e.g., fuc-GMI, GM2 (oncofetal antigen-immunogenic-1; OFA-I-1); GD2 (OFA-I-2), GM3, GD3, and the like. In certain other embodiments, the TAA is alpha-actinin-4, Bage-1, BCR-ABL, Bcr-Abl fusion protein, beta-catenin, CA 125, CA 15-3 (CA 27.29\BCAA), CA 195, CA 242, CA-50, CAM43, Casp-8, cdc27, cdk4, cdkn2a, CEA, coa-1, dek-can fusion protein, EBNA, EF2, Epstein Barr virus antigens, ETV6-AML1 fusion protein, HLA-A2, HLA-A11, hsp70-2, KIAAO205, Mart2, Mum-1, 2, and 3, neo-PAP, myosin class I, OS-9, pml-RARa fusion protein, PTPRK, K-ras, N-ras, triosephosphate isomerase, Gage 3,4,5,6,7, GnTV, Herv-K-mel, Lage-1, NA-88, NY-Eso-1/Lage-2, SP17, SSX-2, TRP2-Int2, gpi00 (Pmel 17), tyrosinase, TRP-1, TRP 2, MAGE-1, MAGE-3, RAGE, GAGE-1, GAGE-2, p15(58), RAGE, SCP-i, Hom/Mel-40, PRAME, p53, H-Ras, HER-2/neu, E2A-PRL, H4-RET, IGH-IGK, MYL-RAR, human papillomavirus (HPV) antigens E6 and E7, TSP-180, MAGE-4, MAGE-5, MAGE-6, pl85erbB2, pl80erbB-3, c-met, nm-23H1, PSA, TAG-72-4, CA 19-9, CA 72-4, CAM 17.1, NuMa, 13-Catenin, Mum-1, p16, TAGE, PSMA (prostate-specific membrane antigen), B cell maturation antigen (BCMA), CT7, telomerase, 43-9F, 5T4, 791Tgp72, 13HCG, BCA225, BTAA, CD68\KPi, CO-029, FGF-5, G250, Ga733 (EpCAM), HTgp-175, M344, MA-50, MG7-Ag, MOV18, NB\70K, NY-CO-1, RCAS1, SDCCAG16, TA-90, TAAL6, TAG72, TLP, TPS, integrin cv3 (CD61), galactin, K Ras (V-Ki-ras2 Kirsten rat sarcoma viral oncogene) or Ral-B.

Other tumor associated antigens are known to those in the art and can be combined with the phDCs provided herein to yield the respective antigen-specific phDCs or can be present in the compositions as disclosed herein.

### II.B Means of Providing the Disease Associated Antigen

II.B.1 Antigen Derived from Disease Causing Particles

The disease associated antigen can be derived from the disease causing particles, e.g. from cells, biopsies, tissue lysates or other particles which comprise disease causing particles. In one embodiment, the disease associated antigen can be derived from the tumor or cancer cells to be treated. For example, a sample of tumor cells can be taken from a subject suffering from a cancer, e.g. a biopsy, or a sample can be taken from a subject suffering from an infectious disease. The sample can further be treated with agents, which are suitable to release antigens such as photoactivatable agents in combination with light (in particular, UV light) such as the combination of 8-MOP and UVA. The treated sample can then be combined with phDCs.

In another example, pathogens can be cultured in a medium. Relevant antigens may be shed into the culture medium by the pathogen and can then be collected for combination with the phDCs. The skilled person is aware of different and further methods known in the art to provide antigens derived from the disease causing particles.

II.B.2 Antigen Expressed from Nucleic Acids

The phDCs can be loaded with a nucleic acid (functional fragments thereof are included) encoding for the at least one disease associated antigen. In one embodiment of the invention, the phDCs are loaded with RNA (functional fragments thereof are included) encoding for the at least one disease associated antigen. In this embodiment, the at least one disease associated antigen corresponds to the at least one antigenic protein. Thus, phDCs are directly combined with RNA (e.g. mRNA). The RNA (e.g. mRNA) may be translated into the respective disease associated antigen (i.e. antigenic protein) after entering the phDCs. The RNA can be a single stranded RNA, an mRNA, a self-amplifying RNA, a circular RNA and/or a synthetic RNA. In a preferred embodiment, the RNA is an mRNA. Optionally, the mRNA is self-amplifying mRNA. The basic components of an mRNA molecule typically include at least one coding region, a 5′ untranslated region (UTR), a 3′ UTR, a 5′ cap and a poly-A tail.

Thus in some embodiments, the at least one disease associated antigen (i.e. antigenic protein) is encoded by an mRNA, which is in a composition with phDCs (first aspect). Put in other words, a vaccine composition is provided herein comprising phDCs and at least one mRNA (which comprises a coding sequence) encoding for at least one antigenic protein.

If a multi-valent composition is desired, the phDCs can be in a composition with multiple mRNAs that encode more than one antigenic protein, such as 2, 3, 4, 5, 6, 7, 8, 9, 10 or more antigenic proteins, from the same or different pathogens or tumors. Alternatively or additionally, a polycistronic mRNA can be designed that can be translated into more than one antigenic protein.

### Coding Region

In one embodiment the mRNA encodes one or more antigenic proteins of the above listed disease causing particles or the mRNA encodes one or more antigenic proteins of the above listed tumor or infectious disease associated antigens. In one embodiment, the mRNA encodes one or more antigenic proteins of the above listed pathogens. In one embodiment, the mRNA encodes one or more antigenic proteins of the above listed viruses, bacteria, fungi, prions or parasites. In one embodiment, the mRNA encodes one or more antigenic proteins of the above listed viruses, optionally coronaviruses. In one embodiment the mRNA encodes one or more antigenic proteins of the above listed disease causing particles. In one embodiment the mRNA encodes one or more antigenic proteins of the above listed tumors or cancers. In one embodiment the mRNA encodes one or more antigenic proteins of the above listed TAAs. In one embodiment, the at least one mRNA encodes for one antigenic protein.

### SARS-CoV-2 Sequences

In one embodiment, the at least one mRNA encodes one or more antigenic proteins derived from SARS-CoV, optionally SARS-CoV-2. Antigenic proteins derived from SARS-CoV or SARS-CoV-2 comprise the spike protein, envelope protein, nucleocapsid protein, membrane protein and/or Orf1ab polyprotein. Thus in one embodiment, the antigenic protein is the spike protein, envelope protein, nucleocapsid protein, membrane protein and/or Orf1ab polyprotein or a fragment of each of the aforementioned. In one embodiment, a fragment comprises at least 10, at least 50, at least 100, at least 200, at least 400 or at least 800 amino acid residues. Due to their surface expression properties, RNA polynucleotides encoding structural proteins are believed to have preferred immunogenic activity and, hence, may be most suitable for the compositions of the invention. In a preferred embodiment, the mRNA encodes one or more antigenic proteins derived from the spike protein or nucleocapsid protein. Even more preferred, the mRNA encodes one or more antigenic proteins derived from the spike protein. The mRNA may encode for an antigenic protein derived from the S1 subunit or the S2 subunit of the spike protein. Expressly included are also variants of the afore-mentioned SARS-CoV-2 proteins. For example, a first generation spike protein variant is referred to as “S-2P” (Pallesen et al. 2017) and contains two proline substitutions at positions 986 and 987 (see e.g. Polack et al. 2020; Bos et al. 2020; Corbett et al. 2020; Wrapp et al. 2020). A second-generation spike construct, termed “HexaPro”, contains four additional prolines at positions 817, 892, 899 and 942. HexaPro is expressed in higher levels than the wild-type spike protein or S-2P and shows improved stability relative to S-2P under low-temperature storage and multiple freeze-thaw cycles (Edwards et al. 2020).

In some embodiments, the mRNA comprises a sequence or part of a sequence selected from the group comprising SEQ ID NO: 1, SEQ ID NO: 2, SEQ ID NO: 3, SEQ ID NO: 4 and/or SEQ ID NO: 5.

In some embodiments, the mRNA comprises a sequence or part of a sequence selected from the group comprising SEQ ID NO: 1, SEQ ID NO: 2, SEQ ID NO: 3, SEQ ID NO: 4 and/or SEQ ID NO: 5 or a sequence having at least 99%, 98%, 97%, 96%, 95%, 90%, 85%, or 80% identity to the selected sequence. In one embodiment, the mRNA comprises a sequence or part of a sequence selected from the group comprising SEQ ID NO: 1, SEQ ID NO: 2, SEQ ID NO: 3, SEQ ID NO: 4 and/or SEQ ID NO: 5 or a sequence having at least 99%, 98%, 97%, 96%, 95%, 90%, 85%, or 80% identity to the selected sequence, wherein the selected RNA sequence is modified with one or more (e.g. all) naturally occurring modified nucleosides as described in the section “Modifications of RNA”. In one embodiment, the naturally occurring modified nucleoside is 1-methylpseudouridine.

The sequences above are based on the reference genome from NCBI (accession number NC_045512.2) and also displayed in Table 1 below. The corresponding amino acid sequences are given in Table 2 below.

In some embodiments, the mRNA comprises a sequence or part of a sequence corresponding to SEQ ID NO: 19 or a sequence having at least 99%, 98%, 97%, 96%, 95%, 90%, 85%, or 80% identity to SEQ ID NO: 19 (Table 3).

Those skilled in the art, reading the present disclosure, will further appreciate that it describes various mRNA constructs comprising a sequence that encodes a full-length SARS-CoV-2 spike protein (e.g., including embodiments in which such encoded SARS-CoV-2 spike protein may comprise at least one or more amino acid substitutions, e.g., proline substitutions as described herein, and/or embodiments in which the mRNA sequence is optimized e.g., for mammalian, e.g., human, subjects, and/or embodiments, in which the mRNA comprises one or more chemical modifications).

### HIV Sequences

In one embodiment, the mRNA encodes one or more antigenic proteins derived from HIV. Antigenic proteins derived from HIV comprise the envelope protein (env), group antigens polyprotein (gag), reverse transcriptase (pol) and/or negative factor protein (nef). Due to their surface expression properties, RNA polynucleotides encoding structural proteins are believed to have preferred immunogenic activity and, hence, may be most suitable for the compositions of the invention. Expressly included are also variants of the afore-mentioned HIV proteins.

In some embodiments, the mRNA comprises a sequence or part of a sequence selected from the group comprising SEQ ID NO: 11, SEQ ID NO: 12, SEQ ID NO: 13 and/or SEQ ID NO: 14.

In some embodiments, the mRNA comprises a sequence or part of a sequence selected from the group comprising SEQ ID NO: 11, SEQ ID NO: 12, SEQ ID NO: 13 and/or SEQ ID NO: 14 or a sequence having at least 99%, 98%, 97%, 96%, 95%, 90%, 85%, or 80% identity to the selected sequence.

The sequences above are based on the reference genome from NCBI (accession number NC_AF033819, HIV-1) and also displayed in Table 1 below. The corresponding amino acid sequences are given in Table 2 below.

Those skilled in the art, reading the present disclosure, will further appreciate that it describes various mRNA constructs comprising a sequence that encodes a full-length HIV structural protein (e.g., including embodiments in which such encoded HIV structural protein may comprise at least one or more amino acid substitutions, and/or embodiments in which the mRNA sequence is optimized e.g., for mammalian, e.g., human, subjects and/or embodiments, in which the mRNA comprises one or more chemical modifications).

TABLE 1

| Transcript | Sequence | SEQ ID NO |
|---|---|---|
| SARS- | AUGUUUGUUUUUCUUGUUUUAUUGCCACUAGUCUCUAGUCAGUGUGUUA | SEQ ID NO: 1 |
| CoV-2 | AUCUUACAACCAGAACUCAAUUACCCCCUGCAUACACUAAUUCUUUCAC | |
| Spike | ACGUGGUGUUUAUUACCCUGACAAAGUUUUCAGAUCCUCAGUUUUACAU | |
| protein | UCAACUCAGGACUUGUUCUUACCUUUCUUUUCCAAUGUUACUUGGUUCC | |
| transcript | AUGCUAUACAUGUCUCUGGGACCAAUGGUACUAAGAGGUUUGAUAACCC | |
| | UGUCCUACCAUUUAAUGAUGGUGUUUAUUUUGCUUCCACUGAGAAGUCU | |
| | AACAUAAUAAGAGGCUGGAUUUUUGGUACUACUUUAGAUUCGAAGACCC | |
| | AGUCCCUACUUAUUGUUAAUAACGCUACUAAUGUUGUUAUUAAAGUCUG | |
| | UGAAUUUCAAUUUUGUAAUGAUCCAUUUUUGGGUGUUUAUUACCACAAA | |
| | AACAACAAAAGUUGGAUGGAAAGUGAGUUCAGAGUUUAUUCUAGUGCG | |
| | AAUAAUUGCACUUUUGAAUAUGUCUCUCAGCCUUUUCUUAUGGACCUUG | |
| | AAGGAAAACAGGGUAAUUUCAAAAAUCUUAGGGAAUUUGUGUUUAAGA | |
| | AUAUUGAUGGUUAUUUUAAAAUAUAUUCUAAGCACACGCCUAUUAAUUU | |
| | AGUGCGUGAUCUCCCUCAGGGUUUUUCGGCUUUAGAACCAUUGGUAGAU | |
| | UUGCCAAUAGGUAUUAACAUCACUAGGUUUCAAACUUUACUUGCUUUAC | |
| | AUAGAAGUUAUUUGACUCCUGGUGAUUCUUCUUCAGGUUGGACAGCUGG | |
| | UGCUGCAGCUUAUUAUGUGGGUUAUCUUCAACCUAGGACUUUUCUAUUA | |
| | AAAUAUAAUGAAAAUGGAACCAUUACAGAUGCUGUAGACUGUGCACUUG | |
| | ACCCUCUCUCAGAAACAAAGUGUACGUUGAAAUCCUUCACUGUAGAAAA | |
| | AGGAAUCUAUCAAACUUCUAACUUUAGAGUCCAACCAACAGAAUCUAUU | |
| | GUUAGAUUUCCUAAUAUUACAAACUUGUGCCCUUUUGGUGAAGUUUUUA | |
| | ACGCCACCAGAUUUGCAUCUGUUUAUGCUUGGAACAGGAAGAGAAUCAG | |
| | CAACUGUGUUGCUGAUUAUUCUGUCCUAUAUAAUUCCGCAUCAUUUUCC | |
| | ACUUUUAAGUGUUAUGGAGUGUCUCCUACUAAAUUAAAUGAUCUCUGCU | |
| | UUACUAAUGUCUAUGCAGAUUCAUUUGUAAUUAGAGGUGAUGAAGUCA | |
| | GACAAAUCGCUCCAGGGCAAACUGGAAAGAUUGCUGAUUAUAAUUAUAA | |
| | AUUACCAGAUGAUUUUACAGGCUGCGUUAUAGCUUGGAAUUCUAACAAU | |
| | CUUGAUUCUAAGGUUGGUGGUAAUUAUAAUUACCUGUAUAGAUUGUUU | |
| | AGGAAGUCUAAUCUCAAACCUUUUGAGAGAGAUAUUUCAACUGAAAUCU | |
| | AUCAGGCCGGUAGCACACCUUGUAAUGGUGUUGAAGGUUUUAAUUGUUA | |
| | CUUUCCUUUACAAUCAUAUGGUUUCCAACCCACUAAUGGUGUUGGUUAC | |
| | CAACCAUACAGAGUAGUAGUACUUUCUUUUGAACUUCUACAUGCACCAG | |
| | CAACUGUUUGUGGACCUAAAAAGUCUACUAAUUUGGUUAAAAACAAAUG | |
| | UGUCAAUUUCAACUUCAAUGGUUUAACAGGCACAGGUGUUCUUACUGAG | |
| | UCUAACAAAAAGUUUCUGCCUUUCCAACAAUUUGGCAGAGACAUUGCUG | |
| | ACACUACUGAUGCUGUCCGUGAUCCACAGACACUUGAGAUUCUUGACAU | |
| | UACACCAUGUUCUUUUGGUGGUGUCAGUGUUAUAACACCAGGAACAAAU | |
| | ACUUCUAACCAGGUUGCUGUUCUUUAUCAGGAUGUUAACUGCACAGAAG | |
| | UCCCUGUUGCUAUUCAUGCAGAUCAACUUACUCCUACUUGGCGUGUUUA | |
| | UUCUACAGGUUCUAAUGUUUUUCAAACACGUGCAGGCUGUUUAAUAGGG | |
| | GCUGAACAUGUCAACAACUCAUAUGAGUGUGACAUACCCAUUGGUGCAG | |
| | GUAUAUGCGCUAGUUAUCAGACUCAGACUAAUUCUCCUCGGCGGGCACG | |
| | UAGUGUAGCUAGUCAAUCCAUCAUUGCCUACACUAUGUCACUUGGUGCA | |
| | GAAAAUUCAGUUGCUUACUCUAAUAACUCUAUUGCCAUACCCACAAAUU | |
| | UUACUAUUAGUGUUACCACAGAAAUUCUACCAGUGUCUAUGACCAAGAC | |
| | AUCAGUAGAUUGUACAAUGUACAUUUGUGGUGAUUCAACUGAAUGCAGC | |
| | AAUCUUUUGUUGCAAUAUGGCAGUUUUUGUACACAAUUAAACCGUGCUU | |
| | UAACUGGAAUAGCUGUUGAACAAGACAAAAACACCCAAGAAGUUUUUGC | |
| | ACAAGUCAAACAAAUUUACAAAACACCACCAAUUAAAGAUUUUGGUGGU | |
| | UUUAAUUUUUCACAAAUAUUACCAGAUCCAUCAAAACCAAGCAAGAGGU | |
| | CAUUUAUUGAAGAUCUACUUUUCAACAAAGUGACACUUGCAGAUGCUGG | |
| | CUUCAUCAAACAAUAUGGUGAUUGCCUUGGUGAUAUUGCUGCUAGAGAC | |
| | CUCAUUUGUGCACAAAAGUUUAACGGCCUUACUGUUUUGCCACCUUUGC | |
| | UCACAGAUGAAAUGAUUGCUCAAUACACUUCUGCACUGUUAGCGGGUAC | |
| | AAUCACUUCUGGUUGGACCUUUGGUGCAGGUGCUGCAUUACAAAUACCA | |
| | UUUGCUAUGCAAAUGGCUUAUAGGUUUAAUGGUAUUGGAGUUACACAG | |
| | AAUGUUCUCUAUGAGAACCAAAAAUUGAUUGCCAACCAAUUUAAUAGUG | |
| | CUAUUGGCAAAAUUCAAGACUCACUUUCUUCCACAGCAAGUGCACUUGG | |
| | AAAACUUCAAGAUGUGGUCAACCAAAAUGCACAAGCUUUAAACACGCUU | |
| | GUUAAACAACUUAGCUCCAAUUUUGGUGCAAUUUCAAGUGUUUUAAAUG | |
| | AUAUCCUUUCACGUCUUGACAAAGUUGAGGCUGAAGUGCAAAUUGAUAG | |
| | GUUGAUCACAGGCAGACUUCAAAGUUUGCAGACAUAUGUGACUCAACAA | |
| | UUAAUUAGAGCUGCAGAAAUCAGAGCUUCUGCUAAUCUUGCUGCUACUA | |
| | AAAUGUCAGAGUGUGUACUUGGACAAUCAAAAAGAGUUGAUUUUUGUG | |
| | GAAAGGGCUAUCAUCUUAUGUCCUUCCCUCAGUCAGCACCUCAUGGUGU | |
| | AGUCUUCUUGCAUGUGACUUAUGUCCCUGCACAAGAAAAGAACUUCACA | |
| | ACUGCUCCUGCCAUUUGUCAUGAUGGAAAAGCACACUUUCCUCGUGAAG | |
| | GUGUCUUUGUUUCAAAUGGCACACACUGGUUUGUAACACAAAGGAAUUU | |
| | UUAUGAACCACAAAUCAUUACUACAGACAACACAUUUGUGUCUGGUAAC | |
| | UGUGAUGUUGUAAUAGGAAUUGUCAACAACACAGUUUAUGAUCCUUUGC | |
| | AACCUGAAUUAGACUCAUUCAAGGAGGAGUUAGAUAAAUAUUUUAAGA | |
| | AUCAUACAUCACCAGAUGUUGAUUUAGGUGACAUCUCUGGCAUUAAUGC | |
| | UUCAGUUGUAAACAUUCAAAAAGAAAUUGACCGCCUCAAUGAGGUUGCC | |
| | AAGAAUUUAAAUGAAUCUCUCAUCGAUCUCCAAGAACUUGGAAAGUAUG | |
| | AGCAGUAUAUAAAAUGGCCAUGGUACAUUUGGCUAGGUUUUAUAGCUGG | |
| | CUUGAUUGCCAUAGUAAUGGUGACAAUUAUGCUUUGCUGUAUGACCAGU | |
| | UGCUGUAGUUGUCUCAAGGGCUGUUGUUCUUGUGGAUCCUGCUGCAAAU | |
| | UUGAUGAAGACGACUCUGAGCCAGUGCUCAAAGGAGUCAAAUUACAUUA | |
| | CACAUAA | |
| SARS- | AUGUACUCAUUCGUUUCGGAAGAGACAGGUACGUUAAUAGUUAAUAGC | SEQ ID NO: 2 |
| CoV-2 | GUACUUCUUUUUCUUGCUUUCGUGGUAUUCUUGCUAGUUACACUAGCCA | |
| Envelope | UCCUUACUGCGCUUCGAUUGUGUGCGUACUGCUGCAAUAUUGUUAACGU | |
| protein | GAGUCUUGUAAAACCUUCUUUUUACGUUUACUCUCGUGUUAAAAAUCU | |
| transcript | GAAUUCUUCUAGAGUUCCUGAUCUUCUGGUCUAA | |
| SARS- | AUGUCUGAUAAUGGACCCCAAAAUCAGCGAAAUGCACCCCGCAUUACGUU | SEQ ID NO: 3 |
| CoV-2 | UGGUGGACCCUCAGAUUCAACUGGCAGUAACCAGAAUGGAGAACGCAGUG | |
| Nucleocapsid | GGGCGCGAUCAAAACAACGUCGGCCCCAAGGUUUACCCAAUAAUACUGCG | |
| protein | UCUUGGUUCACCGCUCUCACUCAACAUGGCAAGGAAGACCUUAAAUUCCC | |
| transcript | UCGAGGACAAGGCGUUCCAAUUAACACCAAUAGCAGUCCAGAUGACCAAA | |
| | UUGGCUACUACCGAAGAGCUACCAGACGAAUUCGUGGUGGUGACGGUAAA | |
| | AUGAAAGAUCUCAGUCCAAGAUGGUAUUUCUACUACCUAGGAACUGGGCC | |
| | AGAAGCUGGACUUCCCUAUGGUGCUAACAAAGACGGCAUCAUAUGGGUUG | |
| | CAACUGAGGGAGCCUUGAAUACACCAAAAGAUCACAUUGGCACCCGCAAU | |
| | CCUGCUAACAAUGCUGCAAUCGUGCUACAACUUCCUCAAGGAACAACAUU | |
| | GCCAAAAGGCUUCUACGCAGAAGGGAGCAGAGGCGGCAGUCAAGCCUCUU | |
| | CUCGUUCCUCAUCACGUAGUCGCAACAGUUCAAGAAAUUCAACUCCAGGC | |
| | AGCAGUAGGGGAACUUCUCCUGCUAGAAUGGCUGGCAAUGGCGGUGAUGC | |
| | UGCUCUUGCUUUGCUGCUGCUUGACAGAUUGAACCAGCUUGAGAGCAAAA | |
| | UGUCUGGUAAAGGCCAACAACAACAAGGCCAAACUGUCACUAAGAAAUCU | |
| | GCUGCUGAGGCUUCUAAGAAGCCUCGGCAAAAACGUACUGCCACUAAAGC | |
| | AUACAAUGUAACACAAGCUUUCGGCAGACGUGGUCCAGAACAAACCCAAG | |
| | GAAAUUUUGGGGACCAGGAACUAAUCAGACAAGGAACUGAUUACAAACA | |
| | UUGGCCGCAAAUUGCACAAUUUGCCCCCAGCGCUUCAGCGUUCUUCGGAA | |
| | UGUCGCGCAUUGGCAUGGAAGUCACACCUUCGGGAACGUGGUUGACCUAC | |
| | ACAGGUGCCAUCAAAUUGGAUGACAAAGAUCCAAAUUUCAAAGAUCAAGU | |
| | CAUUUUGCUGAAUAAGCAUAUUGACGCAUACAAAACAUUCCCACCAACAG | |
| | AGCCUAAAAAGGACAAAAAGAAGAAGGCUGAUGAAACUCAAGCCUUACCG | |
| | CAGAGACAGAAGAAACAGCAAACUGUGACUCUUCUUCCUGCUGCAGAUUU | |
| | GGAUGAUUUCUCCAAACAAUUGCAACAAUCCAUGAGCAGUGCUGACUCAA | |
| | CUCAGGCCUAA | |
| SARS- | AUGGCAGAUUCCAACGGUACUAUUACCGUUGAAGAGCUUAAAAAGCUCCU | SEQ ID NO: 4 |
| CoV-2 | UGAACAAUGGAACCUAGUAAUAGGUUUCCUAUUCCUUACAUGGAUUUGUC | |
| Membrane | UUCUACAAUUUGCCUAUGCCAACAGGAAUAGGUUUUUGUAUAUAAUUAA | |
| protein | GUUAAUUUUCCUCUGGCUGUUAUGGCCAGUAACUUUAGCUUGUUUUGUGC | |
| transcript | UUGCUGCUGUUUACAGAAUAAAUUGGAUCACCGGUGGAAUUGCUAUCGCA | |
| | AUGGCUUGUCUUGUAGGCUUGAUGUGGCUCAGCUACUUCAUUGCUUCUUU | |
| | CAGACUGUUUGCGCGUACGCGUUCCAUGUGGUCAUUCAAUCCAGAAACUA | |
| | ACAUUCUUCUCAACGUGCCACUCCAUGGCACUAUUCUGACCAGACCGCUU | |
| | CUAGAAAGUGAACUCGUAAUCGGAGCUGUGAUCCUUCGUGGACAUCUUCG | |
| | UAUUGCUGGACACCAUCUAGGACGCUGUGACAUCAAGGACCUGCCUAAAG | |
| | AAAUCACUGUUGCUACAUCACGAACGCUUUCUUAUUACAAAUUGGGAGCU | |
| | UCGCAGCGUGUAGCAGGUGACUCAGGUUUUGCUGCAUACAGUCGCUACAG | |
| | GAUUGGCAACUAUAAAUUAAACACAGACCAUUCCAGUAGCAGUGACAAUA | |
| | UUGCUUUGCUUGUACAGUAA | |
| SARS- | AUGGAGAGCCUUGUCCCUGGUUUCAACGAGAAAACACACGUCCAACUCAGUU | SEQ ID NO: 5 |
| CoV-2 | UGCCUGUUUUACAGGUUCGCGACGUGCUCGUACGUGGCUUUGGAGACUCCGU | |
| Orf1ab | GGAGGAGGUCUUAUCAGAGGCACGUCAACAUCUUAAAGAUGGCACUUGUGGC | |
| polyprotein | UUAGUAGAAGUUGAAAAAGGCGUUUUGCCUCAACUUGAACAGCCCUAUGUG | |
| transcript | UUCAUCAAACGUUCGGAUGCUCGAACUGCACCUCAUGGUCAUGUUAUGGUUG | |
| | AGCUGGUAGCAGAACUCGAAGGCAUUCAGUACGGUCGUAGUGGUGAGACACU | |
| | UGGUGUCCUUGUCCCUCAUGUGGGCGAAAUACCAGUGGCUUACCGCAAGGUU | |
| | CUUCUUCGUAAGAACGGUAAUAAAGGAGCUGGUGGCCAUAGUUACGGCGCCG | |
| | AUCUAAAGUCAUUUGACUUAGGCGACGAGCUUGGCACUGAUCCUUAUGAAGA | |
| | UUUUCAAGAAAACUGGAACACUAAACAUAGCAGUGGUGUUACCCGUGAACUC | |
| | AUGCGUGAGCUUAACGGAGGGGCAUACACUCGCUAUGUCGAUAACAACUUCU | |
| | GUGGCCCUGAUGGCUACCCUCUUGAGUGCAUUAAAGACCUUCUAGCACGUGC | |
| | UGGUAAAGCUUCAUGCACUUUGUCCGAACAACUGGACUUUAUUGACACUAAG | |
| | AGGGGUGUAUACUGCUGCCGUGAACAUGAGCAUGAAAUUGCUUGGUACACG | |
| | GAACGUUCUGAAAAGAGCUAUGAAUUGCAGACACCUUUUGAAAUUAAAUUG | |
| | GCAAAGAAAUUUGACACCUUCAAUGGGGAAUGUCCAAAUUUUGUAUUUCCCU | |
| | UAAAUUCCAUAAUCAAGACUAUUCAACCAAGGGUUGAAAAGAAAAAGCUUG | |
| | AUGGCUUUAUGGGUAGAAUUCGAUCUGUCUAUCCAGUUGCGUCACCAAAUGA | |
| | AUGCAACCAAAUGUGCCUUUCAACUCUCAUGAAGUGUGAUCAUUGUGGUGAA | |
| | ACUUCAUGGCAGACGGGCGAUUUUGUUAAAGCCACUUGCGAAUUUUGUGGCA | |
| | CUGAGAAUUUGACUAAAGAAGGUGCCACUACUUGUGGUUACUUACCCCAAAA | |
| | UGCUGUUGUUAAAAUUUAUUGUCCAGCAUGUCACAAUUCAGAAGUAGGACC | |
| | UGAGCAUAGUCUUGCCGAAUACCAUAAUGAAUCUGGCUUGAAAACCAUUCUU | |
| | CGUAAGGGUGGUCGCACUAUUGCCUUUGGAGGCUGUGUGUUCUCUUAUGUU | |
| | GGUUGCCAUAACAAGUGUGCCUAUUGGGUUCCACGUGCUAGCGCUAACAUAG | |
| | GUUGUAACCAUACAGGUGUUGUUGGAGAAGGUUCCGAAGGUCUUAAUGACA | |
| | ACCUUCUUGAAAUACUCCAAAAAGAGAAAGUCAACAUCAAUAUUGUUGGUG | |
| | ACUUUAAACUUAAUGAAGAGAUCGCCAUUAUUUUGGCAUCUUUUUCUGCUUC | |
| | CACAAGUGCUUUUGUGGAAACUGUGAAAGGUUUGGAUUAUAAAGCAUUCAA | |
| | ACAAAUUGUUGAAUCCUGUGGUAAUUUUAAAGUUACAAAAGGAAAAGCUAA | |
| | AAAAGGUGCCUGGAAUAUUGGUGAACAGAAAUCAAUACUGAGUCCUCUUUA | |
| | UGCAUUUGCAUCAGAGGCUGCUCGUGUUGUACGAUCAAUUUUCUCCCGCACU | |
| | CUUGAAACUGCUCAAAAUUCUGUGCGUGUUUUACAGAAGGCCGCUAUAACAA | |
| | UACUAGAUGGAAUUUCACAGUAUUCACUGAGACUCAUUGAUGCUAUGAUGU | |
| | UCACAUCUGAUUUGGCUACUAACAAUCUAGUUGUAAUGGCCUACAUUACAGG | |
| | UGGUGUUGUUCAGUUGACUUCGCAGUGGCUAACUAACAUCUUUGGCACUGUU | |
| | UAUGAAAAACUCAAACCCGUCCUUGAUUGGCUUGAAGAGAAGUUUAAGGAA | |
| | GGUGUAGAGUUUCUUAGAGACGGUUGGGAAAUUGUUAAAUUUAUCUCAACC | |
| | UGUGCUUGUGAAAUUGUCGGUGGACAAAUUGUCACCUGUGCAAAGGAAAUU | |
| | AAGGAGAGUGUUCAGACAUUCUUUAAGCUUGUAAAUAAAUUUUUGGCUUUG | |
| | UGUGCUGACUCUAUCAUUAUUGGUGGAGCUAAACUUAAAGCCUUGAAUUUA | |
| | GGUGAAACAUUUGUCACGCACUCAAAGGGAUUGUACAGAAAGUGUGUUAAA | |
| | UCCAGAGAAGAAACUGGCCUACUCAUGCCUCUAAAAGCCCCAAAAGAAAUUA | |
| | UCUUCUUAGAGGGAGAAACACUUCCCACAGAAGUGUUAACAGAGGAAGUUG | |
| | UCUUGAAAACUGGUGAUUUACAACCAUUAGAACAACCUACUAGUGAAGCUGU | |
| | UGAAGCUCCAUUGGUUGGUACACCAGUUUGUAUUAACGGGCUUAUGUUGCUC | |
| | GAAAUCAAAGACACAGAAAAGUACUGUGCCCUUGCACCUAAUAUGAUGGUAA | |
| | CAAACAAUACCUUCACACUCAAAGGCGGUGCACCAACAAAGGUUACUUUUGG | |
| | UGAUGACACUGUGAUAGAAGUGCAAGGUUACAAGAGUGUGAAUAUCACUUU | |
| | UGAACUUGAUGAAAGGAUUGAUAAAGUACUUAAUGAGAAGUGCUCUGCCUA | |
| | UACAGUUGAACUCGGUACAGAAGUAAAUGAGUUCGCCUGUGUUGUGGCAGA | |
| | UGCUGUCAUAAAAACUUUGCAACCAGUAUCUGAAUUACUUACACCACUGGGC | |
| | AUUGAUUUAGAUGAGUGGAGUAUGGCUACAUACUACUUAUUUGAUGAGUCU | |
| | GGUGAGUUUAAAUUGGCUUCACAUAUGUAUUGUUCUUUCUACCCUCCAGAUG | |
| | AGGAUGAAGAAGAAGGUGAUUGUGAAGAAGAAGAGUUUGAGCCAUCAACUC | |
| | AAUAUGAGUAUGGUACUGAAGAUGAUUACCAAGGUAAACCUUUGGAAUUUG | |
| | GUGCCACUUCUGCUGCUCUUCAACCUGAAGAAGAGCAAGAAGAAGAUUGGUU | |
| | AGAUGAUGAUAGUCAACAAACUGUUGGUCAACAAGACGGCAGUGAGGACAA | |
| | UCAGACAACUACUAUUCAAACAAUUGUUGAGGUUCAACCUCAAUUAGAGAUG | |
| | GAACUUACACCAGUUGUUCAGACUAUUGAAGUGAAUAGUUUUAGUGGUUAU | |
| | UUAAAACUUACUGACAAUGUAUACAUUAAAAAUGCAGACAUUGUGGAAGAA | |
| | GCUAAAAAGGUAAAACCAACAGUGGUUGUUAAUGCAGCCAAUGUUUACCUU | |
| | AAACAUGGAGGAGGUGUUGCAGGAGCCUUAAAUAAGGCUACUAACAAUGCC | |
| | AUGCAAGUUGAAUCUGAUGAUUACAUAGCUACUAAUGGACCACUUAAAGUG | |
| | GGUGGUAGUUGUGUUUUAAGCGGACACAAUCUUGCUAAACACUGUCUUCAU | |
| | GUUGUCGGCCCAAAUGUUAACAAAGGUGAAGACAUUCAACUUCUUAAGAGU | |
| | GCUUAUGAAAAUUUUAAUCAGCACGAAGUUCUACUUGCACCAUUAUUAUCAG | |
| | CUGGUAUUUUUGGUGCUGACCCUAUACAUUCUUUAAGAGUUUGUGUAGAUA | |
| | CUGUUCGCACAAAUGUCUACUUAGCUGUCUUUGAUAAAAAUCUCUAUGACAA | |
| | ACUUGUUUCAAGCUUUUUGGAAAUGAAGAGUGAAAAGCAAGUUGAACAAAA | |
| | GAUCGCUGAGAUUCCUAAAGAGGAAGUUAAGCCAUUUAUAACUGAAAGUAA | |
| | ACCUUCAGUUGAACAGAGAAAACAAGAUGAUAAGAAAAUCAAAGCUUGUGU | |
| | UGAAGAAGUUACAACAACUCUGGAAGAAACUAAGUUCCUCACAGAAAACUUG | |
| | UUACUUUAUAUUGACAUUAAUGGCAAUCUUCAUCCAGAUUCUGCCACUCUUG | |
| | UUAGUGACAUUGACAUCACUUUCUUAAAGAAAGAUGCUCCAUAUAUAGUGG | |
| | GUGAUGUUGUUCAAGAGGGUGUUUUAACUGCUGUGGUUAUACCUACUAAAA | |
| | AGGCUGGUGGCACUACUGAAAUGCUAGCGAAAGCUUUGAGAAAAGUGCCAAC | |
| | AGACAAUUAUAUAACCACUUACCCGGGUCAGGGUUUAAAUGGUUACACUGUA | |
| | GAGGAGGCAAAGACAGUGCUUAAAAAGUGUAAAAGUGCCUUUUACAUUCUA | |
| | CCAUCUAUUAUCUCUAAUGAGAAGCAAGAAAUUCUUGGAACUGUUUCUUGG | |
| | AAUUUGCGAGAAAUGCUUGCACAUGCAGAAGAAACACGCAAAUUAAUGCCUG | |
| | UCUGUGUGGAAACUAAAGCCAUAGUUUCAACUAUACAGCGUAAAUAUAAGG | |
| | GUAUUAAAAUACAAGAGGGUGUGGUUGAUUAUGGUGCUAGAUUUUACUUUU | |
| | ACACCAGUAAAACAACUGUAGCGUCACUUAUCAACACACUUAACGAUCUAAA | |
| | UGAAACUCUUGUUACAAUGCCACUUGGCUAUGUAACACAUGGCUUAAAUUUG | |
| | GAAGAAGCUGCUCGGUAUAUGAGAUCUCUCAAAGUGCCAGCUACAGUUUCUG | |
| | UUUCUUCACCUGAUGCUGUUACAGCGUAUAAUGGUUAUCUUACUUCUUCUUC | |
| | UAAAACACCUGAAGAACAUUUUAUUGAAACCAUCUCACUUGCUGGUUCCUAU | |
| | AAAGAUUGGUCCUAUUCUGGACAAUCUACACAACUAGGUAUAGAAUUUCUU | |
| | AAGAGAGGUGAUAAAAGUGUAUAUUACACUAGUAAUCCUACCACAUUCCACC | |
| | UAGAUGGUGAAGUUAUCACCUUUGACAAUCUUAAGACACUUCUUUCUUUGA | |
| | GAGAAGUGAGGACUAUUAAGGUGUUUACAACAGUAGACAACAUUAACCUCC | |
| | ACACGCAAGUUGUGGACAUGUCAAUGACAUAUGGACAACAGUUUGGUCCAAC | |
| | UUAUUUGGAUGGAGCUGAUGUUACUAAAAUAAAACCUCAUAAUUCACAUGA | |
| | AGGUAAAACAUUUUAUGUUUUACCUAAUGAUGACACUCUACGUGUUGAGGC | |
| | UUUUGAGUACUACCACACAACUGAUCCUAGUUUUCUGGGUAGGUACAUGUCA | |
| | GCAUUAAAUCACACUAAAAAGUGGAAAUACCCACAAGUUAAUGGUUUAACU | |
| | UCUAUUAAAUGGGCAGAUAACAACUGUUAUCUUGCCACUGCAUUGUUAACAC | |
| | UCCAACAAAUAGAGUUGAAGUUUAAUCCACCUGCUCUACAAGAUGCUUAUUA | |
| | CAGAGCAAGGGCUGGUGAAGCUGCUAACUUUUGUGCACUUAUCUUAGCCUAC | |
| | UGUAAUAAGACAGUAGGUGAGUUAGGUGAUGUUAGAGAAACAAUGAGUUAC | |
| | UUGUUUCAACAUGCCAAUUUAGAUUCUUGCAAAAGAGUCUUGAACGUGGUG | |
| | UGUAAAACUUGUGGACAACAGCAGACAACCCUUAAGGGUGUAGAAGCUGUU | |
| | AUGUACAUGGGCACACUUUCUUAUGAACAAUUUAAGAAAGGUGUUCAGAUA | |
| | CCUUGUACGUGUGGUAAACAAGCUACAAAAUAUCUAGUACAACAGGAGUCAC | |
| | CUUUUGUUAUGAUGUCAGCACCACCUGCUCAGUAUGAACUUAAGCAUGGUAC | |
| | AUUUACUUGUGCUAGUGAGUACACUGGUAAUUACCAGUGUGGUCACUAUAA | |
| | ACAUAUAACUUCUAAAGAAACUUUGUAUUGCAUAGACGGUGCUUUACUUAC | |
| | AAAGUCCUCAGAAUACAAAGGUCCUAUUACGGAUGUUUUCUACAAAGAAAAC | |
| | AGUUACACAACAACCAUAAAACCAGUUACUUAUAAAUUGGAUGGUGUUGUU | |
| | UGUACAGAAAUUGACCCUAAGUUGGACAAUUAUUAUAAGAAAGACAAUUCU | |
| | UAUUUCACAGAGCAACCAAUUGAUCUUGUACCAAACCAACCAUAUCCAAACG | |
| | CAAGCUUCGAUAAUUUUAAGUUUGUAUGUGAUAAUAUCAAAUUUGCUGAUG | |
| | AUUUAAACCAGUUAACUGGUUAUAAGAAACCUGCUUCAAGAGAGCUUAAAG | |
| | UUACAUUUUUCCCUGACUUAAAUGGUGAUGUGGUGGCUAUUGAUUAUAAAC | |
| | ACUACACACCCUCUUUUAAGAAAGGAGCUAAAUUGUUACAUAAACCUAUUGU | |
| | UUGGCAUGUUAACAAUGCAACUAAUAAAGCCACGUAUAAACCAAAUACCUGG | |
| | UGUAUACGUUGUCUUUGGAGCACAAAACCAGUUGAAACAUCAAAUUCGUUU | |
| | GAUGUACUGAAGUCAGAGGACGCGCAGGGAAUGGAUAAUCUUGCCUGCGAA | |
| | GAUCUAAAACCAGUCUCUGAAGAAGUAGUGGAAAAUCCUACCAUACAGAAAG | |
| | ACGUUCUUGAGUGUAAUGUGAAAACUACCGAAGUUGUAGGAGACAUUAUAC | |
| | UUAAACCAGCAAAUAAUAGUUUAAAAAUUACAGAAGAGGUUGGCCACACAG | |
| | AUCUAAUGGCUGCUUAUGUAGACAAUUCUAGUCUUACUAUUAAGAAACCUA | |
| | AUGAAUUAUCUAGAGUAUUAGGUUUGAAAACCCUUGCUACUCAUGGUUUAG | |
| | CUGCUGUUAAUAGUGUCCCUUGGGAUACUAUAGCUAAUUAUGCUAAGCCUUU | |
| | UCUUAACAAAGUUGUUAGUACAACUACUAACAUAGUUACACGGUGUUUAAA | |
| | CCGUGUUUGUACUAAUUAUAUGCCUUAUUUCUUUACUUUAUUGCUACAAUU | |
| | GUGUACUUUUACUAGAAGUACAAAUUCUAGAAUUAAAGCAUCUAUGCCGAC | |
| | UACUAUAGCAAAGAAUACUGUUAAGAGUGUCGGUAAAUUUUGUCUAGAGGC | |
| | UUCAUUUAAUUAUUUGAAGUCACCUAAUUUUUCUAAACUGAUAAAUAUUAU | |
| | AAUUUGGUUUUUACUAUUAAGUGUUUGCCUAGGUUCUUUAAUCUACUCAAC | |
| | CGCUGCUUUAGGUGUUUUAAUGUCUAAUUUAGGCAUGCCUUCUUACUGUACU | |
| | GGUUACAGAGAAGGCUAUUUGAACUCUACUAAUGUCACUAUUGCAACCUACU | |
| | GUACUGGUUCUAUACCUUGUAGUGUUUGUCUUAGUGGUUUAGAUUCUUUAG | |
| | ACACCUAUCCUUCUUUAGAAACUAUACAAAUUACCAUUUCAUCUUUUAAAUG | |
| | GGAUUUAACUGCUUUUGGCUUAGUUGCAGAGUGGUUUUUGGCAUAUAUUCU | |
| | UUUCACUAGGUUUUUCUAUGUACUUGGAUUGGCUGCAAUCAUGCAAUUGUU | |
| | UUUCAGCUAUUUUGCAGUACAUUUUAUUAGUAAUUCUUGGCUUAUGUGGUU | |
| | AAUAAUUAAUCUUGUACAAAUGGCCCCGAUUUCAGCUAUGGUUAGAAUGUA | |
| | CAUCUUCUUUGCAUCAUUUUAUUAUGUAUGGAAAAGUUAUGUGCAUGUUGU | |
| | AGACGGUUGUAAUUCAUCAACUUGUAUGAUGUGUUACAAACGUAAUAGAGC | |
| | AACAAGAGUCGAAUGUACAACUAUUGUUAAUGGUGUUAGAAGGUCCUUUUA | |
| | UGUCUAUGCUAAUGGAGGUAAAGGCUUUUGCAAACUACACAAUUGGAAUUG | |
| | UGUUAAUUGUGAUACAUUCUGUGCUGGUAGUACAUUUAUUAGUGAUGAAGU | |
| | UGCGAGAGACUUGUCACUACAGUUUAAAAGACCAAUAAAUCCUACUGACCAG | |
| | UCUUCUUACAUCGUUGAUAGUGUUACAGUGAAGAAUGGUUCCAUCCAUCUUU | |
| | ACUUUGAUAAAGCUGGUCAAAAGACUUAUGAAAGACAUUCUCUCUCUCAUUU | |
| | UGUUAACUUAGACAACCUGAGAGCUAAUAACACUAAAGGUUCAUUGCCUAUU | |
| | AAUGUUAUAGUUUUUGAUGGUAAAUCAAAAUGUGAAGAAUCAUCUGCAAAA | |
| | UCAGCGUCUGUUUACUACAGUCAGCUUAUGUGUCAACCUAUACUGUUACUAG | |
| | AUCAGGCAUUAGUGUCUGAUGUUGGUGAUAGUGCGGAAGUUGCAGUUAAAA | |
| | UGUUUGAUGCUUACGUUAAUACGUUUUCAUCAACUUUUAACGUACCAAUGG | |
| | AAAAACUCAAAACACUAGUUGCAACUGCAGAAGCUGAACUUGCAAAGAAUGU | |
| | GUCCUUAGACAAUGUCUUAUCUACUUUUAUUUCAGCAGCUCGGCAAGGGUUU | |
| | GUUGAUUCAGAUGUAGAAACUAAAGAUGUUGUUGAAUGUCUUAAAUUGUCA | |
| | CAUCAAUCUGACAUAGAAGUUACUGGCGAUAGUUGUAAUAACUAUAUGCUC | |
| | ACCUAUAACAAAGUUGAAAACAUGACACCCCGUGACCUUGGUGCUUGUAUUG | |
| | ACUGUAGUGCGCGUCAUAUUAAUGCGCAGGUAGCAAAAAGUCACAACAUUGC | |
| | UUUGAUAUGGAACGUUAAAGAUUUCAUGUCAUUGUCUGAACAACUACGAAA | |
| | ACAAAUACGUAGUGCUGCUAAAAAGAAUAACUUACCUUUUAAGUUGACAUG | |
| | UGCAACUACUAGACAAGUUGUUAAUGUUGUAACAACAAAGAUAGCACUUAA | |
| | GGGUGGUAAAAUUGUUAAUAAUUGGUUGAAGCAGUUAAUUAAAGUUACACU | |
| | UGUGUUCCUUUUUGUUGCUGCUAUUUUCUAUUUAAUAACACCUGUUCAUGUC | |
| | AUGUCUAAACAUACUGACUUUUCAAGUGAAAUCAUAGGAUACAAGGCUAUU | |
| | GAUGGUGGUGUCACUCGUGACAUAGCAUCUACAGAUACUUGUUUUGCUAACA | |
| | AACAUGCUGAUUUUGACACAUGGUUUAGCCAGCGUGGUGGUAGUUAUACUA | |
| | AUGACAAAGCUUGCCCAUUGAUUGCUGCAGUCAUAACAAGAGAAGUGGGUU | |
| | UUGUCGUGCCUGGUUUGCCUGGCACGAUAUUACGCACAACUAAUGGUGACUU | |
| | UUUGCAUUUCUUACCUAGAGUUUUUAGUGCAGUUGGUAACAUCUGUUACAC | |
| | ACCAUCAAAACUUAUAGAGUACACUGACUUUGCAACAUCAGCUUGUGUUUUG | |
| | GCUGCUGAAUGUACAAUUUUUAAAGAUGCUUCUGGUAAGCCAGUACCAUAU | |
| | UGUUAUGAUACCAAUGUACUAGAAGGUUCUGUUGCUUAUGAAAGUUUACGC | |
| | CCUGACACACGUUAUGUGCUCAUGGAUGGCUCUAUUAUUCAAUUUCCUAACA | |
| | CCUACCUUGAAGGUUCUGUUAGAGUGGUAACAACUUUUGAUUCUGAGUACU | |
| | GUAGGCACGGCACUUGUGAAAGAUCAGAAGCUGGUGUUUGUGUAUCUACUA | |
| | GUGGUAGAUGGGUACUUAACAAUGAUUAUUACAGAUCUUUACCAGGAGUUU | |
| | UCUGUGGUGUAGAUGCUGUAAAUUUACUUACUAAUAUGUUUACACCACUAA | |
| | UUCAACCUAUUGGUGCUUUGGACAUAUCAGCAUCUAUAGUAGCUGGUGGUA | |
| | UUGUAGCUAUCGUAGUAACAUGCCUUGCCUACUAUUUUAUGAGGUUUAGAA | |
| | GAGCUUUUGGUGAAUACAGUCAUGUAGUUGCCUUUAAUACUUUACUAUUCC | |
| | UUAUGUCAUUCACUGUACUCUGUUUAACACCAGUUUACUCAUUCUUACCUGG | |
| | UGUUUAUUCUGUUAUUUACUUGUACUUGACAUUUUAUCUUACUAAUGAUGU | |
| | UUCUUUUUUAGCACAUAUUCAGUGGAUGGUUAUGUUCACACCUUUAGUACCU | |
| | UUCUGGAUAACAAUUGCUUAUAUCAUUUGUAUUUCCACAAAGCAUUUCUAU | |
| | UGGUUCUUUAGUAAUUACCUAAAGAGACGUGUAGUCUUUAAUGGUGUUUCC | |
| | UUUAGUACUUUUGAAGAAGCUGCGCUGUGCACCUUUUUGUUAAAUAAAGAA | |
| | AUGUAUCUAAAGUUGCGUAGUGAUGUGCUAUUACCUCUUACGCAAUAUAAU | |
| | AGAUACUUAGCUCUUUAUAAUAAGUACAAGUAUUUUAGUGGAGCAAUGGAU | |
| | ACAACUAGCUACAGAGAAGCUGCUUGUUGUCAUCUCGCAAAGGCUCUCAAUG | |
| | ACUUCAGUAACUCAGGUUCUGAUGUUCUUUACCAACCACCACAAACCUCUAU | |
| | CACCUCAGCUGUUUUGCAGAGUGGUUUUAGAAAAAUGGCAUUCCCAUCUGGU | |
| | AAAGUUGAGGGUUGUAUGGUACAAGUAACUUGUGGUACAACUACACUUAAC | |
| | GGUCUUUGGCUUGAUGACGUAGUUUACUGUCCAAGACAUGUGAUCUGCACCU | |
| | CUGAAGACAUGCUUAACCCUAAUUAUGAAGAUUUACUCAUUCGUAAGUCUAA | |
| | UCAUAAUUUCUUGGUACAGGCUGGUAAUGUUCAACUCAGGGUUAUUGGACA | |
| | UUCUAUGCAAAAUUGUGUACUUAAGCUUAAGGUUGAUACAGCCAAUCCUAA | |
| | GACACCUAAGUAUAAGUUUGUUCGCAUUCAACCAGGACAGACUUUUUCAGUG | |
| | UUAGCUUGUUACAAUGGUUCACCAUCUGGUGUUUACCAAUGUGCUAUGAGGC | |
| | CCAAUUUCACUAUUAAGGGUUCAUUCCUUAAUGGUUCAUGUGGUAGUGUUG | |
| | GUUUUAACAUAGAUUAUGACUGUGUCUCUUUUUGUUACAUGCACCAUAUGG | |
| | AAUUACCAACUGGAGUUCAUGCUGGCACAGACUUAGAAGGUAACUUUUAUG | |
| | GACCUUUUGUUGACAGGCAAACAGCACAAGCAGCUGGUACGGACACAACUAU | |
| | UACAGUUAAUGUUUUAGCUUGGUUGUACGCUGCUGUUAUAAAUGGAGACAG | |
| | GUGGUUUCUCAAUCGAUUUACCACAACUCUUAAUGACUUUAACCUUGUGGCU | |
| | AUGAAGUACAAUUAUGAACCUCUAACACAAGACCAUGUUGACAUACUAGGAC | |
| | CUCUUUCUGCUCAAACUGGAAUUGCCGUUUUAGAUAUGUGUGCUUCAUUAAA | |
| | AGAAUUACUGCAAAAUGGUAUGAAUGGACGUACCAUAUUGGGUAGUGCUUU | |
| | AUUAGAAGAUGAAUUUACACCUUUUGAUGUUGUUAGACAAUGCUCAGGUGU | |
| | UACUUUCCAAAGUGCAGUGAAAAGAACAAUCAAGGGUACACACCACUGGUUG | |
| | UUACUCACAAUUUUGACUUCACUUUUAGUUUUAGUCCAGAGUACUCAAUGGU | |
| | CUUUGUUCUUUUUUUUGUAUGAAAAUGCCUUUUUACCUUUUGCUAUGGGUA | |
| | UUAUUGCUAUGUCUGCUUUUGCAAUGAUGUUUGUCAAACAUAAGCAUGCAU | |
| | UUCUCUGUUUGUUUUUGUUACCUUCUCUUGCCACUGUAGCUUAUUUUAAUAU | |
| | GGUCUAUAUGCCUGCUAGUUGGGUGAUGCGUAUUAUGACAUGGUUGGAUAU | |
| | GGUUGAUACUAGUUUGUCUGGUUUUAAGCUAAAAGACUGUGUUAUGUAUGC | |
| | AUCAGCUGUAGUGUUACUAAUCCUUAUGACAGCAAGAACUGUGUAUGAUGA | |
| | UGGUGCUAGGAGAGUGUGGACACUUAUGAAUGUCUUGACACUCGUUUAUAA | |
| | AGUUUAUUAUGGUAAUGCUUUAGAUCAAGCCAUUUCCAUGUGGGCUCUUAU | |
| | AAUCUCUGUUACUUCUAACUACUCAGGUGUAGUUACAACUGUCAUGUUUUUG | |
| | GCCAGAGGUAUUGUUUUUAUGUGUGUUGAGUAUUGCCCUAUUUUCUUCAUA | |
| | ACUGGUAAUACACUUCAGUGUAUAAUGCUAGUUUAUUGUUUCUUAGGCUAU | |
| | UUUUGUACUUGUUACUUUGGCCUCUUUUGUUUACUCAACCGCUACUUUAGAC | |
| | UGACUCUUGGUGUUUAUGAUUACUUAGUUUCUACACAGGAGUUUAGAUAUA | |
| | UGAAUUCACAGGGACUACUCCCACCCAAGAAUAGCAUAGAUGCCUUCAAACU | |
| | CAACAUUAAAUUGUUGGGUGUUGGUGGCAAACCUUGUAUCAAAGUAGCCAC | |
| | UGUACAGUCUAAAAUGUCAGAUGUAAAGUGCACAUCAGUAGUCUUACUCUCA | |
| | GUUUUGCAACAACUCAGAGUAGAAUCAUCAUCUAAAUUGUGGGCUCAAUGU | |
| | GUCCAGUUACACAAUGACAUUCUCUUAGCUAAAGAUACUACUGAAGCCUUUG | |
| | AAAAAAUGGUUUCACUACUUUCUGUUUUGCUUUCCAUGCAGGGUGCUGUAG | |
| | ACAUAAACAAGCUUUGUGAAGAAAUGCUGGACAACAGGGCAACCUUACAAGC | |
| | UAUAGCCUCAGAGUUUAGUUCCCUUCCAUCAUAUGCAGCUUUUGCUACUGCU | |
| | CAAGAAGCUUAUGAGCAGGCUGUUGCUAAUGGUGAUUCUGAAGUUGUUCUU | |
| | AAAAAGUUGAAGAAGUCUUUGAAUGUGGCUAAAUCUGAAUUUGACCGUGAU | |
| | GCAGCCAUGCAACGUAAGUUGGAAAAGAUGGCUGAUCAAGCUAUGACCCAAA | |
| | UGUAUAAACAGGCUAGAUCUGAGGACAAGAGGGCAAAAGUUACUAGUGCUA | |
| | UGCAGACAAUGCUUUUCACUAUGCUUAGAAAGUUGGAUAAUGAUGCACUCA | |
| | ACAACAUUAUCAACAAUGCAAGAGAUGGUUGUGUUCCCUUGAACAUAAUACC | |
| | UCUUACAACAGCAGCCAAACUAAUGGUUGUCAUACCAGACUAUAACACAUAU | |
| | AAAAAUACGUGUGAUGGUACAACAUUUACUUAUGCAUCAGCAUUGUGGGAA | |
| | AUCCAACAGGUUGUAGAUGCAGAUAGUAAAAUUGUUCAACUUAGUGAAAUU | |
| | AGUAUGGACAAUUCACCUAAUUUAGCAUGGCCUCUUAUUGUAACAGCUUUAA | |
| | GGGCCAAUUCUGCUGUCAAAUUACAGAAUAAUGAGCUUAGUCCUGUUGCACU | |
| | ACGACAGAUGUCUUGUGCUGCCGGUACUACACAAACUGCUUGCACUGAUGAC | |
| | AAUGCGUUAGCUUACUACAACACAACAAAGGGAGGUAGGUUUGUACUUGCAC | |
| | UGUUAUCCGAUUUACAGGAUUUGAAAUGGGCUAGAUUCCCUAAGAGUGAUG | |
| | GAACUGGUACUAUCUAUACAGAACUGGAACCACCUUGUAGGUUUGUUACAGA | |
| | CACACCUAAAGGUCCUAAAGUGAAGUAUUUAUACUUUAUUAAAGGAUUAAA | |
| | CAACCUAAAUAGAGGUAUGGUACUUGGUAGUUUAGCUGCCACAGUACGUCUA | |
| | CAAGCUGGUAAUGCAACAGAAGUGCCUGCCAAUUCAACUGUAUUAUCUUUCU | |
| | GUGCUUUUGCUGUAGAUGCUGCUAAAGCUUACAAAGAUUAUCUAGCUAGUG | |
| | GGGGACAACCAAUCACUAAUUGUGUUAAGAUGUUGUGUACACACACUGGUAC | |
| | UGGUCAGGCAAUAACAGUUACACCGGAAGCCAAUAUGGAUCAAGAAUCCUUU | |
| | GGUGGUGCAUCGUGUUGUCUGUACUGCCGUUGCCACAUAGAUCAUCCAAAUC | |
| | CUAAAGGAUUUUGUGACUUAAAAGGUAAGUAUGUACAAAUACCUACAACUU | |
| | GUGCUAAUGACCCUGUGGGUUUUACACUUAAAAACACAGUCUGUACCGUCUG | |
| | CGGUAUGUGGAAAGGUUAUGGCUGUAGUUGUGAUCAACUCCGCGAACCCAUG | |
| | CUUCAGUCAGCUGAUGCACAAUCGUUUUUAAACGGGUUUGCGGUGUAAGUGC | |
| | AGCCCGUCUUACACCGUGCGGCACAGGCACUAGUACUGAUGUCGUAUACAGG | |
| | GCUUUUGACAUCUACAAUGAUAAAGUAGCUGGUUUUGCUAAAUUCCUAAAA | |
| | ACUAAUUGUUGUCGCUUCCAAGAAAAGGACGAAGAUGACAAUUUAAUUGAU | |
| | UCUUACUUUGUAGUUAAGAGACACACUUUCUCUAACUACCAACAUGAAGAAA | |
| | CAAUUUAUAAUUUACUUAAGGAUUGUCCAGCUGUUGCUAAACAUGACUUCU | |
| | UUAAGUUUAGAAUAGACGGUGACAUGGUACCACAUAUAUCACGUCAACGUCU | |
| | UACUAAAUACACAAUGGCAGACCUCGUCUAUGCUUUAAGGCAUUUUGAUGAA | |
| | GGUAAUUGUGACACAUUAAAAGAAAUACUUGUCACAUACAAUUGUUGUGAU | |
| | GAUGAUUAUUUCAAUAAAAAGGACUGGUAUGAUUUUGUAGAAAACCCAGAU | |
| | AUAUUACGCGUAUACGCCAACUUAGGUGAACGUGUACGCCAAGCUUUGUUAA | |
| | AAACAGUACAAUUCUGUGAUGCCAUGCGAAAUGCUGGUAUUGUUGGUGUAC | |
| | UGACAUUAGAUAAUCAAGAUCUCAAUGGUAACUGGUAUGAUUUCGGUGAUU | |
| | UCAUACAAACCACGCCAGGUAGUGGAGUUCCUGUUGUAGAUUCUUAUUAUUC | |
| | AUUGUUAAUGCCUAUAUUAACCUUGACCAGGGCUUUAACUGCAGAGUCACAU | |
| | GUUGACACUGACUUAACAAAGCCUUACAUUAAGUGGGAUUUGUUAAAAUAU | |
| | GACUUCACGGAAGAGAGGUUAAAACUCUUUGACCGUUAUUUUAAAUAUUGG | |
| | GAUCAGACAUACCACCCAAAUUGUGUUAACUGUUUGGAUGACAGAUGCAUUC | |
| | UGCAUUGUGCAAACUUUAAUGUUUUAUUCUCUACAGUGUUCCCACCUACAAG | |
| | UUUUGGACCACUAGUGAGAAAAAUAUUUGUUGAUGGUGUUCCAUUUGUAGU | |
| | UUCAACUGGAUACCACUUCAGAGAGCUAGGUGUUGUACAUAAUCAGGAUGU | |
| | AAACUUACAUAGCUCUAGACUUAGUUUUAAGGAAUUACUUGUGUAUGCUGC | |
| | UGACCCUGCUAUGCACGCUGCUUCUGGUAAUCUAUUACUAGAUAAACGCACU | |
| | ACGUGCUUUUCAGUAGCUGCACUUACUAACAAUGUUGCUUUUCAAACUGUCA | |
| | AACCCGGUAAUUUUAACAAAGACUUCUAUGACUUUGCUGUGUCUAAGGGUU | |
| | UCUUUAAGGAAGGAAGUUCUGUUGAAUUAAAACACUUCUUCUUUGCUCAGG | |
| | AUGGUAAUGCUGCUAUCAGCGAUUAUGACUACUAUCGUUAUAAUCUACCAAC | |
| | AAUGUGUGAUAUCAGACAACUACUAUUUGUAGUUGAAGUUGUUGAUAAGUA | |
| | CUUUGAUUGUUACGAUGGUGGCUGUAUUAAUGCUAACCAAGUCAUCGUCAAC | |
| | AACCUAGACAAAUCAGCUGGUUUUCCAUUUAAUAAAUGGGGUAAGGCUAGA | |
| | CUUUAUUAUGAUUCAAUGAGUUAUGAGGAUCAAGAUGCACUUUUCGCAUAU | |
| | ACAAAACGUAAUGUCAUCCCUACUAUAACUCAAAUGAAUCUUAAGUAUGCCA | |
| | UUAGUGCAAAGAAUAGAGCUCGCACCGUAGCUGGUGUCUCUAUCUGUAGUAC | |
| | UAUGACCAAUAGACAGUUUCAUCAAAAAUUAUUGAAAUCAAUAGCCGCCACU | |
| | AGAGGAGCUACUGUAGUAAUUGGAACAAGCAAAUUCUAUGGUGGUUGGCAC | |
| | AACAUGUUAAAAACUGUUUAUAGUGAUGUAGAAAACCCUCACCUUAUGGGU | |
| | UGGGAUUAUCCUAAAUGUGAUAGAGCCAUGCCUAACAUGCUUAGAAUUAUG | |
| | GCCUCACUUGUUCUUGCUCGCAAACAUACAACGUGUUGUAGCUUGUCACACC | |
| | GUUUCUAUAGAUUAGCUAAUGAGUGUGCUCAAGUAUUGAGUGAAAUGGUCA | |
| | UGUGUGGCGGUUCACUAUAUGUUAAACCAGGUGGAACCUCAUCAGGAGAUGC | |
| | CACAACUGCUUAUGCUAAUAGUGUUUUUAACAUUUGUCAAGCUGUCACGGCC | |
| | AAUGUUAAUGCACUUUUAUCUACUGAUGGUAACAAAAUUGCCGAUAAGUAU | |
| | GUCCGCAAUUUACAACACAGACUUUAUGAGUGUCUCUAUAGAAAUAGAGAU | |
| | GUUGACACAGACUUUGUGAAUGAGUUUUACGCAUAUUUGCGUAAACAUUUC | |
| | UCAAUGAUGAUACUCUCUGACGAUGCUGUUGUGUGUUUCAAUAGCACUUAU | |
| | GCAUCUCAAGGUCUAGUGGCUAGCAUAAAGAACUUUAAGUCAGUUCUUUAU | |
| | UAUCAAAACAAUGUUUUUAUGUCUGAAGCAAAAUGUUGGACUGAGACUGAC | |
| | CUUACUAAAGGACCUCAUGAAUUUUGCUCUCAACAUACAAUGCUAGUUAAAC | |
| | AGGGUGAUGAUUAUGUGUACCUUCCUUACCCAGAUCCAUCAAGAAUCCUAGG | |
| | GGCCGGCUGUUUUGUAGAUGAUAUCGUAAAAACAGAUGGUACACUUAUGAU | |
| | UGAACGGUUCGUGUCUUUAGCUAUAGAUGCUUACCCACUUACUAAACAUCCU | |
| | AAUCAGGAGUAUGCUGAUGUCUUUCAUUUGUACUUACAAUACAUAAGAAAG | |
| | CUACAUGAUGAGUUAACAGGACACAUGUUAGACAUGUAUUCUGUUAUGCUU | |
| | ACUAAUGAUAACACUUCAAGGUAUUGGGAACCUGAGUUUUAUGAGGCUAUG | |
| | UACACACCGCAUACAGUCUUACAGGCUGUUGGGGCUUGUGUUCUUUGCAAUU | |
| | CACAGACUUCAUUAAGAUGUGGUGCUUGCAUACGUAGACCAUUCUUAUGUUG | |
| | UAAAUGCUGUUACGACCAUGUCAUAUCAACAUCACAUAAAUUAGUCUUGUCU | |
| | GUUAAUCCGUAUGUUUGCAAUGCUCCAGGUUGUGAUGUCACAGAUGUGACUC | |
| | AACUUUACUUAGGAGGUAUGAGCUAUUAUUGUAAAUCACAUAAACCACCCAU | |
| | UAGUUUUCCAUUGUGUGCUAAUGGACAAGUUUUUGGUUUAUAUAAAAAUAC | |
| | AUGUGUUGGUAGCGAUAAUGUUACUGACUUUAAUGCAAUUGCAACAUGUGA | |
| | CUGGACAAAUGCUGGUGAUUACAUUUUAGCUAACACCUGUACUGAAAGACUC | |
| | AAGCUUUUUGCAGCAGAAACGCUCAAAGCUACUGAGGAGACAUUUAAACUGU | |
| | CUUAUGGUAUUGCUACUGUACGUGAAGUGCUGUCUGACAGAGAAUUACAUC | |
| | UUUCAUGGGAAGUUGGUAAACCUAGACCACCACUUAACCGAAAUUAUGUCUU | |
| | UACUGGUUAUCGUGUAACUAAAAACAGUAAAGUACAAAUAGGAGAGUACAC | |
| | CUUUGAAAAAGGUGACUAUGGUGAUGCUGUUGUUUACCGAGGUACAACAAC | |
| | UUACAAAUUAAAUGUUGGUGAUUAUUUUGUGCUGACAUCACAUACAGUAAU | |
| | GCCAUUAAGUGCACCUACACUAGUGCCACAAGAGCACUAUGUUAGAAUUACU | |
| | GGCUUAUACCCAACACUCAAUAUCUCAGAUGAGUUUUCUAGCAAUGUUGCAA | |
| | AUUAUCAAAAGGUUGGUAUGCAAAAGUAUUCUACACUCCAGGGACCACCUGG | |
| | UACUGGUAAGAGUCAUUUUGCUAUUGGCCUAGCUCUCUACUACCCUUCUGCU | |
| | CGCAUAGUGUAUACAGCUUGCUCUCAUGCCGCUGUUGAUGCACUAUGUGAGA | |
| | AGGCAUUAAAAUAUUUGCCUAUAGAUAAAUGUAGUAGAAUUAUACCUGCAC | |
| | GUGCUCGUGUAGAGUGUUUUGAUAAAUUCAAAGUGAAUUCAACAUUAGAAC | |
| | AGUAUGUCUUUUGUACUGUAAAUGCAUUGCCUGAGACGACAGCAGAUAUAG | |
| | UUGUCUUUGAUGAAAUUUCAAUGGCCACAAAUUAUGAUUUGAGUGUUGUCA | |
| | AUGCCAGAUUACGUGCUAAGCACUAUGUGUACAUUGGCGACCCUGCUCAAUU | |
| | ACCUGCACCACGCACAUUGCUAACUAAGGGCACACUAGAACCAGAAUAUUUC | |
| | AAUUCAGUGUGUAGACUUAUGAAAACUAUAGGUCCAGACAUGUUCCUCGGA | |
| | ACUUGUCGGCGUUGUCCUGCUGAAAUUGUUGACACUGUGAGUGCUUUGGUU | |
| | UAUGAUAAUAAGCUUAAAGCACAUAAAGACAAAUCAGCUCAAUGCUUUAAA | |
| | AUGUUUUAUAAGGGUGUUAUCACGCAUGAUGUUUCAUCUGCAAUUAACAGG | |
| | CCACAAAUAGGCGUGGUAAGAGAAUUCCUUACACGUAACCCUGCUUGGAGAA | |
| | AAGCUGUCUUUAUUUCACCUUAUAAUUCACAGAAUGCUGUAGCCUCAAAGAU | |
| | UUUGGGACUACCAACUCAAACUGUUGAUUCAUCACAGGGCUCAGAAUAUGAC | |
| | UAUGUCAUAUUCACUCAAACCACUGAAACAGCUCACUCUUGUAAUGUAAACA | |
| | GAUUUAAUGUUGCUAUUACCAGAGCAAAAGUAGGCAUACUUUGCAUAAUGU | |
| | CUGAUAGAGACCUUUAUGACAAGUUGCAAUUUACAAGUCUUGAAAUUCCACG | |
| | UAGGAAUGUGGCAACUUUACAAGCUGAAAAUGUAACAGGACUCUUUAAAGA | |
| | UUGUAGUAAGGUAAUCACUGGGUUACAUCCUACACAGGCACCUACACACCUC | |
| | AGUGUUGACACUAAAUUCAAAACUGAAGGUUUAUGUGUUGACAUACCUGGC | |
| | AUACCUAAGGACAUGACCUAUAGAAGACUCAUCUCUAUGAUGGGUUUUAAA | |
| | AUGAAUUAUCAAGUUAAUGGUUACCCUAACAUGUUUAUCACCCGCGAAGAAG | |
| | CUAUAAGACAUGUACGUGCAUGGAUUGGCUUCGAUGUCGAGGGGUGUCAUG | |
| | CUACUAGAGAAGCUGUUGGUACCAAUUUACCUUUACAGCUAGGUUUUUCUAC | |
| | AGGUGUUAACCUAGUUGCUGUACCUACAGGUUAUGUUGAUACACCUAAUAA | |
| | UACAGAUUUUUCCAGAGUUAGUGCUAAACCACCGCCUGGAGAUCAAUUUAAA | |
| | CACCUCAUACCACUUAUGUACAAAGGACUUCCUUGGAAUGUAGUGCGUAUAA | |
| | AGAUUGUACAAAUGUUAAGUGACACACUUAAAAAUCUCUCUGACAGAGUCG | |
| | UAUUUGUCUUAUGGGCACAUGGCUUUGAGUUGACAUCUAUGAAGUAUUUUG | |
| | UGAAAAUAGGACCUGAGCGCACCUGUUGUCUAUGUGAUAGACGUGCCACAUG | |
| | CUUUUCCACUGCUUCAGACACUUAUGCCUGUUGGCAUCAUUCUAUUGGAUUU | |
| | GAUUACGUCUAUAAUCCGUUUAUGAUUGAUGUUCAACAAUGGGGUUUUACA | |
| | GGUAACCUACAAAGCAACCAUGAUCUGUAUUGUCAAGUCCAUGGUAAUGCAC | |
| | AUGUAGCUAGUUGUGAUGCAAUCAUGACUAGGUGUCUAGCUGUCCACGAGU | |
| | GCUUUGUUAAGCGUGUUGACUGGACUAUUGAAUAUCCUAUAAUUGGUGAUG | |
| | AACUGAAGAUUAAUGCGGCUUGUAGAAAGGUUCAACACAUGGUUGUUAAAG | |
| | CUGCAUUAUUAGCAGACAAAUUCCCAGUUCUUCACGACAUUGGUAACCCUAA | |
| | AGCUAUUAAGUGUGUACCUCAAGCUGAUGUAGAAUGGAAGUUCUAUGAUGC | |
| | ACAGCCUUGUAGUGACAAAGCUUAUAAAAUAGAAGAAUUAUUCUAUUCUUA | |
| | UGCCACACAUUCUGACAAAUUCACAGAUGGUGUAUGCCUAUUUUGGAAUUGC | |
| | AAUGUCGAUAGAUAUCCUGCUAAUUCCAUUGUUUGUAGAUUUGACACUAGA | |
| | GUGCUAUCUAACCUUAACUUGCCUGGUUGUGAUGGUGGCAGUUUGUAUGUA | |
| | AAUAAACAUGCAUUCCACACACCAGCUUUUGAUAAAAGUGCUUUUGUUAAUU | |
| | UAAAACAAUUACCAUUUUUCUAUUACUCUGACAGUCCAUGUGAGUCUCAUGG | |
| | AAAACAAGUAGUGUCAGAUAUAGAUUAUGUACCACUAAAGUCUGCUACGUG | |
| | UAUAACACGUUGCAAUUUAGGUGGUGCUGUCUGUAGACAUCAUGCUAAUGA | |
| | GUACAGAUUGUAUCUCGAUGCUUAUAACAUGAUGAUCUCAGCUGGCUUUAGC | |
| | UUGUGGGUUUACAAACAAUUUGAUACUUAUAACCUCUGGAACACUUUUACA | |
| | AGACUUCAGAGUUUAGAAAAUGUGGCUUUUAAUGUUGUAAAUAAGGGACAC | |
| | UUUGAUGGACAACAGGGUGAAGUACCAGUUUCUAUCAUUAAUAACACUGUU | |
| | UACACAAAAGUUGAUGGUGUUGAUGUAGAAUUGUUUGAAAAUAAAACAACA | |
| | UUACCUGUUAAUGUAGCAUUUGAGCUUUGGGCUAAGCGCAACAUUAAACCAG | |
| | UACCAGAGGUGAAAAUACUCAAUAAUUUGGGUGUGGACAUUGCUGCUAAUA | |
| | CUGUGAUCUGGGACUACAAAAGAGAUGCUCCAGCACAUAUAUCUACUAUUGG | |
| | UGUUUGUUCUAUGACUGACAUAGCCAAGAAACCAACUGAAACGAUUUGUGCA | |
| | CCACUCACUGUCUUUUUUGAUGGUAGAGUUGAUGGUCAAGUAGACUUAUUU | |
| | AGAAAUGCCCGUAAUGGUGUUCUUAUUACAGAAGGUAGUGUUAAAGGUUUA | |
| | CAACCAUCUGUAGGUCCCAAACAAGCUAGUCUUAAUGGAGUCACAUUAAUUG | |
| | GAGAAGCCGUAAAAACACAGUUCAAUUAUUAUAAGAAAGUUGAUGGUGUUG | |
| | UCCAACAAUUACCUGAAACUUACUUUACUCAGAGUAGAAAUUUACAAGAAUU | |
| | UAAACCCAGGAGUCAAAUGGAAAUUGAUUUCUUAGAAUUAGCUAUGGAUGA | |
| | AUUCAUUGAACGGUAUAAAUUAGAAGGCUAUGCCUUCGAACAUAUCGUUUA | |
| | UGGAGAUUUUAGUCAUAGUCAGUUAGGUGGUUUACAUCUACUGAUUGGACU | |
| | AGCUAAACGUUUUAAGGAAUCACCUUUUGAAUUAGAAGAUUUUAUUCCUAU | |
| | GGACAGUACAGUUAAAAACUAUUUCAUAACAGAUGCGCAAACAGGUUCAUCU | |
| | AAGUGUGUGUGUUCUGUUAUUGAUUUAUUACUUGAUGAUUUUGUUGAAAUA | |
| | AUAAAAUCCCAAGAUUUAUCUGUAGUUUCUAAGGUUGUCAAAGUGACUAUU | |
| | GACUAUACAGAAAUUUCAUUUAUGCUUUGGUGUAAAGAUGGCCAUGUAGAA | |
| | ACAUUUUACCCAAAAUUACAAUCUAGUCAAGCGUGGCAACCGGGUGUUGCUA | |
| | UGCCUAAUCUUUACAAAAUGCAAAGAAUGCUAUUAGAAAAGUGUGACCUUC | |
| | AAAAUUAUGGUGAUAGUGCAACAUUACCUAAAGGCAUAAUGAUGAAUGUCG | |
| | CAAAAUAUACUCAACUGUGUCAAUAUUUAAACACAUUAACAUUAGCUGUACC | |
| | CUAUAAUAUGAGAGUUAUACAUUUUGGUGCUGGUUCUGAUAAAGGAGUUGC | |
| | ACCAGGUACAGCUGUUUUAAGACAGUGGUUGCCUACGGGUACGCUGCUUGUC | |
| | GAUUCAGAUCUUAAUGACUUUGUCUCUGAUGCAGAUUCAACUUUGAUUGGU | |
| | GAUUGUGCAACUGUACAUACAGCUAAUAAAUGGGAUCUCAUUAUUAGUGAU | |
| | AUGUACGACCCUAAGACUAAAAAUGUUACAAAAGAAAAUGACUCUAAAGAG | |
| | GGUUUUUUCACUUACAUUUGUGGGUUUAUACAACAAAAGCUAGCUCUUGGA | |
| | GGUUCCGUGGCUAUAAAGAUAACAGAACAUUCUUGGAAUGCUGAUCUUUAU | |
| | AAGCUCAUGGGACACUUCGCAUGGUGGACAGCCUUUGUUACUAAUGUGAAUG | |
| | CGUCAUCAUCUGAAGCAUUUUUAAUUGGAUGUAAUUAUCUUGGCAAACCACG | |
| | CGAACAAAUAGAUGGUUAUGUCAUGCAUGCAAAUUACAUAUUUUGGAGGAA | |
| | UACAAAUCCAAUUCAGUUGUCUUCCUAUUCUUUAUUUGACAUGAGUAAAUU | |
| | UCCCCUUAAAUUAAGGGGUACUGCUGUUAUGUCUUUAAAAGAAGGUCAAAU | |
| | CAAUGAUAUGAUUUUAUCUCUUCUUAGUAAAGGUAGACUUAUAAUUAGAGA | |
| | AAACAACAGAGUUGUUAUUUCUAGUGAUGUUCUUGUUAACAACUAA | |
| HIV env | AUGAGAGUGAAGGAGAAAUAUCAGCACUUGUGGAGAUGGGGGUGGAGAUGG | SEQ ID NO: |
| transcript | GGCACCAUGCUCCUUGGGAUGUUGAUGAUCUGUAGUGCUACAGAAAAAUUGU | 11 |
| | GGGUCACAGUCUAUUAUGGGGUACCUGUGUGGAAGGAAGCAACCACCACUCU | |
| | AUUUUGUGCAUCAGAUGCUAAAGCAUAUGAUACAGAGGUACAUAAUGUUUGG | |
| | GCCACACAUGCCUGUGUACCCACAGACCCCAACCCACAAGAAGUAGUAUUGGU | |
| | AAAUGUGACAGAAAAUUUUAACAUGUGGAAAAAUGACAUGGUAGAACAGAUG | |
| | CAUGAGGAUAUAAUCAGUUUAUGGGAUCAAAGCCUAAAGCCAUGUGUAAAAU | |
| | UAACCCCACUCUGUGUUAGUUUAAAGUGCACUGAUUUGAAGAAUGAUACUAA | |
| | UACCAAUAGUAGUAGCGGGAGAAUGAUAAUGGAGAAAGGAGAGAUAAAAAAC | |
| | UGCUCUUUCAAUAUCAGCACAAGCAUAAGAGGUAAGGUGCAGAAAGAAUAUG | |
| | CAUUUUUUUAUAAACUUGAUAUAAUACCAAUAGAUAAUGAUACUACCAGCUA | |
| | UAAGUUGACAAGUUGUAACACCUCAGUCAUUACACAGGCCUGUCCAAAGGUA | |
| | UCCUUUGAGCCAAUUCCCAUACAUUAUUGUGCCCCGGCUGGUUUUGCGAUUC | |
| | UAAAAUGUAAUAAUAAGACGUUCAAUGGAACAGGACCAUGUACAAAUGUCAG | |
| | CACAGUACAAUGUACACAUGGAAUUAGGCCAGUAGUAUCAACUCAACUGCUG | |
| | UUAAAUGGCAGUCUAGCAGAAGAAGAGGUAGUAAUUAGAUCUGUCAAUUUCA | |
| | CGGACAAUGCUAAAACCAUAAUAGUACAGCUGAACACAUCUGUAGAAAUUAA | |
| | UUGUACAAGACCCAACAACAAUACAAGAAAAAGAAUCCGUAUCCAGAGAGGA | |
| | CCAGGGAGAGCAUUUGUUACAAUAGGAAAAAUAGGAAAUAUGAGACAAGCAC | |
| | AUUGUAACAUUAGUAGAGCAAAAUGGAAUAACACUUUAAAACAGAUAGCUAG | |
| | CAAAUUAAGAGAACAAUUUGGAAAUAAUAAAACAAUAAUCUUUAAGCAAUCC | |
| | UCAGGAGGGGACCCAGAAAUUGUAACGCACAGUUUUAAUUGUGGAGGGGAAU | |
| | UUUUCUACUGUAAUUCAACACAACUGUUUAAUAGUACUUGGUUUAAUAGUAC | |
| | UUGGAGUACUGAAGGGUCAAAUAACACUGAAGGAAGUGACACAAUCACCCUC | |
| | CCAUGCAGAAUAAAACAAAUUAUAAACAUGUGGCAGAAAGUAGGAAAAGCAA | |
| | UGUAUGCCCCUCCCAUCAGUGGACAAAUUAGAUGUUCAUCAAAUAUUACAGG | |
| | GCUGCUAUUAACAAGAGAUGGUGGUAAUAGCAACAAUGAGUCCGAGAUCUUC | |
| | AGACCUGGAGGAGGAGAUAUGAGGGACAAUUGGAGAAGUGAAUUAUAUAAA | |
| | UAUAAAGUAGUAAAAAUUGAACCAUUAGGAGUAGCACCCACCAAGGCAAAGA | |
| | GAAGAGUGGUGCAGAGAGAAAAAAGAGCAGUGGGAAUAGGAGCUUUGUUCCU | |
| | UGGGUUCUUGGGAGCAGCAGGAAGCACUAUGGGCGCAGCCUCAAUGACGCUG | |
| | ACGGUACAGGCCAGACAAUUAUUGUCUGGUAUAGUGCAGCAGCAGAACAAUU | |
| | UGCUGAGGGCUAUUGAGGCGCAACAGCAUCUGUUGCAACUCACAGUCUGGGG | |
| | CAUCAAGCAGCUCCAGGCAAGAAUCCUGGCUGUGGAAAGAUACCUAAAGGAU | |
| | CAACAGCUCCUGGGGAUUUGGGGUUGCUCUGGAAAACUCAUUUGCACCACUG | |
| | CUGUGCCUUGGAAUGCUAGUUGGAGUAAUAAAUCUCUGGAACAGAUUUGGAA | |
| | UCACACGACCUGGAUGGAGUGGGACAGAGAAAUUAACAAUUACACAAGCUUA | |
| | AUACACUCCUUAAUUGAAGAAUCGCAAAACCAGCAAGAAAAGAAUGAACAAG | |
| | AAUUAUUGGAAUUAGAUAAAUGGGCAAGUUUGUGGAAUUGGUUUAACAUAA | |
| | CAAAUUGGCUGUGGUAUAUAAAAUUAUUCAUAAUGAUAGUAGGAGGCUUGGU | |
| | AGGUUUAAGAAUAGUUUUUGCUGUACUUUCUAUAGUGAAUAGAGUUAGGCAG | |
| | GGAUAUUCACCAUUAUCGUUUCAGACCCACCUCCCAACCCCGAGGGGACCCGA | |
| | CAGGCCCGAAGGAAUAGAAGAAGAAGGUGGAGAGAGAGACAGAGACAGAUCC | |
| | AUUCGAUUAGUGAACGGAUCCUUGGCACUUAUCUGGGACGAUCUGCGGAGCC | |
| | UGUGCCUCUUCAGCUACCACCGCUUGAGAGACUUACUCUUGAUUGUAACGAG | |
| | GAUUGUGGAACUUCUGGGACGCAGGGGGUGGGAAGCCCUCAAAUAUUGGUGG | |
| | AAUCUCCUACAGUAUUGGAGUCAGGAACUAAAGAAUAGUGCUGUUAGCUUGC | |
| | UCAAUGCCACAGCCAUAGCAGUAGCUGAGGGGACAGAUAGGGUUAUAGAAGU | |
| | AGUACAAGGAGCUUGUAGAGCUAUUCGCCACAUACCUAGAAGAAUAAGACAG | |
| | GGCUUGGAAAGGAUUUUGCUAUAA | |
| HIV gag | AUGGGUGCGAGAGCGUCAGUAUUAAGCGGGGGAGAAUUAGAUCGAUGGGAAA | SEQ ID NO: |
| transcript | AAAUUCGGUUAAGGCCAGGGGGAAAGAAAAAAUAUAAAUUAAAACAUAUAGU | 12 |
| | AUGGGCAAGCAGGGAGCUAGAACGAUUCGCAGUUAAUCCUGGCCUGUUAGAA | |
| | ACAUCAGAAGGCUGUAGACAAAUACUGGGACAGCUACAACCAUCCCUUCAGA | |
| | CAGGAUCAGAAGAACUUAGAUCAUUAUAUAAUACAGUAGCAACCCUCUAUUG | |
| | UGUGCAUCAAAGGAUAGAGAUAAAAGACACCAAGGAAGCUUUAGACAAGAUA | |
| | GAGGAAGAGCAAAACAAAAGUAAGAAAAAAGCACAGCAAGCAGCAGCUGACA | |
| | CAGGACACAGCAAUCAGGUCAGCCAAAAUUACCCUAUAGUGCAGAACAUCCA | |
| | GGGGCAAAUGGUACAUCAGGCCAUAUCACCUAGAACUUUAAAUGCAUGGGUA | |
| | AAAGUAGUAGAAGAGAAGGCUUUCAGCCCAGAAGUGAUACCCAUGUUUUCAG | |
| | CAUUAUCAGAAGGAGCCACCCCACAAGAUUUAAACACCAUGCUAAACACAGU | |
| | GGGGGGACAUCAAGCAGCCAUGCAAAUGUUAAAAGAGACCAUCAAUGAGGAA | |
| | GCUGCAGAAUGGGAUAGAGUGCAUCCAGUGCAUGCAGGGCCUAUUGCACCAG | |
| | GCCAGAUGAGAGAACCAAGGGGAAGUGACAUAGCAGGAACUACUAGUACCCU | |
| | UCAGGAACAAAUAGGAUGGAUGACAAAUAAUCCACCUAUCCCAGUAGGAGAA | |
| | AUUUAUAAAAGAUGGAUAAUCCUGGGAUUAAAUAAAAUAGUAAGAAUGUAU | |
| | AGCCCUACCAGCAUUCUGGACAUAAGACAAGGACCAAAGGAACCCUUUAGAG | |
| | ACUAUGUAGACCGGUUCUAUAAAACUCUAAGAGCCGAGCAAGCUUCACAGGA | |
| | GGUAAAAAAUUGGAUGACAGAAACCUUGUUGGUCCAAAAUGCGAACCCAGAU | |
| | UGUAAGACUAUUUUAAAAGCAUUGGGACCAGCGGCUACACUAGAAGAAAUGA | |
| | UGACAGCAUGUCAGGGAGUAGGAGGACCCGGCCAUAAGGCAAGAGUUUUGGC | |
| | UGAAGCAAUGAGCCAAGUAACAAAUUCAGCUACCAUAAUGAUGCAGAGAGGC | |
| | AAUUUUAGGAACCAAAGAAAGAUUGUUAAGUGUUUCAAUUGUGGCAAAGAAG | |
| | GGCACACAGCCAGAAAUUGCAGGGCCCCUAGGAAAAAGGGCUGUUGGAAAUG | |
| | UGGAAAGGAAGGACACCAAAUGAAAGAUUGUACUGAGAGACAGGCUAAUUUU | |
| | UUAGGGAAGAUCUGGCCUUCCUACAAGGGAAGGCCAGGGAAUUUUCUUCAGA | |
| | GCAGACCAGAGCCAACAGCCCCACCAGAAGAGAGCUUCAGGUCUGGGGUAGA | |
| | GACAACAACUCCCCCUCAGAAGCAGGAGCCGAUAGACAAGGAACUGUAUCCU | |
| | UUAACUUCCCUCAGGUCACUCUUUGGCAACGACCCCUCGUCACAAUAA | |
| HIV pol | UUUUUUAGGGAAGAUCUGGCCUUCCUACAAGGGAAGGCCAGGGAAUUUUCUU | SEQ ID NO: |
| transcript | CAGAGCAGACCAGAGCCAACAGCCCCACCAGAAGAGAGCUUCAGGUCUGGGG | 13 |
| | UAGAGACAACAACUCCCCCUCAGAAGCAGGAGCCGAUAGACAAGGAACUGUA | |
| | UCCUUUAACUUCCCUCAGGUCACUCUUUGGCAACGACCCCUCGUCACAAUAAA | |
| | GAUAGGGGGGCAACUAAAGGAAGCUCUAUUAGAUACAGGAGCAGAUGAUACA | |
| | GUAUUAGAAGAAAUGAGUUUGCCAGGAAGAUGGAAACCAAAAAUGAUAGGGG | |
| | GAAUUGGAGGUUUUAUCAAAGUAAGACAGUAUGAUCAGAUACUCAUAGAAAU | |
| | CUGUGGACAUAAAGCUAUAGGUACAGUAUUAGUAGGACCUACACCUGUCAAC | |
| | AUAAUUGGAAGAAAUCUGUUGACUCAGAUUGGUUGCACUUUAAAUUUUCCCA | |
| | UUAGCCCUAUUGAGACUGUACCAGUAAAAUUAAAGCCAGGAAUGGAUGGCCC | |
| | AAAAGUUAAACAAUGGCCAUUGACAGAAGAAAAAAUAAAAGCAUUAGUAGAA | |
| | AUUUGUACAGAGAUGGAAAAGGAAGGGAAAAUUUCAAAAAUUGGGCCUGAAA | |
| | AUCCAUACAAUACUCCAGUAUUUGCCAUAAAGAAAAAAGACAGUACUAAAUG | |
| | GAGAAAAUUAGUAGAUUUCAGAGAACUUAAUAAGAGAACUCAAGACUUCUGG | |
| | GAAGUUCAAUUAGGAAUACCACAUCCCGCAGGGUUAAAAAAGAAAAAAUCAG | |
| | UAACAGUACUGGAUGUGGGUGAUGCAUAUUUUUCAGUUCCCUUAGAUGAAGA | |
| | CUUCAGGAAGUAUACUGCAUUUACCAUACCUAGUAUAAACAAUGAGACACCA | |
| | GGGAUUAGAUAUCAGUACAAUGUGCUUCCACAGGGAUGGAAAGGAUCACCAG | |
| | CAAUAUUCCAAAGUAGCAUGACAAAAAUCUUAGAGCCUUUUAGAAAACAAAA | |
| | UCCAGACAUAGUUAUCUAUCAAUACAUGGAUGAUUUGUAUGUAGGAUCUGAC | |
| | UUAGAAAUAGGGCAGCAUAGAACAAAAAUAGAGGAGCUGAGACAACAUCUGU | |
| | UGAGGUGGGGACUUACCACACCAGACAAAAAACAUCAGAAAGAACCUCCAUU | |
| | CCUUUGGAUGGGUUAUGAACUCCAUCCUGAUAAAUGGACAGUACAGCCUAUA | |
| | GUGCUGCCAGAAAAAGACAGCUGGACUGUCAAUGACAUACAGAAGUUAGUGG | |
| | GGAAAUUGAAUUGGGCAAGUCAGAUUUACCCAGGGAUUAAAGUAAGGCAAUU | |
| | AUGUAAACUCCUUAGAGGAACCAAAGCACUAACAGAAGUAAUACCACUAACA | |
| | GAAGAAGCAGAGCUAGAACUGGCAGAAAACAGAGAGAUUCUAAAAGAACCAG | |
| | UACAUGGAGUGUAUUAUGACCCAUCAAAAGACUUAAUAGCAGAAAUACAGAA | |
| | GCAGGGGCAAGGCCAAUGGACAUAUCAAAUUUAUCAAGAGCCAUUUAAAAAU | |
| | CUGAAAACAGGAAAAUAUGCAAGAAUGAGGGGUGCCCACACUAAUGAUGUAA | |
| | AACAAUUAACAGAGGCAGUGCAAAAAAUAACCACAGAAAGCAUAGUAAUAUG | |
| | GGGAAAGACUCCUAAAUUUAAACUGCCCAUACAAAAGGAAACAUGGGAAACA | |
| | UGGUGGACAGAGUAUUGGCAAGCCACCUGGAUUCCUGAGUGGGAGUUUGUUA | |
| | AUACCCCUCCCUUAGUGAAAUUAUGGUACCAGUUAGAGAAAGAACCCAUAGU | |
| | AGGAGCAGAAACCUUCUAUGUAGAUGGGGCAGCUAACAGGGAGACUAAAUUA | |
| | GGAAAAGCAGGAUAUGUUACUAAUAGAGGAAGACAAAAAGUUGUCACCCUAA | |
| | CUGACACAACAAAUCAGAAGACUGAGUUACAAGCAAUUUAUCUAGCUUUGCA | |
| | GGAUUCGGGAUUAGAAGUAAACAUAGUAACAGACUCACAAUAUGCAUUAGGA | |
| | AUCAUUCAAGCACAACCAGAUCAAAGUGAAUCAGAGUUAGUCAAUCAAAUAA | |
| | UAGAGCAGUUAAUAAAAAAGGAAAAGGUCUAUCUGGCAUGGGUACCAGCACA | |
| | CAAAGGAAUUGGAGGAAAUGAACAAGUAGAUAAAUUAGUCAGUGCUGGAAUC | |
| | AGGAAAGUACUAUUUUUAGAUGGAAUAGAUAAGGCCCAAGAUGAACAUGAGA | |
| | AAUAUCACAGUAAUUGGAGAGCAAUGGCUAGUGAUUUUAACCUGCCACCUGU | |
| | AGUAGCAAAAGAAAUAGUAGCCAGCUGUGAUAAAUGUCAGCUAAAAGGAGAA | |
| | GCCAUGCAUGGACAAGUAGACUGUAGUCCAGGAAUAUGGCAACUAGAUUGUA | |
| | CACAUUUAGAAGGAAAAGUUAUCCUGGUAGCAGUUCAUGUAGCCAGUGGAUA | |
| | UAUAGAAGCAGAAGUUAUUCCAGCAGAAACAGGGCAGGAAACAGCAUAUUUU | |
| | CUUUUAAAAUUAGCAGGAAGAUGGCCAGUAAAAACAAUACAUACUGACAAUG | |
| | GCAGCAAUUUCACCGGUGCUACGGUUAGGGCCGCCUGUUGGUGGGCGGGAAU | |
| | CAAGCAGGAAUUUGGAAUUCCCUACAAUCCCCAAAGUCAAGGAGUAGUAGAA | |
| | UCUAUGAAUAAAGAAUUAAAGAAAAUUAUAGGACAGGUAAGAGAUCAGGCUG | |
| | AACAUCUUAAGACAGCAGUACAAAUGGCAGUAUUCAUCCACAAUUUUAAAAG | |
| | AAAAGGGGGGAUUGGGGGGUACAGUGCAGGGGAAAGAAUAGUAGACAUAAUA | |
| | GCAACAGACAUACAAACUAAAGAAUUACAAAAACAAAUUACAAAAAUUCAAA | |
| | AUUUUCGGGUUUAUUACAGGGACAGCAGAAAUCCACUUUGGAAAGGACCAGC | |
| | AAAGCUCCUCUGGAAAGGUGAAGGGGCAGUAGUAAUACAAGAUAAUAGUGAC | |
| | AUAAAAGUAGUGCCAAGAAGAAAAGCAAAGAUCAUUAGGGAUUAUGGAAAAC | |
| | AGAUGGCAGGUGAUGAUUGUGUGGCAAGUAGACAGGAUGAGGAUUAG | |
| HIV nef | AUGGGUGGCAAGUGGUCAAAAAGUAGUGUGAUUGGAUGGCCUACUGUAAGGG | SEQ ID NO: |
| transcript | AAAGAAUGAGACGAGCUGAGCCAGCAGCAGAUAGGGUGGGAGCAGCAUCUCG | 14 |
| | AGACCUGGAAAAACAUGGAGCAAUCACAAGUAGCAAUACAGCAGCUACCAAU | |
| | GCUGCUUGUGCCUGGCUAGAAGCACAAGAGGAGGAGGAGGUGGGUUUUCCAG | |
| | UCACACCUCAGGUACCUUUAAGACCAAUGACUUACAAGGCAGCUGUAGAUCU | |
| | UAGCCACUUUUUAAAAGAAAAGGGGGGACUGGAAGGGCUAAUUCACUCCCAA | |
| | AGAAGACAAGAUAUCCUUGAUCUGUGGAUCUACCACACACAAGGCUACUUCC | |
| | CUGAUUAG | |

TABLE 2

| | | SEQ ID |
|---|---|---|
| Protein | Sequence | NO |
| SARS- | MFVFLVLLPLVSSQCVNLTTRTQLPPAYTNSFTRGVYYPDKVFRSSVLHSTQDLFLP | SEQ ID |
| CoV-2 | FFSNVTWFHAIHVSGTNGTKRFDNPVLPFNDGVYFASTEKSNIIRGWIFGTTLDSKT | NO: 6 |
| Spike | QSLLIVNNATNVVIKVCEFQFCNDPFLGVYYHKNNKSWMESEFRVYSSANNCTFEY | |
| protein | VSQPFLMDLEGKQGNFKNLREFVFKNIDGYFKIYSKHTPINLVRDLPQGFSALEPLV | |
| | DLPIGINITRFQTLLALHRSYLTPGDSSSGWTAGAAAYYVGYLQPRTFLLKYNENGT | |
| | ITDAVDCALDPLSETKCTLKSFTVEKGIYQTSNFRVQPTESIVRFPNITNLCPFGEVFN | |
| | ATRFASVYAWNRKRISNCVADYSVLYNSASFSTFKCYGVSPTKLNDLCFTNVYADS | |
| | FVIRGDEVRQIAPGQTGKIADYNYKLPDDFTGCVIAWNSNNLDSKVGGNYNYLYR | |
| | LFRKSNLKPFERDISTEIYQAGSTPCNGVEGFNCYFPLQSYGFQPTNGVGYQPYRVV | |
| | VLSFELLHAPATVCGPKKSTNLVKNKCVNFNFNGLTGTGVLTESNKKFLPFQQFGR | |
| | DIADTTDAVRDPQTLEILDITPCSFGGVSVITPGTNTSNQVAVLYQDVNCTEVPVAIH | |
| | ADQLTPTWRVYSTGSNVFQTRAGCLIGAEHVNNSYECDIPIGAGICASYQTQTNSPR | |
| | RARSVASQSIIAYTMSLGAENSVAYSNNSIAIPTNFTISVTTEILPVSMTKTSVDCTM | |
| | YICGDSTECSNLLLQYGSFCTQLNRALTGIAVEQDKNTQEVFAQVKQIYKTPPIKDF | |
| | GGFNFSQILPDPSKPSKRSFIEDLLFNKVTLADAGFIKQYGDCLGDIAARDLICAQKF | |
| | NGLTVLPPLLTDEMIAQYTSALLAGTITSGWTFGAGAALQIPFAMQMAYRFNGIGV | |
| | TQNVLYENQKLIANQFNSAIGKIQDSLSSTASALGKLQDVVNQNAQALNTLVKQLS | |
| | SNFGAISSVLNDILSRLDKVEAEVQIDRLITGRLQSLQTYVTQQLIRAAEIRASANLA | |
| | ATKMSECVLGQSKRVDFCGKGYHLMSFPQSAPHGVVFLHVTYVPAQEKNFTTAPA | |
| | ICHDGKAHFPREGVFVSNGTHWFVTQRNFYEPQIITTDNTFVSGNCDVVIGIVNNTV | |
| | YDPLQPELDSFKEELDKYFKNHTSPDVDLGDISGINASVVNIQKEIDRLNEVAKNLN | |
| | ESLIDLQELGKYEQYIKWPWYIWLGFIAGLIAIVMVTIMLCCMTSCCSCLKGCCSCG | |
| | SCCKFDEDDSEPVLKGVKLHYT | |
| SARS- | MYSFVSEETGTLIVNSVLLFLAFVVFLLVTLAILTALRLCAYCCNIVNVSLVKPSFYV | SEQ ID |
| CoV-2 | YSRVKNLNSSRVPDLLV | NO: 7 |
| Envelope | | |
| protein | | |
| SARS- | MSDNGPQNQRNAPRITFGGPSDSTGSNQNGERSGARSKQRRPQGLPNNTASWFTAL | SEQ ID |
| CoV-2 | TQHGKEDLKFPRGQGVPINTNSSPDDQIGYYRRATRRIRGGDGKMKDLSPRWYFY | NO: 8 |
| Nucleocapsid | YLGTGPEAGLPYGANKDGIIWVATEGALNTPKDHIGTRNPANNAAIVLQLPQGTTL | |
| protein | PKGFYAEGSRGGSQASSRSSSRSRNSSRNSTPGSSRGTSPARMAGNGGDAALALLL | |
| | LDRLNQLESKMSGKGQQQQGQTVTKKSAAEASKKPRQKRTATKAYNVTQAFGRR | |
| | GPEQTQGNFGDQELIRQGTDYKHWPQIAQFAPSASAFFGMSRIGMEVTPSGTWLTY | |
| | TGAIKLDDKDPNFKDQVILLNKHIDAYKTFPPTEPKKDKKKKADETQALPQRQKKQ | |
| | QTVTLLPAADLDDFSKQLQQSMSSADSTQA | |
| SARS- | MADSNGTITVEELKKLLEQWNLVIGFLFLTWICLLQFAYANRNRFLYIIKLIFLWLL | SEQ ID |
| CoV-2 | WPVTLACFVLAAVYRINWITGGIAIAMACLVGLMWLSYFIASFRLFARTRSMWSFN | NO: 9 |
| Membrane | PETNILLNVPLHGTILTRPLLESELVIGAVILRGHLRIAGHHLGRCDIKDLPKEITVAT | |
| protein | SRTLSYYKLGASQRVAGDSGFAAYSRYRIGNYKLNTDHSSSSDNIALLVQ | |
| SARS- | MESLVPGFNEKTHVQLSLPVLQVRDVLVRGFGDSVEEVLSEARQHLKDGTCGLVE | SEQ ID |
| CoV-2 | VEKGVLPQLEQPYVFIKRSDARTAPHGHVMVELVAELEGIQYGRSGETLGVLVPHV | NO: 10 |
| Orf1ab | GEIPVAYRKVLLRKNGNKGAGGHSYGADLKSFDLGDELGTDPYEDFQENWNTKH | |
| polyprotein | SSGVTRELMRELNGGAYTRYVDNNFCGPDGYPLECIKDLLARAGKASCTLSEQLDF | |
| | IDTKRGVYCCREHEHEIAWYTERSEKSYELQTPFEIKLAKKFDTFNGECPNFVFPLN | |
| | SIIKTIQPRVEKKKLDGFMGRIRSVYPVASPNECNQMCLSTLMKCDHCGETSWQTG | |
| | DFVKATCEFCGTENLTKEGATTCGYLPQNAVVKIYCPACHNSEVGPEHSLAEYHNE | |
| | SGLKTILRKGGRTIAFGGCVFSYVGCHNKCAYWVPRASANIGCNHTGVVGEGSEG | |
| | LNDNLLEILQKEKVNINIVGDFKLNEEIAIILASFSASTSAFVETVKGLDYKAFKQIVE | |
| | SCGNFKVTKGKAKKGAWNIGEQKSILSPLYAFASEAARVVRSIFSRTLETAQNSVR | |
| | VLQKAAITILDGISQYSLRLIDAMMFTSDLATNNLVVMAYITGGVVQLTSQWLTNIF | |
| | GTVYEKLKPVLDWLEEKFKEGVEFLRDGWEIVKFISTCACEIVGGQIVTCAKEIKES | |
| | VQTFFKLVNKFLALCADSIIIGGAKLKALNLGETFVTHSKGLYRKCVKSREETGLLM | |
| | PLKAPKEIIFLEGETLPTEVLTEEVVLKTGDLQPLEQPTSEAVEAPLVGTPVCINGLM | |
| | LLEIKDTEKYCALAPNMMVTNNTFTLKGGAPTKVTFGDDTVIEVQGYKSVNITFEL | |
| | DERIDKVLNEKCSAYTVELGTEVNEFACVVADAVIKTLQPVSELLTPLGIDLDEWS | |
| | MATYYLFDESGEFKLASHMYCSFYPPDEDEEEGDCEEEEFEPSTQYEYGTEDDYQG | |
| | KPLEFGATSAALQPEEEQEEDWLDDDSQQTVGQQDGSEDNQTTTIQTIVEVQPQLE | |
| | MELTPVVQTIEVNSFSGYLKLTDNVYIKNADIVEEAKKVKPTVVVNAANVYLKHG | |
| | GGVAGALNKATNNAMQVESDDYIATNGPLKVGGSCVLSGHNLAKHCLHVVGPNV | |
| | NKGEDIQLLKSAYENFNQHEVLLAPLLSAGIFGADPIHSLRVCVDTVRTNVYLAVF | |
| | DKNLYDKLVSSFLEMKSEKQVEQKIAEIPKEEVKPFITESKPSVEQRKQDDKKIKAC | |
| | VEEVTTTLEETKFLTENLLLYIDINGNLHPDSATLVSDIDITFLKKDAPYIVGDVVQE | |
| | GVLTAVVIPTKKAGGTTEMLAKALRKVPTDNYITTYPGQGLNGYTVEEAKTVLKK | |
| | CKSAFYILPSIISNEKQEILGTVSWNLREMLAHAEETRKLMPVCVETKAIVSTIQRKY | |
| | KGIKIQEGVVDYGARFYFYTSKTTVASLINTLNDLNETLVTMPLGYVTHGLNLEEA | |
| | ARYMRSLKVPATVSVSSPDAVTAYNGYLTSSSKTPEEHFIETISLAGSYKDWSYSGQ | |
| | STQLGIEFLKRGDKSVYYTSNPTTFHLDGEVITFDNLKTLLSLREVRTIKVFTTVDNI | |
| | NLHTQVVDMSMTYGQQFGPTYLDGADVTKIKPHNSHEGKTFYVLPNDDTLRVEAF | |
| | EYYHTTDPSFLGRYMSALNHTKKWKYPQVNGLTSIKWADNNCYLATALLTLQQIE | |
| | LKFNPPALQDAYYRARAGEAANFCALILAYCNKTVGELGDVRETMSYLFQHANLD | |
| | SCKRVLNVVCKTCGQQQTTLKGVEAVMYMGTLSYEQFKKGVQIPCTCGKQATKY | |
| | LVQQESPFVMMSAPPAQYELKHGTFTCASEYTGNYQCGHYKHITSKETLYCIDGAL | |
| | LTKSSEYKGPITDVFYKENSYTTTIKPVTYKLDGVVCTEIDPKLDNYYKKDNSYFTE | |
| | QPIDLVPNQPYPNASFDNFKFVCDNIKFADDLNQLTGYKKPASRELKVTFFPDLNG | |
| | DVVAIDYKHYTPSFKKGAKLLHKPIVWHVNNATNKATYKPNTWCIRCLWSTKPVE | |
| | TSNSFDVLKSEDAQGMDNLACEDLKPVSEEVVENPTIQKDVLECNVKTTEVVGDII | |
| | LKPANNSLKITEEVGHTDLMAAYVDNSSLTIKKPNELSRVLGLKTLATHGLAAVNS | |
| | VPWDTIANYAKPFLNKVVSTTTNIVTRCLNRVCTNYMPYFFTLLLQLCTFTRSTNSR | |
| | IKASMPTTIAKNTVKSVGKFCLEASFNYLKSPNFSKLINIIIWFLLLSVCLGSLIYSTA | |
| | ALGVLMSNLGMPSYCTGYREGYLNSTNVTIATYCTGSIPCSVCLSGLDSLDTYPSLE | |
| | TIQITISSFKWDLTAFGLVAEWFLAYILFTRFFYVLGLAAIMQLFFSYFAVHFISNSW | |
| | LMWLIINLVQMAPISAMVRMYIFFASFYYVWKSYVHVVDGCNSSTCMMCYKRNR | |
| | ATRVECTTIVNGVRRSFYVYANGGKGFCKLHNWNCVNCDTFCAGSTFISDEVARD | |
| | LSLQFKRPINPTDQSSYIVDSVTVKNGSIHLYFDKAGQKTYERHSLSHFVNLDNLRA | |
| | NNTKGSLPINVIVFDGKSKCEESSAKSASVYYSQLMCQPILLLDQALVSDVGDSAEV | |
| | AVKMFDAYVNTFSSTFNVPMEKLKTLVATAEAELAKNVSLDNVLSTFISAARQGF | |
| | VDSDVETKDVVECLKLSHQSDIEVTGDSCNNYMLTYNKVENMTPRDLGACIDCSA | |
| | RHINAQVAKSHNIALIWNVKDFMSLSEQLRKQIRSAAKKNNLPFKLTCATTRQVVN | |
| | VVTTKIALKGGKIVNNWLKQLIKVTLVFLFVAAIFYLITPVHVMSKHTDFSSEIIGYK | |
| | AIDGGVTRDIASTDTCFANKHADFDTWFSQRGGSYTNDKACPLIAAVITREVGFVV | |
| | PGLPGTILRTTNGDFLHFLPRVFSAVGNICYTPSKLIEYTDFATSACVLAAECTIFKD | |
| | ASGKPVPYCYDTNVLEGSVAYESLRPDTRYVLMDGSIIQFPNTYLEGSVRVVTTFDS | |
| | EYCRHGTCERSEAGVCVSTSGRWVLNNDYYRSLPGVFCGVDAVNLLTNMFTPLIQ | |
| | PIGALDISASIVAGGIVAIVVTCLAYYFMRFRRAFGEYSHVVAFNTLLFLMSFTVLCL | |
| | TPVYSFLPGVYSVIYLYLTFYLTNDVSFLAHIQWMVMFTPLVPFWITIAYIICISTKHF | |
| | YWFFSNYLKRRVVFNGVSFSTFEEAALCTFLLNKEMYLKLRSDVLLPLTQYNRYLA | |
| | LYNKYKYFSGAMDTTSYREAACCHLAKALNDFSNSGSDVLYQPPQTSITSAVLQSG | |
| | FRKMAFPSGKVEGCMVQVTCGTTTLNGLWLDDVVYCPRHVICTSEDMLNPNYED | |
| | LLIRKSNHNFLVQAGNVQLRVIGHSMQNCVLKLKVDTANPKTPKYKFVRIQPGQTF | |
| | SVLACYNGSPSGVYQCAMRPNFTIKGSFLNGSCGSVGFNIDYDCVSFCYMHHMELP | |
| | TGVHAGTDLEGNFYGPFVDRQTAQAAGTDTTITVNVLAWLYAAVINGDRWFLNR | |
| | FTTTLNDFNLVAMKYNYEPLTQDHVDILGPLSAQTGIAVLDMCASLKELLQNGMN | |
| | GRTILGSALLEDEFTPFDVVRQCSGVTFQSAVKRTIKGTHHWLLLTILTSLLVLVQST | |
| | QWSLFFFLYENAFLPFAMGIIAMSAFAMMFVKHKHAFLCLFLLPSLATVAYFNMV | |
| | YMPASWVMRIMTWLDMVDTSLSGFKLKDCVMYASAVVLLILMTARTVYDDGAR | |
| | RVWTLMNVLTLVYKVYYGNALDQAISMWALIISVTSNYSGVVTTVMFLARGIVFM | |
| | CVEYCPIFFITGNTLQCIMLVYCFLGYFCTCYFGLFCLLNRYFRLTLGVYDYLVSTQ | |
| | EFRYMNSQGLLPPKNSIDAFKLNIKLLGVGGKPCIKVATVQSKMSDVKCTSVVLLS | |
| | VLQQLRVESSSKLWAQCVQLHNDILLAKDTTEAFEKMVSLLSVLLSMQGAVDINK | |
| | LCEEMLDNRATLQAIASEFSSLPSYAAFATAQEAYEQAVANGDSEVVLKKLKKSLN | |
| | VAKSEFDRDAAMQRKLEKMADQAMTQMYKQARSEDKRAKVTSAMQTMLFTML | |
| | RKLDNDALNNIINNARDGCVPLNIIPLTTAAKLMVVIPDYNTYKNTCDGTTFTYASA | |
| | LWEIQQVVDADSKIVQLSEISMDNSPNLAWPLIVTALRANSAVKLQNNELSPVALR | |
| | QMSCAAGTTQTACTDDNALAYYNTTKGGRFVLALLSDLQDLKWARFPKSDGTGTI | |
| | YTELEPPCRFVTDTPKGPKVKYLYFIKGLNNLNRGMVLGSLAATVRLQAGNATEVP | |
| | ANSTVLSFCAFAVDAAKAYKDYLASGGQPITNCVKMLCTHTGTGQAITVTPEANM | |
| | DQESFGGASCCLYCRCHIDHPNPKGFCDLKGKYVQIPTTCANDPVGFTLKNTVCTV | |
| | CGMWKGYGCSCDQLREPMLQSADAQSFLNRVCGVSAARLTPCGTGTSTDVVYRA | |
| | FDIYNDKVAGFAKFLKTNCCRFQEKDEDDNLIDSYFVVKRHTFSNYQHEETIYNLL | |
| | KDCPAVAKHDFFKFRIDGDMVPHISRQRLTKYTMADLVYALRHFDEGNCDTLKEIL | |
| | VTYNCCDDDYFNKKDWYDFVENPDILRVYANLGERVRQALLKTVQFCDAMRNA | |
| | GIVGVLTLDNQDLNGNWYDFGDFIQTTPGSGVPVVDSYYSLLMPILTLTRALTAES | |
| | HVDTDLTKPYIKWDLLKYDFTEERLKLFDRYFKYWDQTYHPNCVNCLDDRCILHC | |
| | ANFNVLFSTVFPPTSFGPLVRKIFVDGVPFVVSTGYHFRELGVVHNQDVNLHSSRLS | |
| | FKELLVYAADPAMHAASGNLLLDKRTTCFSVAALTNNVAFQTVKPGNFNKDFYDF | |
| | AVSKGFFKEGSSVELKHFFFAQDGNAAISDYDYYRYNLPTMCDIRQLLFVVEVVDK | |
| | YFDCYDGGCINANQVIVNNLDKSAGFPFNKWGKARLYYDSMSYEDQDALFAYTK | |
| | RNVIPTITQMNLKYAISAKNRARTVAGVSICSTMTNRQFHQKLLKSIAATRGATVVI | |
| | GTSKFYGGWHNMLKTVYSDVENPHLMGWDYPKCDRAMPNMLRIMASLVLARKH | |
| | TTCCSLSHRFYRLANECAQVLSEMVMCGGSLYVKPGGTSSGDATTAYANSVFNIC | |
| | QAVTANVNALLSTDGNKIADKYVRNLQHRLYECLYRNRDVDTDFVNEFYAYLRK | |
| | HFSMMILSDDAVVCFNSTYASQGLVASIKNFKSVLYYQNNVFMSEAKCWTETDLT | |
| | KGPHEFCSQHTMLVKQGDDYVYLPYPDPSRILGAGCFVDDIVKTDGTLMIERFVSL | |
| | AIDAYPLTKHPNQEYADVFHLYLQYIRKLHDELTGHMLDMYSVMLINDNTSRYW | |
| | EPEFYEAMYTPHTVLQAVGACVLCNSQTSLRCGACIRRPFLCCKCCYDHVISTSHK | |
| | LVLSVNPYVCNAPGCDVTDVTQLYLGGMSYYCKSHKPPISFPLCANGQVFGLYKN | |
| | TCVGSDNVTDFNAIATCDWTNAGDYILANTCTERLKLFAAETLKATEETFKLSYGI | |
| | ATVREVLSDRELHLSWEVGKPRPPLNRNYVFTGYRVTKNSKVQIGEYTFEKGDYG | |
| | DAVVYRGTTTYKLNVGDYFVLTSHTVMPLSAPTLVPQEHYVRITGLYPTLNISDEFS | |
| | SNVANYQKVGMQKYSTLQGPPGTGKSHFAIGLALYYPSARIVYTACSHAAVDALC | |
| | EKALKYLPIDKCSRIIPARARVECFDKFKVNSTLEQYVFCTVNALPETTADIVVFDEI | |
| | SMATNYDLSVVNARLRAKHYVYIGDPAQLPAPRTLLTKGTLEPEYFNSVCRLMKTI | |
| | GPDMFLGTCRRCPAEIVDTVSALVYDNKLKAHKDKSAQCFKMFYKGVITHDVSSA | |
| | INRPQIGVVREFLTRNPAWRKAVFISPYNSQNAVASKILGLPTQTVDSSQGSEYDYV | |
| | IFTQTTETAHSCNVNRFNVAITRAKVGILCIMSDRDLYDKLQFTSLEIPRRNVATLQA | |
| | ENVTGLFKDCSKVITGLHPTQAPTHLSVDTKFKTEGLCVDIPGIPKDMTYRRLISMM | |
| | GFKMNYQVNGYPNMFITREEAIRHVRAWIGFDVEGCHATREAVGTNLPLQLGFST | |
| | GVNLVAVPTGYVDTPNNTDFSRVSAKPPPGDQFKHLIPLMYKGLPWNVVRIKIVQ | |
| | MLSDTLKNLSDRVVFVLWAHGFELTSMKYFVKIGPERTCCLCDRRATCFSTASDTY | |
| | ACWHHSIGFDYVYNPFMIDVQQWGFTGNLQSNHDLYCQVHGNAHVASCDAIMTR | |
| | CLAVHECFVKRVDWTIEYPIIGDELKINAACRKVQHMVVKAALLADKFPVLHDIGN | |
| | PKAIKCVPQADVEWKFYDAQPCSDKAYKIEELFYSYATHSDKFTDGVCLFWNCNV | |
| | DRYPANSIVCRFDTRVLSNLNLPGCDGGSLYVNKHAFHTPAFDKSAFVNLKQLPFF | |
| | YYSDSPCESHGKQVVSDIDYVPLKSATCITRQNLGGAVCRHHANEYRLYLDAYNM | |
| | MISAGFSLWVYKQFDTYNLWNTFTRLQSLENVAFNVVNKGHFDGQQGEVPVSIIN | |
| | NTVYTKVDGVDVELFENKTTLPVNVAFELWAKRNIKPVPEVKILNNLGVDIAANT | |
| | VIWDYKRDAPAHISTIGVCSMTDIAKKPTETICAPLTVFFDGRVDGQVDLFRNARN | |
| | GVLITEGSVKGLQPSVGPKQASLNGVTLIGEAVKTQFNYYKKVDGVVQQLPETYFT | |
| | QSRNLQEFKPRSQMEIDFLELAMDEFIERYKLEGYAFEHIVYGDFSHSQLGGLHLLI | |
| | GLAKRFKESPFELEDFIPMDSTVKNYFITDAQTGSSKCVCSVIDLLLDDFVEIIKSQD | |
| | LSVVSKVVKVTIDYTEISFMLWCKDGHVETFYPKLQSSQAWQPGVAMPNLYKMQ | |
| | RMLLEKCDLQNYGDSATLPKGIMMNVAKYTQLCQYLNTLTLAVPYNMRVIHFGA | |
| | GSDKGVAPGTAVLRQWLPTGTLLVDSDLNDFVSDADSTLIGDCATVHTANKWDLII | |
| | SDMYDPKTKNVTKENDSKEGFFTYICGFIQQKLALGGSVAIKITEHSWNADLYKLM | |
| | GHFAWWTAFVTNVNASSSEAFLIGCNYLGKPREQIDGYVMHANYIFWRNTNPIQLS | |
| | SYSLFDMSKFPLKLRGTAVMSLKEGQINDMILSLLSKGRLIIRENNRVVISSDVLVN | |
| | N | |
| HIV env | MRVKEKYQHLWRWGWRWGTMLLGMLMICSATEKLWVTVYYGVPVWKEATTTLFCA | SEQ ID |
| protein | SDAKAYDTEVHNVWATHACVPTDPNPQEVVLVNVTENFNMWKNDMVEQMHEDIISL | NO: 15 |
| | WDQSLKPCVKLTPLCVSLKCTDLKNDTNTNSSSGRMIMEKGEIKNCSFNISTSIRGKVQK | |
| | EYAFFYKLDIIPIDNDTTSYKLTSCNTSVITQACPKVSFEPIPIHYCAPAGFAILKCNNKTF | |
| | NGTGPCTNVSTVQCTHGIRPVVSTQLLLNGSLAEEEVVIRSVNFTDNAKTIIVQLNTSVEI | |
| | NCTRPNNNTRKRIRIQRGPGRAFVTIGKIGNMRQAHCNISRAKWNNTLKQIASKLREQF | |
| | GNNKTIIFKQSSGGDPEIVTHSFNCGGEFFYCNSTQLFNSTWFNSTWSTEGSNNTEGSDTI | |
| | TLPCRIKQIINMWQKVGKAMYAPPISGQIRCSSNITGLLLTRDGGNSNNESEIFRPGGGD | |
| | MRDNWRSELYKYKVVKIEPLGVAPTKAKRRVVQREKRAVGIGALFLGFLGAAGSTMG | |
| | AASMTLTVQARQLLSGIVQQQNNLLRAIEAQQHLLQLTVWGIKQLQARILAVERYLKD | |
| | QQLLGIWGCSGKLICTTAVPWNASWSNKSLEQIWNHTTWMEWDREINNYTSLIHSLIEE | |
| | SQNQQEKNEQELLELDKWASLWNWFNITNWLWYIKLFIMIVGGLVGLRIVFAVLSIVNR | |
| | VRQGYSPLSFQTHLPTPRGPDRPEGIEEEGGERDRDRSIRLVNGSLALIWDDLRSLCLFSY | |
| | HRLRDLLLIVTRIVELLGRRGWEALKYWWNLLQYWSQELKNSAVSLLNATAIAVAEGT | |
| | DRVIEVVQGACRAIRHIPRRIRQGLERILL | |
| HIV gag | MGARASVLSGGELDRWEKIRLRPGGKKKYKLKHIVWASRELERFAVNPGLLETSEGCR | SEQ ID |
| protein | QILGQLQPSLQTGSEELRSLYNTVATLYCVHQRIEIKDTKEALDKIEEEQNKSKKKAQQA | NO: 16 |
| | AADTGHSNQVSQNYPIVQNIQGQMVHQAISPRTLNAWVKVVEEKAFSPEVIPMFSALSE | |
| | GATPQDLNTMLNTVGGHQAAMQMLKETINEEAAEWDRVHPVHAGPIAPGQMREPRGS | |
| | DIAGTTSTLQEQIGWMTNNPPIPVGEIYKRWIILGLNKIVRMYSPTSILDIRQGPKEPFRDY | |
| | VDRFYKTLRAEQASQEVKNWMTETLLVQNANPDCKTILKALGPAATLEEMMTACQGV | |
| | GGPGHKARVLAEAMSQVTNSATIMMQRGNFRNQRKIVKCFNCGKEGHTARNCRAPRK | |
| | KGCWKCGKEGHQMKDCTERQANFLGKIWPSYKGRPGNFLQSRPEPTAPPEESFRSGVE | |
| | TTTPPQKQEPIDKELYPLTSLRSLFGNDPSSQ | |
| HIV pol | FFREDLAFLQGKAREFSSEQTRANSPTRRELQVWGRDNNSPSEAGADRQGTVSFNFPQV | SEQ ID |
| protein | TLWQRPLVTIKIGGQLKEALLDTGADDTVLEEMSLPGRWKPKMIGGIGGFIKVRQYDQI | NO: 17 |
| | LIEICGHKAIGTVLVGPTPVNIIGRNLLTQIGCTLNFPISPIETVPVKLKPGMDGPKVKQWP | |
| | LTEEKIKALVEICTEMEKEGKISKIGPENPYNTPVFAIKKKDSTKWRKLVDFRELNKRTQ | |
| | DFWEVQLGIPHPAGLKKKKSVTVLDVGDAYFSVPLDEDFRKYTAFTIPSINNETPGIRYQ | |
| | YNVLPQGWKGSPAIFQSSMTKILEPFRKQNPDIVIYQYMDDLYVGSDLEIGQHRTKIEEL | |
| | RQHLLRWGLTTPDKKHQKEPPFLWMGYELHPDKWTVQPIVLPEKDSWTVNDIQKLVG | |
| | KLNWASQIYPGIKVRQLCKLLRGTKALTEVIPLTEEAELELAENREILKEPVHGVYYDPS | |
| | KDLIAEIQKQGQGQWTYQIYQEPFKNLKTGKYARMRGAHTNDVKQLTEAVQKITTESI | |
| | VIWGKTPKFKLPIQKETWETWWTEYWQATWIPEWEFVNTPPLVKLWYQLEKEPIVGAE | |
| | TFYVDGAANRETKLGKAGYVTNRGRQKVVTLTDTTNQKTELQAIYLALQDSGLEVNIV | |
| | TDSQYALGIIQAQPDQSESELVNQIIEQLIKKEKVYLAWVPAHKGIGGNEQVDKLVSAGI | |
| | RKVLFLDGIDKAQDEHEKYHSNWRAMASDFNLPPVVAKEIVASCDKCQLKGEAMHGQ | |
| | VDCSPGIWQLDCTHLEGKVILVAVHVASGYIEAEVIPAETGQETAYFLLKLAGRWPVKT | |
| | IHTDNGSNFTGATVRAACWWAGIKQEFGIPYNPQSQGVVESMNKELKKIIGQVRDQAE | |
| | HLKTAVQMAVFIHNFKRKGGIGGYSAGERIVDIIATDIQTKELQKQITKIQNFRVYYRDS | |
| | RNPLWKGPAKLLWKGEGAVVIQDNSDIKVVPRRKAKIIRDYGKQMAGDDCVASRQDE | |
| | D | |
| HIV nef | MGGKWSKSSVIGWPTVRERMRRAEPAADRVGAASRDLEKHGAITSSNTAATNAACAW | SEQ ID |
| protein | LEAQEEEEVGFPVTPQVPLRPMTYKAAVDLSHFLKEKGGLEGLIHSQRRQDILDLWIYH | NO: 18 |
| | TQGYFPD | |

TABLE 3

| | | SEQ |
|---|---|---|
| Transcript | Sequence | ID NO |
| Modified Spike | ACAUUUGCUUCUGACACAACUGUGUUCACUAGCAACCUCAAACAGACACCA | SEQ |
| including 3′ | UGUUCGUGUUUCUGGUGCUGCUGCCUCUGGUGAGCUCCCAGUGCGUGAACC | ID |
| and 5′UTR | UGACCACAAGGACCCAGCUGCCCCCUGCCUAUACCAAUUCCUUCACACGGGG | NO: |
| | CGUGUACUAUCCCGACAAGGUGUUUAGAUCUAGCGUGCUGCACUCCACACA | 19 |
| | GGAUCUGUUUCUGCCUUUCUUUUCUAACGUGACCUGGUUCCACGCCAUCCA | |
| | CGUGAGCGGCACCAAUGGCACAAAGCGGUUCGACAAUCCAGUGCUGCCCUU | |
| | UAACGAUGGCGUGUACUUCGCCUCCACCGAGAAGUCUAACAUCAUCAGAGG | |
| | CUGGAUCUUUGGCACCACACUGGACAGCAAGACACAGUCCCUGCUGAUCGU | |
| | GAACAAUGCCACCAACGUGGUCAUCAAGGUGUGCGAGUUCCAGUUUUGUAA | |
| | UGAUCCAUUCCUGGGCGUGUACUAUCACAAGAACAAUAAGUCUUGGAUGGA | |
| | GAGCGAGUUUCGCGUGUAUUCCUCUGCCAACAAUUGCACAUUUGAGUACGU | |
| | GUCCCAGCCCUUCCUGAUGGACCUGGAGGGCAAGCAGGGCAAUUUCAAGAA | |
| | CCUGAGGGAGUUCGUGUUUAAGAAUAUCGAUGGCUACUUCAAGAUCUACUC | |
| | CAAGCACACCCCAAUCAACCUGGUGCGCGACCUGCCACAGGGCUUCUCUGCC | |
| | CUGGAGCCACUGGUGGAUCUGCCCAUCGGCAUCAACAUCACCCGGUUUCAG | |
| | ACACUGCUGGCCCUGCACAGAAGCUACCUGACACCAGGCGACAGCUCCUCU | |
| | GGAUGGACCGCAGGAGCUGCCGCCUACUAUGUGGGCUAUCUGCAGCCCAGG | |
| | ACCUUCCUGCUGAAGUACAACGAGAAUGGCACCAUCACAGACGCCGUGGAU | |
| | UGCGCCCUGGAUCCCCUGUCUGAGACCAAGUGUACACUGAAGAGCUUUACC | |
| | GUGGAGAAGGGCAUCUAUCAGACAAGCAAUUUCAGGGUGCAGCCUACCGAG | |
| | UCCAUCGUGCGCUUUCCCAAUAUCACAAACCUGUGCCCUUUUGGCGAGGUG | |
| | UUCAACGCAACCAGGUUCGCAAGCGUGUACGCAUGGAAUAGGAAGCGCAUC | |
| | UCCAACUGCGUGGCCGACUAUUCUGUGCUGUACAACAGCGCCUCCUUCUCU | |
| | ACCUUUAAGUGCUAUGGCGUGAGCCCCACAAAGCUGAAUGACCUGUGCUUU | |
| | ACCAACGUGUACGCCGAUUCCUUCGUGAUCAGGGGCGACGAGGUGCGCCAG | |
| | AUCGCACCAGGACAGACAGGCAAGAUCGCAGACUACAAUUAUAAGCUGCCU | |
| | GACGAUUUCACCGGCUGCGUGAUCGCCUGGAACUCUAACAAUCUGGAUAGC | |
| | AAAGUGGGCGGCAACUACAAUUAUCUGUACCGGCUGUUUAGAAAGUCUAAU | |
| | CUGAAGCCAUUCGAGAGGGACAUCUCCACAGAGAUCUACCAGGCCGGCUCU | |
| | ACCCCCUGCAAUGGCGUGGAGGGCUUUAACUGUUAUUUCCCUCUGCAGAGC | |
| | UACGGCUUCCAGCCAACAAACGGCGUGGGCUAUCAGCCCUACCGCGUGGUG | |
| | GUGCUGUCUUUUGAGCUGCUGCACGCACCUGCAACAGUGUGCGGACCAAAG | |
| | AAGAGCACCAAUCUGGUGAAGAACAAGUGCGUGAACUUCAACUUCAACGGA | |
| | CUGACCGGAACAGGCGUGCUGACCGAGUCCAACAAGAAGUUCCUGCCUUUU | |
| | CAGCAGUUCGGCAGGGACAUCGCAGAUACCACAGACGCCGUGCGCGACCCU | |
| | CAGACCCUGGAGAUCCUGGAUAUCACACCAUGCUCCUUCGGCGGCGUGUCU | |
| | GUGAUCACACCAGGCACCAAUACAAGCAACCAGGUGGCCGUGCUGUAUCAG | |
| | GACGUGAAUUGUACCGAGGUGCCAGUGGCAAUCCACGCAGAUCAGCUGACC | |
| | CCUACAUGGCGGGUGUACUCUACCGGCAGCAACGUGUUCCAGACAAGAGCA | |
| | GGAUGCCUGAUCGGAGCAGAGCACGUGAACAAUAGCUAUGAGUGCGACAUC | |
| | CCUAUCGGCGCCGGCAUCUGUGCCUCCUACCAGACCCAGACAAACUCCCCAG | |
| | GCAGCGCAAGCUCUGUGGCAAGCCAGUCCAUCAUCGCCUAUACCAUGAGCC | |
| | UGGGCGCCGAGAAUUCCGUGGCCUACUCCAACAAUUCUAUCGCCAUCCCUA | |
| | CCAACUUCACAAUCUCCGUGACCACAGAGAUCCUGCCAGUGAGCAUGACCA | |
| | AGACAUCCGUGGACUGCACAAUGUAUAUCUGUGGCGAUUCCACCGAGUGCU | |
| | CUAACCUGCUGCUGCAGUACGGCUCUUUUUGUACCCAGCUGAAUAGAGCCC | |
| | UGACAGGCAUCGCCGUGGAGCAGGACAAGAACACACAGGAGGUGUUCGCCC | |
| | AGGUGAAGCAGAUCUACAAGACCCCACCCAUCAAGGACUUUGGCGGCUUCA | |
| | ACUUCAGCCAGAUCCUGCCCGAUCCUAGCAAGCCAUCCAAGCGGUCUCCCAU | |
| | CGAGGACCUGCUGUUCAACAAGGUGACCCUGGCCGAUGCCGGCUUCAUCAA | |
| | GCAGUAUGGCGAUUGCCUGGGCGACAUCGCCGCCAGAGACCUGAUCUGUGC | |
| | CCAGAAGUUUAAUGGCCUGACCGUGCUGCCUCCACUGCUGACAGAUGAGAU | |
| | GAUCGCCCAGUACACAUCUGCCCUGCUGGCAGGAACCAUCACAAGCGGAUG | |
| | GACCUUCGGCCCCGGAGCCGCCCUGCAGAUCCCCUUUCCCAUGCAGAUGGCC | |
| | UAUCGGUUCAACGGCAUCGGCGUGACCCAGAAUGUGCUGUACGAGAACCAG | |
| | AAGCUGAUCGCCAAUCAGUUUAACUCCGCCAUCGGCAAGAUCCAGGACUCU | |
| | CUGAGCUCCACACCAAGCGCCCUGGGCAAGCUGCAGGAUGUGGUGAAUCAG | |
| | AACGCCCAGGCCCUGAAUACCCUGGUGAAGCAGCUGUCUAGCAACUUCGGC | |
| | GCCAUCUCCUCUGUGCUGAAUGAUAUCCUGAGCAGGCUGGACCCCCCUGAG | |
| | GCAGAGGUGCAGAUCGACCGGCUGAUCACAGGCAGACUGCAGUCCCUGCAG | |
| | ACCUACGUGACACAGCAGCUGAUCAGGGCAGCAGAGAUCAGGGCAUCUGCC | |
| | AAUCUGGCCGCCACCAAGAUGAGCGAGUGCGUGCUGGGCCAGUCCAAGAGA | |
| | GUGGACUUUUGUGGCAAGGGCUAUCACCUGAUGAGCUUCCCACAGUCCGCC | |
| | CCUCACGGAGUGGUGUUUCUGCACGUGACCUACGUGCCAGCCCAGGAGAAG | |
| | AACUUCACCACAGCACCAGCAAUCUGCCACGAUGGCAAGGCACACUUUCCU | |
| | AGGGAGGGCGUGUUCGUGAGCAACGGCACCCACUGGUUUGUGACACAGCGC | |
| | AAUUUCUACGAGCCACAGAUCAUCACCACAGACAAUACAUUCGUGUCCGGC | |
| | AACUGUGACGUGGUCAUCGGCAUCGUGAACAAUACCGUGUAUGAUCCUCUG | |
| | CAGCCAGAGCUGGACUCUUUUAAGGAGGAGCUGGAUAAGUACUUCAAGAAU | |
| | CACACCAGCCCCGACGUGGAUCUGGGCGACAUCUCUGGCAUCAAUGCCAGC | |
| | GUGGUGAACAUCCAGAAGGAGAUCGACAGGCUGAACGAGGUGGCCAAGAAU | |
| | CUGAACGAGUCCCUGAUCGAUCUGCAGGAGCUGGGCAAGUAUGAGCAGGGA | |
| | UCUGGCUACAUCCCCGAGGCCCCUAGAGAUGGCCAGGCCUACGUGCGGAAGG | |
| | ACGGCGAAUGGGUGCUGCUGAGCACCUUCCUGGGCAGAAGCUACAUCAAGUG | |
| | GCCCUGGUAUAUCUGGCUGGGCUUCAUCGCCGGCCUGAUCGCCAUCGUGAU | |
| | GGUGACCAUCAUGCUGUGCUGUAUGACAAGCUGCUGUUCCUGCCUGAAGGG | |
| | CUGCUGUUCUUGUGGCAGCUGCUGUAAGUUUGAUGAGGACGAUAGCGAGCC | |
| | UGUGCUGAAGGGCGUGAAGCUGCACUACACCGCUCGCUUUCUUGCUGUCCA | |
| | AUUUCUAUUAAAGGUUCCUUUGUUCCCUAAGUCCAACUACUAAACUGGGGG | |
| | AUAUUAUGAAGGGCCUUGAGCAUCUGGAUUCUGCCUAAUAAAAAACAUUUA | |
| | UUUUCAUUGC | |
| Modified OVA | AAGCUUUAAUACGACUCACUAUAGGGAAAUAAGAGAGAAAAGAAGAGUAAGA | SEQ |
| including 3′ | AGAAAUAUAAGAGCCACCAUGGGCAGCAUCGGCGCCGCCAGCAUGGAGUUCU | ID |
| and 5′UTR | GCUUCGACGUGUUCAAGGAGCUGAAGGUGCACCACGCCAACGAGAACAUCUU | NO: |
| | CUACUGCCCCAUCGCCAUCAUGAGCGCCCUGGCCAUGGUGUACCUGGGCGCCA | 20 |
| | AGGACAGCACCCGGACCCAGAUCAACAAGGUGGUGCGGUUCGACAAGCUGCCC | |
| | GGCUUCGGCGACAGCAUCGAGGCCCAGUGCGGCACCAGCGUGAACGUGCACAG | |
| | CAGCCUGCGGGACAUCCUGAACCAGAUCACCAAGCCCAACGACGUGUACAGCU | |
| | UCAGCCUGGCCAGCCGGCUGUACGCCGAGGAGCGGUACCCCAUCCUGCCCGAG | |
| | UACCUGCAGUGCGUGAAGGAGCUGUACCGGGGCGGCCUGGAGCCCAUCAACU | |
| | UCCAGACCGCCGCCGACCAGGCCCGGGAGCUGAUCAACAGCUGGGUGGAGAGC | |
| | CAGACCAACGGCAUCAUCCGGAACGUGCUGCAGCCCAGCAGCGUGGACAGCCA | |
| | GACCGCCAUGGUGCUGGUGAACGCCAUCGUGUUCAAGGGCCUGUGGGAGAAG | |
| | ACCUUCAAGGACGAGGACACCCAGGCCAUGCCCUUCCGGGUGACCGAGCAGGA | |
| | GAGCAAGCCCGUGCAGAUGAUGUACCAGAUCGGCCUGUUCCGGGUGGCCAGC | |
| | AUGGCCAGCGAGAAGAUGAAGAUCCUGGAGCUGCCCUUCGCCAGCGGCACCA | |
| | UGAGCAUGCUGGUGCUGCUGCCCGACGAGGUGAGCGGCCUGGAGCAGCUGGA | |
| | GAGCAUCAUCAACUUCGAGAAGCUGACCGAGUGGACCAGCAGCAACGUGAUG | |
| | GAGGAGCGGAAGAUCAAGGUGUACCUGCCCCGGAUGAAGAUGGAGGAGAAGU | |
| | ACAACCUGACCAGCGUGCUGAUGGCCAUGGGCAUCACCGACGUGUUCAGCAGC | |
| | AGCGCCAACCUGAGCGGCAUCAGCAGCGCCGAGAGCCUGAAGAUCAGCCAGGC | |
| | CGUGCACGCCGCCCACGCCGAGAUCAACGAGGCCGGCCGGGAGGUGGUGGGCA | |
| | GCGCCGAGGCCGGCGUGGACGCCGCCAGCGUGAGCGAGGAGUUCCGGGCCGAC | |
| | CACCCCUUCCUGUUCUGCAUCAAGCACAUCGCCACCAACGCCGUGCUGUUCUU | |
| | CGGCCGGUGCGUGAGCCCCUGAUAGGCUCGCUUUCUUGCUGUCCAAUUUCUA | |
| | UUAAAGGUUCCUUUGUUCCCUAAGUCCAACUACUAAACUGGGGGAUAUUAUG | |
| | AAGGGCCUUGAGCAUCUGGAUUCUGCCUAAUAAAAAACAUUUAUUUUCAUUG | |
| | C | |
| Modified | AAGCUUUAAUACGACUCACUAUAGGGAAAUAAGAGAGAAAAGAAGAGUAAGA | SEQ |
| SIINFEKL | AGAAAUAUAAGAGCCACCAUGUCUAUCAUUAACUUCGAGAAGUUGCUCGCUU | ID |
| including 3′ | UCUUGCUGUCCAAUUUCUAUUAAAGGUUCCUUUGUUCCCUAAGUCCAACUAC | NO: |
| and 5′UTR | UAAACUGGGGGAUAUUAUGAAGGGCCUUGAGCAUCUGGAUUCUGCCUAAUAA | 21 |
| | AAAACAUUUAUUUUCAUUGC | |

### Tumor-Associated Antigen Sequences

In one embodiment, the at least one mRNA encodes one or more antigenic proteins derived from a tumor or cancer. In one embodiment, the mRNA encodes a TAA polypeptide, e.g. an amino acid sequence of a TAA. TAAs comprise Her2, prostate stem cell antigen (PSCA), PSMA (prostate-specific membrane antigen), B cell maturation antigen (BCMA), ERK5, alpha-fetoprotein (AFP), carcinoembryonic antigen (CEA), cancer antigen-125 (CA-125), CA19-9, calretinin, MUC-1, epithelial membrane protein (EMA), epithelial tumor antigen (ETA), tyrosinase, melanoma-associated antigen (MAGE), CD34, CD45, CD99, CD117, chromogranin, cytokeratin, desmin, glial fibrillary acidic protein (GFAP), gross cystic disease fluid protein (GCDFP-15), HMB-45 antigen, protein melan-A (melanoma antigen recognized by T lymphocytes; MART-1), myo-D1, muscle-specific actin (MSA), neurofilament, neuron-specific enolase (NSE), placental alkaline phosphatase, synaptophysin, thyroglobulin, thyroid transcription factor-1, the dimeric form of the pyruvate kinase isoenzyme type M2 (tumor M2-PK), an abnormal ras protein, or an abnormal p53 protein. In certain embodiments, the TAA is CD19, CD22, CD27, CD30, CD70, GD2 (ganglioside G2), EGFRvIII (epidermal growth factor variant III), sperm protein 17 (Sp17), mesothelin, PAP (prostatic acid phosphatase), prostein, TARP (T cell receptor gamma alternate reading frame protein), Trp-p8, or STEAP1 (six-transmembrane epithelial antigen of the prostate 1). In certain embodiments, the TAA is a cancer/testis (CT) antigen, e.g., BAGE, CAGE, CTAGE, FATE, GAGE, HCA661, HOM-TES-85, MAGEA, MAGEB, MAGEC, NA88, NY-ESO-1, NY-SAR-35, OY-TES-1, SPANXB1, SPA17, SSX, SYCP1, or TPTE. In certain other embodiments, the TAA or TSA is alpha-actinin-4, Bage-1, BCR-ABL, Bcr-Abl fusion protein, beta-catenin, CA 125, CA 15-3 (CA 27.29\BCAA), CA 195, CA 242, CA-50, CAM43, Casp-8, cdc27, cdk4, cdkn2a, CEA, coa-1, dek-can fusion protein, EBNA, EF2, Epstein Barr virus antigens, ETV6-AML1 fusion protein, HLA-A2, HLA-A11, hsp70-2, KIAAO205, Mart2, Mum-1, 2, and 3, neo-PAP, myosin class I, OS-9, pml-RARa fusion protein, PTPRK, K-ras, N-ras, triosephosphate isomerase, Gage 3,4,5,6,7, GnTV, Herv-K-mel, Lage-1, NA-88, NY-Eso-1/Lage-2, SP17, SSX-2, TRP2-Int2, gpi00 (Pmel 17), tyrosinase, TRP-1, TRP 2, MAGE-1, MAGE-3, RAGE, GAGE-1, GAGE-2, p15(58), RAGE, SCP-i, Hom/Mel-40, PRAME, p53, H-Ras, HER-2/neu, E2A-PRL, H4-RET, IGH-IGK, MYL-RAR, human papillomavirus (HPV) antigens E6 and E7, TSP-180, MAGE-4, MAGE-5, MAGE-6, pl85erbB2, p180erbB-3, c-met, nm-23H1, PSA, TAG-72-4, CA 19-9, CA 72-4, CAM 17.1, NuMa, 13-Catenin, Mum-1, p16, TAGE, PSMA (prostate-specific membrane antigen), B cell maturation antigen (BCMA), CT7, telomerase, 43-9F, 5T4, 791Tgp72, 13HCG, BCA225, BTAA, CD68\KPi, CO-029, FGF-5, G250, Ga733 (EpCAM), HTgp-175, M344, MA-50, MG7-Ag, MOV18, NB\70K, NY-CO-1, RCAS1, SDCCAG16, TA-90, TAAL6, TAG72, TLP, TPS, integrin cv3 (CD61), galactin, K Ras (V-Ki-ras2 Kirsten rat sarcoma viral oncogene) or Ral-B.

The mRNA which encodes an amino acid sequence of a TAA can be a sequence having at least 99%, 98%, 97%, 96%, 95%, 90%, 85%, or 80% identity to the RNA sequence of the selected TAA.

### Modifications of RNA

In one embodiment, the RNA (e.g. mRNA) is a modified RNA, in particular a stabilized mRNA. In some embodiments, the RNA (e.g. mRNA) may be modified for maximal efficacy, e.g. improved longevity in the cell, transcriptional efficacy, non-immunogenic properties (no toll-receptor induction etc.) and/or structural mRNA-stability.

In one embodiment, the RNA (e.g. mRNA) independently comprises at least one chemical modification. The chemical modification can e.g. be a modified nucleoside. In one embodiment, the chemical modification comprises a naturally occurring modified nucleoside. Naturally occurring modified nucleosides comprise 1-methyladenosine (m¹A), N⁶-methyladenosine (m⁶A), 2′-O-methyladenosine (Am), 5-methylcytidine (m⁵C), 2′-O-methylcytidine (Cm), 2-thiocytidine (s²C), N⁴-acetylcytidine (ac⁴C), 5-formylcytidine (fC), 2′-O-methylguanosine (Gm), inosine (I), pseudouridine (Ψ), 5-methyluridine (m⁵U), 2′-O-methyluridine (Um). 1-methylpseudouridine (m1Ψ), 2-thiouridine (s2U), 4-thiouridine (s4U), 5-methoxyuridine (mo5U), 3-methyluridine (m3U). In one embodiment, the RNA (e.g. mRNA) comprises a modified nucleoside in place of at least one uridine. In one embodiment, the RNA comprises a modified nucleoside in place of each uridine. In one embodiment, the modified nucleoside is independently selected from pseudouridine, N1-methyl-pseudouridine, 5-methyl-uridine and N1-ethylpseudouridine. In one embodiment, the modified nucleoside is a N1-methylpseudouridine modification or a N1-ethylpseudouridine modification. In one embodiment, the modified nucleoside is a N1-methylpseudouridine. For instance, N1-methyl-pseudouridine was found to be superior to several other nucleoside modifications and their combinations in terms of translation capacity. In some embodiments, at least 20%, at least 30%, at least 40%, at least 50%, at least 60%, at least 70%, at least 80%, at least 85%, at least 90%, at least 95%, or 100% of the uridine nucleosides in the mRNA are chemically modified.

Alternatively or additionally to a chemical modification, the sequence of the RNA, in particular mRNA, may be optimized. RNA sequence optimization inter alia comprises codon-optimization, optimization of G/C content and optimization of structural elements (e.g. 5′ cap, 5′ UTR, 3′ UTR and poly(A)-tail).

In some embodiments, the amino acid sequence of the at least one disease associated antigen (or antigenic protein) is encoded by a coding sequence which is codon-optimized and/or the G/C content of which is increased compared to wild type coding sequence. This also includes embodiments, wherein one or more sequence regions of the coding sequence are codon-optimized and/or increased in the G/C content compared to the corresponding sequence regions of the wild type coding sequence. In one embodiment, the codon-optimization and/or the increase in the G/C content preferably does not change the sequence of the encoded amino acid sequence. In some embodiments, the G/C content of the coding region of the RNA is increased by at least 10%, at least 20%, at least 30%, at least 40%, at least 50%, at least 55%, or even more compared to the G/C content of the coding region of the wild type RNA.

In some embodiments, the RNA (e.g. mRNA) may contain one or more optimized structural elements. Structural elements comprise a 5′ cap, 5′ UTR, 3′ UTR and poly(A)-tail. Thus, in one embodiment, the RNA (e.g. mRNA) comprises a 5′ untranslated region (UTR), a 3′ UTR, a 5′ cap and/or a poly(A) tail. In one embodiment, the RNA (e.g. mRNA) contains all of these elements. In some embodiments, the RNA comprises a 5′-UTR and/or a 3′-UTR. In one embodiment, the RNA (e.g. mRNA) comprises a cap. In some embodiments, the RNA (e.g. mRNA) comprises a 3′-poly(A) sequence. In one embodiment, the cap is a Cap1 structure or a m7GpppG cap. In one embodiment, the sequence of the 5′ UTR and/or 3′UTR is optimized. In some embodiments, the mRNA comprises a 5′ or 3′ UTR that is derived from a gene distinct from the sequence encoding the at least one disease associated antigen (or antigenic protein), i.e., the UTR is a heterologous UTR. In some embodiments, the 5′ and/or 3′UTR sequences can be derived from mRNA which are stable (e.g., globin, actin, GAPDH, tubulin, histone, or citric acid cycle enzymes) to increase the stability of the mRNA. For example, as 5′-UTR sequence, the 5′-UTR sequence of the human alpha-globin mRNA, optionally with an optimized ‘Kozak sequence’ to increase translational efficiency may be used. Alternatively, the 5′UTR sequence of a human cytochrome mRNA may be used, e.g. the human cytochrome b-245 alpha mRNA or cytochrome p4502E1 mRNA. As 3′-UTR sequence, a combination of two sequence elements (FI element) derived from the “amino terminal enhancer of split” (AES) mRNA (called F) and the mitochondrial encoded 12S ribosomal RNA (called I) placed between the coding sequence and the poly(A)-tail to assure higher maximum protein levels and prolonged persistence of the mRNA may be used. Alternatively, the 3′-UTR may be two re-iterated 3′-UTRs of the human beta-globin mRNA.

In one embodiment, the poly-A sequence comprises at least 100 nucleotides. In one embodiment, the poly-A sequence comprises at least 150 nucleotides. In one embodiment, the poly-A sequence comprises at least 250 nucleotides. Furthermore, a poly(A)-tail measuring 110 nucleotides in length, consisting of a stretch of 30 adenosine residues, followed by a 10 nucleotide linker sequence (of random nucleotides) and another 70 adenosine residues may be used. This poly(A)-tail sequence enhances RNA stability and translational efficiency. In one embodiment, a poly(A)-tail measures 300 to 800 nucleotides in length.

Furthermore, a secretory signal peptide (sec) may be fused to the antigen-encoding regions preferably in a way that the see is translated as N terminal tag. Sequences coding for short linker peptides predominantly consisting of the amino acids glycine (G) and serine (S), as commonly used for fusion proteins may be used as GS-linkers (glycine-serine-linkers). In other embodiments the RNA (e.g. mRNA) may have one or more AU-rich sequences removed. These sequences, sometimes referred to as AURES are destabilizing sequences found in the 3′UTR. The AURES may be removed from the RNA (e.g. mRNA). Alternatively the AURES may remain in the RNA (e.g. mRNA).

### Lipid Nanoparticles

The at least one disease associated antigen (e.g. at least one mRNA encoding the at least one antigenic protein) can be encapsulated in a lipid nanoparticle (LNP). For example, if the at least one disease associated antigen is an antigenic protein which is encoded by an RNA (e.g. mRNA), the RNA can be encapsulated in a lipid nanoparticle for delivery to the phDCs of the invention. The encapsulated RNA (e.g. mRNA) is combined with the phDCs.

The LNP can comprise 3, 4 or 5 classes of lipids. LNP with three classes of lipids comprise: (1) an ionizable lipid, (2) a PEGylated lipid and (3) a cholesterol-based lipid. LNP with four classes of lipids comprise: (1) an ionizable lipid, (2) a PEGylated lipid, (3) a cholesterol-based lipid and (4) a helper lipid. LNPs with five classes of lipids comprise: (1) an ionizable lipid, (2) a PEGylated lipid, (3) a cholesterol-based lipid, (4) a helper lipid and (5) DSPE-PEG-maleimide or DSPN-PEG-azide. DSPE-PEG-maleimide or DSPN-PEG-azide allow for adding ligands for targeted delivery.

### (1) Ionizable Lipids

An ionizable lipid facilitates mRNA encapsulation and may be a cationic lipid. A cationic lipid affords a positively charged environment at low pH to facilitate efficient encapsulation of the negatively charged mRNA. In one embodiment, the cationic lipid is cKK-E12 ((3,6-bis(4-(bis(2-hydroxydodecyl)amino)butyl)piperazine-2,5-dione); see Dong et al., PNAS (2014) 111(11):3955-60). Other cationic lipids that can be used include those described in Dong et al., 2014.

### (2) PEG Modified Lipid

A PEG modified lipid component provides control over particle size and stability of the nanoparticle. The addition of such components may prevent complex aggregation and provide a means for increasing life-time and increasing the delivery of the LNPs to the phDCs.

Contemplated PEGylated lipids include, but are not limited to, a polyethylene glycol (PEG) chain of up to 5 kDa in length covalently attached to a lipid with alkyl chain(s) of C6-C20 (for example, C8, C10, C12, C14, C16 or C18) length, such as a derivatized ceramide (e.g., N-octanoyl-sphingosine-1-[succinyl(methoxypolyethylene glycol)](C8 PEG ceramide)). In some embodiments, the PEGylated lipid is 1,2-dimyristoyl-rac-glycero-3-methoxypolyethylene glycol (DMG-PEG); 1,2-distearoyl-sn-glycero-3-phosphoethanolamine-polyethylene glycol (DSPE-PEG); 1,2-dilauroyl-sn-glycero-3-phosphoethanolamine-polyethylene glycol (DLPE-PEG); or 1,2-distearayl-rac-glycero-polyethelene glycol (DSG-PEG). In particularly exemplary embodiments, the PEG has a high molecular weight, e.g., 2000-2400 g/mol. In some embodiments, the PEG is PEG2000, also known as PEG-2K. In some embodiments, the PEG-modified lipid is DMG-PEG2000, DSPE-PEG2000, DLPE-PEG2000, DSG-PEG2000 or C8 PEG2000.

### (3) Cholesterol Based Lipid

The cholesterol component provides stability to the lipid bilayer structure within the nanoparticle. In some embodiments, the LNPs comprise one or more cholesterol-based lipids. Suitable cholesterol-based lipids include, for example: N,N-dimethyl-N-ethylcarboxamido-cholesterol, 1,4-bis(3-N-oleylamino-propyl)piperazine, imidazole cholesterol ester, β-sitosterol, fucosterol, stigmasterol, and other modified forms of cholesterol. In some embodiments, the cholesterol-based lipid used in the LNPs is cholesterol.

### (4) Helper Lipids

A helper lipid enhances the structural stability of the LNP and helps the LNP in endosome escape. It improves uptake and release of the RNA (e.g. mRNA) load. The cross-presenting capacity of DCs can be limited by non-specific degradation during endosome maturation. Thus, LNPs providing better endosome escape may be useful in some embodiments (e.g. if the antigen is in the form of, for example, a protein, a peptide or mRNA). In one embodiment, the helper lipid is a non-cationic lipid. In some embodiments, the helper lipid is a zwitterionic lipid, which has fusogenic properties for enhancing uptake and release of the load. Examples of helper lipids are 1,2-dioleoyl-SN-glycero-3-phosphoethanolamine (DOPE); 1,2-distearoyl-sn-glycero-3-phosphocholine (DSPC); 1,2-dioleoyl-sn-glycero-3-phospho-L-serine (DOPS); 1,2-dielaidoyl-sn-glycero-3-phosphocholine (DPOC), dipalmitoylphosphatidylcholine (DPPC), 1,2-dilauroyl-sn-glycero-3-phosphocholine (DLPC), 1,2-Distearoylphosphatidylethanolamine (DSPE), and 1,2-dilauroyl-sn-glycero-3-phosphoethanolamine (DLPE).

In one embodiment, the lipid nanoparticles comprise cKK-E12, cholesterol, C14-PEG 2000-PE and DOPE. In some embodiments, the phDCs may be loaded with different antigens to give rise to multi-valent antigen-specific phDCs. For example, if the antigenic protein is encoded by an mRNA, encapsulated in a LNP, the LNP may carry mRNAs that encode more than one antigenic protein, such as 2, 3, 4, 5, 6, 7, 8, 9, 10 or more antigenic proteins, from the same or different pathogens or from the same or different tumors. For example, the LNP may carry multiple mRNA molecules, each encoding a different antigenic protein. The LNP may also carry a polycistronic mRNA that can be translated into more than one antigenic protein. If the LNP carries different mRNA molecules, there will typically be multiple copies of each mRNA molecule.

### Molar Ratios of the Lipid Components

Specific molar ratios of the above components may be important for the LNPs' effectiveness. The molar ratio of the cationic lipid, the PEGylated lipid, the cholesterol-based lipid, and the helper lipid is A:B:C:D, where A+B+C+D=100%. In some embodiments, the molar ratio of the cationic lipid in the LNPs relative to the total lipids (i.e., A) is 35-50%, optionally 35-45%. In some embodiments, the molar ratio of the PEGylated lipid component relative to the total lipids (i.e., B) is 0.25-2.75%, optionally about 1.5%. In some embodiments, the molar ratio of the cholesterol-based lipid relative to the total lipids (i.e., C) is 20-46.5%. In some embodiments, the molar ratio of the cholesterol-based lipid relative to the total lipids (i.e., C) is about 46.5%. In another embodiment, the molar ratio of the cholesterol-based lipid relative to the total lipids (i.e., C) is about 38.5%. In some embodiments, the molar ratio of the helper lipid relative to the total lipids (i.e., D) is about 10-35%, such as 10-25%, or 16-35% (e.g., 16-32% such as 16%). In some embodiments, the molar ratio of the helper lipid relative to the total lipids (i.e., D) is about 10%. In another embodiment, the molar ratio of the helper lipid relative to the total lipids (i.e., D) is about 16%. In one embodiment, the ratio of the components is 35:2.5:46:16 (A:B:C:D). In another embodiment, the ratio of the components is 50:38.5:1.5:10 (A:B:C:D).

In one embodiment, the lipid nanoparticle comprises cKK-E12, cholesterol, C14-PEG 2000-PE and DOPE. In one embodiment, the lipid nanoparticle comprises cKK-E12, cholesterol, C14-PEG 2000-PE and DOPE at a ratio of 35:46.5:2.5:16.

In some embodiments, the lipid nanoparticle comprises SM102, cholesterol, DMG-PEG-2K, DSPC. In one embodiment, the lipid nanoparticle comprises SM102, cholesterol, DMG-PEG-2K, DSPC at a ratio of 50:38.5:1.5:10.

In some embodiments, the (PEGylated lipid+cholesterol) components have the same molar amount as the helper lipid. In some embodiments, the LNPs contain a molar ratio of the cationic lipid to the helper lipid that is more than 1. To calculate the actual amount of each lipid to be put into an LNP formulation, the molar amount of the cationic lipid is first determined based on a desired N/P ratio, where N is the number of nitrogen atoms in the cationic lipid and P is the number of phosphate groups in the RNA (e.g. mRNA) to be transported by the LNP. Next, the molar amount of each of the other lipids is calculated based on the molar amount of the cationic lipid and the molar ratio selected. These molar amounts are then converted to weights using the molecular weight of each lipid.

The lipid nanoparticles comprising the antigen (e.g. in the form of RNA, particularly mRNA encoding at least one antigenic protein) can be provided frozen. For example, this may be useful if the phDCs and lipid nanoparticles comprising the antigen (e.g. in the form of RNA, particularly mRNA encoding at least one antigenic protein) are provided as a kit.

### Size and Amount of Lipid Nanoparticles

Suitable LNPs may be made in various sizes. In some embodiments, the majority of purified LNPs, i.e., greater than about 50%, 60%, 70%, 80%, 85%, 90%, 95%, 97%, 98%, or 99% of the LNPs, have a size of about 50 to 200 nm (e.g., about 145 nm, about 140 nm, about 135 nm, about 130 nm, about 125 nm, about 120 nm, about 115 nm, about 110 nm, about 105 nm, about 100 nm, about 95 nm, about 90 nm, about 85 nm, or about 80 nm). In some embodiments, substantially all (e.g., greater than 80 or 90%) of the purified lipid nanoparticles have a size of about 70 to 200 nm (e.g., about 145 nm, about 140 nm, about 135 nm, about 130 nm, about 125 nm, about 120 nm, about 115 nm, about 110 nm, about 105 nm, about 100 nm, about 95 nm, about 90 nm, about 85 nm, or about 80 nm). In some embodiments, greater than about 70%, 80%, 85%, 90%, 95%, 97%, 98%, 99% of the LNPs in the present composition have a size ranging from about 85 to 100 nm (e.g., about 45-85 nm, about 50-80 nm, about 55-75 nm, about 60-70 nm) or about 50-70 nm (e.g., 55-65 nm). The smaller sizes are particular suitable for inhalative delivery via nebulization. In some embodiments, the phDCs are combined with at least about 1 μg, 2 μg, 3 μg, 4 μg, 5 μg, 10 μg or 20 μg of encapsulated RNA, particularly mRNA. In some embodiments, the phDCs are combined with about 10 μg of encapsulated RNA, particularly mRNA. In some embodiments, the phDCs are combined with about 20 μg of encapsulated RNA, particularly mRNA.

### Lipoplexes

The at least one disease associated antigen (e.g. at least one mRNA encoding the at least one antigenic protein) can be comprised in a lipoplex. Mixing of RNA (e.g. mRNA) and positive charged liposomes results in the formation of lipoplex particles by spontaneous self assembly. The liposomes typically contain at least two components: a cationic lipid and a neutral lipid. Lipoplexes have extensively be described in the art, see, e.g. Nanomedicine: Nanotechnology, Biology and Medicine, 2009. In one embodiment, the neutral lipid is a helper lipid as defined above. In an exemplary embodiment, the cationic lipid is DOTMA and the neutral lipid is DOPE.

In some embodiments, the molar ratio of the at least one cationic lipid to the at least one neutral lipid is from about 10:0 to about 1:9, about 4:1 to about 1:2, or about 3:1 to about 1:1. In specific embodiments, the molar ratio may be about 3:1, about 2.75:1, about 2.5:1, about 2.25:1, about 2:1, about 1.75:1, about 1.5:1, about 1.25:1, or about 1:1. In an exemplary embodiment, the molar ratio of the at least one cationic lipid to the at least one additional lipid is about 2:1. RNA lipoplex particles described herein have an average diameter that in one embodiment ranges from about 200 nm to about 1000 nm, from about 200 nm to about 800 nm, from about 250 to about 700 nm, from about 400 to about 600 nm, from about 300 nm to about 500 nm, or from about 350 nm to about 400 nm. In specific embodiments, the RNA lipoplex particles have an average diameter of about 200 nm, about 225 nm, about 250 nm, about 275 nm, about 300 nm, about 325 nm, about 350 nm, about 375 nm, about 400 nm, about 425 nm, about 450 nm, about 475 nm, about 500 nm, about 525 nm, about 550 nm, about 575 nm, about 600 nm, about 625 nm, about 650 nm or about 700 nm. In an embodiment, the RNA lipoplex particles have an average diameter that ranges from about 250 nm to about 700 nm. In another embodiment, the RNA lipoplex particles have an average diameter that ranges from about 300 nm to about 500 nm. In an exemplary embodiment, the RNA lipoplex particles have an average diameter of about 400 nm.

In one embodiment, the (mRNA) lipoplex particles comprise at least one cationic lipid and at least one neutral lipid. In one embodiment, the at least one cationic lipid comprises 1,2-di-O-octadecenyl-3-trimethylammonium propane (DOTMA) and/or 1,2-dioleoyl-3-trimethylammonium-propane (DOTAP). In one embodiment, the at least one neutral lipid comprises 1,2-di-(9Z-octadecenoyl)-sn-glycero-3-phosphoethanolamine (DOPE), cholesterol (Chol) and/or 1,2-dioleoyl-sn-glycero-3-phosphocholine (DOPC). In one embodiment, the at least one cationic lipid comprises 1,2-di-O-octadecenyl-3-trimethylammonium propane (DOTMA) and the at least one neutral lipid comprises 1,2-di-(9Z-octadecenoyl)-sn-glycero-3-phosphoethanolamine (DOPE). In one embodiment, the lipoplex particles comprise 1,2-di-O-octadecenyl-3-trimethylammonium propane (DOTMA) and 1,2-di-(9Z-octadecenoyl)-sn-glycero-3-phosphoethanolamine (DOPE). Spleen targeting RNA lipoplex particles are described in WO 2013/143683, herein incorporated by reference. It has been found that lipoplex particles having a net negative charge may be used to preferentially target spleen tissue or spleen cells such as antigen-presenting cells, in particular dendritic cells.

### Oligomers, Polymers or Lipidoids Comprising Oligo(Alkylene Amine) Moieties

The at least one antigen (e.g. at least one mRNA encoding the at least one antigen) can be comprised in oligomers, polymers or lipidoids comprising oligo(alkylene amine) moieties, for example, the characteristic oligo(alkylene amine) moieties as described in PCT/EP2014/063756. In particular, the at least one antigen (e.g. at least one mRNA encoding the at least one antigen) can be comprised in oligomers, polymers or lipidoids as described in PCT/EP2014/063756. One main characteristic of the oligomers, polymers or lipidoids comprising oligo(alkylene amine) moieties is that they contain a following common structural entity of formula (I):

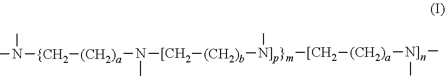

Such oligomers, polymers or lipidoids comprising oligo(alkylene amine) moieties may be selected from:
a) an oligomer or polymer comprising a plurality of groups of formula (II) as a side chain and/or as a terminal group:

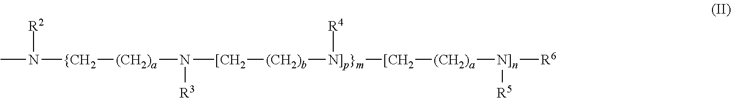

wherein the variables a, b, p, m, n and R² to R⁶ are defined as follows, independently for each group of formula (II) in a plurality of such groups:
a is 1 and b is an integer of 2 to 4; or a is an integer of 2 to 4 and b is 1,
p is 1 or 2,
m is 1 or 2; n is 0 or 1 and m+n is ≥2; and
R² to R⁵ are, independently of each other, selected from hydrogen; a group —CH₂—CH(OH)—R⁷, —CH(R⁷)—CH₂—OH, —CH₂—CH₂—(C═O)—O—R⁷, —CH₂—CH₂—(C═O)—NH—R⁷ or —CH₂—R⁷ wherein R⁷ is selected from C3-C18 alkyl or C3-C18 alkenyl having one C═C double bond; a protecting group for an amino group; —C(NH)—NH₂; a poly(ethylene glycol) chain;
R⁶ is selected from hydrogen; a group —CH₂—CH(OH)—R⁷, —CH(R⁷)—CH₂—OH, —CH₂—CH₂—(C═O)—O—R⁷, —CH₂—CH₂—(C═O)—NH—R⁷ or —CH₂—R⁷ wherein R⁷ is selected from C3-C18 alkyl or C3-C18 alkenyl having one C═C double bond; a protecting group for an amino group; —C(NH)—NH₂; a poly(ethylene glycol) chain; and a receptor ligand, and wherein one or more of the nitrogen atoms indicated in formula (II) may be protonated to provide a cationic group of formula (II);
b) an oligomer or polymer comprising a plurality of groups of formula (III) as repeating units:

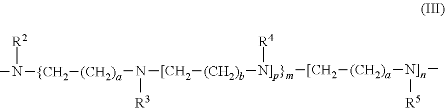

wherein the variables a, b, p, m, n and R² to R⁵ are defined as follows, independently for each group of formula (III) in a plurality of such groups:
a is 1 and b is an integer of 2 to 4; or a is an integer of 2 to 4 and b is 1,
p is 1 or 2,
m is 1 or 2; n is 0 or 1 and m+n is ≥2; and
R² to R⁵ are, independently of each other, selected from hydrogen; a group —CH₂—CH(OH)—R⁷, —CH(R⁷)—CH₂—OH, —CH₂—CH₂—(C═O)—O—R⁷, —CH₂—CH₂—(C═O)—NH—R⁷ or —CH₂—R⁷ wherein R⁷ is selected from C3-C18 alkyl or C3-C18 alkenyl having one C═C double bond; a protecting group for an amino group; —C(NH)—NH₂; and a poly(ethylene glycol) chain;
and wherein one or more of the nitrogen atoms indicated in formula (III) may be protonated to provide a cationic group of formula (III); and
c) a lipidoid having the structure of formula (IV):

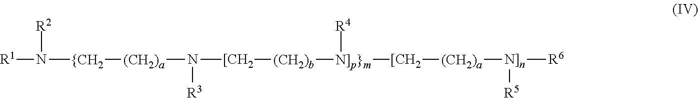

wherein the variables a, b, p, m, n and R² to R⁶ are defined as follows:
a is 1 and b is an integer of 2 to 4; or a is an integer of 2 to 4 and b is 1,
p is 1 or 2,
m is 1 or 2; n is 0 or 1 and m+n is ≥2; and
R¹ to R⁶ are, independently of each other, selected from hydrogen; a group —CH₂—CH(OH)—R⁷, —CH(R⁷)—CH₂—OH, —CH₂—CH₂—(C═O)—O—R⁷, —CH₂—CH₂—(C═O)—NH—R⁷ or —CH₂—R⁷ wherein R⁷ is selected from C3-C18 alkyl or C3-C18 alkenyl having one C═C double bond; a protecting group for an amino group; —C(NH)—NH₂; a poly(ethylene glycol) chain; and a receptor ligand,
provided that at least two residues among R¹ to R⁶ are a group —CH₂—CH(OH)—R⁷, —CH(R⁷)—CH₂—OH, —CH₂—CH₂—(C═O)—O—R⁷, —CH₂—CH₂—(C═O)—NH—R⁷ or —CH₂—R⁷ wherein R⁷ is selected from C3-C18 alkyl or C3-C18 alkenyl having one C═C double bond; and wherein one or more of the nitrogen atoms indicated in formula (IV) may be protonated to provide a cationic lipidoid of formula (IV).

Preferably, oligomers, polymers or lipidoids comprising oligo(alkylene amine) moieties are selected from a) and b), wherein
a) is an oligomer or polymer comprising a plurality of groups of formula (IIa) as a side chain and/or as a terminal group:

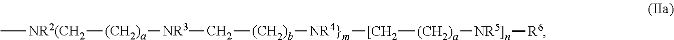

wherein a, b, m, n, and R² to R⁶ are defined as described above, and wherein one or more of the nitrogen atoms indicated in formula (IIa) may be protonated to provide a cationic oligomer or polymer structure; and
b) is an oligomer or polymer comprising a plurality of groups of formula (IIIa) as repeating units:

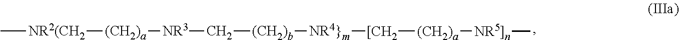

wherein a, b, m, n, and R² to R⁵ are defined as described above, and wherein one or more of the nitrogen atoms indicated in formula (IIIa) may be protonated to provide a cationic oligomer or polymer structure.

Furthermore, lipidoids comprising oligo(alkylene amine) moieties may be selected from a lipidoid having the structure of formula (IVa):

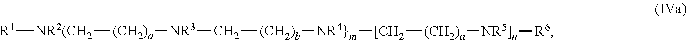

wherein a, b, m, n, and R¹ to R⁶ are defined as described above, and wherein one or more of the nitrogen atoms indicated in formula (IVa) may be protonated to provide a cationic lipidoid.

As to such oligomers, polymers or lipidoids comprising oligo(alkylene amine) moieties, in formula (II), (IIa), (III), (IIIa), (IV) or (IVa) n may be 1; or m may be 1 and n may be 1.

Further, as to such oligomers, polymers or lipidoids comprising oligo(alkylene amine) moieties, in formula (II), (IIa), (III), (IIIa), (IV) or (IVa) a may be 1 and b may be 2; or a may be 2 and b may be 1.

One non-limiting example of such an oligomer, polymer or lipidoid comprising oligo(alkylene amine) moieties is a cationic lipid which was prepared by mixing 100 mg N,N′-Bis(2-aminoethyl)-1,3-propanediamine (0.623 mmol) with 575.07 mg 1,2-Epoxydodecane (3.12 mmol, (N−1) eq. where N is 2× amount of primary amine plus 1× amount secondary amine per oligo(alkylene amine)) and mixed for 96 h at 80° C. under constant shaking. Such an oligomer, polymer or lipidoid is also referred to as lipidoid “C12-(2-3-2)”. An oligomer, polymer or lipidoid comprising oligo(alkylene amine) moieties, in particular a polymer, can be a copolymer, in particular a statistical copolymer. Such a copolymer may be a copolymer which contains a statistical/random arrangement of alkylene amine repeating units of alternating length (e.g. in contrast to a less preferred polymer which contains analogous arrangements of alkylene amine repeating units of non-alternating length).

The copolymer may be a cationic (e.g. protonated) copolymer. Copolymers to be employed are known in the art and are, for example, described in EP 14 19 9439.2, WO 01/00708, EP-A1 1 198 489 and CA-A1 2,377,207.

In particular, the copolymer may be a statistical copolymer comprising a plurality of repeating units (a) independently selected from repeating units of the following formulae (a1) and (a2):

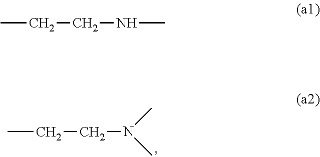

and a plurality of repeating units (b) independently selected from repeating units of the following formulae (b1) to (b4):

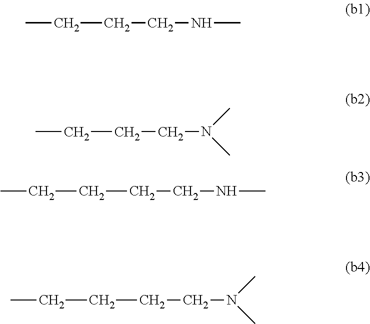

wherein the molar ratio of the sum of the repeating units (a) to the sum of the repeating units (b) lies within the range of 0.7/1.0 to 1.0/0.7, and wherein one or more of the nitrogen atoms of the repeating units (a) and/or (b) contained in the copolymer may be protonated to provide a cationic copolymer.

The copolymer may be a statistical copolymer, wherein any repeating units (a) and any repeating units (b) are statistically distributed in the copolymer macromolecule. It is typically obtained from the copolymerization of a mixture of monomers yielding, during the polymerization reaction, the repeating units (a) with monomers yielding, during the polymerization reaction, the repeating units (b). Preferably, the copolymer is a random copolymer wherein any repeating units (a) and any repeating units (b) are randomly distributed in the polymer macromolecule. Such a copolymer can be a linear, branched or dendritic copolymer. As will be understood by the skilled reader, a repeating unit of the formula (a1), (b1) or (b3) with two valencies (i.e. open bonds to neighboring units) leads to a propagation of the copolymer structure in a linear manner. Thus, a linear copolymer may comprise repeating units of formula (a1) and one or more types of the repeating units of formulae (b1) and (b3), but no repeating units of formula (a2), (b2) or (b4). As will be further understood, the presence of a repeating unit of formula (a2), (b2) or (b4) with three valencies provides a branching point in the copolymer structure. Thus, a branched copolymer comprises one or more types of the repeating units of formulae (a2), (b2) and (b4), and may further comprise one or more types of the repeating units of formulae (a1), (b1) and (b3). Such a copolymer may comprise a plurality of repeating units (a) independently selected from repeating units of formulae (a1) and (a2) defined above, and a plurality of repeating units (b) independently selected from repeating units of formulae (b1) to (b4) defined above. Preferred are copolymers comprising a plurality of repeating units (a) independently selected from repeating units of formulae (a1) and (a2) defined above, and a plurality of repeating units (b) independently selected from repeating units of formulae (b1) and (b2) defined above.

Preferably, such a copolymer is a branched copolymer comprising one or more types of repeating units selected from repeating units (a2), (b2) and (b4), and which optionally further comprises one or more types of the repeating units of formulae (a1), (b1) and (b3), and in particular a copolymer which comprises repeating units of the formula (a2) and one or more type of the repeating units of formulae (b2) and (b4), and which optionally further comprises one or more types of the repeating units of formulae (a1), (b1) and (b3). In line with the above, a more preferred copolymer is thus a branched copolymer which comprises repeating units of the formula (a2) and repeating units of formula (b2), and which optionally further comprises one or more types of the repeating units of formulae (a1) and (b1). In the copolymers, the total number of the repeating units (a) and repeating units (b) is typically 20 or more, preferably 50 or more and more preferably 100 or more. Typically, the total number of the repeating units (a) and repeating units (b) is 10,000 or less, preferably 5,000 or less, more preferably 1,000 or less. Furthermore, it is preferred for the copolymers that the repeating units (a) and (b) account for 80 mol % or more, more preferably 90 mol % or more of all repeating units in the copolymer. Further preferred are copolymers wherein repeating units (a) selected from (a1) and (a2) and repeating units (b) selected from (b1) and (b2) account for 80 mol % or more, more preferably 90 mol % or more of all repeating units in the copolymer. It is most preferred that all of the repeating units in the copolymer are repeating units (a) or (b), in particular that all of the repeating units in the copolymer are repeating units (a) selected from (a1) and (a2) or repeating units (b) selected from (b1) and (b2). The weight average molecular weight of the copolymer, as measured e.g. via size exclusion chromatography relative to linear poly(ethylene oxide) standards, generally ranges from 1,000 to 500,000 Da, preferably from 2,500 to 250,000 Da and more preferably 5,000-50,000 less. The terminal groups of such a copolymer typically comprise one or more types of groups (c) independently selected from groups of the formulae (c1) to (c3) below, preferably from groups of the formulae (c1) and (c2) below:

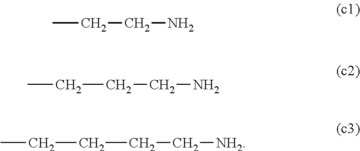

Preferably, the terminal groups in the copolymer consist of one or more types of groups (c) independently selected from groups of the formulae (c1) to (c3) below, preferably from groups of the formulae (c1) and (c2). As will be understood by the skilled person, the number of terminal groups depends on the structure of the copolymer. While a linear copolymer has only two terminals, larger numbers of terminal groups are contained in a branched, in particular in a dendritic copolymer.

As will be further understood, also one or more of the nitrogen atoms of the terminal groups (c) contained in the copolymer may be protonated to provide a cationic copolymer. In the copolymer, the molar ratio of the sum of the repeating units (a) to the sum of the repeating units (b) lies within the range of 0.7/1.0 to 1.0/0.7, and preferably within the range of 0.8/1.0 to 1.0/0.8. This molar ratio can be determined, e.g., via NMR. It will thus be understood that the ratio is usually determined for a plurality of macromolecules of the copolymer, and typically indicates the overall ratio of the sum of repeating units (a) to the sum of repeating units (b) in the plurality of macromolecules. As indicated above, one or more of the nitrogen atoms of the copolymer may be protonated to result in a copolymer in a cationic form, typically an oligocationic or polycationic form. It will be understood that the primary, secondary, or tertiary amino groups in the repeating units (a) or (b) or in the terminal groups (c) can act as proton acceptors, especially in water and aqueous solutions, including physiological fluids. Thus, such copolymers typically have an overall positive charge in an aqueous solution at a pH of below 7.5. An aqueous solution, as referred to herein, is a solution wherein the solvent comprises 50% (vol./vol.) or more, preferably 80 or 90% or more, and most preferably 100% of water. Also, if the compositions are in contact with a physiological fluid having a pH of below 7.5, including e.g. blood and lung fluid, they typically contain repeating units (a) and (b) wherein the nitrogen atoms are protonated. The pK values of the copolymers used in the compositions can be determined by acid-base titration using an automated pK titrator. The net charge at a given pH value can then be calculated e.g. from the Henderson-Hasselbach equation. Any charge may be shared across several of the basic centres and cannot necessarily be attributed to a single point. Typically, in solutions at physiological pH, the copolymers used in the compositions comprise repeating units with amino groups in protonated state and repeating units with amino groups in unprotonated state. However, as will be understood by the skilled reader, the copolymers may also be provided as a dry salt form which contains the copolymer in a cationic form. will be further understood, counterions (anions) for the positive charges of protonated amino groups in compositions comprising the copolymer and nucleic acid, in particular mRNA, are typically provided by anionic moieties contained in the nucleic acid. If the positively charged groups are present in excess compared to the anionic moieties in the nucleic acid, positive charges may be balanced by other anions, in particular those typically encountered in physiological fluids, such as Cl or HCO₃⁻. In line with the above, a preferred copolymer is a random copolymer, wherein 80 mol % or more of all repeating units, more preferably all repeating units, are formed by a plurality of repeating units (a) independently selected from repeating units of the following formulae (a1) and (a2)

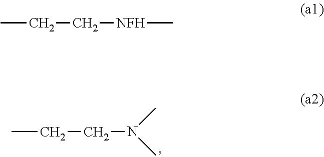

and a plurality of repeating units (b) independently selected from repeating units of the following formulae (b1) and (b2):

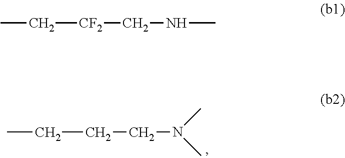

wherein the molar ratio of the sum of the repeating units (a) to the sum of the repeating units (b) lies within the range of 0.7/1.0 to 1.0/0.7, more preferably within the range of 0.8/1.0 to 1.0/0.8; 35 wherein the terminal groups of the copolymer are formed by groups (c) independently selected from groups of the formulae (c1) and (c2);
and wherein one or more of the nitrogen atoms of the repeating units (a) and/or (b) and/or of the terminal groups (c) contained in the copolymer may be protonated to provide a cationic copolymer. It is further preferred that the copolymer is a branched copolymer, comprising units (a2) and (b2), optionally together with units (a1) and/or (b1). Preparation of the copolymers is stated in EP 4 223 306 A2, which is incorporated in its entirety by reference herein. In principle, a lipidoid is a preferred nanoparticle to be employed, in particular as compared to an oligomer and, more particular, to a polymer.

The at least one disease associated antigen may be an antigenic protein which is encoded by an RNA (e.g. mRNA, modified or unmodified), wherein the RNA can be encapsulated in a lipid nanoparticle (LNP) for delivery to the phDCs of the invention. The encapsulated RNA (e.g. mRNA) is combined with the phDCs. The uptake of the RNA (e.g., mRNA, optionally modified mRNA) by phDCs may be monitored or assessed by means known in the art. These means include, but are not limited to, fluorescent LNPs, luminescent LNPs, radioactive LNPs or cascading reactions or detection of expressed antigenic proteins that are encoded by the RNA encapsulated in the LNPs. In one embodiment, the transfection efficacy of phDC, e.g., human phDCs, is assessed by co-incubating phDC generated from isolated PBMCs with mRNA encoding GPI-anchored nanoluciferase (NLuc) (LNP[NLuc]), and observing the luminescent signals. In some embodiments, the spatio-temporal distribution of LNPs is monitored.

### III. The Pharmaceutical Composition or Vaccine Composition

The present invention provides a pharmaceutical composition or vaccine composition, comprising phDCs and at least one RNA (e.g. mRNA), which comprises a coding sequence encoding for at least one antigenic protein. The terms pharmaceutical composition, vaccine and vaccine composition are used synonymously herein. The at least one mRNA may be in any form described under the first aspect and in the detailed description. The pharmaceutical composition of the invention in one embodiment, contains at least 1, at least 10, at least 100, at least 1000, at least 1×10⁴, at least 1×10⁵ or at least 1×10⁶ phDCs. The number of phDCs can e.g. be estimated from the volume of the whole blood, which became apheresed and flow chamber (e.g. plate or bag or hybrid) passed or flow chamber (e.g. plate or bag or hybrid) passed directly. The at least one mRNA can be provided encapsulated in nanoparticles (e.g. LNPs), with the pharmaceutical composition containing at least about 0.1 μg, 0.5 μg, 5 μg, 10 μg, 100 μg, 500 μg or 1000 μg of encapsulated RNA, in particular mRNA. The vaccine composition provides for a pharmaceutically effective amount of antigen-specific phDCs. In some embodiments, a vaccine composition according to the present invention contains at least 1, at least 10, at least 100, at least 1000, at least 1×10⁴, at least 1×10⁵ or at least 1×10⁶ antigen-specific phDCs. In terms of dose sparing, the dose of nanoparticles (e.g. LNPs) comprising RNA, in particular mRNA, can be reduced by at least 10%, at least 20%, at least 30%, at least 40%, at least 50%, at least 60%, at least 70%, at least 80% or at least 90% compared to the dose of nanoparticles (e.g. LNPs) used for a mRNA vaccine not comprising dendritic cells. A typical dose for an mRNA vaccine can be 0.1 to 100 μg, e.g., 1 μg, 5 μg, 10 μg, 30 μg or 50 μg. In one particular embodiment, the phDCs of the invention are combined with a maximum of 4.5 ug of nanoparticles (e.g. LNPs) comprising RNA, in particular mRNA.

The pharmaceutical composition or vaccine composition of the invention optionally comprises a pharmaceutically acceptable carrier and/or diluent. Additionally, the pharmaceutical composition or vaccine composition can comprise adjuvants and/or immuno-modulators to boost the activity of the pharmaceutical composition or vaccine composition and the subject's response. Such adjuvants and/or immuno-modulators are understood by those skilled in the art, and are readily described in available published literature. The pharmaceutical composition of the invention may contain one or more T cell activating agents. As contemplated herein, and depending on the type of vaccine being generated, the production of vaccine can, if desired, be scaled up by culturing cells in bioreactors or fermentors or other such vessels or devices suitable for the growing of cells in bulk. Other steps in vaccine preparation can be individualized to satisfy the requirements of particular vaccines. Such additional steps will be understood by those skilled in the art.

In all embodiments of the invention, the pharmaceutical composition or vaccine composition is used for the prevention of a disease, such as cancer and/or infectious diseases. The pharmaceutical composition or vaccine composition of the invention can induce antigen-specific T-cell and/or high titer antibody responses, thereby eliciting an immune response that is directed to or reactive against the disease (e.g. cancer or infectious disease) associated with the expression of the respective antigen. In some embodiments, the induced or elicited immune response can be a cellular, humoral, or both cellular and humoral immune response. In some embodiments, the induced or elicited cellular immune response includes induction or secretion of interferon-gamma (IFN-γ) and/or tumor necrosis factor alpha (TNF-α). In a particular embodiment, the pharmaceutical composition or vaccine composition acts by one or more of the following: (i) inducing humoral immunity via B cell responses to generate antibodies; (ii) increasing cytotoxic T lymphocytes such as CD8+(CTL) to attack and kill the disease causing particles which express the respective antigen; (iii) increasing T helper cell responses; (iv) increasing inflammatory responses via IFN-γ and/or TNF-α; (v) increasing natural killer cell responses; (vi) increasing central memory T cell and stem-like T cell subsets. In one embodiment, the pharmaceutical composition or vaccine composition acts by all of the aforementioned. In one embodiment, the pharmaceutical composition or vaccine composition acts at least by (ii), (v) and (vi). In one embodiment, the pharmaceutical composition or vaccine composition acts at least by (vi).

In other embodiments, the induced or elicited immune response can reduce or inhibit one or more immune suppression factors that promote the onset of the disease. In case of cancer, the induced or elicited immune response can reduce or inhibit one or more immune suppression factors that promote growth of the tumor or cancer expressing the antigen, for example, but not limited to, factors that downregulate MHC presentation, factors that upregulate antigen-specific regulatory T cells, PD-L1, FasL, cytokines such as IL-10 and TFG-β, tumor associated macrophages, tumor associated fibroblasts, soluble factors produced by immune suppressor cells, CTLA-4, PD-1, MDSCs, MCP-1 and an immune checkpoint molecule. The pharmaceutical composition or vaccine composition can be combined with other pharmaceutical products or compounds. The pharmaceutical composition or vaccine composition of the present invention can be used to prevent a disease such as a cancer or an infectious disease in individuals or it can be used for individuals who might be at risk of developing a certain disease such as cancer or acquiring an infection. The pharmaceutical composition or vaccine composition of the invention may be particularly useful for subjects with an impaired immune system.

In some embodiments, subjects vaccinated with the vaccine composition of the invention experience less adverse effects after vaccination compared to subjects vaccinated with vaccines not comprising dendritic cells, in particular mRNA vaccines (control subjects). Grading of adverse events can be performed, e.g., according to FDA recommendations (U.S. Department of Health and Human Services, Administration, F. and D. & Research, C. for B. E. and. Toxicity grading scale for healthy adult and adolescent volunteers enrolled in preventive vaccine clinical trials. (2007)). In one embodiment, subjects show less fatigue, pain, headache and/or fever than control subjects. For example, grade 3 fatigue, pain, headache or fever according to the FDA Recommendations is observed in less than 20%, less than 10%, less than 5% or 0% of subjects after vaccination (e.g. 7 or 14 days after vaccination) with the pharmaceutical composition or vaccine composition of the invention. Comparison can be made to a control group of subjects vaccinated with a vaccine not comprising DCs or phDCs, in particular an mRNA vaccine.

Depending on the vaccine target, the vaccine can be administered by different routes such as subcutaneous, intravenous, intramuscular, intra-articular, intra-synovial, intrasternal, intrathecal, intra-hepatic, intralesional, intracranial, transdermal, intradermal, intrapulmonal, intraperitoneal, intracardial, intraarterial, or sublingual. Preferred administration routes are intradermal or intramuscular. The composition can also be administered by inhalative administration. In one example, the vaccine composition of the invention is administered intradermally, subcutaneously or intramuscularly to the extremities, arms and legs, of the subjects being treated. In another example, the vaccine composition of the invention is administered intravenously. The vaccine composition of the invention can be administered as a prime and/or boost. In one embodiment, the vaccine composition of the invention is administered as a prime. In one embodiment, the vaccine composition of the invention is administered as a boost. In one embodiment, the vaccine composition of the invention is administered as a prime and a boost.

The present invention provides an article of manufacture, such as a kit, that provides phDCs in one container and the at least one disease associated antigen (e.g. the at least one mRNA encoding the at least one antigenic protein, optionally encapsulated in nanoparticles) in another container. The container may be pre-treated glass or plastic vials or ampules. The article of manufacture may include instructions for use.

### IV. Definitions

Where the term “comprising” is used in the present description and claims, it does not exclude other elements. For the purposes of the present invention, the term “consisting” is considered to be a preferred embodiment of the term “comprising”. If hereinafter a group is defined to comprise at least a certain number of embodiments, this is also to be understood to disclose a group, which preferably consists only of these embodiments.

For the purposes of the present invention, the term “obtained” is considered to be a preferred embodiment of the term “obtainable”. If hereinafter e.g. phDCs are defined to be obtainable by a specific method, this is also to be understood to disclose phDCs, which are obtained by this method.

Where an indefinite or definite article is used when referring to a singular noun, e.g. “a”, “an” or “the”, this includes a plural of that noun unless something else is specifically stated. The term “or” means, and is used interchangeably with, the term “and/or,” unless context clearly indicates otherwise. The terms “about” or “approximately” in the context of the present invention denote an interval of accuracy that the person skilled in the art will understand to still ensure the technical effect of the feature in question. The term typically indicates deviation from the indicated numerical value of ±20%, preferably ±15%, more preferably ±10%, and even more preferably ±5%.

Furthermore, the terms “first”, “second”, “third” or “(a)”, “(b)”, “(c)”, “(d)” or “(i)”, “(ii)”, “(iii)”, “(iv)” etc. and the like in the description and in the claims, are used for distinguishing between similar elements and not necessarily for describing a sequential or chronological order. It is to be understood that the terms so used are interchangeable under appropriate circumstances and that the embodiments of the invention described herein are capable of operation in other sequences than described or illustrated herein. In case the terms “first”, “second”, “third” or “(a)”, “(b)”, “(c)”, “(d)” or “(i)”, “(ii)”, “(iii)”, “(iv)” etc. relate to steps of a method or use or assay there is no time or time interval coherence between the steps unless indicated otherwise, i.e. the steps may be carried out simultaneously or there may be time intervals of seconds, minutes, hours, days, weeks, months or even years between such steps, unless otherwise indicated in the application as set forth herein above or below.

The term “about” means a quantity, level, value, number, frequency, percentage, dimension, size, amount, weight, or length that varies by as much as 30, 25, 20, 15, 30 10, 9, 8, 7, 6, 5, 4, 3, 2 or 1% to a reference quantity, level, value, number, frequency, percentage, dimension, size, amount, weight or length. When the term “about” is used in conjunction with a numerical range, it modifies that range by extending the boundaries above and below the numerical values set forth. The term “about” in the context of the present invention denotes an interval of accuracy that the person skilled in the art will understand to still ensure the technical effect of the feature in question. In general, the term “about” is intended to modify a numerical value above and below the stated value by a variance of ±10%.

Technical terms are used by their common sense. If a specific meaning is conveyed to certain terms, definitions of terms will be given in the following in the context of which the terms are used.

### Nucleic Acids and Proteins

The term “polynucleotide” or “nucleic acid”, as used herein, is intended to include DNA and RNA such as genomic DNA, cDNA, mRNA, recombinantly produced and chemically synthesized molecules. A nucleic acid may be single-stranded or double-stranded. RNA includes in vitro transcribed RNA or synthetic RNA. In one embodiment of all aspects of the invention, the RNA encoding the antigenic protein is expressed in phDCs obtained from monocytes of a donor or the subject to be vaccinated. The loaded phDCs, i.e. phDCs comprising the RNA encoding the antigenic protein, present the antigenic protein to the immune system of the subject.

The nucleic acids described herein may be recombinant and/or isolated molecules. In the present disclosure, the term “RNA” relates to a nucleic acid molecule, which includes ribonucleotide residues. In preferred embodiments, the RNA contains all or a majority of ribonucleotide residues. As used herein, “ribonucleotide” refers to a nucleotide with a hydroxyl group at the 2′-position of a β-D-ribofuranosyl group. RNA encompasses without limitation, double stranded RNA, single stranded RNA, isolated RNA such as partially purified RNA, essentially pure RNA, mRNA, circular RNA, synthetic RNA, recombinantly produced RNA, synthetic RNA, self-amplifying RNA as well as modified RNA (e.g. modified mRNA) that differs from naturally occurring RNA by the addition, deletion, substitution and/or alteration of one or more nucleotides. The RNA species above, e.g. “RNA”, “mRNA”, “self-amplifying RNA”, “modified mRNA” or “synthetic RNA”, always comprise fragments thereof, which are still functional. “Fragment”, can relate to a part of a nucleotide sequence, i.e. a sequence, which is shortened at the 5′ or 3′ end. A fragment of an RNA (e.g. mRNA) sequence comprises e.g. at least 50%, at least 60%, at least 70%, at least 80%, at least 90% of the residues from an RNA sequence. A fragment of an RNA (e.g. mRNA) sequence preferably comprises at least 18, in particular at least 24, at least 36, at least 45, at least 60, at least 90, at least 150, or at least 300 consecutive residues from an RNA sequence.

Alterations may refer to addition of nonnucleotide material to internal RNA nucleotides or to the end(s) of RNA. It is also contemplated herein that nucleotides in RNA (e.g. mRNA) may be non-standard nucleotides, such as chemically synthesized nucleotides, naturally occurring modified nucleotides or deoxynucleotides. For the present disclosure, these altered RNAs are considered analogs of naturally-occurring RNA.

In certain embodiments of the present invention, the RNA is messenger RNA (mRNA) that relates to a RNA transcript, which encodes a peptide or protein. As established in the art, mRNA generally contains a 5′ untranslated region (5′-UTR), a coding region and a 3′ untranslated region (3′-UTR). In some embodiments, the RNA (e.g. mRNA) is produced by in vitro transcription or chemical synthesis. In one embodiment, the mRNA is produced by in vitro transcription using a DNA template where DNA refers to a nucleic acid that contains deoxyribonucleotides. In one embodiment, RNA (e.g. mRNA) is in vitro transcribed RNA (IVT-RNA) and may be obtained by in vitro transcription of an appropriate DNA template. The promoter for controlling transcription can be any promoter for any RNA polymerase.

A DNA template for in vitro transcription may be obtained by cloning of a nucleic acid, in particular cDNA, and introducing it into an appropriate vector for in vitro transcription. The cDNA may be obtained by reverse transcription of RNA.

In one embodiment, the RNA, in particular mRNA, described herein may have modified nucleosides. In some embodiments, the RNA, in particular mRNA, comprises a modified nucleoside in place of at least one (e.g., every) uridine. “Pseudouridine” is one example of a modified nucleoside that is an isomer of uridine, where the uracil is attached to the pentose ring via a carbon-carbon bond instead of a nitrogen carbon glycosidic bond. Another exemplary modified nucleoside is N1-methyl-pseudouridine. Another exemplary modified nucleoside is 5-methyl-uridine. In some embodiments, one or more uridine residues in the RNA (e.g. mRNA) described herein is replaced by a modified uridine residues, e.g., N1-methyl-pseudouridine or 5-methyl-uridine, or a combination thereof. In one embodiment, the RNA comprises other modified nucleosides or comprises further modified nucleosides, e.g., modified cytidine. For example, in one embodiment, in the RNA cytidine is substituted partially or completely, preferably completely, for 5-methyl-cytidine. In one embodiment, the RNA comprises 5-methylcytidine and one or more selected from pseudouridine, N1-methyl-pseudouridine, and 5-methyl-uridine. In one embodiment, the RNA comprises 5-methylcytidine and N1-methyl-pseudouridine. In some embodiments, the RNA comprises 5-methylcytidine in place of each cytidine and N1-methyl-pseudouridine in place of each uridine. In some embodiments, RNA (e.g. mRNA) may comprise more than one type of modified nucleoside. In some embodiments, only a subset of uridine residues is modified. In some embodiments, all uridine residues are modified.

In some embodiments, the modified nucleoside is a modified uridine. Exemplary nucleobases and nucleosides having a modified uracil include pseudouridine (W), pyridin-4-one ribonucleoside, 5-aza-uridine, 6-aza-uridine, 2-thio-5-aza-uridine, 2-thio-uridine (s2U), 4-thio-uridine (s4U), 4-thio-pseudouridine, 2-thio-pseudouridine, 5-hydroxy-uridine (ho5U), 5-aminoallyl-uridine, 5-halo-uridine (e.g., 5-iodo-uridine or 5-bromo-uridine), 3-methyl-uridine (m3U), 5-methoxy-uridine (mo5U), uridine 5-oxyacetic acid (cmo5U), uridine 5-oxyacetic acid methyl ester (mcmo5U), 5-carboxymethyl-uridine (cm5U), 1-carboxymethyl-pseudouridine, 5-carboxyhydroxymethyl-uridine (chm5U), 5-carboxyhydroxymethyl-uridinemethyl ester (mchm5U), 5-methoxycarbonylmethyl-uridine (mcm5U), 5-methoxycarbonylmethyl-2-thio-uridine (mcm5s2U), 5-aminomethyl-2-thio-uridine (nm5s2U), 5-methylaminomethyl-uridine (mnm5U), 5-methylaminomethyl-2-thio-uridine (mnm5s2U), 5-methylaminomethyl-2-seleno-uridine (mnm5se2U), 5-carbamoylmethyl-uridine (ncm5U), 5-carboxymethylaminomethyl-uridine (cmnm5U), 5-carboxymethylaminomethyl-2-thio-uridine (cmnm5s2U), 5-propynyl-uridine, 1-propynyl-pseudouridine, 5-taurinomethyl-uridine (rm5U), 1-taurinomethyl-pseudouridine, 5-taurinomethyl-2-thio-uridine (rm5s2U), 1-taurinomethyl-4-thio-pseudouridine, 5-methyl-uridine (m5U, i.e., having the nucleobase deoxythymine), 1-methyl-pseudouridine (m1ψ), 5-methyl-2-thio-uridine (m5s2U), 1-methyl-4-thio-pseudouridine (m1s4ψ), 4-thio-1-methyl-pseudouridine, 3-methyl-pseudouridine (m3ψ), 2-thio-1-methyl-pseudouridine, 1-methyl-1-deaza-pseudouridine, 2-thio-1-methyl-1-deaza-pseudouridine, dihydrouridine (D), dihydropseudouridine, 5,6-dihydrouridine, 5-methyl-dihydrouridine (m5D), 2-thio-dihydrouridine, 2-thio-dihydropseudouridine, 2-methoxy-uridine, 2-methoxy-4-thio-uridine, 4-methoxy-pseudouridine, 4-methoxy-2-thio-pseudouridine, N1-methyl-pseudouridine, 3-(3-amino-3-carboxypropyl)uridine (acp3U), 1-methyl-3-(3-amino-3-carboxypropyl) pseudouridine (acp3Ψ), 5-(isopentenylaminomethyl)uridine(inm5U), 5-(isopentenylaminomethyl)-2-thiouridine (inm5S2U), α-thio-uridine, 2′-O-methyl-uridine (Um), 5,2′-O-dimethyl-uridine (m5Um), 2′-O-methyl-pseudouridine (ψm), 2-thio-2′-O-methyl-uridine (s2Um), 5-methoxycarbonylmethyl-2′-O-methyl-uridine (mcm5Um), 5-carbamoylmethyl-2′-O-methyl-uridine (ncm5Um), 5-carboxymethylaminomethyl-2′-O-methyl-uridine (cmnm5Um), 3,2′-O-dimethyl-uridine (m3Um), and 5-(isopentenylaminomethyl)-2′-O-methyl-uridine (inm5Um), 1-thio-uridine, deoxythymidine, 2′-F-ara-uridine, 2′-F-uridine, 2′-OH-ara-uridine, 5-(2-carbomethoxyvinyl) uridine, and 5-[3-(1-E-propenylamino)]uridine.

In some embodiments, the modified nucleoside is a modified cytidine. Exemplary nucleobases and nucleosides having a modified cytosine include 5-aza-cytidine, 6-aza-cytidine, pseudoisocytidine, 3-methyl-cytidine (m3C), N4-acetyl-cytidine (ac4C), 5-formyl-cytidine (f5C), N4-methyl-cytidine (m4C), 5-methyl-cytidine (m5C), 5-halo-cytidine (e.g., 5-iodo-cytidine), 5-hydroxymethyl-cytidine (hm5C), 1-methyl-pseudoisocytidine, pyrrolo-cytidine, pyrrolo-pseudoisocytidine, 2-thio-cytidine (s2C), 2-thio-5-methyl-cytidine, 4-thio-pseudoisocytidine, 4-thio-1-methyl-pseudoisocytidine, 4-thio-1-me-thyl-1-deaza-pseudoisocytidine, 1-methyl-1-deaza-pseudoisocytidine, zebularine, 5-aza-zebularine, 5-methyl-zebular-ine, 5-aza-2-thio-zebularine, 2-thio-zebularine, 2-methoxy-cytidine, 2-methoxy-5-methyl-cytidine, 4-methoxy-pseudoi-socytidine, 4-methoxy-1-methyl-pseudoisocytidine, lysidine (k2C), α-thio-cytidine, 2′-O-methyl-cytidine (Cm), 5,2′-O-dimethyl-cytidine (m5Cm), N4-acetyl-2′-O-methyl-cytidine (ac4Cm), N4,2′-O-dimethyl-cytidine (m4Cm), 5-formyl-2′O-me-thyl-cytidine (f5Cm), N4,N4,2′-O-trimethyl-cytidine (m42Cm), 1-thio-cytidine, 2′-F-afa-cytidine, 2′-F-cytidine, and 2′-OH-afa-cytidine.

In some embodiments, the modified nucleoside is a modified adenosine. Exemplary nucleobases and nucleosides having a modified adenine include 2-amino-purine, 2,6-diaminopurine, 2-amino-6-halo-purine (e.g., 2-amino-6-chloro-purine), 6-halo-purine (e.g., 6-chloro-purine), 2-amino-6-methyl-purine, 8-azido-adenosine, 7-deaza-adenine, 7-deaza-8-aza-adenine, 7-deaza-2-amino-purine, 7-deaza-8-aza-2-amino-purine, 7-deaza-2,6-diaminopurine, 7-deaza-8-aza-2,6-diaminopurine, 1-methyl-adenosine (m1A), 2-methyl-adenine (m2A), N6-methyl-adenosine (m6A), 2-methylthio-N6-methyl-adenosine (ms2m6A), N6-isopentenyl-adenosine (i6A), 2-methylthio-N6-isopentenyl-adenosine (ms2i6A), N6-(cis-hydroxyisopentenyl) adenosine (io6A), 2-methylthio-N6-(cis-hydroxyisopentenyl)adenosine (ms2io6A), N6-glyci-nylcarbamoyl-adenosine (g6A), N6-threonylcarbamoyl-adenosine (t6A), N6-methyl-N6-threonylcarbamoyl-adenosine (m6t6A), 2-methylthio-N6-threonylcarbamoyl-adenosine (ms2g6A), N6,N6-dimethyl-adenosine (m62A), N6-hydroxynor-valylcarbamoyl-adenosine (hn6A), 2-methylthio-N6-hydroxynorvalyl-carbamoyl-adenosine (ms2hn6A), N6-acetyl-adeno-sine (ac6A), 7-methyl-adenine, 2-methythio-adenine, 2-methoxy-adenine, α-thio-adenosine, 2′-O-methyl-adenosine (Am), N6,2′-O-dimethyl-adenosine (m6Am), N6,N6,2′-O-trimethyl-adenosine (n62Am), 1,2′-O-dimethyl-adenosine (m1Am), 2′-O-ribosyladenosine (phosphate) (Ar(p)), 2-amino-N6-methyl-purine, 1-thio-adenosine, 8-azido-adenosine, 2′-F-aa-adenosine, 2′-F-adenosine, 2′-OH-ara-adenosine, and N6-(19-amino-pentaoxa-nonadecyl)-adenosine.

In some embodiments, the modified nucleoside is a modified guanidine. Exemplary nucleobases and nucleosides having a modified guanine include inosine (I), 1-methyl-inosine (m11), wyosine (imG), methylmyosine (mimG), 4-demethyl-wyosine (imG-14), isowyosine (imG2), wybutosine (yW), peroxywybutosine (02yW), hydroxywybutosine (OhyW), under-modified hydroxywybutosine (OhyW*), 7-deaza-guanosine, queuosine (Q), epoxyqueuosine (oQ), galactosyl-queuosine(galQ), mannosyl-queuosine (manQ), 7-cyano-7-deaza-guanosine (preQo), 7-aninomethyl-7-deaza-guanosine (preQl), archaeosine (G+), 7-deaza-8-aza-guanosine, 6-thio-guanosine, 6-thio-7-deaza-guanosine, 6-thio-7-deaza-8-a za-gua-nosine, 7-methyl-guanosine (m7G), 6-thio-7-methyl-guanosine, 7-methyl-inosine, 6-methoxy-guanosine, 1-methyl-guanosine (m1G), N2-methyl-guanosine(m2G), N2,N2-dimethyl-guano sine (m22G), N2,7-dimethyl-guanosine (m2,7G),N2, N2,7-dimethyl-guano sine (m2,2,7G), 8-oxo-guanosine, 7-methyl-8-oxo-guanosine, 1-methyl-6-thio-guanosine, N2-methyl-6-thio-guanosine, N2,N2-dimethyl-6-thio-guanosine, α-thio-guanosine, 2′-O-methyl-guanosine (Gm), N2-methyl-2′-O-methyl-guanosine (m2Gm), N2,N2-dimethyl-2′-O-methyl-guanosine (m22Gm), 1-methyl-2′-O-methyl-guanosine (m1Gm), N2,7-dimethyl-2′-O-methyl-guanosine (m2,7Gm), 2′-O-methyl-inosine (Im), 1,2′-O-dimethyl-inosine (m1lm), 2′-O-ribosylguanosine (phosphate) (Gr(p)), 1-thio-guanosine, 06-methyl-guanosine, 2′-F-ara-guanosine, and 2′-F-guano-sine.

In some embodiments, the mRNA comprises a 5′-cap. In one embodiment, the mRNA does not have uncapped 5′-triphosphates. In one embodiment, the mRNA may be modified by a 5′-cap analog. The term “5′-cap” refers to a structure found on the 5′-end of an mRNA molecule and generally consists of a guanosine nucleotide connected to the mRNA via a 5′- to 5′-triphosphate linkage. In one embodiment, this guanosine is methylated at the 7-position. Providing an mRNA with a 5′-cap or 5′-cap analog may be achieved by in vitro transcription, in which the 5′-cap is co-transcriptionally expressed into the mRNA strand, or may be attached to mRNA post-transcriptionally using capping enzymes.

The term “untranslated region” or “UTR” relates to a region in a DNA molecule which is transcribed but is not translated into an amino acid sequence, or to the corresponding region in an RNA molecule, such as an mRNA molecule. An untranslated region (UTR) can be present 5′ (upstream) of an open reading frame (5′-UTR) and/or 3′ (downstream) of an open reading frame (3′-UTR). A 5′-UTR, if present, is located at the 5′ end, upstream of the start codon of a protein-encoding region. A 5′-UTR is downstream of the 5′-cap (if present), e.g. directly adjacent to the 5′-cap. A 3′-UTR, if present, is located at the 3′ end, downstream of the termination codon of a protein-encoding region, but the term “3′-UTR” does preferably not include the poly(A) sequence. Thus, the 3′-UTR is upstream of the poly(A) sequence (if present), e.g. directly adjacent to the poly(A) sequence.

As used herein, the term “poly(A) sequence” or “poly-A tail” refers to an uninterrupted or interrupted sequence of adenylate residues which is typically located at the 3′-end of an RNA molecule (e.g. an mRNA molecule). Poly(A) sequences are known to those of skill in the art and may follow the 3′-UTR in the RNAs (e.g. mRNAs) described herein. An uninterrupted poly(A) sequence is characterized by consecutive adenylate residues. In nature, an uninterrupted poly(A) sequence is typical. RNAs (e.g. mRNAs) according to the invention can have a poly(A) sequence attached to the free 3′-end of the RNA (e.g. mRNA) by a template-independent RNA polymerase after transcription or a poly(A) sequence encoded by DNA and transcribed by a template-dependent RNA polymerase. Poly(A) sequences of about 120 A nucleotides have a beneficial influence on the RNA-levels in transfected eukaryotic cells, as well as on the protein-levels, wherein the protein is translated from an open reading frame that is present upstream (5′) of the poly(A) sequence (Holtkamp et al., 2006, Blood, vol. 108, pp. 4009-4017). The poly(A) sequence may be of any length. In some embodiments, a poly(A) sequence comprises, essentially consists of, or consists of at least 20, at least 30, at least 40, at least 80, or at least 100 and up to 800, up to 400, up to 300, up to 200, or up to 150 A nucleotides. In one embodiment, the poly(A) sequence comprises, essentially consists of, or consists of 300 nucleotides. In this context, “essentially consists of” means that most nucleotides in the poly(A) sequence, typically at least 75%, at least 80%, at least 85%, at least 90%, at least 95%, at least 96%, at least 97%, at least 98%, or at least 99% by number of nucleotides in the poly(A) sequence are A nucleotides, but permits that remaining nucleotides are nucleotides other than A nucleotides, such as U nucleotides (uridylate), G nucleotides (guanylate), or C nucleotides (cytidylate). In this context, “consists of” means that all nucleotides in the poly(A) sequence, i.e., 100% by number of nucleotides in the poly(A) sequence, are A nucleotides. The term “A nucleotide” or “A” refers to adenylate. In some embodiments, a poly(A) sequence is attached during RNA transcription, e.g., during preparation of in vitro transcribed RNA (e.g. mRNA), based on a DNA template comprising repeated dT nucleotides (deoxythymidylate) in the strand complementary to the coding strand. The DNA sequence encoding a poly(A) sequence (coding strand) is referred to as poly(A) cassette.

The term “codon-optimized” refers to the alteration of codons in the coding region of a nucleic acid molecule to reflect the typical codon usage of a host organism without preferably altering the amino acid sequence encoded by the nucleic acid molecule. Within the context of the present invention, coding regions are preferably codon-optimized for optimal expression in a subject to be treated using the RNA (e.g. mRNA) molecules described herein. Codon-optimization is based on the finding that the translation efficiency is also determined by a different frequency in the occurrence of tRNAs in cells. Thus, the sequence of RNA (e.g. mRNA) may be modified such that codons for which frequently occurring tRNAs are available are inserted in place of “rare codons”.

In some embodiments of the invention, the guanosine/cytosine (G/C) content of the coding region of the RNA (e.g. mRNA) described herein is increased compared to the G/C content of the corresponding coding sequence of the wild type RNA, wherein the amino acid sequence encoded by the RNA (e.g. mRNA) is preferably not modified compared to the amino acid sequence encoded by the wild type RNA. This modification of the RNA (e.g. mRNA) sequence is based on the fact that the sequence of any RNA region to be translated is important for efficient translation of that RNA (e.g. mRNA). Sequences having an increased G/C content are more stable than sequences having an increased A (adenosine)/U (uracil) content. In respect to the fact that several codons code for one and the same amino acid (so-called degeneration of the genetic code), the most favourable codons for the stability can be determined (so-called alternative codon usage). Depending on the amino acid to be encoded by the RNA, there are various possibilities for modification of the RNA (e.g. mRNA) sequence, compared to its wild type sequence. In particular, codons which contain A and/or U nucleotides can be modified by substituting these codons by other codons, which code for the same amino acids but contain no A and/or U or contain a lower content of A and/or U nucleotides.

In the context of the present invention, the term “transcription” relates to a process, wherein the genetic code in a DNA sequence is transcribed into RNA (e.g. mRNA). Subsequently, the RNA (e.g. mRNA) may be translated into peptide or protein.

According to the present invention, the term “transcription” comprises “in vitro transcription”, wherein the term “in vitro transcription” relates to a process wherein RNA, in particular mRNA, is in vitro synthesized in a cell-free system, preferably using appropriate cell extracts. Preferably, cloning vectors are applied for the generation of transcripts. These cloning vectors are generally designated as transcription vectors and are according to the present invention encompassed by the term “vector”.

With respect to RNA (e.g. mRNA), the term “expression” or “translation” relates to the process in the ribosomes of a cell by which a strand of mRNA directs the assembly of a sequence of amino acids to make a peptide or protein. In one embodiment, after combination of the RNA (e.g. mRNA) described herein, e.g., formulated as RNA (e.g. mRNA) lipid particles, with phDCs, at least a portion of the RNA (e.g. mRNA) is delivered to the phDCs. In one embodiment, the RNA (e.g. mRNA) is translated by the phDCs to produce the peptide or protein it encodes. RNA (e.g. mRNA) particles such as RNA (e.g. mRNA) lipid particles described herein may be used for delivering RNA to phDCs.

“Encoding” refers to the inherent property of specific sequences of nucleotides in a polynucleotide, such as a gene, a cDNA, RNA or an mRNA, to serve as templates for synthesis of other polymers and macromolecules in biological processes having either a defined sequence of nucleotides (i.e., rRNA, tRNA and mRNA) or a defined sequence of amino acids and the biological properties resulting therefrom. Thus, a RNA (e.g. mRNA) sequence may encode a protein (e.g. antigen) if translation of the RNA (e.g. mRNA) occurs in a cell.

Terms such as “reduce”, “effectively reduce”, “decrease”, “inhibit” or “impair” as used herein relate to an overall reduction or the ability to cause an overall reduction, preferably of at least 5%, at least 10%, at least 20%, at least 50%, at least 75% or even more, in the level. These terms include a complete or essentially complete inhibition, i.e., a reduction to zero or essentially to zero. Terms such as “increase”, “enhance” or “exceed” preferably relate to an increase or enhancement by at least 10%, at least 20%, at least 30%, at least 40%, at least 50%, at least 80%, at least 100%, at least 200%, at least 500%, or even more.

According to the disclosure, the term “peptide” comprises oligo- and polypeptides and refers to substances which comprise about two or more, about 3 or more, about 4 or more, about 6 or more, about 8 or more, about 10 or more, about 13 or more, about 16 or more, about 20 or more, and up to about 50, about 100 or about 150, consecutive amino acids linked to one another via peptide bonds. The term “protein” or “polypeptide” refers to large peptides, in particular peptides having at least about 150 amino acids, but the terms “peptide”, “protein” and “polypeptide” are used herein as synonyms if not mentioned otherwise.

“Fragment”, can relate to a part of an antigen such as a disease associated antigen, an amino acid sequence, an antigenic protein, an RNA (including the RNA species listed above) and in particular mRNA.

If fragment relates to an amino acid sequence (e.g. antigenic protein), the term refers to a part of an amino acid sequence, i.e. a sequence which represents the amino acid sequence (e.g. of the antigenic protein) shortened at the N-terminus and/or C-terminus. A fragment shortened at the C-terminus (N-terminal fragment) is obtainable e.g. by translation of a truncated open reading frame that lacks the 3′-end of the open reading frame. A fragment shortened at the N-terminus (C-terminal fragment) is obtainable e.g. by translation of a truncated open reading frame that lacks the 5′-end of the open reading frame, as long as the truncated open reading frame comprises a start codon that serves to initiate translation. A fragment of an amino acid sequence comprises e.g. at least 50%, at least 60%, at least 70%, at least 80%, at least 90% of the amino acid residues from an amino acid sequence. A fragment of an amino acid sequence preferably comprises at least 6, in particular at least 8, at least 12, at least 15, at least 20, at least 30, at least 50, or at least 100 consecutive amino acids from an amino acid sequence.

By “variant” herein is meant an amino acid sequence that differs from a parent amino acid sequence by virtue of at least one amino acid modification. The parent amino acid sequence may be a naturally occurring or wild type (WT) amino acid sequence, or may be a modified version of a wild type amino acid sequence. Preferably, the variant amino acid sequence has at least one amino acid modification compared to the parent amino acid sequence, e.g., from 1 to about 20 amino acid modifications, from 1 to about 10 or from 1 to about 5 amino acid modifications compared to the parent.

By “wild type” or “WT” or “native” herein is meant an amino acid sequence that is found in nature, including allelic variations. A wild type amino acid sequence, peptide or protein has an amino acid sequence that has not been intentionally modified.

“Variants” of an amino acid sequence (peptide, protein or polypeptide) comprise amino acid insertion variants, amino acid addition variants, amino acid deletion variants and/or amino acid substitution variants. The term “variant” includes all mutants, splice variants, posttranslationally modified variants, conformations, isoforms, allelic variants, species variants, and species homologs, in particular those which are naturally occurring. The term “variant” includes, in particular, fragments of an amino acid sequence. Preferably the degree of similarity (or identity) between a given amino acid sequence and an amino acid sequence which is a variant of said given amino acid sequence will be at least about 60%, 70%, 80%, 81%, 82%, 83%, 84%, 85%, 86%, 87%, 88%, 89%, 90%, 91%, 92%, 93%, 94%, 95%, 96%, 97%, 98%, or 99%. The alignment for determining sequence similarity, preferably sequence identity can be done with art known tools, preferably using the best sequence alignment, for example, using Align, using standard settings, preferably EM-BOSS::needle, Matrix: Blosum62, and the like. “Sequence similarity” indicates the percentage of amino acids that either are identical or that represent conservative amino acid substitutions.

“Sequence identity” between two amino acid sequences indicates the percentage of amino acids that are identical between the sequences. “Sequence identity” between two nucleic acid sequences indicates the percentage of nucleotides that are identical between the sequences. Percentage identity is obtained by determining the number of identical positions at which the sequences to be compared correspond, dividing this number by the number of positions compared (e.g., the number of positions in the reference sequence) and multiplying this result by 100.

In one embodiment, a fragment or “variant” is preferably a “functional fragment” or “functional variant”. The term “functional fragment” or “functional variant” relates to any fragment or variant exhibiting one or more functional properties identical or similar to those of the unfragmented agent (e.g. unfragmented amino acid sequence, unfragmented RNA sequence, unfragmented mRNA sequence, etc.) from which it is derived, i.e., it is functionally equivalent. With respect to antigens or antigenic proteins, one particular function is one or more immunogenic (“antigenic” synonymous herein) activities displayed by the amino acid sequence from which the fragment or variant is derived. The term “functional fragment” or “functional variant”, as used herein, in particular refers to a variant molecule or sequence that comprises a sequence that is altered by one or more amino acids or nucleotides compared to the parent molecule or sequence and that is still capable of fulfilling one or more of the functions of the parent molecule or sequence, e.g., inducing an immune response (i.e. being antigenic). In one embodiment, the modifications in the amino acid sequence of the parent molecule or sequence do not significantly affect or alter the characteristics of the molecule or sequence. In different embodiments, the function of the functional fragment or functional variant may be reduced but still significantly present, e.g., immunogenicity of the functional fragment or variant may be at least 50%, at least 60%, at least 70%, at least 80%, or at least 90% of the parent agent, molecule or sequence. However, in other embodiments, immunogenicity of the functional fragment or functional variant may be enhanced compared to the parent molecule or sequence.

### Composition and Administration

As used herein, the term “vaccine” refers to a composition that induces an immune response upon inoculation into a subject. In some embodiments, the induced immune response provides protective immunity. Different types of vaccines inter alia include live, attenuated vaccines, inactivated vaccines, toxoid vaccines or subunit and conjugate vaccines (also referred to as conventional vaccines) and mRNA vaccines. Conventional vaccines such mRNA vaccines within the meaning herein do not include DCs, phDCs in particular.

For the vaccine composition of the invention to be useful as a vaccine, an immune response against the disease associated antigen (e.g. antigenic protein) must be induced in a cell, tissue or subject (e.g., a human). In some embodiments, the vaccine composition of the invention induces an immune response against the at least one disease associated antigen (e.g. antigenic protein) in a cell, tissue or subject (e.g., a human). In some instances, the vaccine composition of the invention induces a protective immune response in a mammal. The vaccine composition of the invention is administered prophylactically (i.e., to prevent a disease or disorder or to alleviate the course of the disease or disorder. However, vaccination occurs prior to the initiation of a pathologic event or contact with an infectious agent, e.g. prior to infection with an infectious disease or prior to initiation of a tumor). The vaccine composition of the invention may be administered to a subject being at risk of (or susceptible to) developing a disease or disorder. Such subjects may be identified using standard clinical methods. In the context of the present invention, prophylactic administration occurs prior to the initiation of a pathologic event or contact with an infectious agent, such that a disease or disorder is prevented or alternatively delayed in its progression or alleviated in its symptoms. In the context of the field of medicine, the term “prevent” encompasses any activity, which reduces the burden of mortality or morbidity from disease. In some embodiments, administration of the vaccine composition of the invention may be performed by single administration or boosted by multiple administrations. If it is referred to administration as “initial” or “prime” herein, this corresponds to a first administration. Any subsequent administration is referred to as a “boost”. A booster administration is typically performed at least 14, at least 21, at least 28, at least 35, at least 42, at least 49 or at least 56 or more days after a prime administration.

The present invention contemplates an immune response that may be protective, preventive, and/or prophylactic. As used herein, “induces/stimulates (or inducing/stimulating) an immune response” may indicate that no immune response against a particular antigen was present before induction/stimulation or it may indicate that there was a basal level of immune response against a particular antigen before induction, which was enhanced after induction. Therefore, “induces/stimulates (or inducing/stimulating) an immune response” includes “enhances (or enhancing) an immune response”. The terms “immunization” or “vaccination” describe the process of administering an antigen to an individual with the purpose of stimulating an immune response for prophylactic reasons.

In contrast to vaccination, the term “treatment” or “therapeutic treatment” includes treatment of a subject (e.g. a mammal, such as a human) or a cell to alter the current course of the subject or cell. Thus, (therapeutic) treatment refers to a subject or cell, which is diagnosed, has been previously been diagnosed or which is determined to have the disease or disorder (as opposed to prevention). (Therapeutic) treatment is performed subsequent to the initiation of a pathologic event or contact with an infectious agent.

### Immunity

As used herein, the term “antigen” or “antigenic protein” refers to a compound, composition, or substance that can stimulate the production of antibodies or a T cell response in an animal, including compositions that are injected or absorbed into an animal. An antigen or antigenic protein reacts with the products of specific humoral or cellular immunity, including those induced by heterologous immunogens. The term antigen is used interchangeably with the term “immunogen”. The term “antigen” or “antigenic protein” include all related antigenic epitopes. The terms “antigen”, “antigenic molecule/protein” or “immunogen” include fragments thereof that are still capable of acting as an antigen. The term “antigenic protein” is used interchangeably with “immunogen” or “disease-associated antigen” throughout the present application. An “antigenic protein” comprises a number of amino acids ranging from small peptides to large proteins, e.g. 4 to 2000 amino acids or more, 4 to 1800 amino acids, 4 to 1600 amino acids or 4 to 1400 amino acids. In one embodiment, the antigenic protein, for which the RNA (e.g. mRNA) encodes, comprises at least 100, at least 200, at least 300, at least 400, at least 500, at least 600, at least 700, at least 800, at least 900, at least 1000, at least 1200, at least 2000 or at least 3000 amino acids. In one embodiment, the antigenic protein, for which the RNA (e.g. mRNA) encodes, comprises at least 50 amino acids. In one embodiment, the antigenic protein, for which the RNA (e.g. mRNA) encodes, comprises at least 500 amino acids. In one embodiment, the antigenic protein, for which the RNA (e.g. mRNA) encodes, comprises at least 1000 amino acids. In one embodiment, the antigenic protein comprises 100 to 1500 amino acids. In one embodiment, the antigenic protein comprises 200 to 1300 amino acids. In one embodiment, the antigenic protein comprises 400 to 1300 amino acids.

“Epitope” refers to a site on an antigen to which B and/or T cells respond.

As used herein, the term “immunogenicity” refers to the ability of a substance, a cell or a part thereof, such as an antigen or antigenic protein, to provoke an immune response in the body of a human or animal.

“Cell-mediated immunity”, “cellular immunity”, “cellular immune response”, or similar terms are meant to include a cellular response directed to cells characterized by expression of an antigen or antigenic protein, in particular characterized by presentation of an antigen with class I or class II MHC. The cellular response relates to immune effector cells, in particular to cells called T cells or T lymphocytes which act as either “helpers” or “killers”. The helper T cells (also termed CD4+ T cells) play a central role by regulating the immune response and the killer cells (also termed cytotoxic T cells, cytolytic T cells, CD8+ T cells or CTLs) kill diseased cells such as virus-infected cells, preventing the production of more diseased cells.

“Humoral immunity” or “humoral immune response” is the aspect of immunity that is mediated by macromolecules found in extracellular fluids such as secreted antibodies, complement proteins, and certain antimicrobial peptides. It contrasts with cell-mediated immunity. Its aspects involving antibodies are often called antibody-mediated immunity. Humoral immunity refers to antibody production and the accessory processes that accompany it, including: Th2 activation and cytokine production, germinal center formation and isotype switching, affinity maturation and memory cell generation.

“Dendritic cells,” also referred to herein as “DCs,” are antigen-presenting immune cells that process antigenic material and present it to other cells of the immune system, most notably to T cells. DCs function to capture and process antigens. When DCs endocytose antigens, they process the antigens into smaller fragments, generally peptides, that are displayed on the DC surface, where they are presented to, for example, antigen-specific T cells through MHC molecules. After uptake of antigens, DCs migrate to the lymph nodes. During maturation, DCs can be prompted by various signals, including signaling through Toll-like receptors (TLR), to express co-stimulatory signals that induce cognate effector T cells (Teff) to become activated and to proliferate, thereby initiating a T-cell mediated immune response to the antigen. Alternatively, DCs can present an antigen to antigen-specific T cells without providing co-stimulatory signals (or while providing co-inhibitory signals), such that Teff are not properly activated. Such presentation can cause, for example, death or anergy of T cells recognizing the antigen, or can induce the generation and/or expansion of regulatory T cells (Treg). The term “dendritic cells” includes differentiated dendritic cells, immature, and mature dendritic cells. These cells can be characterized by expression of certain cell surface markers (e.g., CD11c, MHC class II, and at least low levels of CD80 and CD86), CD11b, CD304 (BDCA4)). In some embodiments, DCs express CD8, CD103, CD1d, etc. Other DCs can be identified by the absence of lineage markers such as CD3, CD14, CD19, CD56, etc. In addition, dendritic cells can be characterized functionally by their capacity to stimulate alloresponses and mixed lymphocyte reactions (MLR). In one example, phDCs that are combined with a SARS-CoV-2 derived spike protein antigen (antigenic protein) or an mRNA encoding therefore, will process and present spike protein associated antigens, i.e. the phDCs will be antigen-specific to the spike protein.

The term “AB serum” or “human AB serum” relates to a well known in the art cell culture reagent for some human cell types providing growth factors, vitamins, nutrients as well as trace elements and transport factors. Human AB serum is collected from healthy volunteer male donors of the AB serotype.

The term “FBS” relates to a widely-used growth supplement for cell culture media. It typically has a high content of embryonic growth-promoting factors.

### Diseases

The terms “disease” or “disorder” refer to any disease or disorder which implicates an antigen or antigenic protein, e.g. a disease which is characterized by the presence of an antigen or antigenic protein. The disease can, e.g., be an infectious disease or a tumor disease (cancer). As mentioned above, the antigen may be a disease-associated antigen, such as a viral antigen or a tumor antigen. In one embodiment, a disease involving an antigen is a disease involving cells expressing an antigen, preferably on the cell surface. In the context of the present invention, diseases are caused by disease causing particles. The concentration of disease causing particles in a subject can be determined by any suitable means known to the skilled person. For example, the viral load may be determined by quantitative PCR and expressed as copy number per ng tissue. If suitable, expression markers may also be used for the quantification of disease causing particles.

The term “hyper-proliferative diseases” encompasses tumors and cancer. The term “tumor” refers to a disease in which some of the body's cells grow uncontrollably and spread to other parts of the body. Tumors can be cancerous or not cancerous (benign). In the context of the present invention, the terms tumor and cancer are used interchangeably. Cancerous tumors spread into, or invade, nearby tissues and can travel to distant places in the body to form new tumors (also called metastasis). Cancerous tumors may also be called malignant tumors. Many cancers form solid tumors, but cancers of the blood, such as leukemias, do not. Benign tumors do not spread into, or invade, nearby tissues. Blood cancer includes leukemia, lymphoma, myelodysplastic syndromes (MDS), myeloproliferative disorder (MPD), multiple myeloma and all subtypes thereof.

The term “infectious disease” refers to any disease which can be transmitted from individual to individual or from organism to organism, and is caused by a microbial agent (e.g. common cold). Infectious diseases are known in the art and include, for example, a viral disease, a bacterial disease, a fungal disease, a prion disease or a parasitic disease, which diseases are caused by a virus, a bacterium, a fungus, a prion, and a parasite, respectively. In this regard, the infectious disease can be, for example, hepatitis, sexually transmitted diseases (e.g. chlamydia or gonorrhea), tuberculosis, HIV/acquired immune deficiency syndrome (AIDS), diphtheria, hepatitis B, hepatitis C, cholera, severe acute respiratory syndrome (SARS), Covid-19, the bird flu, and influenza.

“Corona disease 2019” or COVID-19 is a contagious disease caused by severe acute respiratory syndrome coronavirus-2 (SARS-CoV-2). The outbreak of SARS-CoV-2 that causes atypical pneumonia has raged in China since mid-December 2019, and has developed to be a public health emergency of international concern. SARS-CoV-2 (MN908947.3) belongs to betacoronavirus lineage B. It has at least 70% sequence similarity to SARS-CoV. In particular COVID-19 refers to a disease as defined in the current international classification of diseases (ICD-11, World Health Organisation, Version: 09/2020). More particularly, COVID-19 is used to denote the disease, diagnosed clinically, epidemiologically or otherwise, irrespective of whether laboratory testing is conclusive, inconclusive or not available. In general, coronaviruses have four structural proteins, namely, envelope (E), membrane (M), nucleocapsid (N), and spike (S). The E and M proteins have important functions in the viral assembly, and the N protein is necessary for viral RNA synthesis. The critical glycoprotein S is responsible for virus binding and entry into target cells. The S protein is synthesized as a singlechain inactive precursor that is cleaved by furin-like host proteases in the producing cell into two noncovalently associated subunits, S1 and S2. The S1 subunit contains the receptor-binding domain (RBD), which recognizes the host-cell receptor. The S2 subunit contains the fusion peptide, two heptad repeats, and a trans-membrane domain, all of which are required to mediate fusion of the viral and host-cell membranes by undergoing a large conformational rearrangement. The S1 and S2 subunits trimerize to form a large prefusion spike. The S precursor protein of SARS-CoV-2 can be proteolytically cleaved into S1 (685 aa) and S2 (588 aa) subunits. The S1 subunit consists of the receptor-binding domain (RBD), which mediates virus entry into sensitive cells through the host angiotensin-converting enzyme 2 (ACE2) receptor. Preventing COVID-19 may include preventing at least one of lung fibrosis, interstitial pneumonia, acute lung injury (ALI), acute respiratory distress syndrome (ARDS), aveolar damage, kidney injury, vasculopathy, cardiac injury, acute myocardial injury, chronic damage to the cardiovascular system, thrombosis and venous thromboembolism, in a subject with COVID-19. In a specific embodiment lung fibrosis, interstitial pneumonia, acute lung injury (ALI), acute respiratory distress syndrome (ARDS), kidney injury, such as proteinuria and acute kidney injury, and vasculopathy are triggered by COVID-19. SARS-CoV-2 is used to denote all variants of a virus, according to ICTV belonging to realm Riboviria, kingdom Orthornavirae, phylum Pisuviricota, class Pisoniviricetes, order Nidovirales, family Coronaviridae, genus Betacoronavirus, subgenus Sarbecovirus, species Severe acute respiratory syndrome-related coronavirus, strain Severe acute respiratory syndrome coronavirus 2 (SARS-CoV-2).

The term “HIV” refers to human immunodeficiency viruses. HIV are a member of the genus Lentivirus, part of the family Retroviridae. Two types of HIV, HIV-1 and HIV-2, have been characterized. HIV-1 is the virus that was initially discovered and termed both lymphadenopathy associated virus (LAV) and human T-lymphotropic virus 3 (HTLV-III). HIV-1 is more virulent and more infective than HIV-2, and is the cause of the majority of HIV infections globally. HIV infect humans and cause acquired immunodeficiency syndrome (AIDS) over time. AIDS manifests in progressive failure of the immune system, allowing life-threatening opportunistic infections and cancers to thrive.

As used herein, the term “animal” or “mammal” encompasses all mammals, including humans. Preferably, the mammal of the present invention is a human subject. In one embodiment, the subject is a human.

The terms “individual” and “subject” are used herein interchangeably. They refer to a human or another mammal (e.g. mouse, rat, rabbit, dog, cat, cattle, swine, sheep, horse or primate) that can be afflicted with or is susceptible to a disease or disorder. In many embodiments, the individual is a human being. Unless otherwise stated, the terms “individual” and “subject” do not denote a particular age, and thus encompass adults, elderlies, children, and newborns. In some embodiments, the term “subject” includes humans of age of at least 50, at least 55, at least 60, at least 65, at least 70, or older. In some embodiments, the term “subject” includes humans of age of at least 65, such as 65 to 80, 65 to 75, or 65 to 70. In some embodiments of the present invention, the “individual” or “subject” is a “patient”.

The term “patient” means an individual or subject for treatment or preventive treatment. The present invention relates to the preventive treatment, i.e. vaccination.

The invention is now described with respect to some specific examples which, however, are for illustrative purposes and not to be construed in a limiting manner.

The invention further relates to the following embodiments:

1. A vaccine composition comprising a pharmaceutically effective amount of physiological DCs (phDCs) and at least one mRNA, which comprises a coding sequence encoding for at least one antigenic protein.

2. The vaccine composition according to 1, further comprising a pharmaceutically acceptable carrier or diluent.

3. The vaccine composition according to 1 or 2, wherein said at least one mRNA is comprised in lipid nanoparticles.

4. The vaccine composition according to any one of 1 to 3, wherein said at least one mRNA comprises at least one modified nucleoside.

5. The vaccine composition according to 4, wherein said at least one modified nucleoside is a modified uridine, optionally wherein all uridine residues are replaced with modified uridine.

6. The vaccine composition according to 5, wherein said modified uridine is N1-methyl-pseudouridine.

7. The vaccine composition according to any of 1 to 6, wherein said at least one mRNA is partially modified with N1-methyl-pseudouridine.

8. The vaccine composition according to any of 1 to 6, wherein said at least one mRNA is fully modified with N1-methyl-pseudouridine.

9. The vaccine composition according to any of 1 to 8, wherein said at least one mRNA comprises a 5′ UTR, a 3′ UTR, a 5′ cap and/or a poly(A) tail.

10. The vaccine composition according to any of 1 to 9, wherein said at least one mRNA comprises a sequence which is optimized; optionally, wherein the sequence of said 5′ UTR, 3′UTR and/or coding sequence for the antigenic protein is optimized.

11. The vaccine composition according to any of 1 to 10, wherein said lipid nanoparticles comprise a cationic lipid, a polyethylene glycol (PEG) modified lipid, a cholesterol-based lipid and/or a non-cationic lipid.

12. The vaccine composition according to 11, wherein said cationic lipid is present at a molar ratio between 30% and 40%, said PEG-modified lipid is present at a molar ratio between 1.5% and 4.0%, said cholesterol-based lipid is present at a molar ratio between 40% and 52%, and said non-cationic lipid is present at a molar ratio between 11% and 21%, wherein all the molar ratios are relative to the total lipid content of the LNP.

13. The vaccine composition according to 11 or 12, wherein the cationic lipid is a cKK-E12 lipid.

14. The vaccine composition according to any of 1 to 13, wherein said at least one antigenic protein is associated with a disease.

15. The vaccine composition according to 14, wherein said antigenic protein associated with a disease is an infectious disease associated antigenic protein.

16. The vaccine composition according to 15, wherein said infectious disease associated antigenic protein is a viral antigenic protein, a bacterial antigenic protein, a fungal antigenic protein, a prion antigenic protein or a parasite antigenic protein.

17. The vaccine composition according to 16, wherein said viral antigenic protein is a coronavirus antigenic protein or human immunodeficiency virus (HIV) antigenic protein.

18. The vaccine composition according to 17, wherein said coronavirus antigenic protein is derived from the spike protein, envelope protein, nucleocapsid protein, membrane protein and/or Orf1ab polyprotein.

19. The vaccine composition according to 17 or 18, wherein said coronavirus antigenic protein is derived from the spike protein.

20. The vaccine composition according to any of 17 to 19, wherein said coronavirus antigenic protein is a betacoronavirus antigenic protein.

21. The vaccine composition according to 20, wherein said betacoronavirus antigenic protein is a SARS-CoV-2 antigenic protein.

22. The vaccine composition according to any of 1 to 21, wherein said at least one mRNA comprises a nucleotide sequence which is at least 75%, 85%, 90%, 95%, 98% or 99% or 100% identical to SEQ ID NO: 1, SEQ ID NO: 2, SEQ ID NO: 3, SEQ ID NO: 4 or SEQ ID NO: 5.

23. The vaccine composition according to any of 1 to 22, wherein said at least one mRNA comprises a nucleotide sequence which is at least 75%, 85%, 90%, 95%, 98% or 99% or 100% identical to SEQ ID NO: 1.

24. The vaccine composition according to any of 1 to 21, wherein said at least one mRNA comprises a nucleotide sequence which is at least 75%, 85%, 90%, 95%, 98% or 99% or 100% identical to SEQ ID NO: 19.

25. The vaccine composition according to 16, wherein said bacterial antigenic protein is an antigenic protein of Borrelia spp. or Mycobacteria spp.

26. The vaccine composition according to 16, wherein said fungal antigenic protein is an antigenic protein of Cryptococcus neoformans, Histoplasma capsulatum, Coccidioides immitis, Blastomyces dermatitidis, Chlamydia trachomatis or Candida albicans.

27. The vaccine composition according to 16, wherein said parasite antigenic protein is an antigenic protein of Plasmodium malariae.

28. The vaccine composition according to 14, wherein said antigenic protein associated with a disease is a tumor associated antigenic protein.

29. The vaccine composition according to 28, wherein said tumor associated antigenic protein is a blood cancer antigenic protein.

30. The vaccine composition according to 29, wherein said blood cancer antigenic protein is a leukemia antigenic protein, lymphoma antigenic protein or myeloma antigenic protein.

31. The vaccine composition according to 28, wherein said tumor associated antigenic protein is a solid tumor antigenic protein.

32. The vaccine composition according to 31, wherein said solid tumor antigenic protein is a melanoma antigenic protein, endometrial cancer antigenic protein, kidney cancer antigenic protein, brain cancer antigenic protein, cervical cancer antigenic protein, liver cancer antigenic protein, head and neck cancer antigenic protein, gastrointestinal cancer antigenic protein, lymph node cancer antigenic protein, pancreas cancer antigenic protein, ear, nose and throat (ENT) cancer antigenic protein, breast cancer antigenic protein, prostate cancer antigenic protein, ovarian cancer antigenic protein or lung cancer antigenic protein.

33. The vaccine composition according to any of 1 to 32, wherein the phDCs are obtainable by subjecting monocytes to a physical force.

34. The vaccine composition according to 33, wherein said physical force is applied by passing said monocytes through a flow chamber.

35. The vaccine composition according to 34, wherein said flow chamber is a plate or flexible bag, optionally a flexible plastic bag.

36. The vaccine composition according to 33 to 35, wherein said monocytes are autologous.

37. The vaccine composition of any one of 1 to 36 for use in a prophylactic method of preventing or reducing a disease in a subject, said method comprising administering the vaccine composition to said subject.

38. The vaccine composition of any one of 15 to 27 for use in a prophylactic method of preventing or reducing an infectious disease in a subject, said method comprising administering the vaccine composition to said subject.

39. The vaccine composition of any one of 16 to 24 for use in a prophylactic method of preventing or reducing a viral disease in a subject, said method comprising administering the vaccine composition to said subject.

40. The vaccine composition of any one of 17 to 24 for use in a prophylactic method of preventing or reducing a coronavirus disease, preferably coronavirus disease 2019 (Covid-19), in a subject, said method comprising administering the vaccine composition to said subject.

41. The vaccine composition of any one of 28 to 32 for use in a prophylactic method of preventing or reducing a tumor or a cancer in a subject, said method comprising administering the vaccine composition to said subject.

42. The vaccine composition of any one of 29 or 30 for use in a prophylactic method of preventing or reducing a blood cancer in a subject, said method comprising administering the vaccine composition to said subject.

43. The vaccine composition of any one of 31 or 32 for use in a prophylactic method of preventing or reducing a solid tumor in a subject, said method comprising administering the vaccine composition to said subject.

44. The vaccine composition for use according to any one of 37 to 43, wherein the administration of the vaccine composition is intradermal, intravenous or intramuscular.

45. The vaccine composition for use according to any one of 37 to 43, wherein the administration of the vaccine composition is inhalative.

46. The vaccine composition for use according to any one of 37 to 45, wherein the vaccine composition is administered once or multiple times.

47. The vaccine composition for use according to any one of 37 to 46, wherein at least two doses are administered to the subject.

48. The vaccine composition for use according to any one of 37 to 47, wherein said at least two doses are administered to the subject with an interval of 2 to 6 weeks between each dose, optionally 2 weeks, 3 weeks, 4 weeks, 5 weeks or 6 weeks between each dose.

49. A method for preparing antigen-specific physiologic dendritic cells (phDCs), the method comprising combining phDCs obtained from a donor with at least one mRNA encoding at least one antigenic protein.

50. The method according to embodiment 49, wherein said phDCs are generated by subjecting monocytes obtained from said donor to a physical force.

51. The method according to embodiment 49 or 50, wherein said physical force is applied by passing said monocytes obtained from said donor through a flow chamber.

52. The method according to any of embodiments 49 to 51, wherein the method comprises a further step of incubating said at least one mRNA with said phDCs.

53. The method according to embodiment 52 wherein the incubation step is performed for at least 0.5 h, 1 h, 2 h, 3 h, 4 h, 5 h, 6 h, 8 h, 12 h or 24 h.

54. The method according to any of claims 49 to 53, wherein said monocytes obtained from said donor are derived from an extracorporeal blood sample or PBMCs from said donor.

55. The method according to any of embodiments 49 to 54, wherein said flow chamber is a plate.

56. The method according to any of embodiments 49 to 55, wherein said flow chamber is a bag, optionally a flexible bag or plastic bag.

57. The method according to embodiment 56, wherein said plastic bag is made of a material comprising polyolefin, polyethylene, fluoropolymer, polyvinyl chloride, ethylene-vinyl acetate-copolymer, ethylene vinyl alcohol, polyvinylidene fluoride, and/or other plastic comprising materials approved for medical use.

58. The method according to any of embodiments 49 to 57, wherein said at least one mRNA comprises at least one modified nucleoside.

59. The method according to embodiment 58, wherein said at least one modified nucleoside is a substitution of some or all uridine residues with at least one modified uridine, optionally wherein some or all uridine residues are substituted with N1-methyl-pseudouridine.

60. The method according to embodiment 59, wherein said some or all modified uridine residues comprise one or more uniquely modified uridine residues.

61. The method according to embodiment 60, wherein said one or more uniquely modified uridine residues comprise N1-methyl-pseudouridine.

62. The method according to any of embodiments 49 to 61, wherein said at least one mRNA is partially modified with N1-methyl-pseudouridine.

63. The method according to any of embodiments 49 to 61, wherein said at least one mRNA is fully modified with N1-methyl-pseudouridine.

64. The method according to any of embodiments 49 to 63, wherein said at least one mRNA comprises a 5′ UTR, a 3′ UTR, a 5′ cap and/or a poly(A) tail.

65. The method according to embodiment 64, wherein said 5′ cap is a Cap1 structure or a m7GpppG cap.

66. The method according to any of embodiments 49 to 65, wherein said mRNA comprises a coding sequence, which encodes the antigenic protein.

67. The method according to any of embodiments 49 to 66, wherein said at least one mRNA comprises a sequence which is optimized; preferably, wherein the sequence of the said 5′ UTR, 3′UTR, and/or coding sequence for the antigenic protein is optimized.

68. The method according to any of embodiments 49 to 67, wherein said at least one mRNA encoding the at least one antigenic protein is provided in a lipid nanoparticle.

69. The method according to embodiment 68, wherein said lipid nanoparticle comprises a cationic lipid, a PEG-modified lipid, a cholesterol and/or a non-cationic lipid.

70. The method according to any of embodiments 49 to 69, wherein said antigenic protein is associated with a disease, optionally an infectious disease associated antigenic protein or a tumor associated antigenic protein.

71. The method according to embodiment 70, wherein said infectious disease associated antigenic protein is a viral antigenic protein, a bacterial antigenic protein, a fungal antigenic protein, a prion antigenic protein or a parasite antigenic protein.

72. The method according to embodiment 71, wherein said viral antigenic protein is a coronavirus or HIV antigenic protein.

73. The method according to embodiment 72, wherein said coronavirus antigenic protein is a SARS-CoV antigenic protein, optionally a SARS-CoV-2 antigenic protein.

74. The method according to embodiment 73, wherein said SARS-CoV or SARS-CoV-2 antigenic protein is derived from a spike protein, envelope protein, nucleocapsid protein, membrane protein and/or Orf1ab polyprotein.

75. The method according to embodiment 71, wherein said bacterial antigenic protein is an antigenic protein of Borrelia spp. or Mycobacteria spp.

76. The method according to embodiment 71, wherein said fungal antigenic protein is an antigenic protein of Cryptococcus neoformans, Histoplasma capsulatum, Coccidioides immitis, Blastomyces dermatitidis, Chlamydia trachomatis or Candida albicans.

77. The method according to embodiment 71, wherein said parasite antigenic protein is an antigenic protein of Plasmodium malariae.

78. The method according to embodiment 70, wherein said tumor associated antigenic protein is a leukemia antigenic protein, melanoma antigenic protein, lymphoma antigenic protein, endometrial cancer antigenic protein, kidney cancer antigenic protein, brain cancer antigenic protein, cervical cancer antigenic protein, liver cancer antigenic protein, head and neck cancer antigenic protein, gastrointestinal cancer antigenic protein, lymph node cancer antigenic protein, pancreas cancer antigenic protein, ear, nose and throat (ENT) cancer antigenic protein, breast cancer antigenic protein, prostate cancer antigenic protein, ovarian cancer antigenic protein or lung cancer antigenic protein.

79. The method according to any of embodiments 49 to 74, wherein said mRNA comprises a nucleotide sequence which is at least 75%, 85%, 90%, 95%, 98% or 99% or a 100% identical to a sequence or a part of a sequence selected from SEQ ID NO: 1 (spike protein transcript), SEQ ID NO: 2 (envelope protein transcript), SEQ ID NO: 3 (nucleocapsid protein transcript), SEQ ID NO: 4 (membrane protein transcript) and/or SEQ ID NO: 5 (Orf1ab polyprotein transcript).

80. Antigen-specific phDCs obtainable by a method according to any of embodiments 49 to 79.

81. A vaccine composition comprising a pharmaceutically effective amount of antigen-specific phDCs and at least one mRNA, which encodes for at least one antigenic protein.

82. The vaccine composition according to embodiment 81, further comprising a pharmaceutically acceptable carrier or diluent.

83. The vaccine composition according to embodiment 81 or 82, wherein said mRNA is comprised in lipid nanoparticles.

84. The vaccine composition according to any one of embodiments 81 to 83, wherein said at least one mRNA comprises at least one modified nucleoside.

85. The vaccine composition according to embodiment 84, wherein said at least one modified nucleoside is a substitution of some or all uridine residues.

86. The vaccine composition according to embodiment 85, wherein said some or all modified uridine residues comprise one or more uniquely modified uridine residues.

87. The vaccine composition according to embodiment 85 or 86, wherein said one or more modified uridine residues comprise N1-methyl-pseudouridine.

88. The vaccine composition according to any of embodiments 81 to 87, wherein said at least one mRNA is partially modified with N1-methyl-pseudouridine.

89. The vaccine composition according to any of embodiments 81 to 87, wherein said at least one mRNA is fully modified with N1-methyl-pseudouridine.

90. The vaccine composition according to any of embodiments 81 to 89, wherein said at least one mRNA comprises a 5′ UTR, a 3′ UTR, a 5′ cap and/or a poly(A) tail.

91. The vaccine composition according to embodiment 90, wherein said 5′ cap is a Cap1 structure or a m7GpppG cap.

92. The vaccine composition according to any of embodiments 81 to 91, wherein the mRNA comprises a coding sequence encoding the at least one antigenic protein.

93. The vaccine composition according to any of embodiments 81 to 92, wherein said at least one mRNA comprises a sequence which is optimized; optionally, wherein the sequence of said 5′ UTR, 3′UTR and/or coding sequence for the antigenic protein is optimized.

94. The vaccine composition according to any of embodiments 83 to 93, wherein said lipid nanoparticles comprise a cationic lipid, a PEG-modified lipid, a cholesterol and/or a non-cationic lipid.

95. The vaccine composition according to any of embodiments 81 to 94, wherein said antigenic protein is associated with a disease, optionally an infectious disease associated antigenic protein or a tumor associated antigenic protein.

96. The vaccine composition according to embodiment 95, wherein said infectious disease associated antigenic protein is a viral antigenic protein, a bacterial antigenic protein, a fungal antigenic protein, a prion antigenic protein or a parasite antigenic protein.

97. The vaccine composition according to embodiment 96, wherein said viral antigenic protein is a coronavirus or HIV antigenic protein, preferably a SARS-CoV-2 antigenic protein.

98. The vaccine composition according to embodiment 97, wherein said SARS-CoV-2 antigenic protein is derived from the spike protein, envelope protein, nucleocapsid protein, membrane protein and/or Orf1ab polyprotein.

99. The vaccine composition according to embodiment 96, wherein said bacterial antigenic protein is an antigenic protein of Borrelia spp. or Mycobacteria spp.

100. The vaccine composition according to embodiment 96, wherein said fungal antigenic protein is an antigenic protein of Cryptococcus neoformans, Histoplasma capsulatum, Coccidioides immitis, Blastomyces dermatitidis, Chlamydia trachomatis or Candida albicans.

101. The vaccine composition according to embodiment 96, wherein said parasite antigenic protein is an antigenic protein of Plasmodium malariae.

102. The vaccine composition according to embodiment 96, wherein said tumor associated antigenic proteinic protein is a leukemia antigenic protein, melanoma antigenic protein, lymphoma antigenic protein, endometrial cancer antigenic protein, kidney cancer antigenic protein, brain cancer antigenic protein, cervical cancer antigenic protein, liver cancer antigenic protein, head and neck cancer antigenic protein, gastrointestinal cancer antigenic protein, lymph node cancer antigenic protein, pancreas cancer antigenic protein, ear, nose and throat (ENT) cancer antigenic protein, breast cancer antigenic protein, prostate cancer antigenic protein, ovarian cancer antigenic protein or lung cancer antigenic protein.

103. The vaccine composition according to any of embodiments 81 to 102, wherein said at least one mRNA comprises a nucleotide sequence which is at least 75%, 85%, 90%, 95%, 98% or 99% or 100% identical to a sequence or a part of a sequence selected from the group comprising SEQ ID NO: 1 (spike protein transcript), SEQ ID NO: 2 (envelope protein transcript), SEQ ID NO: 3 (nucleocapsid protein transcript), SEQ ID NO: 4 (membrane protein transcript) and/or SEQ ID NO: 5 (Orf1ab polyprotein transcript).

104. The vaccine composition according to any of embodiments 81 to 103, wherein said phDCs, before combining them with said at least one mRNA, are generated by subjecting monocytes obtained from a donor to a physical force.

105. The vaccine composition according to embodiment 104, wherein said physical force is applied by passing said monocytes obtained from said donor through a flow chamber.

106. The vaccine composition according to embodiment 104 or 105, wherein said monocytes obtained from said donor are derived from an extracorporeal blood sample or PBMCs from said donor.

107. The vaccine composition according to any of embodiments 104 to 106, wherein said flow chamber is a plate.

108. The vaccine composition according to any of embodiments 104 to 106, wherein said flow chamber is a bag, optionally a flexible bag or plastic bag.

109. The vaccine composition according to any of embodiments 104 to 108, wherein the method comprises a further step of incubating said at least one mRNA with said phDCs.

110. The vaccine composition according to embodiment 109, wherein the incubation step is performed for at least 0.5 h, 1 h, 2 h, 3 h, 4 h, 5 h, 6 h, 8 h, 12 h or 24 h.

111. The antigen-specific phDCs of embodiment 80 or the vaccine composition of any one of claims 81 to 110 for use in a prophylactic method of preventing or reducing a disease in a subject, said method comprising administering the antigen-specific phDCs or vaccine composition to said subject.

112. The antigen-specific phDCs or the vaccine composition for use according to embodiment 111, wherein the administration of the vaccine composition is subcutaneous, intravenous, intramuscular, intra-articular, intra-synovial, intrasternal, intrathecal, intra-hepatic, intralesional, intracranial, transdermal, intradermal, intrapulmonal, intraperitoneal, intracardial, intraarterial, intranasal or sublingual; preferably, intradermal, intravenous or intramuscular.

113. The antigen-specific phDCs or the vaccine composition for use according to embodiment 111 or 112, wherein said disease is caused by disease causing particles, optionally wherein the disease causing particles are viruses, bacteria, fungi, parasites and/or tumor cells.

114. The antigen-specific phDCs or the vaccine composition for use according to embodiment 113, wherein the concentration of said disease causing particles is lower upon an infection in the subject as compared to a concentration of disease causing particles in a second infected subject, which had not been administered the vaccine composition.

115. The antigen-specific phDCs or the vaccine composition for use according to embodiment 113, wherein the concentration of said disease causing particles is lower upon an infection in the subject as compared to a concentration of disease causing particles in a second infected subject which had been vaccinated against the same disease causing particle with a different vaccine.

116. The antigen-specific phDCs or the vaccine composition for use according to embodiment 115, wherein said different vaccine does not comprise dendritic cells.

117. The antigen-specific phDCs or the vaccine composition for use according to embodiment 115 or 116, wherein said different vaccine is an RNA vaccine.

118. The antigen-specific phDCs or the vaccine composition for use according to embodiment 115 or 116, wherein said concentration of the disease causing particles is lower by at least 10%, 20%, 30%, 40%, 50%, 60%, 70%, 80%, 90%, 95%, 98% or 99% in the subject as compared to the concentration of the disease causing particles in the second subject.

119. The antigen-specific phDCs or the vaccine composition for use according to embodiment 113, wherein said concentration of the disease causing particles is lower in the subject systemically upon infection.

120. The antigen-specific phDCs or the vaccine composition for use according to embodiment 113, wherein said concentration of the disease causing particles is lower locally, optionally wherein the concentration of the disease causing particles is lower in the brain.

121. The antigen-specific phDCs or the vaccine composition for use according to any of embodiments 111 to 120, wherein said disease in the subject is an infectious disease.

122. The antigen-specific phDCs or the vaccine composition for use according to any of embodiments 111 to 120, wherein said disease in the subject is a viral disease.

123. The antigen-specific phDCs or the vaccine composition for use according to any of embodiments 111 to 120, wherein said disease in the subject is Covid-19.

124. The antigen-specific phDCs or the vaccine composition for use according to any of embodiments 111 to 113, wherein said disease in the subject is a tumor.

125. The antigen-specific phDCs or the vaccine composition for use according to any of embodiments 111 to 113, wherein said disease in the subject is a blood cancer.

126. Use of a vaccine composition according to any of embodiments 113 to 125 for vaccination.

127. A kit comprising physiologic dendritic cells (phDCs) and at least one mRNA, wherein said at least one mRNA encodes for at least one antigenic protein.

128. The kit according to embodiment 127, wherein said phDCs are obtained by passing monocytes obtained from a donor through a flow chamber.

129. The kit according to embodiment 127 or 128, wherein said at least one mRNA encodes for at least one viral antigenic protein.

130. The kit according to embodiment 127 or 128, wherein said at least one mRNA encodes for at least one tumor associated antigenic protein.

131. The kit according to embodiment 129, wherein said viral antigenic protein is a coronavirus antigenic protein, preferably a SARS-CoV-2 antigenic protein.

132. The kit according to embodiment 131, wherein said SARS-CoV-2 antigenic protein is derived from the spike protein, envelope protein, nucleocapsid protein, membrane protein and/or Orf1ab polyprotein.

133. The kit according to any of embodiments 127 to 132, wherein said at least one mRNA is formulated in a lipid nanoparticle.

134. A method of preventing or reducing a disease in a subject, said method comprising administering the antigen-specific phDCs of embodiment 80 or vaccine composition according to any of embodiments 81 to 110 to the subject.

135. The method according to embodiment 134, wherein the administration of the vaccine composition is subcutaneous, intravenous, intramuscular, intra-articular, intra-synovial, intrasternal, intrathecal, intra-hepatic, intralesional, intracranial, transdermal, intradermal, intrapulmonal, intraperitoneal, intracardial, intraarterial, or sublingual; preferably, intradermal, intravenous or intramuscular.

136. The method according to embodiment 134 or 135, wherein the disease is caused by disease causing particles, optionally wherein the disease causing particles are viruses, bacteria, fungi, parasites and/or tumor cells.

137. The method according to embodiment 136, wherein the concentration of the disease causing particles is lower upon an infection in the subject as compared to a concentration of disease causing particles in a second infected subject, which had not been administered the vaccine composition.

138. The method according to embodiment 137, wherein the concentration of disease causing particles is lower upon an infection in the subject as compared to a concentration of disease causing particles in a second infected subject which had been vaccinated against the same disease causing particle with a different vaccine.

139. The method according to embodiment 137 or 138, wherein the concentration of the disease causing particles is lower by at least 10%, 20%, 30%, 40%, 50%, 60%, 70%, 80%, 90%, 95%, 98% or 99% in the subject as compared to the concentration of the disease causing particles in the second subject.

140. The method according to embodiment 136, wherein the concentration of the disease causing particles is lower in the subject systemically upon infection.

141. The method according to embodiment 136, wherein the concentration of the disease causing particles is lower locally, optionally wherein the concentration of the disease causing particles is lower in the brain.

142. The method according to any of embodiments 134 to 141, wherein the disease in the subject is an infectious disease.

143. The method according to any of embodiments 134 to 141, wherein the disease in the subject is a viral disease.

144. The method according to any of embodiments 134 to 141, wherein the disease in the subject is Covid-19.

145. The method according to any of embodiments 134 to 136, wherein the disease in the subject is a tumor.

146. The method according to any of embodiments 134 to 136, wherein the disease in the subject is a blood cancer.

### EXAMPLES

### Example 1. Preparation of phDC Transduced with LNP Particles

### Isolation of Murine Peripheral Blood Mononuclear Cells (PBMC)

Peripheral blood (100-200 uL/mouse) is collected from experimental and control mice (as well as any additional bleeder mice if needed) as required for the experiment; eg, on day 1 and day 7 for preventive vaccination and booster studies, or twice a week for the duration of treatment for therapeutic tumor vaccination studies. Whole blood is collected into 1:100 5,000 U/mL heparin (McKesson Packaging Services). Platelet-containing peripheral blood mononuclear cells (PBMC) are isolated from peripheral whole blood via Lympholyte M gradient separation (Cedarlane Labs). Autologous serum is collected from a separate cohort of syngeneic donor mice and reserved for subsequent steps (overnight culture).

### Transimmunization Chamber

The miniaturized ECP device suitable for work in animal models, called the Transimmunization (TI) chamber, was designed and created for Dr. Edelson's laboratory by Transimmune AG in collaboration with Fraunhofer Institute for Biomedical Engineering, Saarland, Germany. The sterile polystyrene TI chamber has the external dimensions of 25*75 mm, with the flow path of 18*66 mm, and the flow passage height of 290+/−15 um.

### PBMC TI Treatment Protocol

Isolated murine platelet-containing PBMC are resuspended in fetal bovine serum (FBS). The cells are then is incubated in the TI chamber for 1 hour at 37 C. This step allows for platelet-activating plasma protein deposition in the chamber, and platelet adherence to the coated chamber surfaces, as confirmed by light microscopy. The cells are subsequently passed through the TI chamber using a syringe pump, at a rate of 0.09 mL/min. Following plate passage, cells are collected, and the TI chamber washed with 100% FBS at 0.49 mL/min while being physically perturbed by flicking or tapping the plate surface to help detach any adherent cells from the chamber. The collected cells are washed and cultured overnight at standard conditions in RPMI without phenol red (Gibco) supplemented with 15% autologous mouse serum and 1% penicillin/streptomycin/L-glutamine (Invitrogen).

mRNA-Containing LNP Transfection of phDC

The desired amount of LNP (cKK-E12, cholesterol, C14-PEG 2000-PE and DOPE at a ratio of 35:46.5:2.5:16 (Santangelo laboratory, Emory University) containing mRNA for the antigen of interest (such as SARS-CoV-2 Spike protein, or a sample tumor antigen), is added directly to the overnight phDC culture, at the time of setting up the culture. In our experience, LNP amounts can vary from 20 ug to 10 ng, depending on the LNP and antigen used.

phDC Re-Introduction into Experimental Animals

The following day, LNP-transfected cells are harvested by scraping, washed, resuspended in sterile PBS (Gibco), and administered intravenously at 100 uL/animal via the retro-orbital plexus.

### Example 2. PhDC Internalization of Viral Antigen-Containing LNP

### Methods

phDC are prepared from healthy C57BL/6 donor mouse blood by standard protocol described in Example 1. To the plate-passed PBMC in culture is added the desired amount of LNP containing mRNA for the viral antigen of interest (such as SARS-CoV-2 Spike protein mRNA), in this Example 20 ug/mL of cKK-E12 based LNP containing SARS-CoV-2 Spike protein mRNA is used (LNP prepared and provided by the Santangelo laboratory, Emory University, mRNA sequence corresponds to SEQ ID NO: 19). Plate-passed PBMC are incubated with the LNP overnight, to allow for LNP uptake and antigen expression.

Antigen expression is monitored after overnight incubation by using an appropriate fluorescently labeled detection antibody (anti-human SARS-CoV-2 Spike protein antibody, kindly provided by Santangelo laboratory, Emory University), as well as any necessary antibodies to identify cells of interest (eg CD11b⁺, Ly6G⁻ for murine phDC within the plate-passed PBMC; CD11b Biolegend clone M1/70; Ly6G Biolegend clone 1A8).

Antibody binding to phDC can be detected by either flow cytometry (Cytoflex cytometer, analysis using FlowJo v10 software) or by confocal microscopy.

### Results

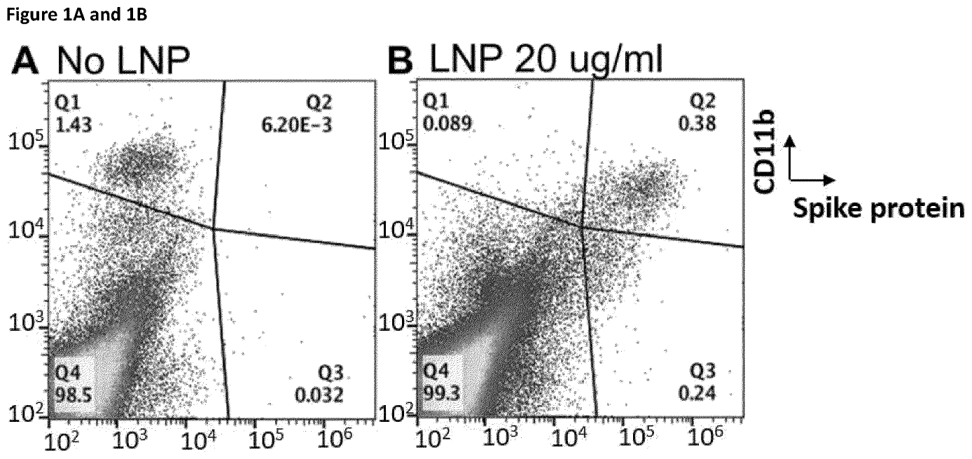

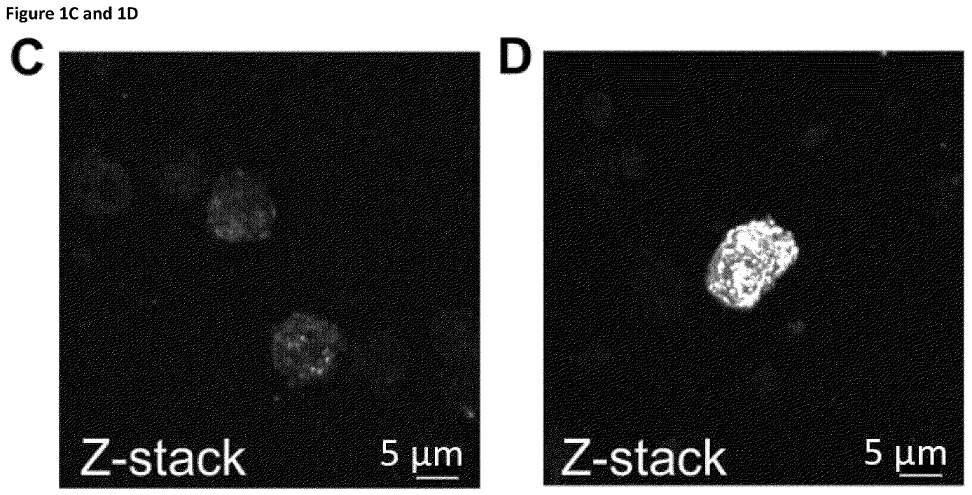

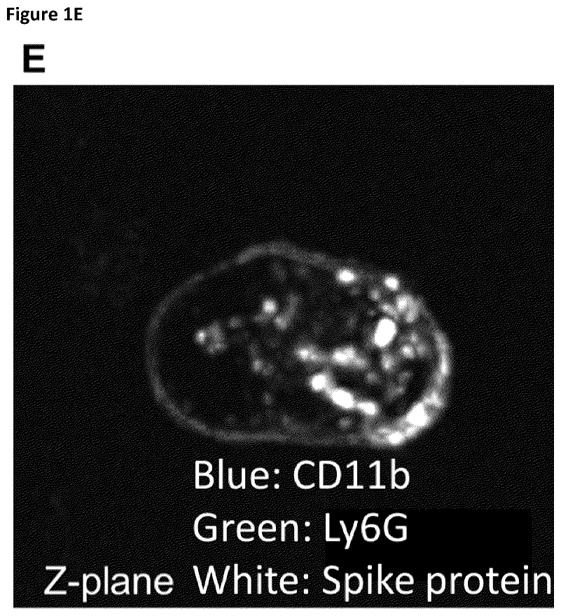

Murine phDC, among all other immune cell subsets contained in the PBMC, specifically internalize the cKK-E12 LNP, and efficiently express Spike protein encoded by mRNA contained in such LNP. FACS analysis shows Spike protein positivity specifically in CD11b+ phDCs, as compared to cultures without LNP transfection (FIG. 1 A, B). This is confirmed by confocal microscopy analysis, showing Spike protein cell surface and cytoplasmic expression specifically in CD11b⁺Ly6G⁻ phDCs (projected Z-stack images, FIG. 1 C, D; Z-plane slice image, FIG. 1E.

### Example 3. Therapeutic phDC Administration in the EG7-OVA Lymphoma Mouse Model Using Tumor Antigen mRNA-Containing LNP (Ovalbumin (OVA) Antigen)

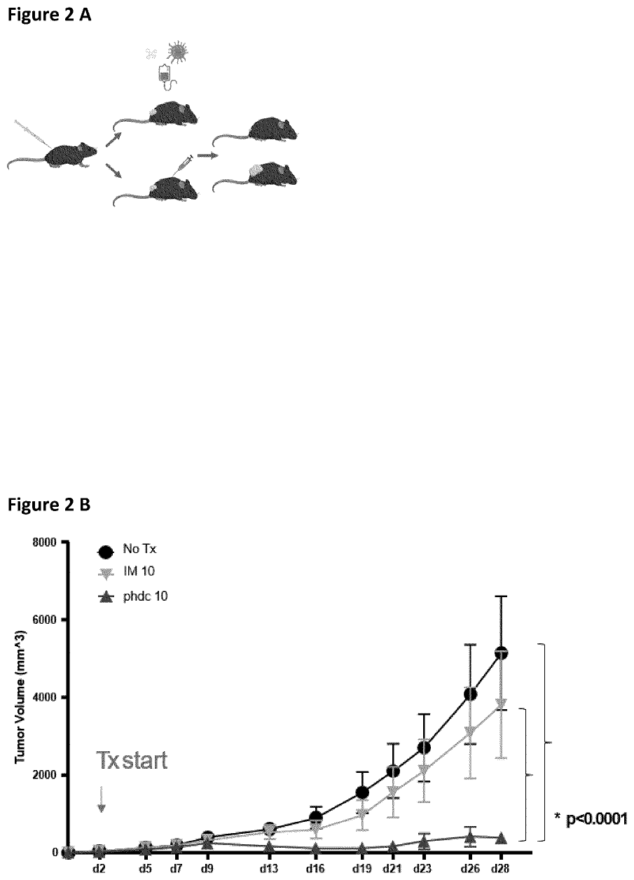

The experimental design is schematically described in FIG. 2A.

For tumor induction, 3×10⁶ EG7-OVA tumor cells are injected subcutaneously in 100 mL into the right flanks of recipient wild-type C57BL/6J mice. Therapeutic treatment is initiated on day 2 following tumor implantation. For each treatment, phDC are prepared from the blood of respective experimental group (200 uL blood collected per animal) by standard protocol described in Example 1. Blood from groups not treated with phDC is discarded.

To the plate-passed PBMC in culture is added 10 ng (0.5 ug/kg of LNP, dose equivalent to COVID-19 Pfizer mRNA LNP vaccine in current human vaccine setting) of cKK-E12 based LNP containing mRNA for the tumor antigen of interest, here the model antigen ovalbumin (OVA) that has been introduced into the EL4 lymphoma cells to produce the EG7-OVA tumor model (SEQ ID NO: 20, also used for all following experiments relating to OVA mRNA containing LNPs). All mRNA and LNPs prepared and provided by the Santangelo laboratory, Emory University.

After overnight incubation, cells co-cultured with LNP containing OVA mRNA are collected, and injected intravenously in a 100 uL volume of sterile PBS into the retro-orbital plexus of the “phDC”-treated experimental group animals. At the same time, an equivalent dose of LNP containing OVA mRNA in 10-50 ul volume of sterile PBS is injected into the thigh of each animal in the intramuscularly (“IM”)-treated experimental group. The animals in the “Untreated” (“No Tx”) group do not receive any therapy.

Treatment is repeated twice a week until the end of the experiment, which is determined by the speed of largest tumor growth up to the maximal limit permitted by the animal care facility. Mice are usually bled for phDC therapeutic manufacture on Mondays and Thursdays, with phDC or i.m. treatments taking place on Tuesdays and Fridays. Typically, 5-6 bi-weekly therapeutic immunization treatments can be carried out.

Tumor volume is monitored via biweekly measurement of perpendicular tumor diameters and height using a caliper, and tumor volume calculated as (tumor length×width×height)/2.

Splenocytes are harvested at the end of the experiment (Day 30 post tumor inoculation) from all treatment and control groups for further analysis, to characterize the resulting anti-tumor immune response. Elispot analysis: CD8+ splenic T cells from treated mice (negative selection using a Miltenyi CD8 T cell isolation kit) are immediately placed into an 18 hr IFN-g Elispot assay at 1*10⁵ cells per well in the presence or absence of 10 ug/mL SIINFEKL peptide. Antigen (SIINFEKL)-specific CD8 T cell evaluation: CD8 (BioRad clone KT1.5) and specific H2d-SIINFEKL dextramer staining (Immudex) followed by flow cytometry (Cytoflex). Tem (effector)/Tcm (central memory) type phenotype analysis: staining for CD44 (Biolegend clone IM7) and CD62L (Biolegend clone MEL-14) expression on antigen-specific T cells followed by flow cytometry (Cytoflex). Stem-like T cell analysis: staining for IL7Ra (Biolegend clone A7R34) and SCA-1 (Biolegend clone E13-161.7) expression on antigen-specific T cells followed by flow cytometry (Cytoflex). T cell exhaustion marker evaluation: PD-1 (Biolegend clone 29F.1A12) expression on antigen-specific T cells followed by flow cytometry (Cytoflex).

### Results

Compared to the control untreated (“No Tx”) group, therapeutic phDC treatment using the clinically relevant dose of 10 ng OVA mRNA-containing LNPs (0.5 ug/kg of LNP, dose equivalent to COVID-19 Pfizer mRNA LNP vaccine in current human vaccine setting) successfully controlled the growth of EG7-OVA tumors, while direct intramuscular administration of the same amount of OVA mRNA-containing LNPs (“IM” group) did not significantly alter the kinetics of tumor growth (FIG. 2B).

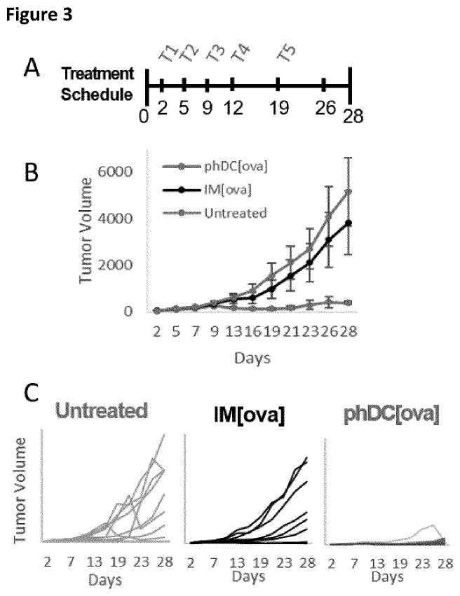

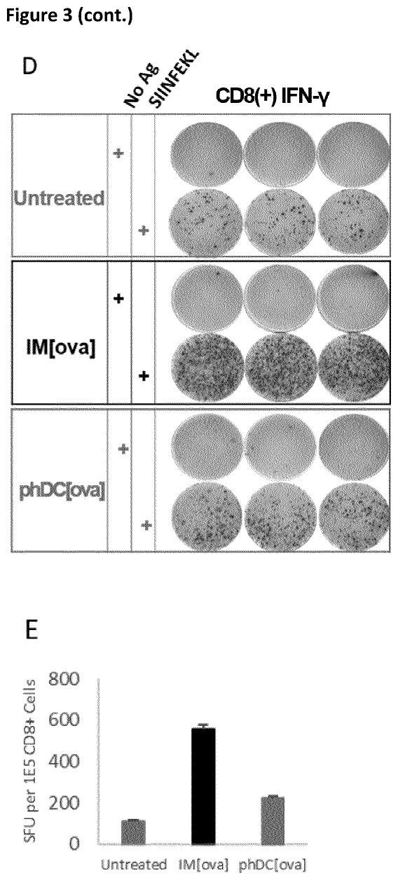

To better understand the therapeutic effect of the phDC vs the IM vaccination methods, splenocytes were collected at the end of the experiment (Day 30 post tumor inoculation) and further analyzed (FIG. 3A-C, experiment schematic). Overall reactivity against the OVA immunodominant peptide SIINFEKL, measured by IFNg Elispot analysis of splenocytes from the different groups, showed some response in the control untreated group as expected of an OVA-expressing tumor-bearing mouse; a stronger response in the phDC-treated group, and, intriguingly, the strongest response in the IM-vaccinated group (FIG. 3D-E), even though this response was clinically unproductive (FIG. 2B).

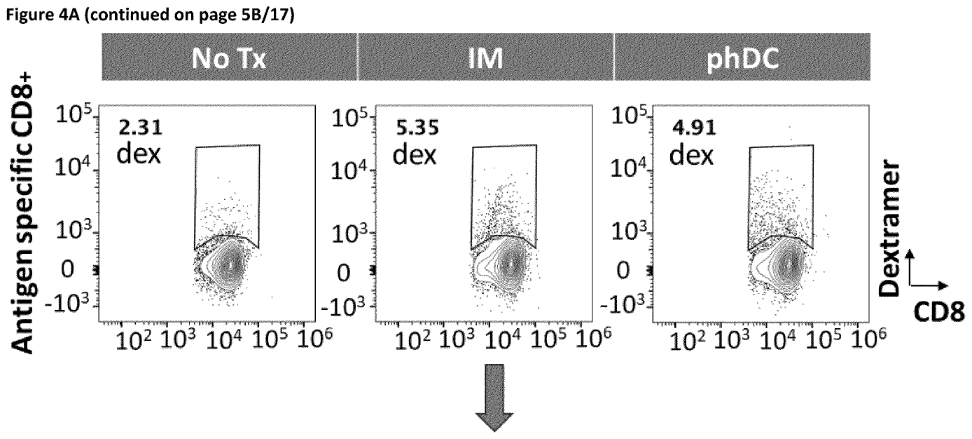

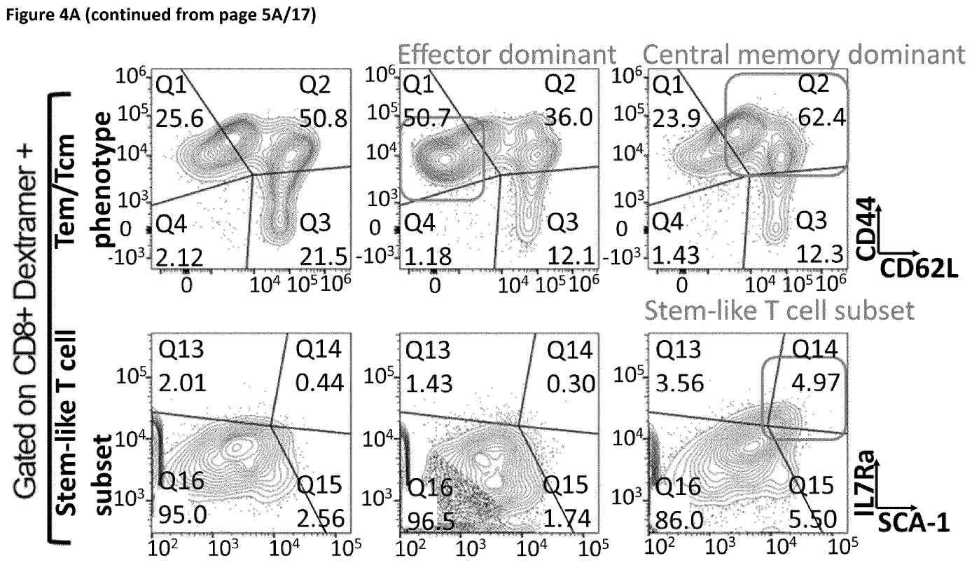

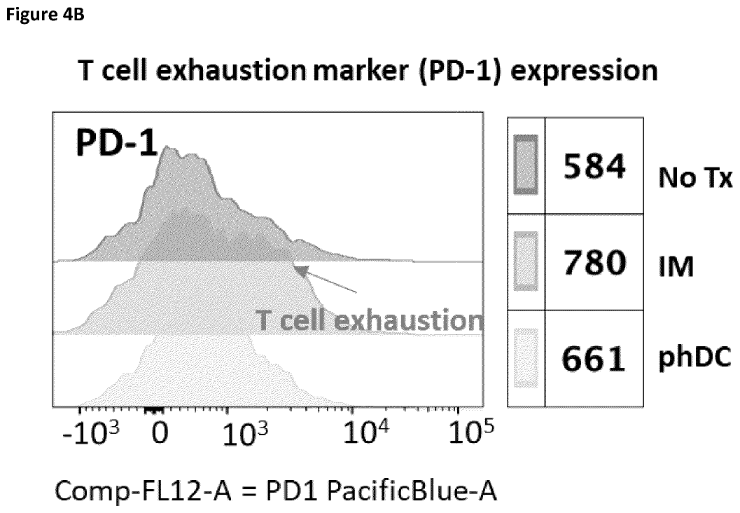

It thus became apparent that it was not the overall quantity of the model tumor antigen OVA-specific T cell response that controls tumor growth. To account for the strong therapeutic effect of phDC treatment, it therefore had to be the quality of the T cell response generated. The quality of the T cell response at Day 30 in the splenocytes of tumor-bearing treated mice was further investigated. This analysis demonstrated that antigen-specific T cells in phDC-treated mice showed a phenotype characterized by the presence of central memory T cell and stem-like T cell subsets (FIG. 4A) that have been linked to productive anti-cancer immune responses in human and mouse studies. In contrast, the T cells from the IM-vaccinated group showed a strong effector T cell-dominant phenotype (FIG. 4A), which is consistent with the strong response seen in the IFNg Elispot assay (FIG. 3D-E).

This effector-like response generated by IM vaccination appears to be clinically unproductive in a tumor setting; perhaps due to the possibly exhausted state of such effector T cells, as demonstrated by their high levels of PD1 expression compared to antigen-specific T cells from phDC-treated animals (FIG. 4B).

### Example 4. Analysis of Spontaneous Immune Response in Prophylactic phDC Vaccination with LNP Containing Ovalbumin (OVA) Sample Antigen mRNA

### Methods

For each treatment, phDC are prepared from the blood of healthy C57BL/6 mice from respective experimental group (200 uL blood collected per animal) by standard protocol described in Example 1. Blood from groups not treated with phDC is discarded.

To the plate-passed PBMC in culture is added 6 ug of cKK-E12 based LNP containing mRNA for the tumor antigen of interest, here the model antigen ovalbumin (OVA). All LNPs prepared and provided by the Santangelo laboratory, Emory University.

Plate-passed PBMC are incubated with the LNP overnight, to allow for LNP uptake and antigen expression. On the following day, the PBMC are collected and prepared as described for Example 1, and administered intravenously at 100 uL/animal via the retro-orbital plexus to the “phDC [ova]”-vaccinated experimental group. In parallel, an equivalent dose of LNP containing OVA mRNA is injected in 10-50 ul volume of sterile PBS into the thigh of each animal in the intramuscularly (“IM [ova]”)-vaccinated experimental group. The animals in the “Untreated” group do not receive any therapy.

Mice are bled on experiment Days −8 and −1, and prophylactically vaccinated with either phDC or with intramuscular LNP on Days −7 and 0. On Days 0, 5, and 13 post vaccination, whole undifferentiated splenocytes are collected for analysis of the spontaneous immune response by IFNg Elispot. Elispot analysis: whole undifferentiated splenocytes from all experimental groups are immediately placed into an 18 hr IFN-g Elispot assay at 1*10⁵ cells per well in the absence of any additional stimulation. To further identify cells responsible for IFNg production, some Day 13 samples were further fractionated into CD8 T cells (negative selection using a Miltenyi CD8 T cell isolation kit), or into NK cells (positive selection using Miltenyi NK1.1 cell isolation kit).

### Results

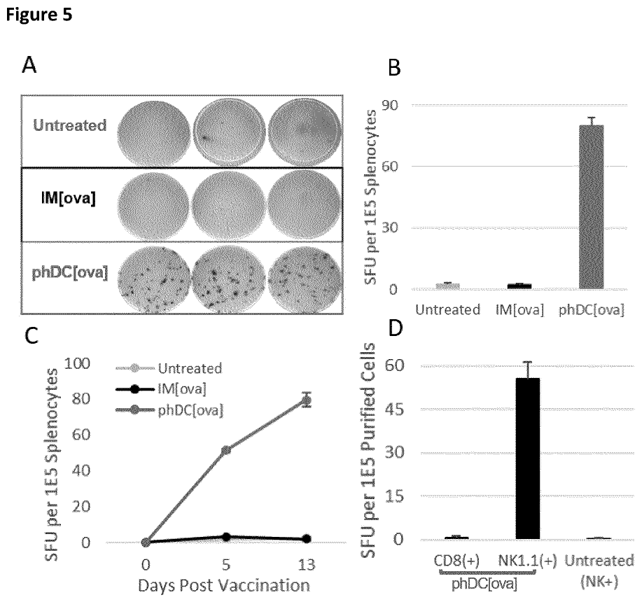

Interestingly, animals prophylactically vaccinated and boosted with phDC, but not with intramuscular LNP injection, demonstrate spontaneous IFNg production in the absence of any additional stimulation (FIG. 5A-C). This spontaneous response is detectable at least as long as 13 days post vaccination (FIG. 5C).

Further dissection of the source of this spontaneous IFNg signal, by isolation of either CD8 T cells or NK cells, identified NK cells as the IFNg-secreting cell subset. This suggests that phDC vaccination not only initiates the antigen-specific T and B cell responses (FIGS. 4, 5), but also broadly engages the innate immune system, such as NK cells.

### Example 5. Prophylactic phDC Vaccination in the EG7-OVA Lymphoma Mouse Model Using Tumor Antigen mRNA-Containing LNP (Ovalbumin (OVA) Antigen)

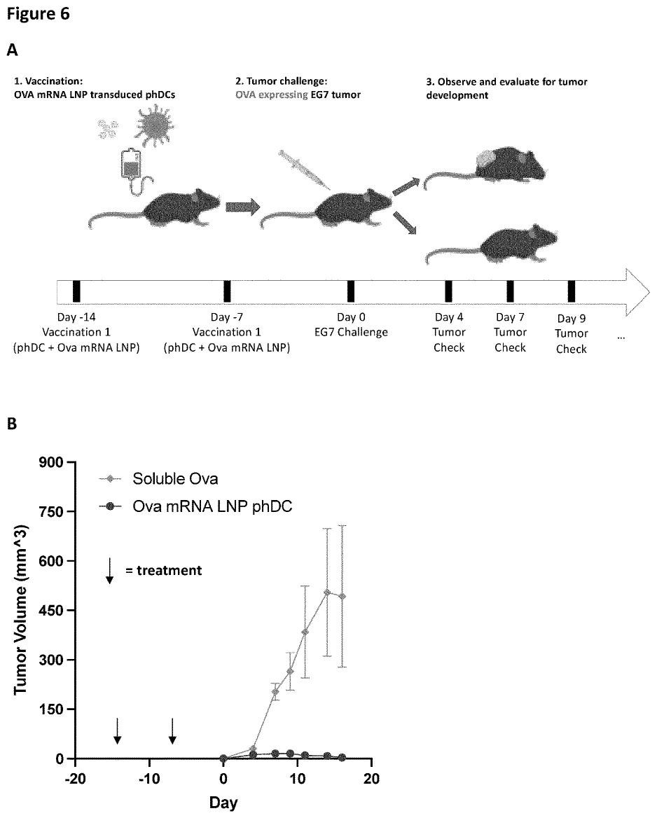

The experimental design is schematically described in FIG. 6A.

For each vaccination treatment, phDC are prepared from the blood of respective experimental group (200 uL blood collected per animal) by standard protocol described in Example 1. Blood from groups not treated with phDC is discarded.

For the initial vaccination (Day −14), to the plate-passed PBMC in culture is added 1 ug of cKK-E12 based LNP containing mRNA for the tumor antigen of interest, here the model antigen ovalbumin (OVA) that has been introduced into the EL4 lymphoma cells to produce the EG7-OVA tumor model. All LNPs prepared and provided by the Santangelo laboratory, Emory University. After overnight incubation, cells co-cultured with LNP containing OVA mRNA are collected, and injected intravenously in a 100 uL volume of sterile PBS into the retro-orbital plexus of the “phDC”-treated experimental group animals. At the same time, 50 ug of soluble OVA protein in 100 uL volume of sterile PBS is injected into the retro-orbital plexus of the “Soluble Ova”-treated experimental group animals.

The initial vaccination treatment is followed 1 week later (Day −7) by a booster vaccination, carried out in the identical manner as that described above.

For tumor challenge at Day 0, 1×10⁶ EG7-OVA tumor cells are injected subcutaneously in 100 mL into the right flanks of mice from all treatment groups. Tumor volume is monitored via biweekly measurement of perpendicular tumor diameters and height using a caliper, and tumor volume calculated as (tumor length×width×height)/2.

### Results

Prophylactic phDC vaccination using OVA mRNA-containing LNPs successfully prevented the growth of EG7-OVA tumors, while vaccination with soluble OVA (“Soluble Ova” group) did not protect against EG7-OVA tumor challenge (FIG. 6B).

### Experiment 6. Detection of OVA Protein in OVA mRNA LNP Transduced phDC

### Methods

phDC are prepared from healthy C57BL/6 donor mouse blood by standard protocol described in Experiment 1. To the plate-passed PBMC in culture is added the desired amount of LNP containing mRNA for antigen of interest, such as 1 ug/mL of cKK-E12 based LNP containing OVA or SARS-CoV-2 Spike protein mRNA (LNP prepared and provided by the Santangelo laboratory, Emory University). Plate-passed PBMC are incubated with the LNP overnight, to allow for LNP uptake and antigen expression.

Antigen expression is monitored after overnight incubation by staining the cells intracellularly with fluorescently labeled anti-OVA antibody (Rockland Immunochemicals), as well as any necessary antibodies to identify cells of interest (eg CD11b+ for murine phDC within the plate-passed PBMC; CD11b Biolegend clone M1/70).

Antibody binding to phDC can be detected by flow cytometry (Cytoflex cytometer, analysis using FlowJo v10 software).

### Results

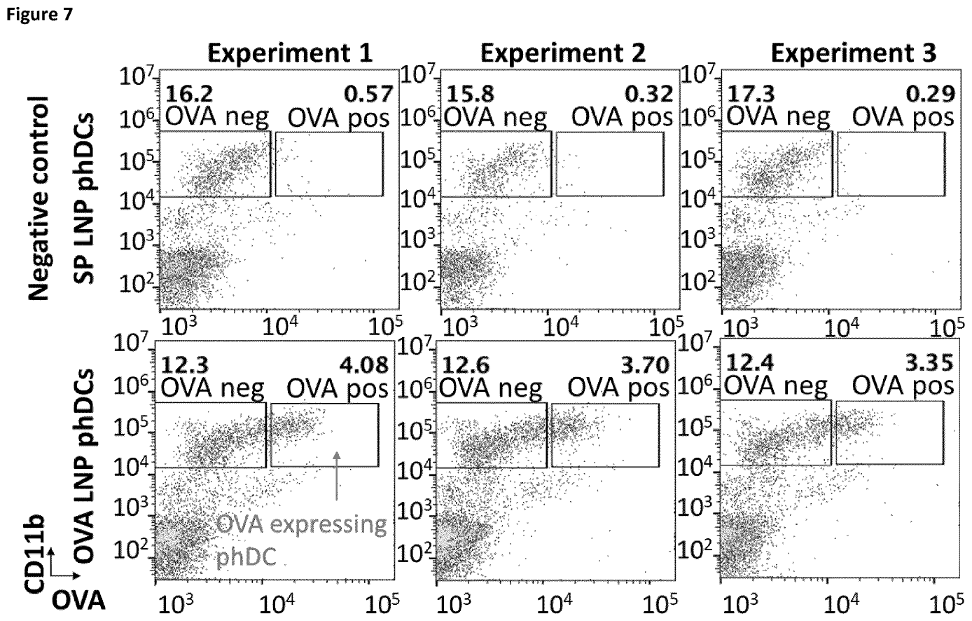

Murine phDC, among all other immune cell subsets contained in the PBMC, specifically express OVA protein encoded by mRNA contained in cKK-E12 LNP. FACS analysis shows OVA protein positivity specifically in CD11b+ phDCs, as compared to cultures with mock (Spike protein) LNP transfection (FIG. 7).

### Experiment 7. Detection of SIINFEKL Bound MHC I (H-2Kb) on Mouse phDC Loaded with Soluble OVA, OVA Expressing Tumor Cells or OVA mRNA LNPs

### Methods

phDC are prepared from healthy C57BL/6 donor mouse blood by standard protocol described in Experiment 1. To the plate-passed PBMC in culture is added the antigen source, such as soluble OVA protein (10 ug/mL; 50 ug/mL); EG7-OVA tumor cells treated with 8-MOP/UVA (200 ng/mL 8-MOP, UVADEX, Therakos; 2 or 4 J/cm2 UVA); or LNP containing OVA protein mRNA (1 ug/mL or 5 ug/mL of cKK-E12 based LNP) (LNP prepared and provided by the Santangelo laboratory, Emory University). Plate-passed PBMC are incubated with the respective antigen sources overnight, to allow for antigen uptake and/or expression, and processing.

Antigenic OVA peptide SIINFEKL presentation on phDC H-2Kb MHCI molecules is detected after overnight incubation by staining the cells with fluorescently labeled 25.D1 TCR-like antibody (Biolegend, clone 25-D1.16), as well as any necessary antibodies to identify cells of interest (eg CD11b+, Ly6G⁻ for murine phDC within the plate-passed PBMC; CD11b Biolegend clone M1/70; Ly6G Biolegend clone 1A8). Antibody binding to phDC can be detected by flow cytometry (Cytoflex cytometer, analysis using FlowJo v10 software).

### Results

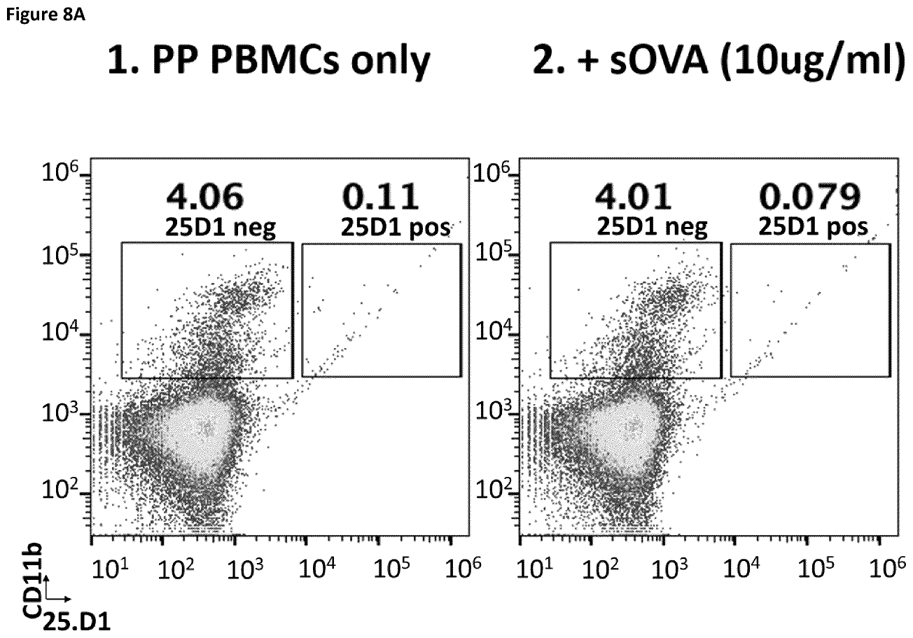

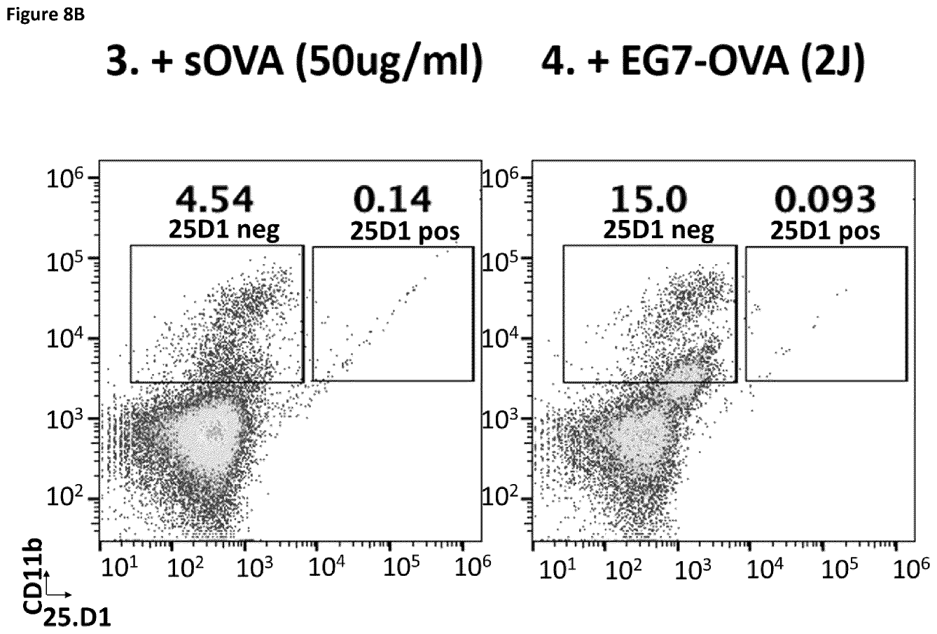

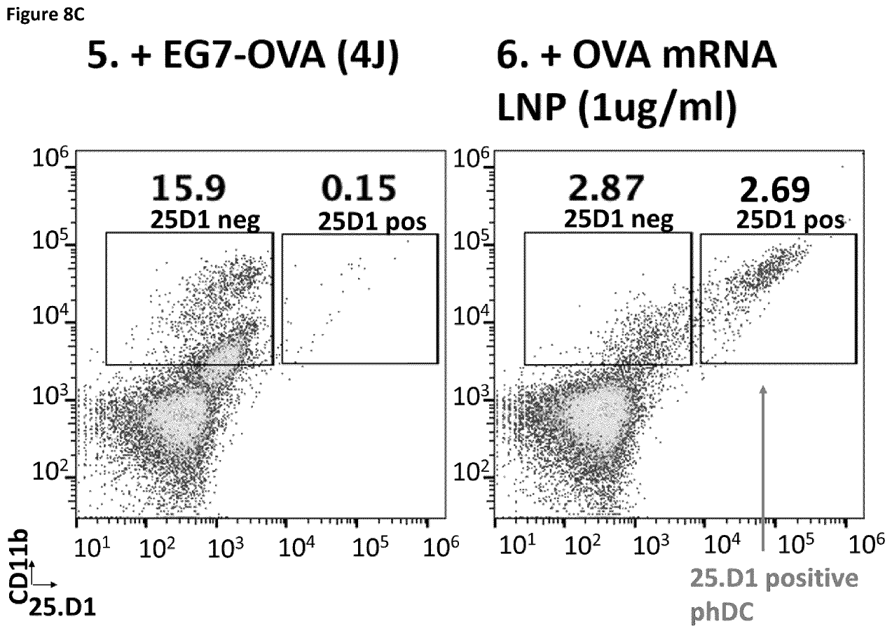

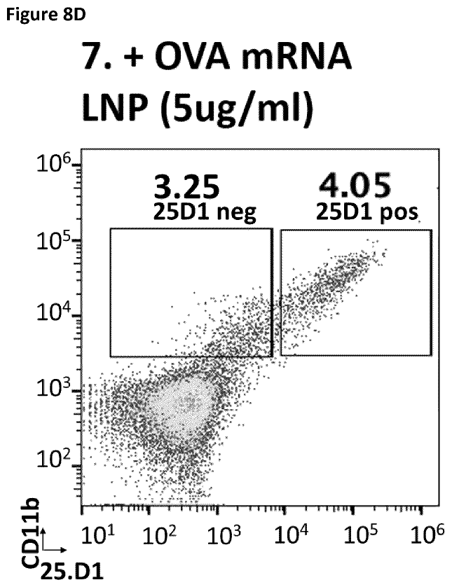

Murine phDC, among all other immune cell subsets contained in the PBMC, specifically process and present antigenic OVA SIINFEKL peptide in their MHCI molecules. Antigenic peptide presentation is significantly higher when OVA protein is expressed via mRNA LNP, as opposed to delivery via soluble OVA protein, or dying tumor cells expressing OVA protein (FIG. 8).

### Experiment 8. Time Kinetics of Surface SIINFEKL-MHC I Complex Expression (Via 25.D1 Ab Stain) in phDC Transduced with OVA mRNA LNP

### Methods

phDC are prepared from healthy C57BL/6 donor mouse blood by standard protocol described in Experiment 1. To the plate-passed PBMC in culture is added the desired amount of LNP containing mRNA for antigen of interest, such as 1 ug/mL of cKK-E12 based LNP containing OVA protein mRNA (LNP prepared and provided by the Santangelo laboratory, Emory University). Plate-passed PBMC are incubated with the LNP for 2, 4, 6, 8, 12, or 20 hrs, then washed and fixed for staining.

Antigenic OVA peptide SIINFEKL expression on phDC H-2Kb MHCI molecules is detected after 2-20 hr incubation by staining the cells with fluorescently labeled 25.D1 TCR-like antibody (Biolegend, clone 25-D1.16), as well as any necessary antibodies to identify cells of interest (eg CD11b+ for murine phDC within the plate-passed PBMC; CD11b Biolegend clone M1/70).

Antibody binding to phDC can be detected by flow cytometry, and antibody-positive cell percentages/mean fluorescence intensity (MFI) of positive cells quantitated (Cytoflex cytometer, analysis using FlowJo v10 software).

### Results

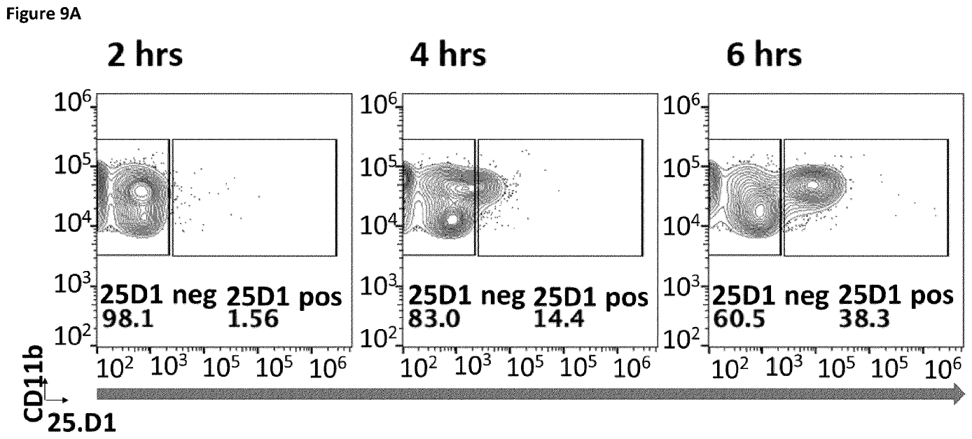

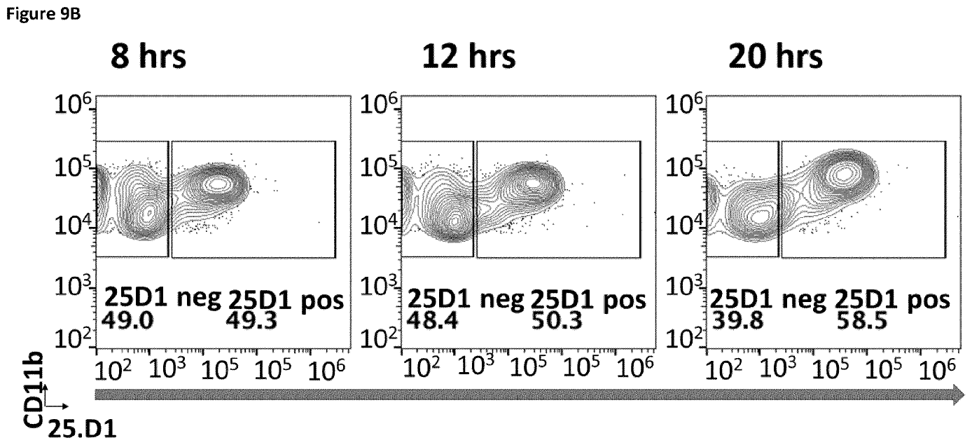

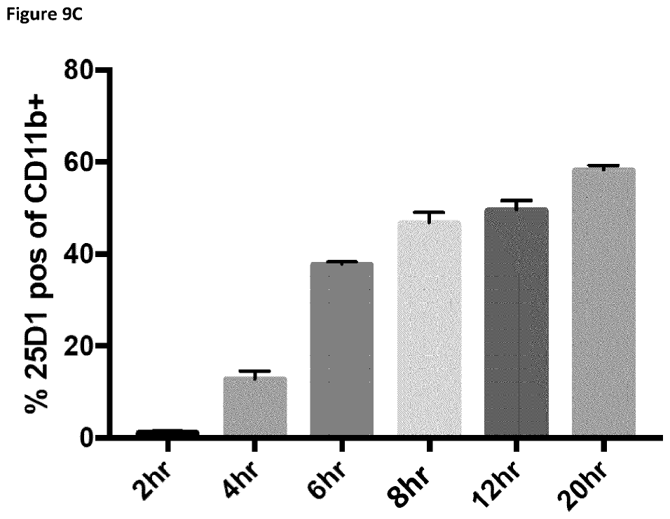

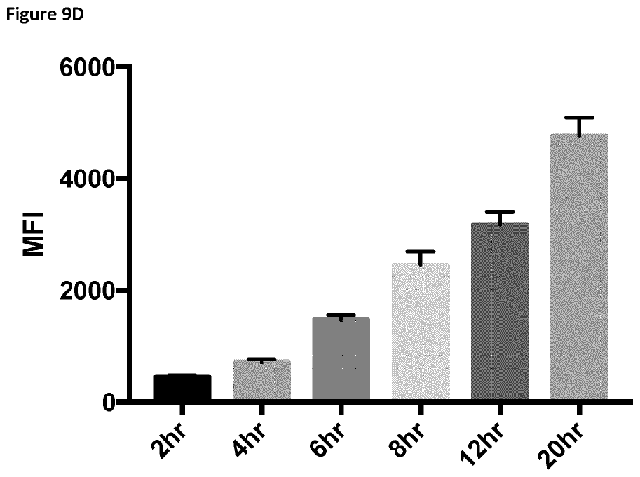

The percentage of phDC displaying antigenic OVA SIINFEKL peptide bound to MHCI molecules on their surface, as well as the amount of SIINFEKL/MHCI per cell, increases with phDC incubation time with OVA mRNA LNP (FIG. 9). The 25.D1 expression level is shown in CD11b+ cells at various time points (FACS plot, upper panel). Percentage of 25.D1 positive cells and MFI level of 25.D1 in CD11b+ subset is shown in the bar graph (lower panel).

### Experiment 9. Detection of SIINFEKL Bound MHC I (H-2Kb) on Mouse phDC or BMDC Transduced with OVA mRNA LNP (at Reduced LNP Concentration)

### Methods

phDC are prepared from healthy C57BL/6 donor mouse blood by standard protocol described in Experiment 1. BMDC were cultured from healthy C57BL/6 donor mouse bone marrow according to standard protocol. Briefly, bone marrow cells were plated in cell culture media containing GM-CSF (20 ng/ml); after 5 days, nonadherent cells were washed and replated in fresh GM-CSF-supplemented media and incubated for 48 hours more; subsequently nonadherent BMDC cells were removed, washed, and used immediately for experiments.

To the plate-passed PBMC or BMDC in culture is added the desired amount of LNP containing mRNA for antigen of interest, such as 0.1 ug/mL of cKK-E12 based LNP containing OVA protein mRNA (LNP prepared and provided by the Santangelo laboratory, Emory University). Plate-passed PBMC or cultured BMDC are incubated with the LNP overnight, to allow for LNP uptake and antigen expression.

Antigenic OVA peptide SIINFEKL expression on phDC H-2Kb MHCI molecules is detected after incubation by staining the cells with fluorescently labeled 25.D1 TCR-like antibody (Biolegend, clone 25-D1.16), as well as any necessary antibodies to identify cells of interest (eg CD11b+ for murine phDC within the plate-passed PBMC; CD11b Biolegend clone M1/70; CD11c for murine BMDC, Biolegend clone N418).

Antibody binding to phDC or BMDC can be detected by flow cytometry (Cytoflex cytometer, analysis using FlowJo v10 software).

### Results

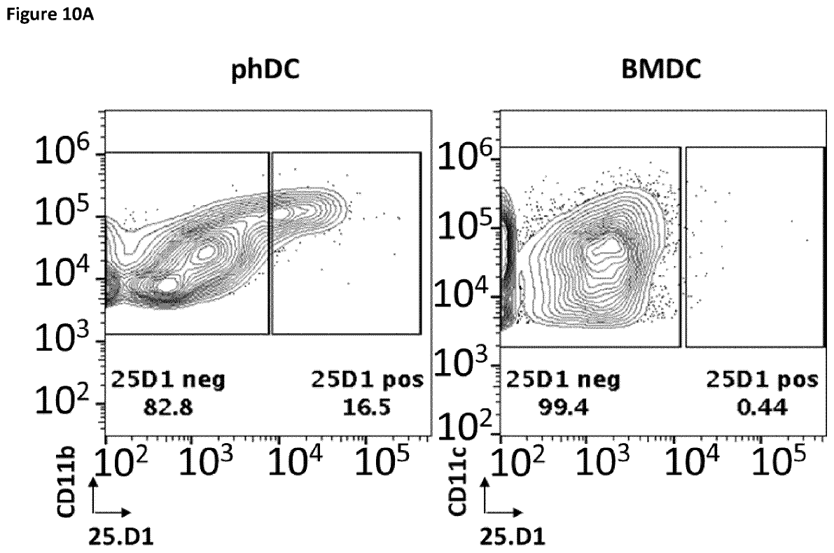

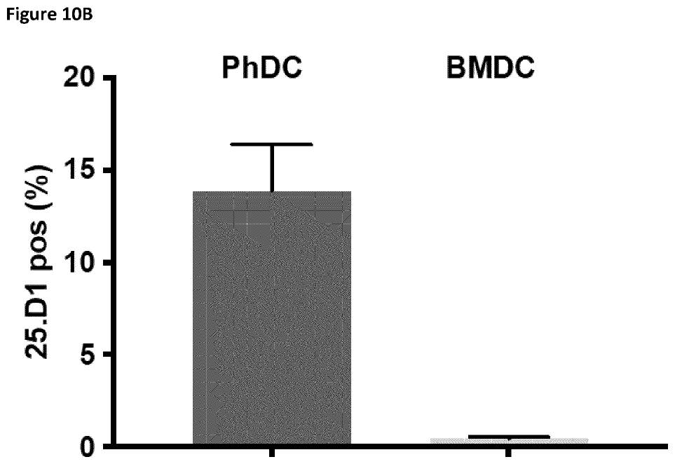

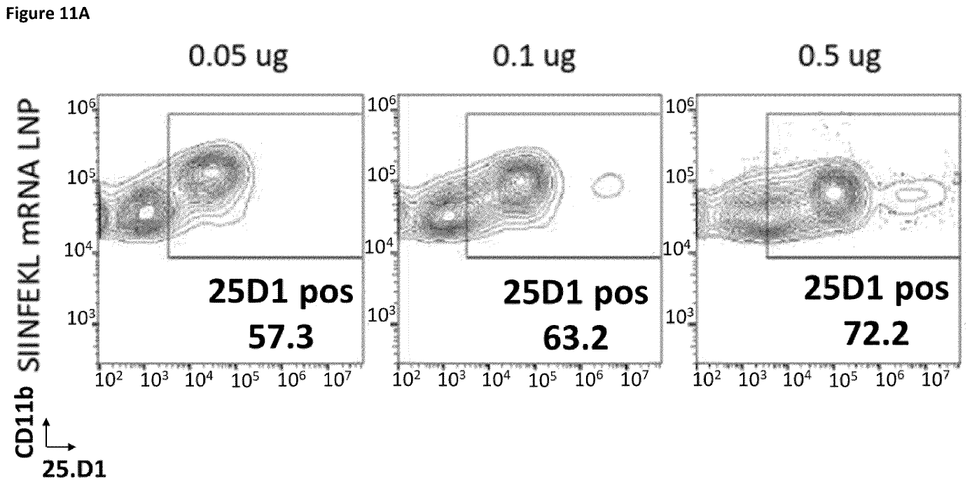

The percentage of phDC displaying antigenic OVA SIINFEKL peptide bound to MHCI molecules on their surface was significantly higher than the percentage of BMDC displaying the same, suggesting that phDC are superior at either transfection with OVA mRNA LNP, or antigen expression and processing, or all of the above, from LNP mRNA antigen source (FIG. 10).

### Experiment 10. Surface 25.D1 Stain in phDC Transduced with SIINFEKL Peptide mRNA Vs OVA Protein mRNA

### Methods

phDC are prepared from healthy C57BL/6 donor mouse blood by standard protocol described in Experiment 1. To the plate-passed PBMC in culture is added the desired amount of LNP containing mRNA for antigen of interest, such as 1 ug/mL of cKK-E12 based LNP containing OVA protein mRNA, or 0.05, 0.1 ug, 0.5 ug, 1 ug, or 5 ug of LNP containing immunogenic OVA SIINFEKL peptide mRNA (LNP prepared and provided by the Santangelo laboratory, Emory University, SEQ ID NO: 21). Plate-passed PBMC are incubated with the LNP overnight, to allow for LNP uptake and antigen expression.

Antigenic OVA peptide SIINFEKL expression on phDC H-2Kb MHCI molecules is detected after overnight incubation by staining the cells with fluorescently labeled 25.D1 TCR-like antibody (Biolegend, clone 25-D1.16), as well as any necessary antibodies to identify cells of interest (eg CD11b+ for murine phDC within the plate-passed PBMC; CD11b Biolegend clone M1/70).

Antibody binding to phDC or BMDC can be detected by flow cytometry (Cytoflex cytometer, analysis using FlowJo v10 software).

### Results

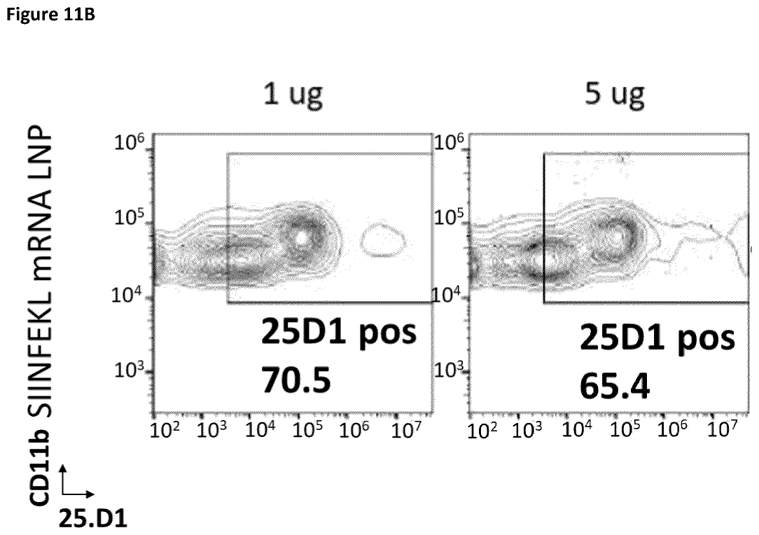

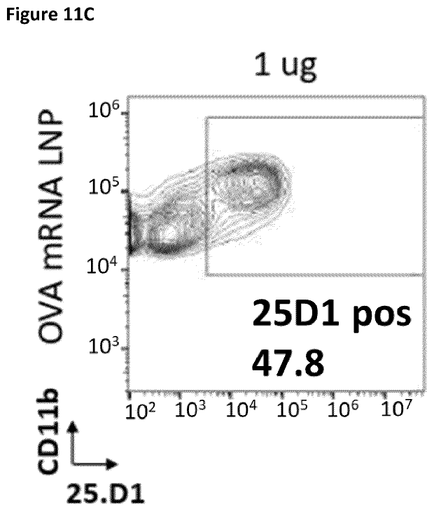

The percentage of phDC displaying antigenic OVA SIINFEKL peptide bound to MHCI molecules on their surface is higher at the same LNP concentration (1 ug/mL) for phDC transduced with SIINFEKL peptide mRNA LNP, than for phDC transduced with OVA protein mRNA LNP (FIG. 11). phDC transduction with SIINFEKL peptide mRNA LNP is also efficient at much lower LNP concentrations, displaying surface antigen/MHCI complexes with as little as 0.05 ug/mL LNP used. Together, these data show that loading of phDC with immunogenic peptides alone, rather than with whole proteins, as antigens, can be highly efficient.

### Experiment 11. OT1 Proliferation Assay: PhDC Pulsed with Titrating Amounts of LNP or Soluble OVA Protein Cultured with OT1 T Cells

### Methods

phDC are prepared from healthy C57BL/6 donor mouse blood by standard protocol described in Experiment 1. To the plate-passed PBMC in culture is added the antigen source, such as soluble OVA protein (50 ug/mL; 200 ug/mL); or LNP containing OVA protein mRNA (1, 0.1, 0.01, or 0.001 ug/mL of cKK-E12 based LNP) (LNP prepared and provided by the Santangelo laboratory, Emory University). Plate-passed PBMC are incubated with the respective antigen sources overnight, to allow for antigen uptake and/or expression, and processing.

After overnight incubation, antigen-loaded cells are harvested and cocultured for 3 days at 2*10⁵ cells/mL (of note, phDC are not purified; the cell number is for total PBMC, of which 3-10% are CD11+ phDC; phDC cell numbers within PBMC are therefore at most ~2*10⁴ cells/mL) under standard conditions in 96-well plates with 1*10⁵ cells/mL CFSE-labeled OVA-specific OT1 CD8 T cells (isolated from spleens of C57Bl/6-Tg (TcraTcrb)1100Mjb/J mice that recognize OVA peptide residues 257 to 264 in the context of H-2Kb, Jackson Laboratory). At the end of coculture, cells are stained with anti-CD8 antibody to identify antigen-reactive T cells (Biolegend, clone 53-6.7).

Antigen-specific CD8 T cell proliferation, as indicated by CFSE dilution in OT1 CD8+ T cells, can be assessed by flow cytometry (Cytoflex cytometer, analysis using FlowJo v10 software).

### Results

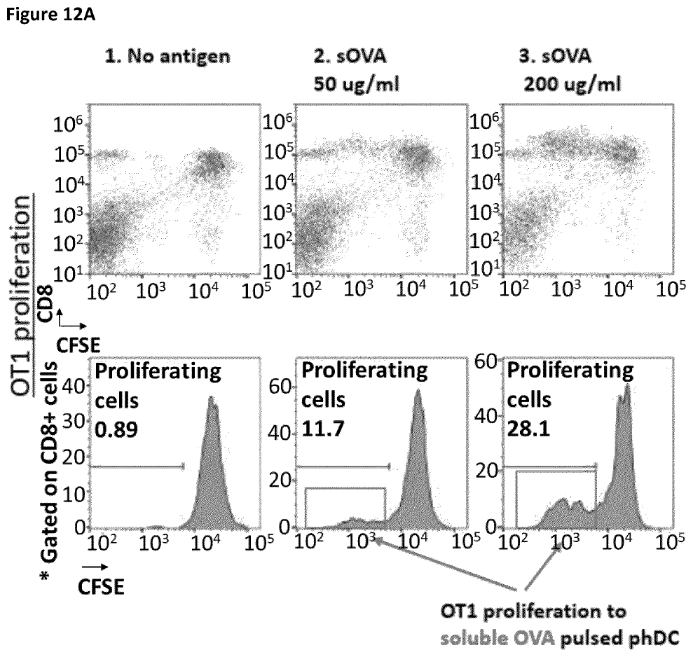

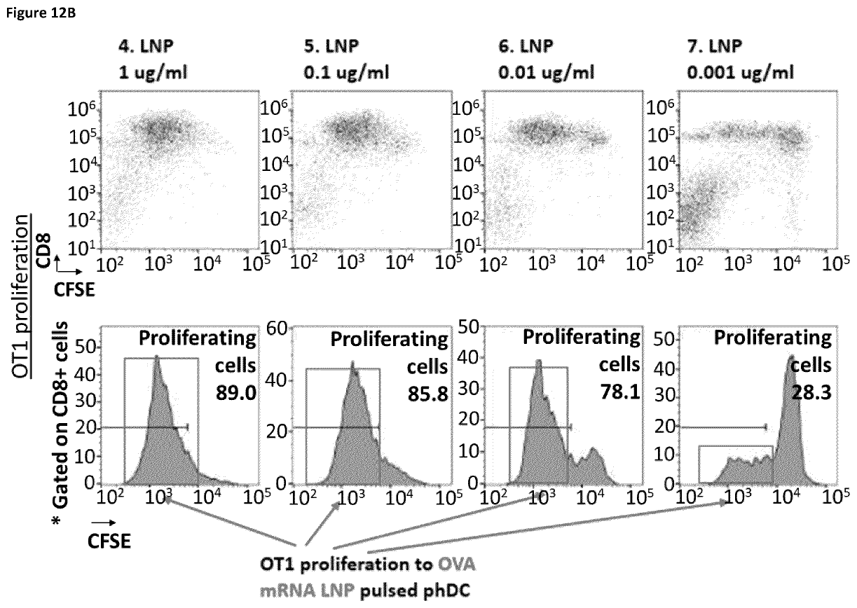

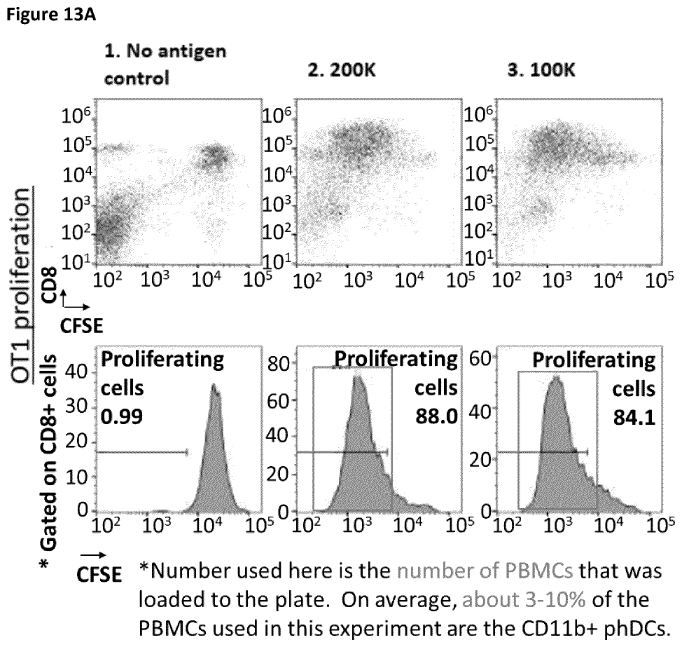

The percentage of proliferated OVA-reactive OT1 CD8 T cells when cultured with OVA protein mRNA LNP-transduced phDC is very high, even at the lowest LNP concentration (0.001 ug/mL, FIG. 12). This demonstrates that LNP-transduced phDC are extremely potent at stimulating antigen-specific CD8 T cell responses. Furthermore, phDC transduction with the lowest LNP concentration (0.001 ug/mL) was at least as potent at stimulating CD8 T cell response as phDC loading with the highest soluble OVA protein concentration (200 ng/mL), suggesting that mRNA LNP are a significantly superior antigen source.

### Experiment 12. OT1 Proliferation Assay: OT1 T Cells Cultured with Titrating Amount of phDC (at 1 μg/mL LNP)

### Methods

phDC are prepared from healthy C57BL/6 donor mouse blood by standard protocol described in Experiment 1. To the plate-passed PBMC in culture is added the antigen source, such as LNP containing OVA protein mRNA (1 ug/mL of cKK-E12 based LNP) (LNP prepared and provided by the Santangelo laboratory, Emory University). Plate-passed PBMC are incubated with the respective antigen sources overnight, to allow for antigen uptake and/or expression, and processing.

After overnight incubation, cells are harvested and cocultured for 3 days at 2*10⁵, 1*10⁵, 5*10⁴, 2.5*10⁴, or 1.2*10⁴ cells/mL (of note, phDC are not purified; the cell number is for total PBMC, of which 3-10% are CD11+ phDC; phDC cell numbers within PBMC are therefore at most ~2*10⁴, 1*10⁴, 5*10³, 2.5*10³, or 1.2*10³ cells/mL) under standard conditions in 96-well plates with 1*10⁵ cells/mL CFSE-labeled OVA-specific OT1 CD8 T cells (isolated from spleens of C57Bl/6-Tg (TcraTcrb)1100Mjb/J mice that recognize OVA peptide residues 257 to 264 in the context of H-2Kb, Jackson Laboratory). At the end of coculture, cells are stained with anti-CD8 antibody to identify antigen-reactive T cells (Biolegend, clone 53-6.7).

CD8 T cell proliferation as indicated by CFSE dilution in CD8+ T cells can be assessed by flow cytometry (Cytoflex cytometer, analysis using FlowJo v10 software).

### Results

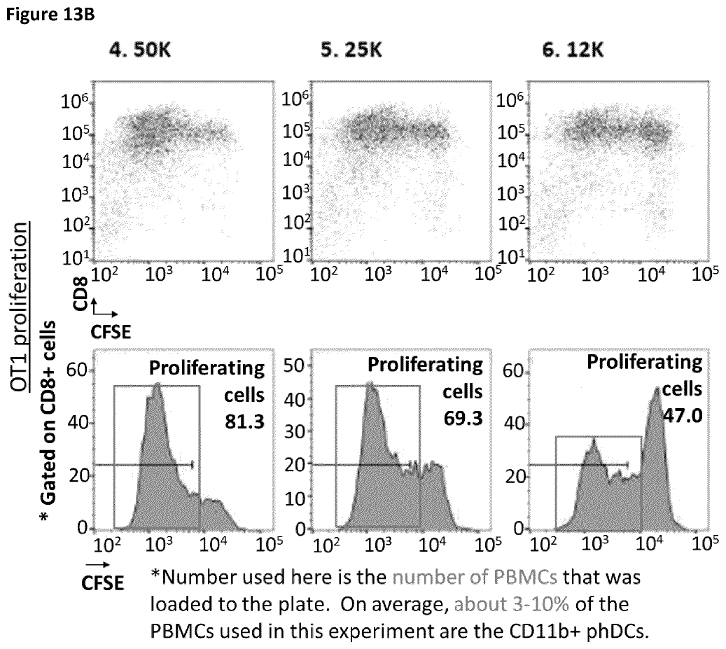

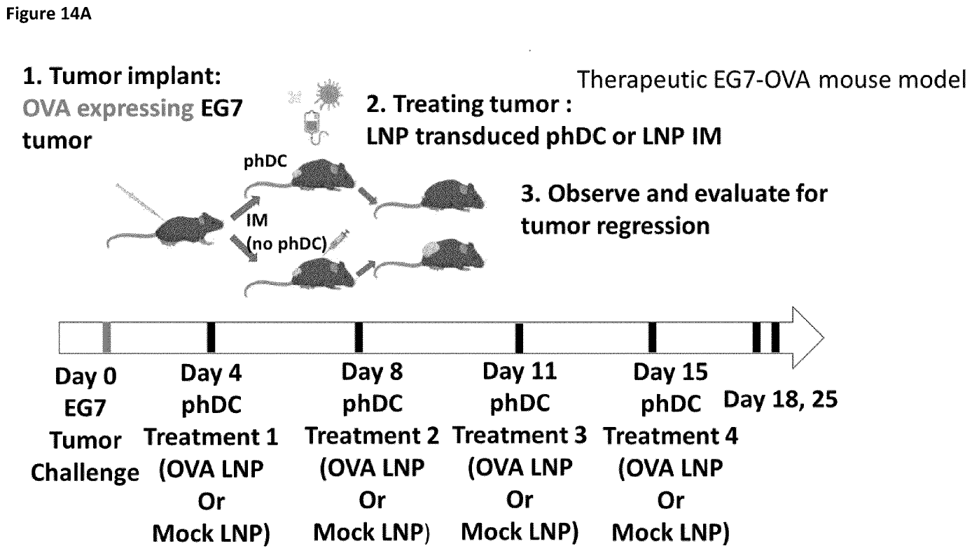

The percentage of proliferated OVA-reactive OT1 CD8 T cells when cultured with titrating amounts of OVA protein mRNA LNP-transduced phDC remains notably high, with as few as 1,200 phDC per mL (120 cells per well in a 96-well plate) retaining the ability to stimulate proliferation in up to 47% of OT1 CD8 T cells (FIG. 13). This again indicates that mRNA LNP-transduced phDC are extremely potent and efficient stimulators of antigen-specific CD8 T cell response.

### Experiment 13. Therapeutic phDC Administration in the EG7-OVA Lymphoma Mouse Model Using Tumor Antigen mRNA-Containing LNP (Ovalbumin (OVA) Antigen)

### Methods

The experimental protocol is as described for Experiment 3, with the sole difference that control mice, instead of remaining untreated, received bi-weekly intramuscular injection of the same amount (10 ng/mouse) of mock mRNA LNP (expressing SARS-CoV-2 Spike protein, irrelevant in the EG7-OVA tumor model setting).

The experimental groups therefore are:
Mock (SARS-CoV-2 Spike protein) mRNA LNP intramuscular vaccination
OVA mRNA LNP intramuscular vaccination
OVA mRNA LNP phDC retro-orbital vaccination

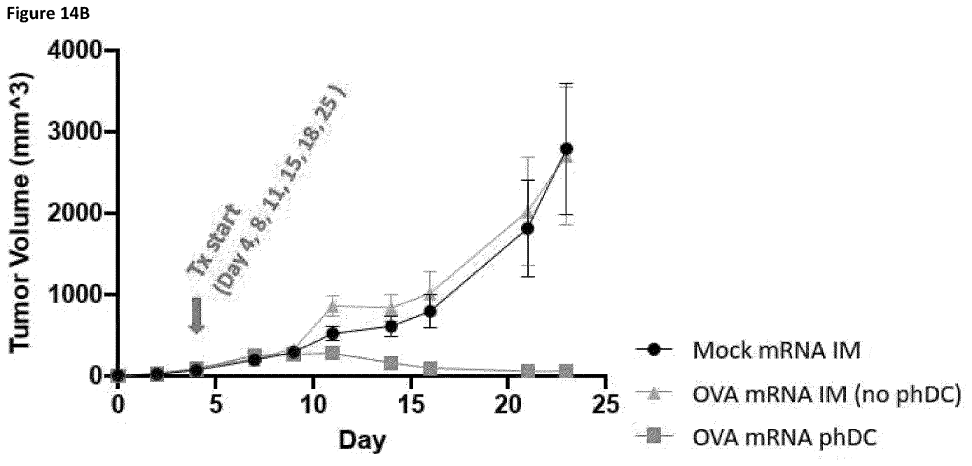

With treatments administered on days 4, 8, 11, 15, 18, and 25 post EG7-OVA tumor implantation (FIG. 14 a).

### Results

Compared to the control mock-treated (“Mock mRNA IM”) group, therapeutic phDC administration (“OVA mRNA phDC”) using the clinically relevant dose of 10 ng OVA mRNA-containing LNPs (0.5 ug/kg of LNP, dose equivalent to COVID-19 Pfizer mRNA LNP vaccine in current human vaccine setting) successfully controlled the growth of EG7-OVA tumors, while direct intramuscular administration of the same amount of OVA mRNA-containing LNPs (“OVA mRNA IM (no phDC)” group) did not significantly alter the kinetics of tumor growth (FIG. 14 b).

### Experiment 14. SIINFEKL Tetramer Analysis and Adoptive T Cell Transfer

### Methods

Splenocytes are harvested at the end of Experiment 13 described above (were harvested at the end of the in vivo tumor monitoring period (Day 32 after tumor inoculation) from all treatment and control groups for further analysis, to characterize the resulting anti-tumor immune response.

Antigen (SIINFEKL)-specific CD8 T cell evaluation: CD8 (BioRad, clone KT1.5) and specific H2d-SIINFEKL dextramer (Immudex) staining of splenocytes isolated above, followed by flow cytometry and analysis (Cytoflex).

Adoptive T cell transfer: CD3+ T cells isolated from splenocytes of the three experimental groups of Experiment 13 (Miltenyi T cell isolation kit) are transferred intravenously at 1.5*10⁷ T cells/mouse into antigen-naiive C57BL/6 mice, freshly inoculated with EG7-OVA tumors (tumor inoculation as described for Experiment 3), and tumor development was monitored over the course of 19 days.

### Results

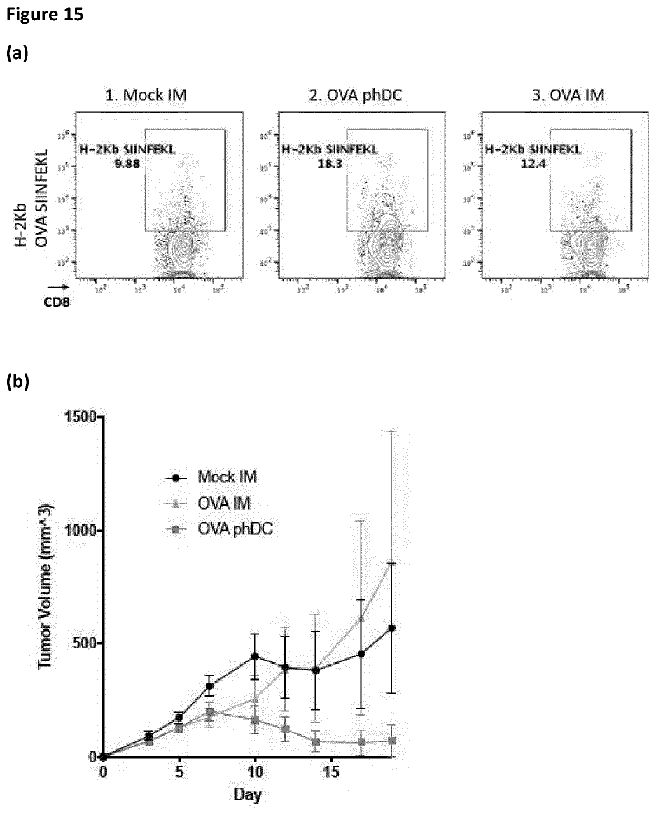

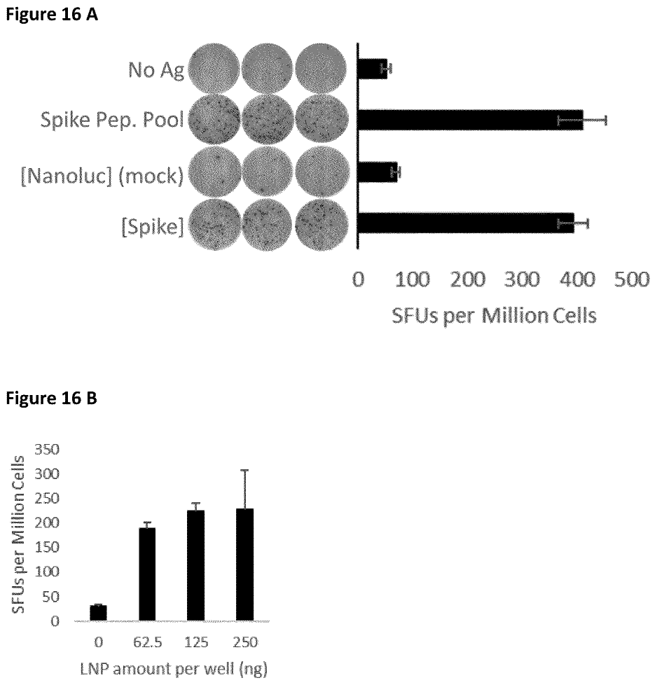

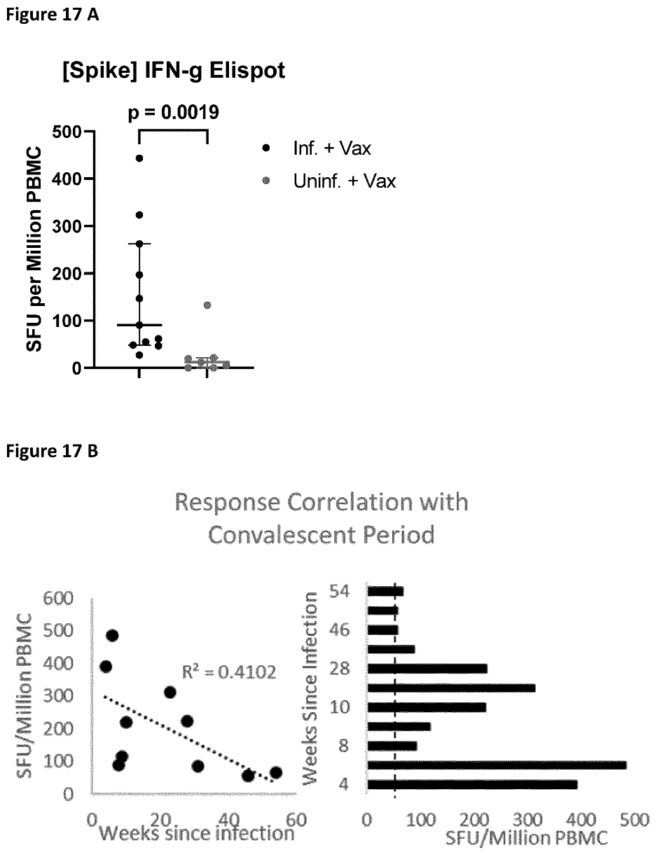

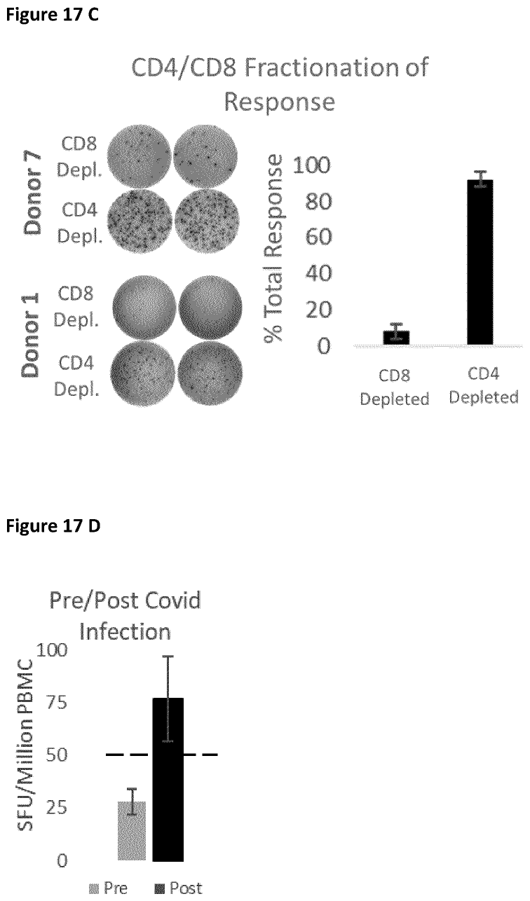

Therapeutic OVA protein mRNA LNP-transduced phDC vaccination (“OVA phDC”) and intramuscular OVA protein mRNA LNP vaccination (“OVA IM”) both successfully induced OVA antigen-specific T cells, as measured by percentage of H-2Kb SIINFEKL tetramer-positive CD8 T cells (FIG. 15 a). Mock-treated animals (“Mock IM”) also showed some tetramer positivity, reflecting the presence of background, natural T immunity in EG7-OVA tumor bearing mice.

However, despite the presence of tumor-reactive T cells in all groups, only T cells isolated from the spleens of mice vaccinated with OVA protein mRNA LNP-transduced phDC (“OVA phDC”) conferred protective immunity against EG7-OVA tumors in antigen-naiive, unvaccinated mice (FIG. 15 b). This indicates that only phDC therapeutic vaccination provides true anti-tumor immunity.

### Experiment 15: Spike Protein ELISpot Detection of SARS-CoV-2 Reactive T Cells

Spike Protein mRNA-Containing LNP Transduction of Human phDC

Plate-passed PBMC containing nascent phDC from healthy human donors, produced per methods described in Experiment 1, are plated at 5*10⁵ cells per well, in triplicate per experimental group, in human IFNγ ELISpot plates. To each experimental well is added 62.5-250 ng/well of cKK-E12 based LNP (cKK-E12, cholesterol, C14-PEG 2000-PE and DOPE at a ratio of 35:46.5:2.5:16) containing Spike protein mRNA (LNPs prepared and provided by the Santangelo laboratory, Emory University, RNA sequence corresponds to SEQ ID NO: 19) in 200 μL of culture medium consisting of RPMI without phenol red (Gibco) supplemented with 15% autologous human plasma and 1% penicillin/streptomycin/L-glutamine (Invitrogen). Positive control wells are treated with an overlapping pool of Spike Class I & II peptides (Miltenyi Biotec, PepTivatorSARS-CoV-2 Prot-S Complete). Negative control wells are treated with 62.5-250 ng/well of cKK-E12 based LNP containing an irrelevant (eg Nanoluciferase) protein mRNA.

### Elispot Assay Read-Out

After overnight incubation, wells are washed and IFNγ spots are detected with biotinylated anti-human IFNγ mAb, streptavidin-ALP and BCIP/NBT-plus substrate per manufacturer's protocol (MAbTech, 3420-2AST-2). Spot forming units (SFU) for each experimental condition are quantitated using an ELISpot plate reader and ELISpot 6.0 iSpot software (Autoimmun Diagnostika GmbH, Strasburg, Germany). By convention, an ELISpot signal is deemed positive if it is above 50 SFU/million cells, and/or the experimental group signal is at least 2-fold higher than the control (mock) group signal.

### CD4 and CD8 T Cell Depletions

To test the contribution of T cell subsets to cytokine production, CD4 or CD8 T cells may be selectively depleted from PBMC after plate passage, but prior to overnight incubation with LNPs in Elispot wells. T cells are depleted using standard depletion kits (Miltenyi; CellSep).

### Results

FIG. 16A: An example of a phDC ELISpot using PBMC isolated from a single human donor, 4 weeks post SARS-CoV2 infection. IFNγ release was significantly elevated in the presence of phDC [Spike], when compared to negative controls. This demonstrates detection of human Spike specific T cells via mRNA transduced phDC in a Covid convalescent donor.

FIG. 16B: An example of a phDC ELISpot dose response using PBMC from a single human donor, 4 weeks post SARS-CoV2 infection. IFNγ release increased to levels above 150 SFU/million when LNP [Spike] antigen was added at 62.5 ng/well. T cell activation improved further at higher doses but showed evidence of a response plateau at 250 ng/well. This demonstrates phDC LNP Spike dose response detectable at range as low as 62.5 ng/well.

FIG. 17A: 18 previously vaccinated human donors were screened in phDC ELISpot against SARS-CoV-2 Spike antigen. Donors were separated into two cohorts based on whether they were previously infected with SARS-CoV-2 (black) or not (blue). Statistical analysis utilized an unpaired, two-tailed, Mann-Whitney U test. This demonstrates that human phDC transduced with LNP [Spike] can differentiate T cell responses associated with natural immunity from the response associated with vaccination alone.

FIG. 17B: 11 previously vaccinated and convalescent human donors were screened in phDC ELISpot against SARS-CoV-2 Spike antigen. Dotted line represents the standard threshold cutoff for positive response (50 SFU/Million cells). This demonstrates that 1) the response strength is generally negatively correlated w/convalescent period; 2) the phDC induced IFNγ response is durable and detectable out to 1 year post Covid infection.

FIG. 17C: Vaccinated and convalescent donors' plate passed PBMC were depleted of either CD8 or CD4 T cells prior to incorporation into the standard 18 hr phDC [Spike] ELISpot IFNγ assay. Depletion of CD8 T cells eliminates the majority of phDC [Spike] response, while CD4 depletion has minimal effect. This demonstrates that convalescent phDC [Spike] IFNγ response is largely driven by CD8+ T cells.

FIG. 17D: An example of a phDC ELISpot using PBMC isolated from a single human donor prior to, and six weeks post, SARS-CoV2 infection. IFNγ release increased above the positive threshold of 50 SFU/Million cells following infection. This demonstrates the phDC induced IFNγ T cell response increases following natural Covid infection, thus showing the feasibility and utility of longitudinal immune monitoring.

1. A vaccine composition comprising a pharmaceutically effective amount of physiological DCs (phDCs) and at least one mRNA, which comprises a coding sequence encoding for at least one antigenic protein.

2. The vaccine composition according to claim 1, wherein said at least one mRNA is comprised in lipid nanoparticles.

3. The vaccine composition according to claim 1 or 2, wherein said at least one mRNA is modified with N1-methyl-pseudouridine in place of uridine.

4. The vaccine composition according to any of claims 1 to 3, wherein the lipid nanoparticles comprise a cKK-E12 lipid, a SM-102 lipid or a MC3 lipid.

5. The vaccine composition according to any of claims 1 to 4, wherein the phDCs are obtainable by subjecting monocytes obtained from a donor to a shear force by passing said monocytes through a flow chamber.

6. The vaccine composition according to any of claims 1 to 5, wherein said at least one antigenic protein is an infectious disease associated antigenic protein, optionally a viral antigenic protein, a bacterial antigenic protein, a fungal antigenic protein, a prion antigenic protein or a parasite antigenic protein.

7. The vaccine composition according to claim 6, wherein said viral antigenic protein is a coronavirus antigenic protein or human immunodeficiency virus (HIV) antigenic protein.

8. The vaccine composition according to any one of claims 1 to 7, wherein said at least one antigenic protein is exogenous to the phDCs.

9. The vaccine composition according to any of claims 1 to 6, wherein said at least one mRNA comprises a nucleotide sequence which is at least 75%, 85%, 90%, 95%, 98% or 99% or 100% identical to SEQ ID NO: 1.

10. The vaccine composition according to any of claims 1 to 7, wherein said at least one mRNA comprises a nucleotide sequence which is at least 75%, 85%, 90%, 95%, 98% or 99% or 100% identical to SEQ ID NO: 19.

11. The vaccine composition according to any one of claims 1 to 9, wherein said at least one mRNA is heterologous mRNA or exogenous mRNA to said phDCs.

12. The vaccine composition according to any one of claims 1 to 10, wherein the mRNA is at least partially comprised in the phDCs.

13. The vaccine composition according to claim 11, wherein substantially all of the mRNA is comprised in the phDCs.

14. The vaccine composition according to claim 12, wherein the composition comprises unincorporated mRNA.

15. The vaccine composition according to any one of claims 1 to 10, wherein the mRNA is at least partially not incorporated into the phDCs.

16. The vaccine composition according to any one of 14, wherein more than 30%, 40%, 50%, 60%, 70%, 80%, or 90% of the mRNA is not incorporated into the phDCs.

17. The vaccine composition according to any of claims 1 to 16 wherein said at least one antigenic protein is a tumor associated antigenic protein.

18. The vaccine composition according to claim 17, wherein said tumor associated antigenic protein is a blood cancer antigenic protein.

19. The vaccine composition according to any one of claims 6 to 16 for use in a prophylactic method of preventing or reducing an infectious disease, preferably a viral disease, in a subject, said method comprising administering the vaccine composition to said subject.

20. The vaccine composition according to any one of claims 7 to 16 for use in a prophylactic method of preventing or reducing a coronavirus disease, preferably coronavirus disease 2019 (Covid-19), in a subject, said method comprising administering the vaccine composition to said subject.

21. The vaccine composition according to any of claims 17 or 18 for use in a prophylactic method of preventing or reducing a tumor or a cancer in a subject, said method comprising administering the vaccine composition to said subject.

22. The vaccine composition according to claim 18 for use in a prophylactic method of preventing or reducing a blood cancer in a subject, said method comprising administering the vaccine composition to said subject.


SEQUENCE LISTING (22 sequences)

SEQ ID NO: 1 - RNA, 3822 nt, SARS-CoV-2
  source 1..3822: mol_type = mRNA
atgtttgtttttcttgttttattgccactagtctctagtcagtgtgttaatcttacaaccagaactcaattaccccctgcatacactaattctttcacacgtggtgtttattaccctgacaaagttttcagatcctcagttttacattcaactcaggacttgttcttacctttcttttccaatgttacttggttccatgctatacatgtctctgggaccaatggtactaagaggtttgataaccctgtcctaccatttaatgatggtgtttattttgcttccactgagaagtctaacataataagaggctggatttttggtactactttagattcgaagacccagtccctacttattgttaataacgctactaatgttgttattaaagtctgtgaatttcaattttgtaatgatccatttttgggtgtttattaccacaaaaacaacaaaagttggatggaaagtgagttcagagtttattctagtgcgaataattgcacttttgaatatgtctctcagccttttcttatggaccttgaaggaaaacagggtaatttcaaaaatcttagggaatttgtgtttaagaatattgatggttattttaaaatatattctaagcacacgcctattaatttagtgcgtgatctccctcagggtttttcggctttagaaccattggtagatttgccaataggtattaacatcactaggtttcaaactttacttgctttacatagaagttatttgactcctggtgattcttcttcaggttggacagctggtgctgcagcttattatgtgggttatcttcaacctaggacttttctattaaaatataatgaaaatggaaccattacagatgctgtagactgtgcacttgaccctctctcagaaacaaagtgtacgttgaaatccttcactgtagaaaaaggaatctatcaaacttctaactttagagtccaaccaacagaatctattgttagatttcctaatattacaaacttgtgcccttttggtgaagtttttaacgccaccagatttgcatctgtttatgcttggaacaggaagagaatcagcaactgtgttgctgattattctgtcctatataattccgcatcattttccacttttaagtgttatggagtgtctcctactaaattaaatgatctctgctttactaatgtctatgcagattcatttgtaattagaggtgatgaagtcagacaaatcgctccagggcaaactggaaagattgctgattataattataaattaccagatgattttacaggctgcgttatagcttggaattctaacaatcttgattctaaggttggtggtaattataattacctgtatagattgtttaggaagtctaatctcaaaccttttgagagagatatttcaactgaaatctatcaggccggtagcacaccttgtaatggtgttgaaggttttaattgttactttcctttacaatcatatggtttccaacccactaatggtgttggttaccaaccatacagagtagtagtactttcttttgaacttctacatgcaccagcaactgtttgtggacctaaaaagtctactaatttggttaaaaacaaatgtgtcaatttcaacttcaatggtttaacaggcacaggtgttcttactgagtctaacaaaaagtttctgcctttccaacaatttggcagagacattgctgacactactgatgctgtccgtgatccacagacacttgagattcttgacattacaccatgttcttttggtggtgtcagtgttataacaccaggaacaaatacttctaaccaggttgctgttctttatcaggatgttaactgcacagaagtccctgttgctattcatgcagatcaacttactcctacttggcgtgtttattctacaggttctaatgtttttcaaacacgtgcaggctgtttaataggggctgaacatgtcaacaactcatatgagtgtgacatacccattggtgcaggtatatgcgctagttatcagactcagactaattctcctcggcgggcacgtagtgtagctagtcaatccatcattgcctacactatgtcacttggtgcagaaaattcagttgcttactctaataactctattgccatacccacaaattttactattagtgttaccacagaaattctaccagtgtctatgaccaagacatcagtagattgtacaatgtacatttgtggtgattcaactgaatgcagcaatcttttgttgcaatatggcagtttttgtacacaattaaaccgtgctttaactggaatagctgttgaacaagacaaaaacacccaagaagtttttgcacaagtcaaacaaatttacaaaacaccaccaattaaagattttggtggttttaatttttcacaaatattaccagatccatcaaaaccaagcaagaggtcatttattgaagatctacttttcaacaaagtgacacttgcagatgctggcttcatcaaacaatatggtgattgccttggtgatattgctgctagagacctcatttgtgcacaaaagtttaacggccttactgttttgccacctttgctcacagatgaaatgattgctcaatacacttctgcactgttagcgggtacaatcacttctggttggacctttggtgcaggtgctgcattacaaataccatttgctatgcaaatggcttataggtttaatggtattggagttacacagaatgttctctatgagaaccaaaaattgattgccaaccaatttaatagtgctattggcaaaattcaagactcactttcttccacagcaagtgcacttggaaaacttcaagatgtggtcaaccaaaatgcacaagctttaaacacgcttgttaaacaacttagctccaattttggtgcaatttcaagtgttttaaatgatatcctttcacgtcttgacaaagttgaggctgaagtgcaaattgataggttgatcacaggcagacttcaaagtttgcagacatatgtgactcaacaattaattagagctgcagaaatcagagcttctgctaatcttgctgctactaaaatgtcagagtgtgtacttggacaatcaaaaagagttgatttttgtggaaagggctatcatcttatgtccttccctcagtcagcacctcatggtgtagtcttcttgcatgtgacttatgtccctgcacaagaaaagaacttcacaactgctcctgccatttgtcatgatggaaaagcacactttcctcgtgaaggtgtctttgtttcaaatggcacacactggtttgtaacacaaaggaatttttatgaaccacaaatcattactacagacaacacatttgtgtctggtaactgtgatgttgtaataggaattgtcaacaacacagtttatgatcctttgcaacctgaattagactcattcaaggaggagttagataaatattttaagaatcatacatcaccagatgttgatttaggtgacatctctggcattaatgcttcagttgtaaacattcaaaaagaaattgaccgcctcaatgaggttgccaagaatttaaatgaatctctcatcgatctccaagaacttggaaagtatgagcagtatataaaatggccatggtacatttggctaggttttatagctggcttgattgccatagtaatggtgacaattatgctttgctgtatgaccagttgctgtagttgtctcaagggctgttgttcttgtggatcctgctgcaaatttgatgaagacgactctgagccagtgctcaaaggagtcaaattacattacacataa

SEQ ID NO: 2 - RNA, 228 nt, Severe acute respiratory syndrome-related coronavirus
  source 1..228: mol_type = mRNA
atgtactcattcgtttcggaagagacaggtacgttaatagttaatagcgtacttctttttcttgctttcgtggtattcttgctagttacactagccatccttactgcgcttcgattgtgtgcgtactgctgcaatattgttaacgtgagtcttgtaaaaccttctttttacgtttactctcgtgttaaaaatctgaattcttctagagttcctgatcttctggtctaa

SEQ ID NO: 3 - RNA, 1260 nt, Severe acute respiratory syndrome-related coronavirus
  source 1..1260: mol_type = mRNA
atgtctgataatggaccccaaaatcagcgaaatgcaccccgcattacgtttggtggaccctcagattcaactggcagtaaccagaatggagaacgcagtggggcgcgatcaaaacaacgtcggccccaaggtttacccaataatactgcgtcttggttcaccgctctcactcaacatggcaaggaagaccttaaattccctcgaggacaaggcgttccaattaacaccaatagcagtccagatgaccaaattggctactaccgaagagctaccagacgaattcgtggtggtgacggtaaaatgaaagatctcagtccaagatggtatttctactacctaggaactgggccagaagctggacttccctatggtgctaacaaagacggcatcatatgggttgcaactgagggagccttgaatacaccaaaagatcacattggcacccgcaatcctgctaacaatgctgcaatcgtgctacaacttcctcaaggaacaacattgccaaaaggcttctacgcagaagggagcagaggcggcagtcaagcctcttctcgttcctcatcacgtagtcgcaacagttcaagaaattcaactccaggcagcagtaggggaacttctcctgctagaatggctggcaatggcggtgatgctgctcttgctttgctgctgcttgacagattgaaccagcttgagagcaaaatgtctggtaaaggccaacaacaacaaggccaaactgtcactaagaaatctgctgctgaggcttctaagaagcctcggcaaaaacgtactgccactaaagcatacaatgtaacacaagctttcggcagacgtggtccagaacaaacccaaggaaattttggggaccaggaactaatcagacaaggaactgattacaaacattggccgcaaattgcacaatttgcccccagcgcttcagcgttcttcggaatgtcgcgcattggcatggaagtcacaccttcgggaacgtggttgacctacacaggtgccatcaaattggatgacaaagatccaaatttcaaagatcaagtcattttgctgaataagcatattgacgcatacaaaacattcccaccaacagagcctaaaaaggacaaaaagaagaaggctgatgaaactcaagccttaccgcagagacagaagaaacagcaaactgtgactcttcttcctgctgcagatttggatgatttctccaaacaattgcaacaatccatgagcagtgctgactcaactcaggcctaa

SEQ ID NO: 4 - RNA, 669 nt, Severe acute respiratory syndrome-related coronavirus
  source 1..669: mol_type = mRNA
atggcagattccaacggtactattaccgttgaagagcttaaaaagctccttgaacaatggaacctagtaataggtttcctattccttacatggatttgtcttctacaatttgcctatgccaacaggaataggtttttgtatataattaagttaattttcctctggctgttatggccagtaactttagcttgttttgtgcttgctgctgtttacagaataaattggatcaccggtggaattgctatcgcaatggcttgtcttgtaggcttgatgtggctcagctacttcattgcttctttcagactgtttgcgcgtacgcgttccatgtggtcattcaatccagaaactaacattcttctcaacgtgccactccatggcactattctgaccagaccgcttctagaaagtgaactcgtaatcggagctgtgatccttcgtggacatcttcgtattgctggacaccatctaggacgctgtgacatcaaggacctgcctaaagaaatcactgttgctacatcacgaacgctttcttattacaaattgggagcttcgcagcgtgtagcaggtgactcaggttttgctgcatacagtcgctacaggattggcaactataaattaaacacagaccattccagtagcagtgacaatattgctttgcttgtacagtaa

SEQ ID NO: 5 - RNA, 21290 nt, Severe acute respiratory syndrome-related coronavirus
  source 1..21290: mol_type = mRNA
atggagagccttgtccctggtttcaacgagaaaacacacgtccaactcagtttgcctgttttacaggttcgcgacgtgctcgtacgtggctttggagactccgtggaggaggtcttatcagaggcacgtcaacatcttaaagatggcacttgtggcttagtagaagttgaaaaaggcgttttgcctcaacttgaacagccctatgtgttcatcaaacgttcggatgctcgaactgcacctcatggtcatgttatggttgagctggtagcagaactcgaaggcattcagtacggtcgtagtggtgagacacttggtgtccttgtccctcatgtgggcgaaataccagtggcttaccgcaaggttcttcttcgtaagaacggtaataaaggagctggtggccatagttacggcgccgatctaaagtcatttgacttaggcgacgagcttggcactgatccttatgaagattttcaagaaaactggaacactaaacatagcagtggtgttacccgtgaactcatgcgtgagcttaacggaggggcatacactcgctatgtcgataacaacttctgtggccctgatggctaccctcttgagtgcattaaagaccttctagcacgtgctggtaaagcttcatgcactttgtccgaacaactggactttattgacactaagaggggtgtatactgctgccgtgaacatgagcatgaaattgcttggtacacggaacgttctgaaaagagctatgaattgcagacaccttttgaaattaaattggcaaagaaatttgacaccttcaatggggaatgtccaaattttgtatttcccttaaattccataatcaagactattcaaccaagggttgaaaagaaaaagcttgatggctttatgggtagaattcgatctgtctatccagttgcgtcaccaaatgaatgcaaccaaatgtgcctttcaactctcatgaagtgtgatcattgtggtgaaacttcatggcagacgggcgattttgttaaagccacttgcgaattttgtggcactgagaatttgactaaagaaggtgccactacttgtggttacttaccccaaaatgctgttgttaaaatttattgtccagcatgtcacaattcagaagtaggacctgagcatagtcttgccgaataccataatgaatctggcttgaaaaccattcttcgtaagggtggtcgcactattgcctttggaggctgtgtgttctcttatgttggttgccataacaagtgtgcctattgggttccacgtgctagcgctaacataggttgtaaccatacaggtgttgttggagaaggttccgaaggtcttaatgacaaccttcttgaaatactccaaaaagagaaagtcaacatcaatattgttggtgactttaaacttaatgaagagatcgccattattttggcatctttttctgcttccacaagtgcttttgtggaaactgtgaaaggtttggattataaagcattcaaacaaattgttgaatcctgtggtaattttaaagttacaaaaggaaaagctaaaaaaggtgcctggaatattggtgaacagaaatcaatactgagtcctctttatgcatttgcatcagaggctgctcgtgttgtacgatcaattttctcccgcactcttgaaactgctcaaaattctgtgcgtgttttacagaaggccgctataacaatactagatggaatttcacagtattcactgagactcattgatgctatgatgttcacatctgatttggctactaacaatctagttgtaatggcctacattacaggtggtgttgttcagttgacttcgcagtggctaactaacatctttggcactgtttatgaaaaactcaaacccgtccttgattggcttgaagagaagtttaaggaaggtgtagagtttcttagagacggttgggaaattgttaaatttatctcaacctgtgcttgtgaaattgtcggtggacaaattgtcacctgtgcaaaggaaattaaggagagtgttcagacattctttaagcttgtaaataaatttttggctttgtgtgctgactctatcattattggtggagctaaacttaaagccttgaatttaggtgaaacatttgtcacgcactcaaagggattgtacagaaagtgtgttaaatccagagaagaaactggcctactcatgcctctaaaagccccaaaagaaattatcttcttagagggagaaacacttcccacagaagtgttaacagaggaagttgtcttgaaaactggtgatttacaaccattagaacaacctactagtgaagctgttgaagctccattggttggtacaccagtttgtattaacgggcttatgttgctcgaaatcaaagacacagaaaagtactgtgcccttgcacctaatatgatggtaacaaacaataccttcacactcaaaggcggtgcaccaacaaaggttacttttggtgatgacactgtgatagaagtgcaaggttacaagagtgtgaatatcacttttgaacttgatgaaaggattgataaagtacttaatgagaagtgctctgcctatacagttgaactcggtacagaagtaaatgagttcgcctgtgttgtggcagatgctgtcataaaaactttgcaaccagtatctgaattacttacaccactgggcattgatttagatgagtggagtatggctacatactacttatttgatgagtctggtgagtttaaattggcttcacatatgtattgttctttctaccctccagatgaggatgaagaagaaggtgattgtgaagaagaagagtttgagccatcaactcaatatgagtatggtactgaagatgattaccaaggtaaacctttggaatttggtgccacttctgctgctcttcaacctgaagaagagcaagaagaagattggttagatgatgatagtcaacaaactgttggtcaacaagacggcagtgaggacaatcagacaactactattcaaacaattgttgaggttcaacctcaattagagatggaacttacaccagttgttcagactattgaagtgaatagttttagtggttatttaaaacttactgacaatgtatacattaaaaatgcagacattgtggaagaagctaaaaaggtaaaaccaacagtggttgttaatgcagccaatgtttaccttaaacatggaggaggtgttgcaggagccttaaataaggctactaacaatgccatgcaagttgaatctgatgattacatagctactaatggaccacttaaagtgggtggtagttgtgttttaagcggacacaatcttgctaaacactgtcttcatgttgtcggcccaaatgttaacaaaggtgaagacattcaacttcttaagagtgcttatgaaaattttaatcagcacgaagttctacttgcaccattattatcagctggtatttttggtgctgaccctatacattctttaagagtttgtgtagatactgttcgcacaaatgtctacttagctgtctttgataaaaatctctatgacaaacttgtttcaagctttttggaaatgaagagtgaaaagcaagttgaacaaaagatcgctgagattcctaaagaggaagttaagccatttataactgaaagtaaaccttcagttgaacagagaaaacaagatgataagaaaatcaaagcttgtgttgaagaagttacaacaactctggaagaaactaagttcctcacagaaaacttgttactttatattgacattaatggcaatcttcatccagattctgccactcttgttagtgacattgacatcactttcttaaagaaagatgctccatatatagtgggtgatgttgttcaagagggtgttttaactgctgtggttatacctactaaaaaggctggtggcactactgaaatgctagcgaaagctttgagaaaagtgccaacagacaattatataaccacttacccgggtcagggtttaaatggttacactgtagaggaggcaaagacagtgcttaaaaagtgtaaaagtgccttttacattctaccatctattatctctaatgagaagcaagaaattcttggaactgtttcttggaatttgcgagaaatgcttgcacatgcagaagaaacacgcaaattaatgcctgtctgtgtggaaactaaagccatagtttcaactatacagcgtaaatataagggtattaaaatacaagagggtgtggttgattatggtgctagattttacttttacaccagtaaaacaactgtagcgtcacttatcaacacacttaacgatctaaatgaaactcttgttacaatgccacttggctatgtaacacatggcttaaatttggaagaagctgctcggtatatgagatctctcaaagtgccagctacagtttctgtttcttcacctgatgctgttacagcgtataatggttatcttacttcttcttctaaaacacctgaagaacattttattgaaaccatctcacttgctggttcctataaagattggtcctattctggacaatctacacaactaggtatagaatttcttaagagaggtgataaaagtgtatattacactagtaatcctaccacattccacctagatggtgaagttatcacctttgacaatcttaagacacttctttctttgagagaagtgaggactattaaggtgtttacaacagtagacaacattaacctccacacgcaagttgtggacatgtcaatgacatatggacaacagtttggtccaacttatttggatggagctgatgttactaaaataaaacctcataattcacatgaaggtaaaacattttatgttttacctaatgatgacactctacgtgttgaggcttttgagtactaccacacaactgatcctagttttctgggtaggtacatgtcagcattaaatcacactaaaaagtggaaatacccacaagttaatggtttaacttctattaaatgggcagataacaactgttatcttgccactgcattgttaacactccaacaaatagagttgaagtttaatccacctgctctacaagatgcttattacagagcaagggctggtgaagctgctaacttttgtgcacttatcttagcctactgtaataagacagtaggtgagttaggtgatgttagagaaacaatgagttacttgtttcaacatgccaatttagattcttgcaaaagagtcttgaacgtggtgtgtaaaacttgtggacaacagcagacaacccttaagggtgtagaagctgttatgtacatgggcacactttcttatgaacaatttaagaaaggtgttcagataccttgtacgtgtggtaaacaagctacaaaatatctagtacaacaggagtcaccttttgttatgatgtcagcaccacctgctcagtatgaacttaagcatggtacatttacttgtgctagtgagtacactggtaattaccagtgtggtcactataaacatataacttctaaagaaactttgtattgcatagacggtgctttacttacaaagtcctcagaatacaaaggtcctattacggatgttttctacaaagaaaacagttacacaacaaccataaaaccagttacttataaattggatggtgttgtttgtacagaaattgaccctaagttggacaattattataagaaagacaattcttatttcacagagcaaccaattgatcttgtaccaaaccaaccatatccaaacgcaagcttcgataattttaagtttgtatgtgataatatcaaatttgctgatgatttaaaccagttaactggttataagaaacctgcttcaagagagcttaaagttacatttttccctgacttaaatggtgatgtggtggctattgattataaacactacacaccctcttttaagaaaggagctaaattgttacataaacctattgtttggcatgttaacaatgcaactaataaagccacgtataaaccaaatacctggtgtatacgttgtctttggagcacaaaaccagttgaaacatcaaattcgtttgatgtactgaagtcagaggacgcgcagggaatggataatcttgcctgcgaagatctaaaaccagtctctgaagaagtagtggaaaatcctaccatacagaaagacgttcttgagtgtaatgtgaaaactaccgaagttgtaggagacattatacttaaaccagcaaataatagtttaaaaattacagaagaggttggccacacagatctaatggctgcttatgtagacaattctagtcttactattaagaaacctaatgaattatctagagtattaggtttgaaaacccttgctactcatggtttagctgctgttaatagtgtcccttgggatactatagctaattatgctaagccttttcttaacaaagttgttagtacaactactaacatagttacacggtgtttaaaccgtgtttgtactaattatatgccttatttctttactttattgctacaattgtgtacttttactagaagtacaaattctagaattaaagcatctatgccgactactatagcaaagaatactgttaagagtgtcggtaaattttgtctagaggcttcatttaattatttgaagtcacctaatttttctaaactgataaatattataatttggtttttactattaagtgtttgcctaggttctttaatctactcaaccgctgctttaggtgttttaatgtctaatttaggcatgccttcttactgtactggttacagagaaggctatttgaactctactaatgtcactattgcaacctactgtactggttctataccttgtagtgtttgtcttagtggtttagattctttagacacctatccttctttagaaactatacaaattaccatttcatcttttaaatgggatttaactgcttttggcttagttgcagagtggtttttggcatatattcttttcactaggtttttctatgtacttggattggctgcaatcatgcaattgtttttcagctattttgcagtacattttattagtaattcttggcttatgtggttaataattaatcttgtacaaatggccccgatttcagctatggttagaatgtacatcttctttgcatcattttattatgtatggaaaagttatgtgcatgttgtagacggttgtaattcatcaacttgtatgatgtgttacaaacgtaatagagcaacaagagtcgaatgtacaactattgttaatggtgttagaaggtccttttatgtctatgctaatggaggtaaaggcttttgcaaactacacaattggaattgtgttaattgtgatacattctgtgctggtagtacatttattagtgatgaagttgcgagagacttgtcactacagtttaaaagaccaataaatcctactgaccagtcttcttacatcgttgatagtgttacagtgaagaatggttccatccatctttactttgataaagctggtcaaaagacttatgaaagacattctctctctcattttgttaacttagacaacctgagagctaataacactaaaggttcattgcctattaatgttatagtttttgatggtaaatcaaaatgtgaagaatcatctgcaaaatcagcgtctgtttactacagtcagcttatgtgtcaacctatactgttactagatcaggcattagtgtctgatgttggtgatagtgcggaagttgcagttaaaatgtttgatgcttacgttaatacgttttcatcaacttttaacgtaccaatggaaaaactcaaaacactagttgcaactgcagaagctgaacttgcaaagaatgtgtccttagacaatgtcttatctacttttatttcagcagctcggcaagggtttgttgattcagatgtagaaactaaagatgttgttgaatgtcttaaattgtcacatcaatctgacatagaagttactggcgatagttgtaataactatatgctcacctataacaaagttgaaaacatgacaccccgtgaccttggtgcttgtattgactgtagtgcgcgtcatattaatgcgcaggtagcaaaaagtcacaacattgctttgatatggaacgttaaagatttcatgtcattgtctgaacaactacgaaaacaaatacgtagtgctgctaaaaagaataacttaccttttaagttgacatgtgcaactactagacaagttgttaatgttgtaacaacaaagatagcacttaagggtggtaaaattgttaataattggttgaagcagttaattaaagttacacttgtgttcctttttgttgctgctattttctatttaataacacctgttcatgtcatgtctaaacatactgacttttcaagtgaaatcataggatacaaggctattgatggtggtgtcactcgtgacatagcatctacagatacttgttttgctaacaaacatgctgattttgacacatggtttagccagcgtggtggtagttatactaatgacaaagcttgcccattgattgctgcagtcataacaagagaagtgggttttgtcgtgcctggtttgcctggcacgatattacgcacaactaatggtgactttttgcatttcttacctagagtttttagtgcagttggtaacatctgttacacaccatcaaaacttatagagtacactgactttgcaacatcagcttgtgttttggctgctgaatgtacaatttttaaagatgcttctggtaagccagtaccatattgttatgataccaatgtactagaaggttctgttgcttatgaaagtttacgccctgacacacgttatgtgctcatggatggctctattattcaatttcctaacacctaccttgaaggttctgttagagtggtaacaacttttgattctgagtactgtaggcacggcacttgtgaaagatcagaagctggtgtttgtgtatctactagtggtagatgggtacttaacaatgattattacagatctttaccaggagttttctgtggtgtagatgctgtaaatttacttactaatatgtttacaccactaattcaacctattggtgctttggacatatcagcatctatagtagctggtggtattgtagctatcgtagtaacatgccttgcctactattttatgaggtttagaagagcttttggtgaatacagtcatgtagttgcctttaatactttactattccttatgtcattcactgtactctgtttaacaccagtttactcattcttacctggtgtttattctgttatttacttgtacttgacattttatcttactaatgatgtttcttttttagcacatattcagtggatggttatgttcacacctttagtacctttctggataacaattgcttatatcatttgtatttccacaaagcatttctattggttctttagtaattacctaaagagacgtgtagtctttaatggtgtttcctttagtacttttgaagaagctgcgctgtgcacctttttgttaaataaagaaatgtatctaaagttgcgtagtgatgtgctattacctcttacgcaatataatagatacttagctctttataataagtacaagtattttagtggagcaatggatacaactagctacagagaagctgcttgttgtcatctcgcaaaggctctcaatgacttcagtaactcaggttctgatgttctttaccaaccaccacaaacctctatcacctcagctgttttgcagagtggttttagaaaaatggcattcccatctggtaaagttgagggttgtatggtacaagtaacttgtggtacaactacacttaacggtctttggcttgatgacgtagtttactgtccaagacatgtgatctgcacctctgaagacatgcttaaccctaattatgaagatttactcattcgtaagtctaatcataatttcttggtacaggctggtaatgttcaactcagggttattggacattctatgcaaaattgtgtacttaagcttaaggttgatacagccaatcctaagacacctaagtataagtttgttcgcattcaaccaggacagactttttcagtgttagcttgttacaatggttcaccatctggtgtttaccaatgtgctatgaggcccaatttcactattaagggttcattccttaatggttcatgtggtagtgttggttttaacatagattatgactgtgtctctttttgttacatgcaccatatggaattaccaactggagttcatgctggcacagacttagaaggtaacttttatggaccttttgttgacaggcaaacagcacaagcagctggtacggacacaactattacagttaatgttttagcttggttgtacgctgctgttataaatggagacaggtggtttctcaatcgatttaccacaactcttaatgactttaaccttgtggctatgaagtacaattatgaacctctaacacaagaccatgttgacatactaggacctctttctgctcaaactggaattgccgttttagatatgtgtgcttcattaaaagaattactgcaaaatggtatgaatggacgtaccatattgggtagtgctttattagaagatgaatttacaccttttgatgttgttagacaatgctcaggtgttactttccaaagtgcagtgaaaagaacaatcaagggtacacaccactggttgttactcacaattttgacttcacttttagttttagtccagagtactcaatggtctttgttcttttttttgtatgaaaatgcctttttaccttttgctatgggtattattgctatgtctgcttttgcaatgatgtttgtcaaacataagcatgcatttctctgtttgtttttgttaccttctcttgccactgtagcttattttaatatggtctatatgcctgctagttgggtgatgcgtattatgacatggttggatatggttgatactagtttgtctggttttaagctaaaagactgtgttatgtatgcatcagctgtagtgttactaatccttatgacagcaagaactgtgtatgatgatggtgctaggagagtgtggacacttatgaatgtcttgacactcgtttataaagtttattatggtaatgctttagatcaagccatttccatgtgggctcttataatctctgttacttctaactactcaggtgtagttacaactgtcatgtttttggccagaggtattgtttttatgtgtgttgagtattgccctattttcttcataactggtaatacacttcagtgtataatgctagtttattgtttcttaggctatttttgtacttgttactttggcctcttttgtttactcaaccgctactttagactgactcttggtgtttatgattacttagtttctacacaggagtttagatatatgaattcacagggactactcccacccaagaatagcatagatgccttcaaactcaacattaaattgttgggtgttggtggcaaaccttgtatcaaagtagccactgtacagtctaaaatgtcagatgtaaagtgcacatcagtagtcttactctcagttttgcaacaactcagagtagaatcatcatctaaattgtgggctcaatgtgtccagttacacaatgacattctcttagctaaagatactactgaagcctttgaaaaaatggtttcactactttctgttttgctttccatgcagggtgctgtagacataaacaagctttgtgaagaaatgctggacaacagggcaaccttacaagctatagcctcagagtttagttcccttccatcatatgcagcttttgctactgctcaagaagcttatgagcaggctgttgctaatggtgattctgaagttgttcttaaaaagttgaagaagtctttgaatgtggctaaatctgaatttgaccgtgatgcagccatgcaacgtaagttggaaaagatggctgatcaagctatgacccaaatgtataaacaggctagatctgaggacaagagggcaaaagttactagtgctatgcagacaatgcttttcactatgcttagaaagttggataatgatgcactcaacaacattatcaacaatgcaagagatggttgtgttcccttgaacataatacctcttacaacagcagccaaactaatggttgtcataccagactataacacatataaaaatacgtgtgatggtacaacatttacttatgcatcagcattgtgggaaatccaacaggttgtagatgcagatagtaaaattgttcaacttagtgaaattagtatggacaattcacctaatttagcatggcctcttattgtaacagctttaagggccaattctgctgtcaaattacagaataatgagcttagtcctgttgcactacgacagatgtcttgtgctgccggtactacacaaactgcttgcactgatgacaatgcgttagcttactacaacacaacaaagggaggtaggtttgtacttgcactgttatccgatttacaggatttgaaatgggctagattccctaagagtgatggaactggtactatctatacagaactggaaccaccttgtaggtttgttacagacacacctaaaggtcctaaagtgaagtatttatactttattaaaggattaaacaacctaaatagaggtatggtacttggtagtttagctgccacagtacgtctacaagctggtaatgcaacagaagtgcctgccaattcaactgtattatctttctgtgcttttgctgtagatgctgctaaagcttacaaagattatctagctagtgggggacaaccaatcactaattgtgttaagatgttgtgtacacacactggtactggtcaggcaataacagttacaccggaagccaatatggatcaagaatcctttggtggtgcatcgtgttgtctgtactgccgttgccacatagatcatccaaatcctaaaggattttgtgacttaaaaggtaagtatgtacaaatacctacaacttgtgctaatgaccctgtgggttttacacttaaaaacacagtctgtaccgtctgcggtatgtggaaaggttatggctgtagttgtgatcaactccgcgaacccatgcttcagtcagctgatgcacaatcgtttttaaacgggtttgcggtgtaagtgcagcccgtcttacaccgtgcggcacaggcactagtactgatgtcgtatacagggcttttgacatctacaatgataaagtagctggttttgctaaattcctaaaaactaattgttgtcgcttccaagaaaaggacgaagatgacaatttaattgattcttactttgtagttaagagacacactttctctaactaccaacatgaagaaacaatttataatttacttaaggattgtccagctgttgctaaacatgacttctttaagtttagaatagacggtgacatggtaccacatatatcacgtcaacgtcttactaaatacacaatggcagacctcgtctatgctttaaggcattttgatgaaggtaattgtgacacattaaaagaaatacttgtcacatacaattgttgtgatgatgattatttcaataaaaaggactggtatgattttgtagaaaacccagatatattacgcgtatacgccaacttaggtgaacgtgtacgccaagctttgttaaaaacagtacaattctgtgatgccatgcgaaatgctggtattgttggtgtactgacattagataatcaagatctcaatggtaactggtatgatttcggtgatttcatacaaaccacgccaggtagtggagttcctgttgtagattcttattattcattgttaatgcctatattaaccttgaccagggctttaactgcagagtcacatgttgacactgacttaacaaagccttacattaagtgggatttgttaaaatatgacttcacggaagagaggttaaaactctttgaccgttattttaaatattgggatcagacataccacccaaattgtgttaactgtttggatgacagatgcattctgcattgtgcaaactttaatgttttattctctacagtgttcccacctacaagttttggaccactagtgagaaaaatatttgttgatggtgttccatttgtagtttcaactggataccacttcagagagctaggtgttgtacataatcaggatgtaaacttacatagctctagacttagttttaaggaattacttgtgtatgctgctgaccctgctatgcacgctgcttctggtaatctattactagataaacgcactacgtgcttttcagtagctgcacttactaacaatgttgcttttcaaactgtcaaacccggtaattttaacaaagacttctatgactttgctgtgtctaagggtttctttaaggaaggaagttctgttgaattaaaacacttcttctttgctcaggatggtaatgctgctatcagcgattatgactactatcgttataatctaccaacaatgtgtgatatcagacaactactatttgtagttgaagttgttgataagtactttgattgttacgatggtggctgtattaatgctaaccaagtcatcgtcaacaacctagacaaatcagctggttttccatttaataaatggggtaaggctagactttattatgattcaatgagttatgaggatcaagatgcacttttcgcatatacaaaacgtaatgtcatccctactataactcaaatgaatcttaagtatgccattagtgcaaagaatagagctcgcaccgtagctggtgtctctatctgtagtactatgaccaatagacagtttcatcaaaaattattgaaatcaatagccgccactagaggagctactgtagtaattggaacaagcaaattctatggtggttggcacaacatgttaaaaactgtttatagtgatgtagaaaaccctcaccttatgggttgggattatcctaaatgtgatagagccatgcctaacatgcttagaattatggcctcacttgttcttgctcgcaaacatacaacgtgttgtagcttgtcacaccgtttctatagattagctaatgagtgtgctcaagtattgagtgaaatggtcatgtgtggcggttcactatatgttaaaccaggtggaacctcatcaggagatgccacaactgcttatgctaatagtgtttttaacatttgtcaagctgtcacggccaatgttaatgcacttttatctactgatggtaacaaaattgccgataagtatgtccgcaatttacaacacagactttatgagtgtctctatagaaatagagatgttgacacagactttgtgaatgagttttacgcatatttgcgtaaacatttctcaatgatgatactctctgacgatgctgttgtgtgtttcaatagcacttatgcatctcaaggtctagtggctagcataaagaactttaagtcagttctttattatcaaaacaatgtttttatgtctgaagcaaaatgttggactgagactgaccttactaaaggacctcatgaattttgctctcaacatacaatgctagttaaacagggtgatgattatgtgtaccttccttacccagatccatcaagaatcctaggggccggctgttttgtagatgatatcgtaaaaacagatggtacacttatgattgaacggttcgtgtctttagctatagatgcttacccacttactaaacatcctaatcaggagtatgctgatgtctttcatttgtacttacaatacataagaaagctacatgatgagttaacaggacacatgttagacatgtattctgttatgcttactaatgataacacttcaaggtattgggaacctgagttttatgaggctatgtacacaccgcatacagtcttacaggctgttggggcttgtgttctttgcaattcacagacttcattaagatgtggtgcttgcatacgtagaccattcttatgttgtaaatgctgttacgaccatgtcatatcaacatcacataaattagtcttgtctgttaatccgtatgtttgcaatgctccaggttgtgatgtcacagatgtgactcaactttacttaggaggtatgagctattattgtaaatcacataaaccacccattagttttccattgtgtgctaatggacaagtttttggtttatataaaaatacatgtgttggtagcgataatgttactgactttaatgcaattgcaacatgtgactggacaaatgctggtgattacattttagctaacacctgtactgaaagactcaagctttttgcagcagaaacgctcaaagctactgaggagacatttaaactgtcttatggtattgctactgtacgtgaagtgctgtctgacagagaattacatctttcatgggaagttggtaaacctagaccaccacttaaccgaaattatgtctttactggttatcgtgtaactaaaaacagtaaagtacaaataggagagtacacctttgaaaaaggtgactatggtgatgctgttgtttaccgaggtacaacaacttacaaattaaatgttggtgattattttgtgctgacatcacatacagtaatgccattaagtgcacctacactagtgccacaagagcactatgttagaattactggcttatacccaacactcaatatctcagatgagttttctagcaatgttgcaaattatcaaaaggttggtatgcaaaagtattctacactccagggaccacctggtactggtaagagtcattttgctattggcctagctctctactacccttctgctcgcatagtgtatacagcttgctctcatgccgctgttgatgcactatgtgagaaggcattaaaatatttgcctatagataaatgtagtagaattatacctgcacgtgctcgtgtagagtgttttgataaattcaaagtgaattcaacattagaacagtatgtcttttgtactgtaaatgcattgcctgagacgacagcagatatagttgtctttgatgaaatttcaatggccacaaattatgatttgagtgttgtcaatgccagattacgtgctaagcactatgtgtacattggcgaccctgctcaattacctgcaccacgcacattgctaactaagggcacactagaaccagaatatttcaattcagtgtgtagacttatgaaaactataggtccagacatgttcctcggaacttgtcggcgttgtcctgctgaaattgttgacactgtgagtgctttggtttatgataataagcttaaagcacataaagacaaatcagctcaatgctttaaaatgttttataagggtgttatcacgcatgatgtttcatctgcaattaacaggccacaaataggcgtggtaagagaattccttacacgtaaccctgcttggagaaaagctgtctttatttcaccttataattcacagaatgctgtagcctcaaagattttgggactaccaactcaaactgttgattcatcacagggctcagaatatgactatgtcatattcactcaaaccactgaaacagctcactcttgtaatgtaaacagatttaatgttgctattaccagagcaaaagtaggcatactttgcataatgtctgatagagacctttatgacaagttgcaatttacaagtcttgaaattccacgtaggaatgtggcaactttacaagctgaaaatgtaacaggactctttaaagattgtagtaaggtaatcactgggttacatcctacacaggcacctacacacctcagtgttgacactaaattcaaaactgaaggtttatgtgttgacatacctggcatacctaaggacatgacctatagaagactcatctctatgatgggttttaaaatgaattatcaagttaatggttaccctaacatgtttatcacccgcgaagaagctataagacatgtacgtgcatggattggcttcgatgtcgaggggtgtcatgctactagagaagctgttggtaccaatttacctttacagctaggtttttctacaggtgttaacctagttgctgtacctacaggttatgttgatacacctaataatacagatttttccagagttagtgctaaaccaccgcctggagatcaatttaaacacctcataccacttatgtacaaaggacttccttggaatgtagtgcgtataaagattgtacaaatgttaagtgacacacttaaaaatctctctgacagagtcgtatttgtcttatgggcacatggctttgagttgacatctatgaagtattttgtgaaaataggacctgagcgcacctgttgtctatgtgatagacgtgccacatgcttttccactgcttcagacacttatgcctgttggcatcattctattggatttgattacgtctataatccgtttatgattgatgttcaacaatggggttttacaggtaacctacaaagcaaccatgatctgtattgtcaagtccatggtaatgcacatgtagctagttgtgatgcaatcatgactaggtgtctagctgtccacgagtgctttgttaagcgtgttgactggactattgaatatcctataattggtgatgaactgaagattaatgcggcttgtagaaaggttcaacacatggttgttaaagctgcattattagcagacaaattcccagttcttcacgacattggtaaccctaaagctattaagtgtgtacctcaagctgatgtagaatggaagttctatgatgcacagccttgtagtgacaaagcttataaaatagaagaattattctattcttatgccacacattctgacaaattcacagatggtgtatgcctattttggaattgcaatgtcgatagatatcctgctaattccattgtttgtagatttgacactagagtgctatctaaccttaacttgcctggttgtgatggtggcagtttgtatgtaaataaacatgcattccacacaccagcttttgataaaagtgcttttgttaatttaaaacaattaccatttttctattactctgacagtccatgtgagtctcatggaaaacaagtagtgtcagatatagattatgtaccactaaagtctgctacgtgtataacacgttgcaatttaggtggtgctgtctgtagacatcatgctaatgagtacagattgtatctcgatgcttataacatgatgatctcagctggctttagcttgtgggtttacaaacaatttgatacttataacctctggaacacttttacaagacttcagagtttagaaaatgtggcttttaatgttgtaaataagggacactttgatggacaacagggtgaagtaccagtttctatcattaataacactgtttacacaaaagttgatggtgttgatgtagaattgtttgaaaataaaacaacattacctgttaatgtagcatttgagctttgggctaagcgcaacattaaaccagtaccagaggtgaaaatactcaataatttgggtgtggacattgctgctaatactgtgatctgggactacaaaagagatgctccagcacatatatctactattggtgtttgttctatgactgacatagccaagaaaccaactgaaacgatttgtgcaccactcactgtcttttttgatggtagagttgatggtcaagtagacttatttagaaatgcccgtaatggtgttcttattacagaaggtagtgttaaaggtttacaaccatctgtaggtcccaaacaagctagtcttaatggagtcacattaattggagaagccgtaaaaacacagttcaattattataagaaagttgatggtgttgtccaacaattacctgaaacttactttactcagagtagaaatttacaagaatttaaacccaggagtcaaatggaaattgatttcttagaattagctatggatgaattcattgaacggtataaattagaaggctatgccttcgaacatatcgtttatggagattttagtcatagtcagttaggtggtttacatctactgattggactagctaaacgttttaaggaatcaccttttgaattagaagattttattcctatggacagtacagttaaaaactatttcataacagatgcgcaaacaggttcatctaagtgtgtgtgttctgttattgatttattacttgatgattttgttgaaataataaaatcccaagatttatctgtagtttctaaggttgtcaaagtgactattgactatacagaaatttcatttatgctttggtgtaaagatggccatgtagaaacattttacccaaaattacaatctagtcaagcgtggcaaccgggtgttgctatgcctaatctttacaaaatgcaaagaatgctattagaaaagtgtgaccttcaaaattatggtgatagtgcaacattacctaaaggcataatgatgaatgtcgcaaaatatactcaactgtgtcaatatttaaacacattaacattagctgtaccctataatatgagagttatacattttggtgctggttctgataaaggagttgcaccaggtacagctgttttaagacagtggttgcctacgggtacgctgcttgtcgattcagatcttaatgactttgtctctgatgcagattcaactttgattggtgattgtgcaactgtacatacagctaataaatgggatctcattattagtgatatgtacgaccctaagactaaaaatgttacaaaagaaaatgactctaaagagggttttttcacttacatttgtgggtttatacaacaaaagctagctcttggaggttccgtggctataaagataacagaacattcttggaatgctgatctttataagctcatgggacacttcgcatggtggacagcctttgttactaatgtgaatgcgtcatcatctgaagcatttttaattggatgtaattatcttggcaaaccacgcgaacaaatagatggttatgtcatgcatgcaaattacatattttggaggaatacaaatccaattcagttgtcttcctattctttatttgacatgagtaaatttccccttaaattaaggggtactgctgttatgtctttaaaagaaggtcaaatcaatgatatgattttatctcttcttagtaaaggtagacttataattagagaaaacaacagagttgttatttctagtgatgttcttgttaacaactaa

SEQ ID NO: 6 - protein, 1273 aa, Severe acute respiratory syndrome-related coronavirus
MFVFLVLLPLVSSQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSPRRARSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDKVEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLHYT

SEQ ID NO: 7 - protein, 75 aa, Severe acute respiratory syndrome-related coronavirus
MYSFVSEETGTLIVNSVLLFLAFVVFLLVTLAILTALRLCAYCCNIVNVSLVKPSFYVYSRVKNLNSSRVPDLLV

SEQ ID NO: 8 - protein, 419 aa, Severe acute respiratory syndrome-related coronavirus
MSDNGPQNQRNAPRITFGGPSDSTGSNQNGERSGARSKQRRPQGLPNNTASWFTALTQHGKEDLKFPRGQGVPINTNSSPDDQIGYYRRATRRIRGGDGKMKDLSPRWYFYYLGTGPEAGLPYGANKDGIIWVATEGALNTPKDHIGTRNPANNAAIVLQLPQGTTLPKGFYAEGSRGGSQASSRSSSRSRNSSRNSTPGSSRGTSPARMAGNGGDAALALLLLDRLNQLESKMSGKGQQQQGQTVTKKSAAEASKKPRQKRTATKAYNVTQAFGRRGPEQTQGNFGDQELIRQGTDYKHWPQIAQFAPSASAFFGMSRIGMEVTPSGTWLTYTGAIKLDDKDPNFKDQVILLNKHIDAYKTFPPTEPKKDKKKKADETQALPQRQKKQQTVTLLPAADLDDFSKQLQQSMSSADSTQA

SEQ ID NO: 9 - protein, 222 aa, Severe acute respiratory syndrome-related coronavirus
MADSNGTITVEELKKLLEQWNLVIGFLFLTWICLLQFAYANRNRFLYIIKLIFLWLLWPVTLACFVLAAVYRINWITGGIAIAMACLVGLMWLSYFIASFRLFARTRSMWSFNPETNILLNVPLHGTILTRPLLESELVIGAVILRGHLRIAGHHLGRCDIKDLPKEITVATSRTLSYYKLGASQRVAGDSGFAAYSRYRIGNYKLNTDHSSSSDNIALLVQ

SEQ ID NO: 10 - protein, 7096 aa, Severe acute respiratory syndrome-related coronavirus [sequence omitted]

SEQ ID NO: 11 - RNA, 2571 nt, Human immunodeficiency virus 1
  source 1..2571: mol_type = mRNA
atgagagtgaaggagaaatatcagcacttgtggagatgggggtggagatggggcaccatgctccttgggatgttgatgatctgtagtgctacagaaaaattgtgggtcacagtctattatggggtacctgtgtggaaggaagcaaccaccactctattttgtgcatcagatgctaaagcatatgatacagaggtacataatgtttgggccacacatgcctgtgtacccacagaccccaacccacaagaagtagtattggtaaatgtgacagaaaattttaacatgtggaaaaatgacatggtagaacagatgcatgaggatataatcagtttatgggatcaaagcctaaagccatgtgtaaaattaaccccactctgtgttagtttaaagtgcactgatttgaagaatgatactaataccaatagtagtagcgggagaatgataatggagaaaggagagataaaaaactgctctttcaatatcagcacaagcataagaggtaaggtgcagaaagaatatgcatttttttataaacttgatataataccaatagataatgatactaccagctataagttgacaagttgtaacacctcagtcattacacaggcctgtccaaaggtatcctttgagccaattcccatacattattgtgccccggctggttttgcgattctaaaatgtaataataagacgttcaatggaacaggaccatgtacaaatgtcagcacagtacaatgtacacatggaattaggccagtagtatcaactcaactgctgttaaatggcagtctagcagaagaagaggtagtaattagatctgtcaatttcacggacaatgctaaaaccataatagtacagctgaacacatctgtagaaattaattgtacaagacccaacaacaatacaagaaaaagaatccgtatccagagaggaccagggagagcatttgttacaataggaaaaataggaaatatgagacaagcacattgtaacattagtagagcaaaatggaataacactttaaaacagatagctagcaaattaagagaacaatttggaaataataaaacaataatctttaagcaatcctcaggaggggacccagaaattgtaacgcacagttttaattgtggaggggaatttttctactgtaattcaacacaactgtttaatagtacttggtttaatagtacttggagtactgaagggtcaaataacactgaaggaagtgacacaatcaccctcccatgcagaataaaacaaattataaacatgtggcagaaagtaggaaaagcaatgtatgcccctcccatcagtggacaaattagatgttcatcaaatattacagggctgctattaacaagagatggtggtaatagcaacaatgagtccgagatcttcagacctggaggaggagatatgagggacaattggagaagtgaattatataaatataaagtagtaaaaattgaaccattaggagtagcacccaccaaggcaaagagaagagtggtgcagagagaaaaaagagcagtgggaataggagctttgttccttgggttcttgggagcagcaggaagcactatgggcgcagcctcaatgacgctgacggtacaggccagacaattattgtctggtatagtgcagcagcagaacaatttgctgagggctattgaggcgcaacagcatctgttgcaactcacagtctggggcatcaagcagctccaggcaagaatcctggctgtggaaagatacctaaaggatcaacagctcctggggatttggggttgctctggaaaactcatttgcaccactgctgtgccttggaatgctagttggagtaataaatctctggaacagatttggaatcacacgacctggatggagtgggacagagaaattaacaattacacaagcttaatacactccttaattgaagaatcgcaaaaccagcaagaaaagaatgaacaagaattattggaattagataaatgggcaagtttgtggaattggtttaacataacaaattggctgtggtatataaaattattcataatgatagtaggaggcttggtaggtttaagaatagtttttgctgtactttctatagtgaatagagttaggcagggatattcaccattatcgtttcagacccacctcccaaccccgaggggacccgacaggcccgaaggaatagaagaagaaggtggagagagagacagagacagatccattcgattagtgaacggatccttggcacttatctgggacgatctgcggagcctgtgcctcttcagctaccaccgcttgagagacttactcttgattgtaacgaggattgtggaacttctgggacgcagggggtgggaagccctcaaatattggtggaatctcctacagtattggagtcaggaactaaagaatagtgctgttagcttgctcaatgccacagccatagcagtagctgaggggacagatagggttatagaagtagtacaaggagcttgtagagctattcgccacatacctagaagaataagacagggcttggaaaggattttgctataa

SEQ ID NO: 12 - RNA, 1503 nt, Human immunodeficiency virus 1
  source 1..1503: mol_type = mRNA
atgggtgcgagagcgtcagtattaagcgggggagaattagatcgatgggaaaaaattcggttaaggccagggggaaagaaaaaatataaattaaaacatatagtatgggcaagcagggagctagaacgattcgcagttaatcctggcctgttagaaacatcagaaggctgtagacaaatactgggacagctacaaccatcccttcagacaggatcagaagaacttagatcattatataatacagtagcaaccctctattgtgtgcatcaaaggatagagataaaagacaccaaggaagctttagacaagatagaggaagagcaaaacaaaagtaagaaaaaagcacagcaagcagcagctgacacaggacacagcaatcaggtcagccaaaattaccctatagtgcagaacatccaggggcaaatggtacatcaggccatatcacctagaactttaaatgcatgggtaaaagtagtagaagagaaggctttcagcccagaagtgatacccatgttttcagcattatcagaaggagccaccccacaagatttaaacaccatgctaaacacagtggggggacatcaagcagccatgcaaatgttaaaagagaccatcaatgaggaagctgcagaatgggatagagtgcatccagtgcatgcagggcctattgcaccaggccagatgagagaaccaaggggaagtgacatagcaggaactactagtacccttcaggaacaaataggatggatgacaaataatccacctatcccagtaggagaaatttataaaagatggataatcctgggattaaataaaatagtaagaatgtatagccctaccagcattctggacataagacaaggaccaaaggaaccctttagagactatgtagaccggttctataaaactctaagagccgagcaagcttcacaggaggtaaaaaattggatgacagaaaccttgttggtccaaaatgcgaacccagattgtaagactattttaaaagcattgggaccagcggctacactagaagaaatgatgacagcatgtcagggagtaggaggacccggccataaggcaagagttttggctgaagcaatgagccaagtaacaaattcagctaccataatgatgcagagaggcaattttaggaaccaaagaaagattgttaagtgtttcaattgtggcaaagaagggcacacagccagaaattgcagggcccctaggaaaaagggctgttggaaatgtggaaaggaaggacaccaaatgaaagattgtactgagagacaggctaattttttagggaagatctggccttcctacaagggaaggccagggaattttcttcagagcagaccagagccaacagccccaccagaagagagcttcaggtctggggtagagacaacaactccccctcagaagcaggagccgatagacaaggaactgtatcctttaacttccctcaggtcactctttggcaacgacccctcgtcacaataa

SEQ ID NO: 13 - RNA, 3012 nt, Human immunodeficiency virus 1
  source 1..3012: mol_type = mRNA
ttttttagggaagatctggccttcctacaagggaaggccagggaattttcttcagagcagaccagagccaacagccccaccagaagagagcttcaggtctggggtagagacaacaactccccctcagaagcaggagccgatagacaaggaactgtatcctttaacttccctcaggtcactctttggcaacgacccctcgtcacaataaagataggggggcaactaaaggaagctctattagatacaggagcagatgatacagtattagaagaaatgagtttgccaggaagatggaaaccaaaaatgatagggggaattggaggttttatcaaagtaagacagtatgatcagatactcatagaaatctgtggacataaagctataggtacagtattagtaggacctacacctgtcaacataattggaagaaatctgttgactcagattggttgcactttaaattttcccattagccctattgagactgtaccagtaaaattaaagccaggaatggatggcccaaaagttaaacaatggccattgacagaagaaaaaataaaagcattagtagaaatttgtacagagatggaaaaggaagggaaaatttcaaaaattgggcctgaaaatccatacaatactccagtatttgccataaagaaaaaagacagtactaaatggagaaaattagtagatttcagagaacttaataagagaactcaagacttctgggaagttcaattaggaataccacatcccgcagggttaaaaaagaaaaaatcagtaacagtactggatgtgggtgatgcatatttttcagttcccttagatgaagacttcaggaagtatactgcatttaccatacctagtataaacaatgagacaccagggattagatatcagtacaatgtgcttccacagggatggaaaggatcaccagcaatattccaaagtagcatgacaaaaatcttagagccttttagaaaacaaaatccagacatagttatctatcaatacatggatgatttgtatgtaggatctgacttagaaatagggcagcatagaacaaaaatagaggagctgagacaacatctgttgaggtggggacttaccacaccagacaaaaaacatcagaaagaacctccattcctttggatgggttatgaactccatcctgataaatggacagtacagcctatagtgctgccagaaaaagacagctggactgtcaatgacatacagaagttagtggggaaattgaattgggcaagtcagatttacccagggattaaagtaaggcaattatgtaaactccttagaggaaccaaagcactaacagaagtaataccactaacagaagaagcagagctagaactggcagaaaacagagagattctaaaagaaccagtacatggagtgtattatgacccatcaaaagacttaatagcagaaatacagaagcaggggcaaggccaatggacatatcaaatttatcaagagccatttaaaaatctgaaaacaggaaaatatgcaagaatgaggggtgcccacactaatgatgtaaaacaattaacagaggcagtgcaaaaaataaccacagaaagcatagtaatatggggaaagactcctaaatttaaactgcccatacaaaaggaaacatgggaaacatggtggacagagtattggcaagccacctggattcctgagtgggagtttgttaatacccctcccttagtgaaattatggtaccagttagagaaagaacccatagtaggagcagaaaccttctatgtagatggggcagctaacagggagactaaattaggaaaagcaggatatgttactaatagaggaagacaaaaagttgtcaccctaactgacacaacaaatcagaagactgagttacaagcaatttatctagctttgcaggattcgggattagaagtaaacatagtaacagactcacaatatgcattaggaatcattcaagcacaaccagatcaaagtgaatcagagttagtcaatcaaataatagagcagttaataaaaaaggaaaaggtctatctggcatgggtaccagcacacaaaggaattggaggaaatgaacaagtagataaattagtcagtgctggaatcaggaaagtactatttttagatggaatagataaggcccaagatgaacatgagaaatatcacagtaattggagagcaatggctagtgattttaacctgccacctgtagtagcaaaagaaatagtagccagctgtgataaatgtcagctaaaaggagaagccatgcatggacaagtagactgtagtccaggaatatggcaactagattgtacacatttagaaggaaaagttatcctggtagcagttcatgtagccagtggatatatagaagcagaagttattccagcagaaacagggcaggaaacagcatattttcttttaaaattagcaggaagatggccagtaaaaacaatacatactgacaatggcagcaatttcaccggtgctacggttagggccgcctgttggtgggcgggaatcaagcaggaatttggaattccctacaatccccaaagtcaaggagtagtagaatctatgaataaagaattaaagaaaattataggacaggtaagagatcaggctgaacatcttaagacagcagtacaaatggcagtattcatccacaattttaaaagaaaaggggggattggggggtacagtgcaggggaaagaatagtagacataatagcaacagacatacaaactaaagaattacaaaaacaaattacaaaaattcaaaattttcgggtttattacagggacagcagaaatccactttggaaaggaccagcaaagctcctctggaaaggtgaaggggcagtagtaatacaagataatagtgacataaaagtagtgccaagaagaaaagcaaagatcattagggattatggaaaacagatggcaggtgatgattgtgtggcaagtagacaggatgaggattag

SEQ ID NO: 14 - RNA, 372 nt, Human immunodeficiency virus 1
  source 1..372: mol_type = mRNA
atgggtggcaagtggtcaaaaagtagtgtgattggatggcctactgtaagggaaagaatgagacgagctgagccagcagcagatagggtgggagcagcatctcgagacctggaaaaacatggagcaatcacaagtagcaatacagcagctaccaatgctgcttgtgcctggctagaagcacaagaggaggaggaggtgggttttccagtcacacctcaggtacctttaagaccaatgacttacaaggcagctgtagatcttagccactttttaaaagaaaaggggggactggaagggctaattcactcccaaagaagacaagatatccttgatctgtggatctaccacacacaaggctacttccctgattag

SEQ ID NO: 15 - protein, 856 aa, Human immunodeficiency virus 1
MRVKEKYQHLWRWGWRWGTMLLGMLMICSATEKLWVTVYYGVPVWKEATTTLFCASDAKAYDTEVHNVWATHACVPTDPNPQEVVLVNVTENFNMWKNDMVEQMHEDIISLWDQSLKPCVKLTPLCVSLKCTDLKNDTNTNSSSGRMIMEKGEIKNCSFNISTSIRGKVQKEYAFFYKLDIIPIDNDTTSYKLTSCNTSVITQACPKVSFEPIPIHYCAPAGFAILKCNNKTFNGTGPCTNVSTVQCTHGIRPVVSTQLLLNGSLAEEEVVIRSVNFTDNAKTIIVQLNTSVEINCTRPNNNTRKRIRIQRGPGRAFVTIGKIGNMRQAHCNISRAKWNNTLKQIASKLREQFGNNKTIIFKQSSGGDPEIVTHSFNCGGEFFYCNSTQLFNSTWFNSTWSTEGSNNTEGSDTITLPCRIKQIINMWQKVGKAMYAPPISGQIRCSSNITGLLLTRDGGNSNNESEIFRPGGGDMRDNWRSELYKYKVVKIEPLGVAPTKAKRRVVQREKRAVGIGALFLGFLGAAGSTMGAASMTLTVQARQLLSGIVQQQNNLLRAIEAQQHLLQLTVWGIKQLQARILAVERYLKDQQLLGIWGCSGKLICTTAVPWNASWSNKSLEQIWNHTTWMEWDREINNYTSLIHSLIEESQNQQEKNEQELLELDKWASLWNWFNITNWLWYIKLFIMIVGGLVGLRIVFAVLSIVNRVRQGYSPLSFQTHLPTPRGPDRPEGIEEEGGERDRDRSIRLVNGSLALIWDDLRSLCLFSYHRLRDLLLIVTRIVELLGRRGWEALKYWWNLLQYWSQELKNSAVSLLNATAIAVAEGTDRVIEVVQGACRAIRHIPRRIRQGLERILL

SEQ ID NO: 16 - protein, 500 aa, Human immunodeficiency virus 1
MGARASVLSGGELDRWEKIRLRPGGKKKYKLKHIVWASRELERFAVNPGLLETSEGCRQILGQLQPSLQTGSEELRSLYNTVATLYCVHQRIEIKDTKEALDKIEEEQNKSKKKAQQAAADTGHSNQVSQNYPIVQNIQGQMVHQAISPRTLNAWVKVVEEKAFSPEVIPMFSALSEGATPQDLNTMLNTVGGHQAAMQMLKETINEEAAEWDRVHPVHAGPIAPGQMREPRGSDIAGTTSTLQEQIGWMTNNPPIPVGEIYKRWIILGLNKIVRMYSPTSILDIRQGPKEPFRDYVDRFYKTLRAEQASQEVKNWMTETLLVQNANPDCKTILKALGPAATLEEMMTACQGVGGPGHKARVLAEAMSQVTNSATIMMQRGNFRNQRKIVKCFNCGKEGHTARNCRAPRKKGCWKCGKEGHQMKDCTERQANFLGKIWPSYKGRPGNFLQSRPEPTAPPEESFRSGVETTTPPQKQEPIDKELYPLTSLRSLFGNDPSSQ

SEQ ID NO: 17 - protein, 1003 aa, Human immunodeficiency virus 1
FFREDLAFLQGKAREFSSEQTRANSPTRRELQVWGRDNNSPSEAGADRQGTVSFNFPQVTLWQRPLVTIKIGGQLKEALLDTGADDTVLEEMSLPGRWKPKMIGGIGGFIKVRQYDQILIEICGHKAIGTVLVGPTPVNIIGRNLLTQIGCTLNFPISPIETVPVKLKPGMDGPKVKQWPLTEEKIKALVEICTEMEKEGKISKIGPENPYNTPVFAIKKKDSTKWRKLVDFRELNKRTQDFWEVQLGIPHPAGLKKKKSVTVLDVGDAYFSVPLDEDFRKYTAFTIPSINNETPGIRYQYNVLPQGWKGSPAIFQSSMTKILEPFRKQNPDIVIYQYMDDLYVGSDLEIGQHRTKIEELRQHLLRWGLTTPDKKHQKEPPFLWMGYELHPDKWTVQPIVLPEKDSWTVNDIQKLVGKLNWASQIYPGIKVRQLCKLLRGTKALTEVIPLTEEAELELAENREILKEPVHGVYYDPSKDLIAEIQKQGQGQWTYQIYQEPFKNLKTGKYARMRGAHTNDVKQLTEAVQKITTESIVIWGKTPKFKLPIQKETWETWWTEYWQATWIPEWEFVNTPPLVKLWYQLEKEPIVGAETFYVDGAANRETKLGKAGYVTNRGRQKVVTLTDTTNQKTELQAIYLALQDSGLEVNIVTDSQYALGIIQAQPDQSESELVNQIIEQLIKKEKVYLAWVPAHKGIGGNEQVDKLVSAGIRKVLFLDGIDKAQDEHEKYHSNWRAMASDFNLPPVVAKEIVASCDKCQLKGEAMHGQVDCSPGIWQLDCTHLEGKVILVAVHVASGYIEAEVIPAETGQETAYFLLKLAGRWPVKTIHTDNGSNFTGATVRAACWWAGIKQEFGIPYNPQSQGVVESMNKELKKIIGQVRDQAEHLKTAVQMAVFIHNFKRKGGIGGYSAGERIVDIIATDIQTKELQKQITKIQNFRVYYRDSRNPLWKGPAKLLWKGEGAVVIQDNSDIKVVPRRKAKIIRDYGKQMAGDDCVASRQDED

SEQ ID NO: 18 - protein, 123 aa, Human immunodeficiency virus 1
MGGKWSKSSVIGWPTVRERMRRAEPAADRVGAASRDLEKHGAITSSNTAATNAACAWLEAQEEEEVGFPVTPQVPLRPMTYKAAVDLSHFLKEKGGLEGLIHSQRRQDILDLWIYHTQGYFPD

SEQ ID NO: 19 - RNA, 4097 nt, synthetic construct
  source 1..4097: mol_type = other RNA
  modified_base 1..4097: mod_base = m1f
acatttgcttctgacacaactgtgttcactagcaacctcaaacagacaccatgttcgtgtttctggtgctgctgcctctggtgagctcccagtgcgtgaacctgaccacaaggacccagctgccccctgcctataccaattccttcacacggggcgtgtactatcccgacaaggtgtttagatctagcgtgctgcactccacacaggatctgtttctgcctttcttttctaacgtgacctggttccacgccatccacgtgagcggcaccaatggcacaaagcggttcgacaatccagtgctgccctttaacgatggcgtgtacttcgcctccaccgagaagtctaacatcatcagaggctggatctttggcaccacactggacagcaagacacagtccctgctgatcgtgaacaatgccaccaacgtggtcatcaaggtgtgcgagttccagttttgtaatgatccattcctgggcgtgtactatcacaagaacaataagtcttggatggagagcgagtttcgcgtgtattcctctgccaacaattgcacatttgagtacgtgtcccagcccttcctgatggacctggagggcaagcagggcaatttcaagaacctgagggagttcgtgtttaagaatatcgatggctacttcaagatctactccaagcacaccccaatcaacctggtgcgcgacctgccacagggcttctctgccctggagccactggtggatctgcccatcggcatcaacatcacccggtttcagacactgctggccctgcacagaagctacctgacaccaggcgacagctcctctggatggaccgcaggagctgccgcctactatgtgggctatctgcagcccaggaccttcctgctgaagtacaacgagaatggcaccatcacagacgccgtggattgcgccctggatcccctgtctgagaccaagtgtacactgaagagctttaccgtggagaagggcatctatcagacaagcaatttcagggtgcagcctaccgagtccatcgtgcgctttcccaatatcacaaacctgtgcccttttggcgaggtgttcaacgcaaccaggttcgcaagcgtgtacgcatggaataggaagcgcatctccaactgcgtggccgactattctgtgctgtacaacagcgcctccttctctacctttaagtgctatggcgtgagccccacaaagctgaatgacctgtgctttaccaacgtgtacgccgattccttcgtgatcaggggcgacgaggtgcgccagatcgcaccaggacagacaggcaagatcgcagactacaattataagctgcctgacgatttcaccggctgcgtgatcgcctggaactctaacaatctggatagcaaagtgggcggcaactacaattatctgtaccggctgtttagaaagtctaatctgaagccattcgagagggacatctccacagagatctaccaggccggctctaccccctgcaatggcgtggagggctttaactgttatttccctctgcagagctacggcttccagccaacaaacggcgtgggctatcagccctaccgcgtggtggtgctgtcttttgagctgctgcacgcacctgcaacagtgtgcggaccaaagaagagcaccaatctggtgaagaacaagtgcgtgaacttcaacttcaacggactgaccggaacaggcgtgctgaccgagtccaacaagaagttcctgccttttcagcagttcggcagggacatcgcagataccacagacgccgtgcgcgaccctcagaccctggagatcctggatatcacaccatgctccttcggcggcgtgtctgtgatcacaccaggcaccaatacaagcaaccaggtggccgtgctgtatcaggacgtgaattgtaccgaggtgccagtggcaatccacgcagatcagctgacccctacatggcgggtgtactctaccggcagcaacgtgttccagacaagagcaggatgcctgatcggagcagagcacgtgaacaatagctatgagtgcgacatccctatcggcgccggcatctgtgcctcctaccagacccagacaaactccccaggcagcgcaagctctgtggcaagccagtccatcatcgcctataccatgagcctgggcgccgagaattccgtggcctactccaacaattctatcgccatccctaccaacttcacaatctccgtgaccacagagatcctgccagtgagcatgaccaagacatccgtggactgcacaatgtatatctgtggcgattccaccgagtgctctaacctgctgctgcagtacggctctttttgtacccagctgaatagagccctgacaggcatcgccgtggagcaggacaagaacacacaggaggtgttcgcccaggtgaagcagatctacaagaccccacccatcaaggactttggcggcttcaacttcagccagatcctgcccgatcctagcaagccatccaagcggtctcccatcgaggacctgctgttcaacaaggtgaccctggccgatgccggcttcatcaagcagtatggcgattgcctgggcgacatcgccgccagagacctgatctgtgcccagaagtttaatggcctgaccgtgctgcctccactgctgacagatgagatgatcgcccagtacacatctgccctgctggcaggaaccatcacaagcggatggaccttcggccccggagccgccctgcagatcccctttcccatgcagatggcctatcggttcaacggcatcggcgtgacccagaatgtgctgtacgagaaccagaagctgatcgccaatcagtttaactccgccatcggcaagatccaggactctctgagctccacaccaagcgccctgggcaagctgcaggatgtggtgaatcagaacgcccaggccctgaataccctggtgaagcagctgtctagcaacttcggcgccatctcctctgtgctgaatgatatcctgagcaggctggacccccctgaggcagaggtgcagatcgaccggctgatcacaggcagactgcagtccctgcagacctacgtgacacagcagctgatcagggcagcagagatcagggcatctgccaatctggccgccaccaagatgagcgagtgcgtgctgggccagtccaagagagtggacttttgtggcaagggctatcacctgatgagcttcccacagtccgcccctcacggagtggtgtttctgcacgtgacctacgtgccagcccaggagaagaacttcaccacagcaccagcaatctgccacgatggcaaggcacactttcctagggagggcgtgttcgtgagcaacggcacccactggtttgtgacacagcgcaatttctacgagccacagatcatcaccacagacaatacattcgtgtccggcaactgtgacgtggtcatcggcatcgtgaacaataccgtgtatgatcctctgcagccagagctggactcttttaaggaggagctggataagtacttcaagaatcacaccagccccgacgtggatctgggcgacatctctggcatcaatgccagcgtggtgaacatccagaaggagatcgacaggctgaacgaggtggccaagaatctgaacgagtccctgatcgatctgcaggagctgggcaagtatgagcagggatctggctacatccccgaggcccctagagatggccaggcctacgtgcggaaggacggcgaatgggtgctgctgagcaccttcctgggcagaagctacatcaagtggccctggtatatctggctgggcttcatcgccggcctgatcgccatcgtgatggtgaccatcatgctgtgctgtatgacaagctgctgttcctgcctgaagggctgctgttcttgtggcagctgctgtaagtttgatgaggacgatagcgagcctgtgctgaagggcgtgaagctgcactacaccgctcgctttcttgctgtccaatttctattaaaggttcctttgttccctaagtccaactactaaactgggggatattatgaagggccttgagcatctggattctgcctaataaaaaacatttattttcattgc

SEQ ID NO: 20 - RNA, 1366 nt, synthetic construct
  source 1..1366: mol_type = other RNA
  modified_base 1..1366: mod_base = m1f
aagctttaatacgactcactatagggaaataagagagaaaagaagagtaagaagaaatataagagccaccatgggcagcatcggcgccgccagcatggagttctgcttcgacgtgttcaaggagctgaaggtgcaccacgccaacgagaacatcttctactgccccatcgccatcatgagcgccctggccatggtgtacctgggcgccaaggacagcacccggacccagatcaacaaggtggtgcggttcgacaagctgcccggcttcggcgacagcatcgaggcccagtgcggcaccagcgtgaacgtgcacagcagcctgcgggacatcctgaaccagatcaccaagcccaacgacgtgtacagcttcagcctggccagccggctgtacgccgaggagcggtaccccatcctgcccgagtacctgcagtgcgtgaaggagctgtaccggggcggcctggagcccatcaacttccagaccgccgccgaccaggcccgggagctgatcaacagctgggtggagagccagaccaacggcatcatccggaacgtgctgcagcccagcagcgtggacagccagaccgccatggtgctggtgaacgccatcgtgttcaagggcctgtgggagaagaccttcaaggacgaggacacccaggccatgcccttccgggtgaccgagcaggagagcaagcccgtgcagatgatgtaccagatcggcctgttccgggtggccagcatggccagcgagaagatgaagatcctggagctgcccttcgccagcggcaccatgagcatgctggtgctgctgcccgacgaggtgagcggcctggagcagctggagagcatcatcaacttcgagaagctgaccgagtggaccagcagcaacgtgatggaggagcggaagatcaaggtgtacctgccccggatgaagatggaggagaagtacaacctgaccagcgtgctgatggccatgggcatcaccgacgtgttcagcagcagcgccaacctgagcggcatcagcagcgccgagagcctgaagatcagccaggccgtgcacgccgcccacgccgagatcaacgaggccggccgggaggtggtgggcagcgccgaggccggcgtggacgccgccagcgtgagcgaggagttccgggccgaccaccccttcctgttctgcatcaagcacatcgccaccaacgccgtgctgttcttcggccggtgcgtgagcccctgataggctcgctttcttgctgtccaatttctattaaaggttcctttgttccctaagtccaactactaaactgggggatattatgaagggccttgagcatctggattctgcctaataaaaaacatttattttcattgc

SEQ ID NO: 21 - RNA, 228 nt, synthetic construct
  source 1..228: mol_type = other RNA
  modified_base 1..228: mod_base = m1f
aagctttaatacgactcactatagggaaataagagagaaaagaagagtaagaagaaatataagagccaccatgtctatcattaacttcgagaagttgctcgctttcttgctgtccaatttctattaaaggttcctttgttccctaagtccaactactaaactgggggatattatgaagggccttgagcatctggattctgcctaataaaaaacatttattttcattgc

SEQ ID NO: 22 - protein, 8 aa, synthetic construct
SIINFEKL